(19) United States
(12) Patent Application Publication
(10) Pub. No.: US 2023/0159989 A1
(43) Pub. Date: May 25, 2023

(54) MULTIPLEXED FLUORESCENCE IN SITU HYBRIDIZATION METHOD CAPABLE OF RAPID DETECTION OF BILLIONS OF TARGETS

(71) Applicant: Kanvas Biosciences, Inc., Monmouth Junction, NJ (US)

(72) Inventors: Philip S. Burnham, Monmouth Junction, NJ (US); Hannah Bronson, Monmouth Junction, NJ (US); Matthew P. Cheng, Monmouth Junction, NJ (US); Hao Shi, Monmouth Junction, NJ (US); Prateek Sehgal, Monmouth Junction, NJ (US); Gregory T. Booth, Monmouth Junction, NJ (US); Iwijn De Vlaminck, Monmouth Junction, NJ (US)

(73) Assignee: Kanvas Biosciences, Inc., Monmouth Junction, NJ (US)

(21) Appl. No.: 18/058,171

(22) Filed: Nov. 22, 2022

Related U.S. Application Data
(60) Provisional application No. 63/339,291, filed on May 6, 2022.
(60) Provisional application No. 63/282,947, filed on Nov. 24, 2021.

(51) Int. Cl. C12Q 1/6816

(52) U.S. Cl. CPC C12Q 1/6816

(57) ABSTRACT

The present disclosure provides multiplexed methods, and constructs made to be used in said methods, for characterizing microbes from a biological sample to both rapidly identify the microbe and characterize drug susceptibility or resistance and perform microbial taxa identification and nucleic acid target detection at high multiplexity. The methods can also be used to predict future microbe drug susceptibility or resistance.

### CROSS-REFERENCE TO RELATED APPLICATIONS

This application claims the benefit of U.S. Provisional Application No. 63/282,947, filed on Nov. 24, 2021, and U.S. Provisional Application No. 63/339,291, filed on May 6, 2022. The entire contents of the aforementioned applications are incorporated herein by reference in their entireties.

### SEQUENCE LISTING

This application includes and incorporates by reference in its entirety a Sequence Listing XML in the required .xml format. The Sequence Listing XML file that has been electronically filed contains the information of the nucleotide and/or amino acid sequences disclosed in the patent application using the symbols and format in accordance with the requirements of 37 C.F.R. §§ 1.832 through 1.834.

The Sequence Listing XML filed herewith serves as the electronic copy required by § 1.834(b)(1).

The Sequence Listing XML is identified as follows: “KANVAS_003_SEQ_LIST.xml” (1649 kilo bytes in size), which was created on Nov. 22, 2022.

### TECHNICAL FIELD

This disclosure relates to methods for highly-multiplexed, rapid detection of nucleotides in samples, and constructs to be used in said methods.

### BACKGROUND

Microbes, both individually and in communities (i.e. microbiomes), play a large role in human health and disease. Conventional methods to study biologically and clinically relevant aspects of these microbes, including antimicrobial resistance, suffer from long turnaround times and are limited in the number of taxa and genetic elements they can profile. As a result, researchers are left with an incomplete understanding of microbiota in their native biological contexts. In addition, clinicians are faced with diagnostic delays that are detrimental to patient care, which increases the risk of patient morbidity and mortality.

### SUMMARY

Antimicrobial resistance is an emerging threat to global public health. Current tests available in clinical laboratories are time-consuming and limited in scope for antimicrobial resistance profile measurement. Timely and accurate information on pathogen identity and their associated antimicrobial susceptibility profile is critical in helping clinicians treat patients with shorter response time and higher precision. In addition, many other microbial phenotypes, such as persistence, tolerance, motility, hyphae formation, spore formation, and quorum sensing, can provide useful biological and clinical information, but are difficult to measure using standard sequencing techniques. The present disclosure provides methods for microbial identification and rapid antimicrobial susceptibility profile measurement or other microbial phenotype measurements. These methods combine a short period of culturing with known concentrations of antimicrobial drugs, or other alterations to the environment, with a highly multiplexed fluorescence readout to distinguish cellular taxonomic identity and susceptibility to different classes of antimicrobials or other relevant microbial phenotypes. This approach will enable a rapid and cost-effective test that can be deployed in clinical settings for fast diagnosis of infectious agents and proper selection of antimicrobial drugs for treatment.

The present disclosure provides methods that combine single-cell imaging, single-molecule imaging, microfluidic technologies, and phenotypic antimicrobial susceptibility testing to enable rapid identification of microbial species, current antimicrobial susceptibility profile, and future antimicrobial susceptibility profile, directly from patient samples. The present disclosure also provides methods that enable the detection of millions or billions of potential nucleic acid based targets in a single assay.

The present disclosure provides methods that can rapidly identify microbial species, genera, families, orders, classes, and phyla associated with a particular tissue or specimen. In further embodiments, the present disclosure provides methods to rapidly determine any antimicrobial drugs or compounds the identified microbial species is susceptible to or to which the microbial species may become susceptible in the future.

In some aspects, the present disclosure provides methods of characterizing a microbial cell from a biological sample, the method comprising a) directly inoculating the microbe onto a device; b) identifying the microbe; and c) detecting susceptibility to one or more antimicrobial agents.

In some aspects, the present disclosure provides methods of characterizing a microbial cell from a biological sample, the method comprising a) directly inoculating the microbe onto a device; b) identifying the microbe; and c) detecting future susceptibility to one or more antimicrobial agents.

In some embodiments, the sample is not subjected to culturing before the microbe is inoculated onto the device. In some embodiments, the microbe in the sample is cultured for one to 12 cell divisions before it is inoculated onto the device. In some embodiments, the microbe in the sample is cultured for one to numerous cell divisions before it is inoculated onto the device. The number of cell divisions depends on the species doubling time, which can be variable.

In some embodiments, the microbe is identified by in situ hybridization. In some embodiments, the microbe is identified by fluorescence in situ hybridization (FISH). In some embodiments, the fluorescence in situ hybridization is high-phylogenetic-resolution fluorescence in situ hybridization (HiPR-FISH).

In some embodiments, the microbe is further characterized via live-cell imaging or growth dynamics calculation while in situ hybridization is performed.

In some embodiments, the microbe is identified by hybridization of a bar-coded probe a 16S ribosomal RNA sequence in the microbe, 5S ribosomal RNA sequence in the microbe, and/or 23S ribosomal RNA sequence in the microbe. In some embodiments, the in situ hybridization is multiplexed. In some embodiments, the susceptibility to one or more microbial agents is determined by measuring the minimum inhibitory concentration of the microbe when exposed to an antimicrobial agent. In some embodiments, the susceptibility to one or more microbial agents is determined by measuring microbial cell metabolism when the microbe is exposed to an antimicrobial agent. In some embodiments, microbial cell metabolism is measured by determining the concentration of dissolved carbon dioxide, oxygen consumption of microbes in the sample, expression of genes involved in cell division and/or growth, or expression of stress response genes. In some embodiments, microbial cell susceptibility is determined by a live/dead stain. In some embodiments, wherein microbial cell susceptibility is determined by cell number. In some embodiments, microbial cell susceptibility is determined by detecting the presence or absence of one or more antimicrobial genes in the microbial cell. In some embodiments, microbial cell susceptibility is determined by detecting the presence or absence of one or more gene mutations associated with the development of antimicrobial resistance or susceptibility in the microbial cell. In some embodiments, future microbial cell susceptibility is determined by detecting the presence or absence of one or more antimicrobial genes in the microbial cell. In some embodiments, future microbial cell susceptibility is determined by detecting the presence or absence of one or more gene mutations associated with the development of antimicrobial resistance or susceptibility in the microbial cell.

In some embodiments, wherein the one or more gene mutations associated with the development of antimicrobial resistance or susceptibility is selected from deletions, duplications, single nucleotide polymorphisms (SNPs), frame-shift mutations, inversions, insertions, and/or nucleotide substitutions. In some embodiments, the one or more antimicrobial genes is selected from: genes encoding multidrug resistance proteins (e.g. PDR1, PDR3, PDR7, PDR9), ABC transporters (e.g. SNQ2, STE6, PDR5, PDR10, PDR11, YOR1), membrane associated transporters (GAS1, D4405), soluble proteins (e.g. G3PD), RNA polymerase, rpoB, gyrA, gyrB, 16S RNA, 23S rRNA, NADPH nitroreductase, sul2, strAB, tetAR, aac3-iid, aph, sph, cmy-2, floR, tetB; aadA, aac3-VIa, and sul1. In some embodiments, the presence or absence of one or more antimicrobial genes, or the gene mutation associated with the development of antimicrobial resistance or susceptibility in the microbial cell is detected using in situ hybridization. In some embodiments, the presence or absence of one or more antimicrobial genes, or the gene mutation associated with the development of antimicrobial resistance or susceptibility in the microbial cell is detected using fluorescence in situ hybridization (FISH). In some embodiments, the fluorescence in situ hybridization is high-phylogenetic-resolution fluorescence in situ hybridization (HiPR-FISH).

In some embodiments, the identification of the microbial cell and the detection of susceptibility or future susceptibility to one or more antimicrobial agents occurs sequentially.

In some embodiments, the identification of the microbial cell and the detection of susceptibility or future susceptibility to one or more antimicrobial agents occurs simultaneously.

In some embodiments, the identification of the microbial cell and the detection of susceptibility or future susceptibility to one or more antimicrobial agents occurs in parallel.

In some embodiments, the biological sample is obtained from a patient. In some embodiments, the biological sample is obtained from a patient diagnosed with or believed to be suffering from an infection or disorder. In some embodiments, the disease or disorder is an infection. In some embodiments, the infection is a bacterial, viral, fungal, or parasitic infections. In some embodiments, the bacterial infection is selected from Mycobacterium, Streptococcus, Staphylococcus, Shigella, Campylobacter, Salmonella, Clostridium, Corynebacterium, Pseudomonas, Neisseria, Listeria, Vibrio, Bordetella, E. coli (including pathogenic E. coli), Pseudomonas aeruginosa, Enterobacter cloacae, Mycobacterium tuberculosis, Staphylococcus aureus, Helicobacter pylori, Legionella, Acinetobacter baumannii, Citrobacter freundii, Citrobacter koseri, Enterobacter cloacae, Enterococcus faecalis, Enterococcus faecium, Escherichia coli, Klebsiella oxytoca, Klebsiella pneumoniae, Proteus mirabilis, Proteus vulgaris, Pseudomonas aeruginosa, Serratia marcescens, Staphylococcus aureus, Staphylococcus saprophyticus, and Streptococcus agalactiae, or a combination thereof. In some embodiments, the viral infection is selected from Helicobacter pylori, infectious haematopoietic necrosis virus (IHNV), Parvovirus B19, Herpes Simplex Virus, Varicella-zoster virus, Cytomegalovirus, Epstein-Barr virus, Hepatitis A virus, Hepatitis B virus, Hepatitis C virus, Hepatitis D virus, Hepatitis E virus, Measles virus, Mumps virus, Rubella virus, Human Immunodeficiency Virus (HIV), Influenza virus, Rhinovirus, Rotavirus A, Rotavirus B, Rotavirus C, Respiratory Syncytial Virus (RSV), Varicella zoster, Poliovirus, Norovirus, Zika Virus, Dengue Virus, Rabies Virus, Newcastle Disease Virus, and White Spot Syndrome Virus, or a combination thereof. In some embodiments, the fungal infection is selected from Aspergillus, Candida, Pneumocystis, Blastomyces, Coccidioides, Cryptococcus, and Histoplasma, or a combination thereof. In some embodiments, the parasitic infection is selected from Plasmodium (i.e. P. falciparum, P. malariae, P. ovale, P. knowlesi, and P. vivax), Trypanosoma, Toxoplasma, Giardia, and Leishmania, Cryptosporidium, helminthic parasites: Trichuris spp. (whipworms), Enterobius spp. (pinworms), Ascaris spp. (roundworms), Ancylostoma spp. and Necator spp. (hookworms), Strongyloides spp. (threadworms), Dracunculus spp. (Guinea worms), Onchocerca spp. and Wuchereria spp. (filarial worms), Taenia spp., Echinococcus spp., and Diphyllobothrium spp. (human and animal cestodes), Fasciola spp. (liver flukes) and Schistosoma spp. (blood flukes), or a combination thereof.

In some embodiments, the biological sample is selected from bronchoalveolar lavage fluid (BAL), blood, serum, plasma, urine, cerebrospinal fluid, pleural fluid, synovial fluid, ocular fluid, peritoneal fluid, amniotic fluid, gastric fluid, lymph fluid, interstitial fluid, tissue homogenate, cell extracts, saliva, sputum, stool, physiological secretions, tears, mucus, sweat, milk, semen, seminal fluid, vaginal secretions, fluid from ulcers and other surface eruptions, blisters, and abscesses, and extracts of tissues including biopsies of normal, malignant, and suspect tissues or any other constituents of the body which may contain the microorganism of interest. In some embodiments, the biological sample is a human oral microbiome sample. In some embodiments, the biological sample is a whole organism.

In another aspect, a method for analyzing a sample can include:
contacting at least one encoding probe with the sample to produce a first complex, wherein each encoding probe comprises a targeting sequence, a first landing pad sequence, and a second landing pad sequence;
adding at least one first emissive readout probe to the first complex, wherein the first emissive readout probe comprises a label and a sequence complementary to the first landing pad sequence;
acquiring one or more emission spectra from the first emissive readout probe;
adding an exchange probe to the sample, wherein the exchange probe comprises a 100% complementary sequence to the first emissive readout probe sequence,
hybridizing the exchange probe to the first emissive readout probe to form a second complex;
removing the second complex from the sample,
adding at least one second emissive readout probe to the first complex, wherein the second emissive readout probe comprises a label and a sequence complementary to the second landing pad sequence;
acquiring one or more emission spectra from the second emissive readout probe;
repeating the aforementioned steps for at least one different encoding probe;
determining the spectra of “signal” (e.g., puncta, blobs) and assigning them to a species of interest; and
decoding the spectra into a single, targeted transcript through means of signal deconvolution, error correction, comparison to reference standards.

In certain embodiments, the first emissive readout probe sequence can be the same length as the first landing pad sequence.

In certain embodiments, the first emissive readout probe sequence can be at least 2 nucleotides longer than the first landing pad sequence.

In certain embodiments, the second emissive readout probe sequence can be the same length as the second landing pad sequence.

In certain embodiments, the second emissive readout probe sequence can be at least 2 nucleotides longer than the second landing pad sequence.

In another aspect, a method for analyzing a sample can include:
generating a set of probes, wherein each probe comprises:
(i) a targeting sequence;
(ii) a first landing pad sequence; and
(iii) a second landing pad sequence;
contacting the set of probes with the sample to permit hybridization of the probes to nucleotides present in the sample to produce a complex;
adding a first set of emissive readout probes to the complex, wherein each emissive readout probe comprises:
(i) a label, and
(ii) a sequence complementary to the first or second landing pad sequence;
acquiring one or more emission spectra from the first emissive readout probe;
adding a set of exchange probes to the sample, wherein each exchange probe comprises a 100% complementary sequence to the first emissive readout probe sequences,
hybridizing the exchange probes to the first emissive readout probes to form a second complex;
removing the second complex from the sample, adding a second set of emissive readout probes to the complex, wherein each emissive readout probe comprises:
(i) a label, and
(ii) a sequence complementary to the first or second landing pad sequence;
acquiring one or more emission spectra from the second emissive readout probe;
determining the spectra of “signal” (e.g., puncta, blobs) and assigning them to a species of interest; and
decoding the spectra into a single, targeted transcript through means of signal deconvolution, error correction, comparison to reference standards.

In certain embodiments, the emissive readout probe sequence can be the same length as the first or second landing pad sequence.

In certain embodiments, the emissive readout probe sequence can be at least 2 nucleotides longer than the first or second landing pad sequence.

In another aspect, a construct can include:
a targeting sequence that is a region of interest on a nucleotide;
a first landing pad sequence;
a second landing pad sequence, wherein the second landing pad sequence is different from the first landing pad sequence;
a first emissive readout probe comprising a first label and a sequence complimentary to the first landing pad sequence;
an exchange probe comprising a 100% complementary sequence to the first emissive readout probe sequences; and
a second emissive readout probe comprising a second label and a sequence complimentary to the second landing pad sequence.

In certain embodiments, the first emissive readout probe sequence can be the same length as the first landing pad sequence.

In certain embodiments, the first emissive readout probe sequence can be at least 2 nucleotides longer than the first landing pad sequence.

In certain embodiments, the second emissive readout probe sequence can be the same length as the second landing pad sequence.

In certain embodiments, the second emissive readout probe sequence can be at least 2 nucleotides longer than the second landing pad sequence.

In another aspect, a library of constructs comprising a plurality of barcoded probes, wherein each barcoded probe comprises:
a targeting sequence that is a region of interest on a nucleotide;
a first landing pad sequence;
a second landing pad sequence, wherein the second landing pad sequence is different from the first landing pad sequence;
a first emissive readout probe comprising a first label and a sequence complimentary to the first landing pad sequence;
an exchange probe comprising a 100% complementary sequence to the first emissive readout probe sequences; and
a second emissive readout probe comprising a second label and a sequence complimentary to the second landing pad sequence.

In certain embodiments, the first emissive readout probe sequence can be the same length as the first landing pad sequence.

In certain embodiments, the first emissive readout probe sequence can be at least 2 nucleotides longer than the first landing pad sequence.

In certain embodiments, the second emissive readout probe sequence can be the same length as the second landing pad sequence.

In certain embodiments, the second emissive readout probe sequence can be at least 2 nucleotides longer than the second landing pad sequence.

In another aspect, a method for analyzing a bacterial sample can include:
contacting at least one encoding probe with the sample to produce a first complex, wherein each encoding probe comprises a targeting sequence, a first landing pad sequence, and a second landing pad sequence;
adding at least one first emissive readout probe to the first complex, wherein the first emissive readout probe comprises a label and a sequence complementary to the first landing pad sequence;
detecting the first emissive readout probe with a confocal microscope;
adding an exchange probe to the sample, wherein the exchange probe comprises a 100% complementary sequence to the first emissive readout probe sequence,
hybridizing the exchange probe to the first emissive readout probe to form a second complex;
removing the second complex from the sample,
adding at least one second emissive readout probe to the first complex, wherein the second emissive readout probe comprises a label and a sequence complementary to the second landing pad sequence;
detecting the second emissive readout probe with a confocal microscope;
repeating the aforementioned steps for at least one different encoding probe;
determining the spectra of “signal” (e.g., puncta, blobs) and assigning them to a bacterium; and
decoding the spectra into a single, targeted transcript through means of signal deconvolution, error correction, comparison to reference standards.

In certain embodiments, the first emissive readout probe sequence can be the same length as the first landing pad sequence.

In certain embodiments, the first emissive readout probe sequence can be at least 2 nucleotides longer than the first landing pad sequence.

In certain embodiments, the second emissive readout probe sequence can be the same length as the second landing pad sequence.

In certain embodiments, the second emissive readout probe sequence can be at least 2 nucleotides longer than the second landing pad sequence.

In another aspect, a method for analyzing a bacterial sample can include:
generating a set of probes, wherein each probe comprises:
(i) a targeting sequence;
(ii) a first landing pad sequence; and
(iii) a second landing pad sequence;
contacting the set of probes with the sample to permit hybridization of the probes to nucleotides present in the sample to produce a complex;
adding a first set of emissive readout probes to the complex, wherein each emissive readout probe comprises:
(i) a label, and
(ii) a sequence complementary to the first or second landing pad sequence;
detecting the first set of emissive readout probes in the sample with a confocal microscope;
adding a set of exchange probes to the sample, wherein each exchange probe comprises a 100% complementary sequence to the first emissive readout probe sequences,
hybridizing the exchange probes to the first emissive readout probes to form a second complex;
removing the second complex from the sample,
adding a second set of emissive readout probes to the complex, wherein each emissive readout probe comprises:
(i) a label, and
(ii) a sequence complementary to the first or second landing pad sequence;
detecting the second set of emissive readout probes in the sample with a confocal microscope;
determining the spectra of “signal” (e.g., puncta, blobs) and assigning them to a bacterium; and
decoding the spectra into a single, targeted transcript through means of signal deconvolution, error correction, comparison to reference standards.

In certain embodiments, the emissive readout probe sequence can be the same length as the first or second landing pad sequence.

In certain embodiments, the emissive readout probe sequence can be at least 2 nucleotides longer than the first or second landing pad sequence.

Other aspects, embodiments, and features will be apparent from the following description, the drawings, and the claims.

### BRIEF DESCRIPTION OF THE FIGURES

FIGS. 1A-1B shows an exemplary method of rapid phenotypic profiling of antibiotic resistance followed by microbial identification using imaging. FIG. 1A shows an experimental set-up. FIG. 1B shows an example of a binary barcoding and spectral imaging approach for highly multiplexed labeling of microbes for taxonomic identification.

FIG. 2 shows an experimental work-flow for HiPR-FISH to identify a microbe in a sample and characterize a drug-resistance phenotype.

FIG. 3 shows E. coli detected in urine samples obtained from three different patients. HiPR-FISH was performed directly on three patient samples, each with over 100,000 CFU/mL of E. coli. The images were collected from the first emission channel after excitation of a 561 nm laser (in agreement with dye corresponding to the readout probe used, Alexa546).

FIG. 4 shows a HiPR-FISH panel identifying species including A. baumannii, C. freundii, S. saprophyticus, and a mixture of A. baumannii and C. freundii. Maximum merged emission images from different laser excitation wavelengths (first three columns). The fourth column is a false-colored, merged image of 405 nm (blue), 488 nm (green), and 561 nm (red). The fifth column is a close-up of the white boxes in column 4.

FIG. 5 shows the ability of HiPR-FISH to report drug susceptibility and minimum inhibitory concentration (MIC), and determine antimicrobial resistance or susceptibility. A comparison of the first and last time points, after several hours growth on a HiPR-FISH chip, for several concentrations of meropenem for carbapenem-resistant and carbapenem-susceptible K. pneumonia. The carbapenem-resistant K. pneumoniae grows beyond 2 μg/mL (* denotes MIC) in agreement with Clinical and Laboratory Standards Institute criteria.

FIG. 6 shows the ability of HiPR-FISH to detect fastidious and slow growing organisms in a synthetic mixture of fixed and digested Candida species. HiPR-FISH probes were designed to detect C. tropicalis (blue), C. glabrata (orange), and C. albicans (green) (colors not shown).

FIGS. 7A-7C shows gene expression measurements enable rapid detection of stress response in HiPR-FISH compatible manner. FIG. 7A shows a schematic of an ultrarapid gene expression measurement assay that can be performed in 2 hours with only 5 minutes of exposure to stress. The results of the 2 hour assay, with E. coli rRNA and heat-shock response gene clpB mRNA are shown in E. coli grown at 30° C. (FIG. 7B) and shocked at 46° C. (FIG. 7C) for 5 minutes. Scale bars=20 μm.

FIG. 8 shows a schematic of HiPR-Swap.

FIG. 9 shows probe stripping and signal recovery in HiPR-Swap. Fixed monomicrobial stock of E. coli, Klebsiella pneumoniae, and Pseudomonas aeruginosa, were hybridized with species-specific encoding probes and individual readout probes in a single step (left column of images). Exchange buffer, with exchange probes for each readout, was added and incubated overnight to remove the readout probes (middle column). Signals for each species were recovered by adding back readout probes without encoding probes (right column).

FIG. 10 shows speed of stripping readout probes in HiPR-Swap. HiPR-Swap samples of E. coli, Klebsiella pneumoniae, and Pseudomonas aeruginosa, from FIG. 9 were imaged after 5 days (column “After 5 days”). Exchange buffer, with exchange probes for each readout, was added and incubated for 1 hour to remove the readout probes (column “Strip—1 hr”).

FIG. 11 shows samples of E. coli, Klebsiella pneumoniae, and Pseudomonas aeruginosa, from FIG. 10 were imaged after stripping overnight (column “Strip—overnight”). Signals for each species were recovered by adding different readout probes of green color without encoding probes (column “Swap—R #-488”).

FIG. 12 shows probe stripping and swapping reaction in a single step. Top panel shows single step strip and swap reaction and bottom panel shows sequential strip and swap reaction. Fixed synthetic mixtures of E. coli and Pseudomonas aeruginosa (P. aeru; P. aeruginosa) were hybridized with species-specific encoding probes and Eubacterium probes (conjugated with Rhodamine Red-X fluorophore). First row of each panel shows the Eubacterium signal. In round 1, only E. coli is hybridized with its readout probes for both the single step and sequential step conditions (left column second row of each panel). In round 2 of the single step condition, exchange buffer containing exchange probes for E. coli and readout probes for P. aeruginosa was added and incubated for 2 hours (top panel middle column second row). In round 2 of the sequential step condition, exchange buffer containing exchange probes for E. coli was added and incubated for 2 hours (bottom panel middle column second row). In round 3 of the single step condition, exchange buffer containing readout probes for E. coli and exchange probes for P. aeruginosa was added and incubated for 2 hours (top panel last column second row). In round 3 of sequential step condition, exchange buffer containing readout probes for P. aeruginosa was added and incubated for 2 hours (bottom panel last column second row).

FIG. 13 shows real time measurement of single step strip and swap reaction of HiPR-Swap. Fixed synthetic mixtures of E. coli and Pseudomonas aeruginosa (P. aeru, P. aeruginosa) were hybridized with species-specific encoding probes. Before performing single step stripping and swapping reaction, only E. coli is hybridized with its readout probes (Image: “Before”). While keeping the sample under the microscope, exchange buffer containing exchange probes for E. coli and readout probes for P. aeruginosa was added and acquisition of images was started (Image: 0 min, 2 min, 4 min, 8 min, 12 min).

FIG. 14 shows an overview of Example 11. Fixed monomicrobial stocks of E. coli are plated in different wells and encoded with a unique set of 24 probes that have several readout bits (at least one on-bit per round). After encoding, the bacteria undergo: washing of encoding probes, probe exchange with the addition of readout and exchange probes, wash of readout and exchange probes, and imaging. Each round will yield a potentially non-unique 10 bit (sub-) barcode. The readouts exchanges were performed in four rounds, with the first and final round having identical readout probes used for a recovery check. After the final round a full barcode (30 bits) can be generated.

FIG. 15 shows a basic design and concept for HiPR-Swap, in situ. A unique set of 30 readout probes were designed that can be used with a standard 10-bit system described herein. To achieve this, each oligo sequence on the readout probe is unique, but each fluorophore is used three times. Readout probes with the same fluorophore must be used in different rounds to achieve accurate barcode interpretation. As an example of their use, a schematic of bacteria and the encoding scheme is shown. The bacteria is encoded with probes targeting the rRNA and with flanking landing pads (colored) that correspond to the reverse complement of the intended readout probe. In each round a set of 10 readout probes (and possibly 10 exchange probes) are added to determine a sub-barcode for the round. After each round, the readout probes are removed from the specimen and a new batch is added. After all rounds are complete, classification is performed to determine the round-barcode for each cell. The round barcodes are then concatenated to determine the full barcode.

FIGS. 16A-16B provide a summary of classification accuracy for Example 11. Barcode classification was performed for each cell in each round. Each well was encoded with a unique barcode (legend in bottom right). FIG. 16A: The accuracy was defined as the number of cells with round-barcodes exactly matching the encoding (Match=TRUE) divided by the number with any difference from encoding (Match=FALSE). For each well, in each round, over 2000 bacterial cells were classified. A single cell was misclassified in round 3 of well 1. FIG. 16B: A fourth round of exchange was performed to restore the original, round 1 barcode in each well and again performed classification. The accuracy of classification for round 1 and round 4 for each well is shown.

FIG. 17 illustrates that bacteria fluorescence matches expected barcode. In each well a mask for the most abundant barcode applied to the maximum spectral projection. Fluorescent bacteria only appear in channels corresponding to the “1” bit.

FIG. 18 shows a field of view in tissue for three different rounds of HiPR-Swap to detect microbial taxa at the phylum level. For each round, the colors corresponding to the phyla present in the round are shown. Large speckled blue (color not shown) objects at the bottom of each image are DAPI-stained nuclei in the host epithelium. Insets with bacteria are shown in white boxes. Outline phylum names indicate low abundance taxa.

FIG. 19 shows a field of view in tissue for three different rounds of HiPR-Swap to detect microbial taxa at the species level. For each round, individual species were encoded with a single bit. Large speckled blue objects at the top right of each image are DAPI-stained nuclei in the host epithelium. Color change between images indicates signal exchange from the HiPR-Swap assay.

### DETAILED DESCRIPTION

It is to be appreciated that certain aspects, modes, embodiments, variations, and features of the present methods and compositions are described below in various levels of detail in order to provide a substantial understanding of the present disclosure.

### Definitions

Where values are described as ranges, endpoints are included. Furthermore, it will be understood that such disclosure includes the disclosure of all possible sub-ranges within such ranges, as well as specific numerical values that fall within such ranges irrespective of whether a specific numerical value or specific sub-range is expressly stated.

“5′-end” and “3′-end” refers to the directionality, e.g., the end-to-end orientation of a nucleotide polymer (e.g., DNA). The 5′-end of a polynucleotide is the end of the polynucleotide that has the fifth carbon.

The term “about,” as used herein, refers to +/−10% of a recited value.

“Complementary” refers to the topological compatibility or matching together of interacting surfaces of two nucleotides as understood by those of skill in the art. Thus, two sequences are “complementary” to one another if they are capable of hybridizing to one another to form a stable anti-parallel, double-stranded nucleic acid structure. A first nucleotide is complementary to a second nucleotide if the nucleotide sequence of the first nucleotide is substantially identical to the nucleotide sequence of the nucleotide binding partner of the second nucleotide, or if the first nucleotide can hybridize to the second nucleotide under stringent hybridization conditions. Thus, the nucleotide whose sequence is 5′-TATAC-3′ is complementary to a nucleotide whose sequence is 5′-GTATA-3′.

“Nucleotides,” “Nucleic acids,” “polynucleotide” or “oligonucleotide” refer to a polymeric-form of DNA and/or RNA (e.g., ribonucleotides, deoxyribonucleotides, or analogs thereof) of any length; e.g., a sequence of two or more ribonucleotides or deoxyribonucleotides. As used herein, the term “nucleotides” includes double- and single-stranded DNA, as well as double- and single-stranded RNA; it also includes modified and unmodified forms of a nucleotide (modifications to and of a nucleotide, for example, can include methylation, phosphorylation, and/or capping). In some embodiments, a nucleotide can be one of the following: a gene or gene fragment; genomic DNA; genomic DNA fragment; exon; intron; messenger RNA (mRNA); transfer RNA (tRNA); ribosomal RNA (rRNA); ribozyme; cDNA; recombinant nucleotide; branched nucleotide; plasmid; vector; isolated DNA of any sequence; isolated RNA of any sequence; any DNA described herein, any RNA described herein, primer or amplified copy of any of the foregoing.

In some embodiments, nucleotides can have any three-dimensional structure and may perform any function, known or unknown. The structure of nucleotides can also be referenced to by their 5′- or 3′-end or terminus, which indicates the directionality of the nucleotide sequence. Adjacent nucleotides in a single-strand of nucleotides are typically joined by a phosphodiester bond between their 3′ and 5′ carbons. However, different internucleotide linkages could also be used, such as linkages that include a methylene, phosphoramidate linkages, etc. This means that the respective 5′ and 3′ carbons can be exposed at either end of the nucleotide sequence, which may be called the 5′ and 3′ ends or termini. The 5′ and 3′ ends can also be called the phosphoryl (PO₄) and hydroxyl (OH) ends, respectively, because of the chemical groups attached to those ends. The term “nucleotides” also refers to both double- and single-stranded molecules.

In some embodiments, nucleotides can include modified nucleotides, such as methylated nucleotides and nucleotide analogs (including nucleotides with non-natural bases, nucleotides with modified natural bases such as aza- or deaza-purines, etc.). If present, modifications to the nucleotide structure can be imparted before or after assembly of the nucleotide sequence.

In some embodiments, the sequence of nucleotides can be interrupted by non-nucleotide components. One or more ends of the nucleotides can be protected or otherwise modified to prevent that end from interacting in a particular way (e.g. forming a covalent bond) with other nucleotides.

In some embodiments, nucleotides can be composed of a specific sequence of four nucleotide bases: adenine (A); cytosine (C); guanine (G); and thymine (T). Uracil (U) can also be present, for example, as a natural replacement for thymine when the nucleotide is RNA. Uracil can also be used in DNA. Thus, the term “sequence” refers to the alphabetical representation of nucleotides or any nucleic acid molecule, including natural and non-natural bases.

When used in terms of length, for example 20 nt, “nt” refers to nucleotides.

As used herein a “taxon” refers to a group of one or more populations of an organism or organisms. In some embodiments, a “taxon” refers to a phylum, a class, an order, a family, a genus, a species, or a train. In some embodiments, the disclosure includes providing a list of taxa of microorganisms. In some embodiments, the list of taxa of microorganisms is selected from a list of phyla, a list of classes, a list of orders, a list of families, a list of genera, or a list of species, of microorganisms.

In analysis of a sample, a species can be a target of interest. For example, a species can include a taxonomic species.

In the event of any term having an inconsistent definition between this application and a referenced document, the term is to be interpreted as defined herein.

The development of antimicrobial resistance among infectious organisms is an emerging problem in patient treatment. Some microbial organisms have even become resistant to multiple classes of antimicrobials, leading to increasing incidences of potentially fatal infections that cannot be treated with available antimicrobials. In some cases, microbes possessing more than one antimicrobial gene may only begin expressing one or more of these genes after exposure to antimicrobials. Currently, microbiology laboratories in hospitals and clinics rely on culturing bacteria from patient samples before species identification or antimicrobial susceptibility testing, however culturing bacteria is time-consuming and labor-intensive. Furthermore, many microorganisms are not readily culturable.

In a typical clinical lab workflow, the culturing step involves plating patient samples on an agar substrate and waiting for individual bacterium to grow into macroscopic colonies, each containing 10 to 100 million cells. Depending on the species of bacteria, this process can take between 24 hours to several days, which leads to a significant time delay from sampling to diagnosis. In addition, culturing bacteria requires a technician to prepare the culture plates by hand and evaluate bacterial growth by eye. Both of these factors add unnecessary hands-on time for the technician, and further increase the amount of time required for diagnosis. In certain classes of diseases such as sepsis, a delayed diagnosis can mean life or death for the patient.

The key innovative step of this method is to implement parallel single-cell imaging for microbial identification and characterization, which identification of microbial genera and species, and assessment of growth under different antimicrobial conditions directly on individual microbial cells or small colonies of cells, without the need to wait for cells to grow and divide into colonies containing millions to billions of cells.

Cell division events or microbial morphology changes can be monitored via iterative imaging of the sample during culture, or at the conclusion of culturing and following fixation, to measure microbial growth and stress in a solution with a given concentration of antimicrobials. Because the observation of only a few cell division events is sufficient to assess susceptibility or resistance of the microbial species to an antimicrobial agent, this technique can provide definitive results in less time than a complete cell cycle. This process is orders of magnitude faster than current techniques. For example, in a population of 1000 asynchronously dividing cells, the mean waiting time for the next division event to occur is 1/1000 of the duration of the typical cell cycle. For example, if the bacteria is E. coli, with an average cell division time of 20 minutes, the next event occurs after roughly one second. The parallel observation of many (thousands and more) cells also enables the construction of division time distribution for accurate determination of growth rate over a time duration of one or few cell cycles. In some embodiments, the cells may be allowed to grow for a defined period of time. After the growth period, the samples can be fixed and observed on a microscope. In some cases, growth is measured by counting the number of micro colonies present in the sample. In other embodiments, the cells may be observed on a microscope while they are growing. In some embodiments, after acquiring the necessary growth and stress data, the sample can be fixed directly and parallel single-cell imaging performed to read out the species identity of the microorganism of interest. This may be followed up with single molecule imaging to measure the presence of genes that may indicate current or future susceptibility to antimicrobials. The micro-colony level or single-cell level observation will drastically cut down the time required to go from sample to diagnosis, requiring on average a few (e.g. one, two, or three) cell divisions to occur before the readout step, and will provide clinicians with actionable information earlier than any existing technology. Furthermore, the present methods provide clinicians with the antimicrobial susceptibility information needed to deploy targeted antimicrobials and enable precise treatments tailored for each individual case, thereby reducing the spread of multi-drug resistance among microbial populations. A live/dead stain (e.g. viability dye) can also be incorporated in unused spectral channels, to distinguish single, living microorganisms which did not divide over the course of the assay from those that are dead.

In addition to antimicrobial susceptibility, other microbial phenotype measurements can be combined with HiPR-FISH species identification and quantification. In some embodiments, the tolerance or persistence of microbial cells in the presence of environmental stress can be determined by measuring the gene expression levels for stress response genes (e.g. RpoS, RpoN, and/or RpoE, which encodes the sigma factor that regulates the response to conditions of stress). In some embodiments, motility or chemotaxis measurements can be combined with HiPR-FISH to identify cellular motility in a taxa-specific fashion. In some embodiments, the production of reactive oxygen species (ROS), which play important roles in promoting microbial tolerance to environmental stress, can be measured and linked to the species identity of each cell. In some embodiments, the expression of Type 3 Secretion System (T3 SS) genes, which are used by certain pathogens to infect host cells and evade host immune response, can be measured and linked to species identity. In some embodiments, the expression of Type IV Secretion System (T4SS), which is related to the prokaryotic conjugation machinery and is involved in transport of proteins and DNA across the cell membrane, can be measured and linked to species identity. In some embodiments, the expression of quorum sensing genes, which are important in modulating collective behavior of communities containing many microbial cells, can be measured and linked to species identity. In some embodiments, the expression of genes related to biofilm formation can be measured and linked to species identity. In some embodiments, microbial cells can be subjected to a phage to identify phage-susceptible microbial species.

### Single Cell Imaging

In some embodiments, the present disclosure is directed to a method that achieves high phylogenetic resolution by taking advantage of the abundance of existing ribosomal subunit sequence information, such as the 16S ribosomal RNA sequence information, and a highly multiplexed binary encoding scheme. In some embodiments, each taxon from a list of taxa of microorganisms is probed with a custom designed taxon-specific targeting sequence, flanked by a subset of n unique encoding sequences. In some embodiments, each taxon is assigned a unique n-bit binary word, where 1 or 0 at the i^{th} bit indicates the taxon-specific targeting sequence is flanked or not flanked by the i^{h} encoding sequence. In some embodiments, a mixture of n decoding probes, each complementary to one of the n encoding sequences and conjugated to a unique label, is allowed to hybridize to their complementary encoding sequences. In some embodiments, the spectrum of labels for each cell is then detected using spectral imaging techniques. In some embodiments, the barcode identity for each cell can then be assigned using a support vector machine, using spectra of cells encoded with known barcodes or using computationally simulated spectra as training data.

In some embodiments, each taxon from a list of taxa of microorganisms is assigned a unique n-bit binary code selected from a plurality of unique n-bit binary codes, where n is an integer greater than 1.

A “binary code” refers to a representation of taxa using a string made up of a plurality of “0” and “1” from the binary number system. The binary code is made up of a pattern of n binary digits (n-bits), where n is an integer representing the number of labels used. The bigger the number n, the greater number of taxa can be represented using the binary code. For example, a binary code of eight bits (an 8-bit binary code, using 8 different labels) can represent up to 255 (2⁸−1) possible taxa. (One is subtracted from the total possible number of codes because no taxon is assigned a code of all zeros “00000000.” A code of all zeros would mean no decoding sequence, and thus no label, is attached. In other words, there are no non-labeled taxa.) Similarly, a binary code of ten bits (a 10-bit binary code) can represent up to 1023 (2¹⁰−1) possible taxa. In some embodiments a binary code may be translated into and represented by a decimal number. For example, the 10-bit binary code “0001100001” can also be represented as the decimal number “97.”

Each digit in a unique binary code represents whether a readout probe and the fluorophore corresponding to that readout probe are present for the selected species. In some embodiments, each digit in the binary code corresponds to a Readout probe (from Readout probe 1 (R1) through Readout probe n (Rn) in an n-bit coding scheme). In a specific embodiment, the n is 10 and the digits of an n-bit code correspond to R1 through R10. In some embodiments, the fluorophores that correspond to R1 through Rn are determined arbitrarily. For example, if n is 10, R1 can correspond to an Alexa 488 fluorophore, R2 can correspond to an Alexa 546 fluorophore, R3 can correspond to a 6-ROX (6-Carboxy-X-Rhodamine, or Rhodamine Red X) fluorophore, R4 can correspond to a Pacific Green fluorophore, R5 can correspond to a Pacific Blue fluorophore, R6 can correspond to an Alexa 610 fluorophore, R7 can correspond to an Alexa 647 fluorophore, R8 can correspond to a DyLight-510-LS fluorophore, R9 can correspond to an Alexa 405 fluorophore, and R10 can correspond to an Alexa532 fluorophore. Other n-bit and readout probes combinations are also contemplated herein. In some embodiments, other fluorophores including, but not limited to Hydroxycoumarin, methoxycoumarin, Cy2, FAM, Flourescein FITC, Alexa 430, R-phycoerythrin (PE), Tamara, Cy3.5 581, Rox, Alexa fluor 568, Red 613, Texas Red, Alexa fluor 594, Alexa fluor 633, Alexa fluor 660, Alexa fluor 680, Cy5, Cy 5.5, Cy 7, and Allophycocyanin are used in the n-bit encoding system.

In some embodiments, the n-bit binary code is between a 2-bit binary code and 50-bit binary code, a 2-bit binary code and 40-bit binary code, or 2-bit binary code and 30-bit binary code. In some embodiments, the n-bit binary code is selected from the group consisting of 2-bit binary code, 3-bit binary code, 4-bit binary code, 5-bit binary code, 6-bit binary code, 7-bit binary code, 8-bit binary code, 9-bit binary code, 10-bit binary-code, 11-bit binary code, 12-bit binary code, 13-bit binary code, 14-bit binary code, 15-bit binary code, 16-bit binary code, 17-bit binary code, 18-bit binary code, 19-bit binary code, 20-bit binary code, 21-bit binary code, 22-bit binary code, 23-bit binary code, 24-bit binary code, 25-bit binary code, 26-bit binary code, 27-bit binary code, 28 bit binary code, 29-bit binary code, 30-bit binary code, 31-bit binary code, 32-bit binary code, 33-bit binary code, 34-bit binary code, 35-bit binary code, 36-bit binary code, 37-bit binary code, 38 bit binary code, 39-bit binary code, 40-bit binary code, 41-bit binary code, 42-bit binary code, 43-bit binary code, 44-bit binary code, 45-bit binary code, 46-bit binary code, 47-bit binary code, 48 bit binary code, 49-bit binary code, and 50-bit binary code.

Encoding Probes

In some embodiments, the gene for a ribosomal subunit is used as a marker for phylogenetic placement. In some embodiments, 16S rRNA gene is used as a marker for phylogenetic placement. In some embodiments, methods of the present disclosure comprise multiplexed in-situ hybridization of encoding probes targeting taxon-specific segments of multiple unique 16S rRNA genes present in a microorganism population. In some embodiments, the 5S and/or 23S rRNA are used independently or in conjunction with 16S rRNA as a marker for phylogenetic placement. In some embodiments, if non-bacterial microorganisms are targeted, other rRNA may be targeted.

In some embodiments, a set of ending probes comprises subsets of encoding probes, wherein each subset targets a specific taxon. In some embodiments, a subset of encoding probes contains one unique targeting sequence specific to a taxon; that is, the encoding probes within a subset share a common targeting sequence specific to a taxon. In some embodiments, a subset of encoding probes contains multiple unique targeting sequences, each unique targeting sequence being specific to the same taxon as other targeting sequences within the same subset.

Targeting Sequences

In some embodiments, each encoding probe comprises a targeting sequence which is substantially complementary to a taxon-specific 16S rRNA sequence. By “substantially complementary” it is meant that the nucleic add fragment is capable of hybridizing to at least one nucleic acid strand or duplex even if less than all nucleobases base pair with a counterpart nucleobase. In certain embodiments, a “substantially complementary” nucleic add contains at least one sequence in which about 70%, about 71%, about 72%, about 73%, about 74%, about 75%, about 76%, about 77%, about 77%, 8%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range therein, of the nucleobase sequence is capable of basepairing with at least one single or double stranded nucleic acid molecule during hybridization.

In some embodiments, the targeting sequence is designed to have a predicted melting temperature of between about 45° C. and about 65° C. or between about 55° C. and about 65° C. As used herein, the term “about” refers to an approximately ±10% variation from a given value. In some embodiments, the predicted melting temperature of the targeting sequence is 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C. or 65° C. In some embodiments, the targeting sequence has a GC content of about 55%, 60%, 65% or 70%.

In some embodiments, the taxon-specific targeting sequence in an encoding probe is designed as follows. At first, 16S sequences from a plurality of microorganisms are grouped by taxon and sequence similarity and a consensus sequence is generated for each taxon. In some embodiments, a targeting sequence specific for a consensus sequence is at least 10 nucleotides to at least 100 nucleotides long. In some embodiments, a targeting sequence specific for a consensus sequence is at least 15 nucleotides long, at least 16 nucleotides long, at least 17 nucleotides long, at least 18 nucleotides long, at least 19 nucleotides long, at least 20 nucleotides long, at least 21 nucleotides long, at least 22 nucleotides long, at least 23 nucleotides long, at least 24 nucleotides long, at least 25 nucleotides long, at least 26 nucleotides long, at least 27 nucleotides long, at least 28 nucleotides long, at least 29 nucleotides long, at least 30 nucleotides long, at least 35 nucleotides long, at least 40 nucleotides long, at least 45 nucleotides long, or at least 50 nucleotides long. In some embodiments, the candidate targeting sequence is aligned against a catalog of all full-length 16S rRNA sequences of a list of microorganisms. In a specific embodiment, the alignment is performed using Blastn (NCBI). In a specific embodiment, the alignment is performed using BWA. In a specific embodiment, the alignment is performed using bowtie. In a specific embodiment, the alignment is performed using bowtie2. In some embodiments, a maximum continuous homology (MCH) score, defined as the maximum number of continuous bases that are shared between the query and the target sequence, is calculated for each blast hit. In some embodiments, only candidate targeting sequences having blast hits to the consensus sequence above a threshold MCH score are considered significant and used for further analysis. In some embodiments, a blast on-target rate, defined as the ratio between the number of correct blast hits and the total number of significant blast hits, is calculated for each candidate targeting sequence having a significant BLAST hit. In some embodiments, any candidate targeting sequence with a blast on-target rate of less than 1 is excluded from the probe set to avoid ambiguity, and the remaining candidate targeting sequences are used as targeting sequences in encoding probe synthesis.

In some embodiments, the targeting sequence of an encoding probe is designed using publicly-available 16S rRNA sequence data. In some embodiments, the targeting sequence of an encoding probe is designed using publicly-available 23S rRNA sequence data. In some embodiments, the targeting sequence of an encoding probe is designed using publicly-available 5S rRNA sequence data. In some embodiments, the targeting sequence of an encoding probe design is designed using custom catalogues of 16S rRNA sequences. In some embodiments, the targeting sequence of an encoding probe design is designed using custom catalogues of 23S rRNA sequences. In some embodiments, the targeting sequence of an encoding probe design is designed using custom catalogues of 5S rRNA sequences. In some embodiments, the targeting sequence of an encoding probe is designed using publicly-available 16S-5S rRNA sequence data. In some embodiments, the targeting sequence of an encoding probe is designed using publicly-available 16S-5S-23S rRNA sequence data. In some embodiments, the targeting sequence of an encoding probe design is designed using custom catalogues of 16S-5S rRNA sequences. In some embodiments, the targeting sequence of an encoding probe design is designed using custom catalogues of 16S-5S-23S rRNA sequences. In a specific embodiment, high-quality, full-length 16S sequences are obtained by circular consensus sequencing (SMRT-CCS). In a specific embodiment, high-quality, full-length 16S sequences are obtained by Nanopore sequencing.

In some embodiments, SMRT-CCS of a 16S ribosomal sequence involves isolating ribosomal DNA from a microorganism. In a specific embodiment, DNA isolation is achieved using QIAamp DNA Mini Kit. In a specific embodiment, DNA isolation is achieved using DNeasy PowerSoil Pro Kit. In some embodiments, ribosomal DNA is amplified using universal primers. In some embodiments, the amplified ribosomal DNA is purified, and sequenced. In a specific embodiment, sequencing is performed on a PacBio Sequel instrument. In a specific embodiment, sequencing is performed on a PacBio Sequel IIe instrument. In a specific embodiment, sequencing is performed on a Nanopore MinION instrument. In a specific embodiment, sequencing is performed on a Nanopore GridION instrument. In a specific embodiment, sequencing is performed on a Nanopore PromethION instrument. In some embodiments, sequence data is processed to create a circular consensus sequence with a threshold of 99% accuracy. In a specific embodiment, the sequence data processing is achieved using rDnaTools. In some embodiments, the circular consensus sequences are used for probe design. In some embodiments, to increase the sequence design space, and to improve identification of closely related species, the workflow uses a full 16S-23S rRNA region. In some embodiments, to increase the sequence design space, and to improve identification of closely related species, the workflow uses a full 16S-5S-23S rRNA region.

In some embodiments, the targeting sequence of an encoding probe is designed using a database that is relevant for a system. In a specific embodiment, the system is the gut microbiome. In some embodiments, the targeting sequence of an encoding probe is designed using a database that is relevant for a disease or infection.

Spacers

In some embodiments, a targeting sequence in an encoding probe is concatenated on both ends with 3 nucleotide (3-nt) spacers. In some embodiments, the 3-nt spacers comprise a random string of three nucleotides. In some embodiments, the 3-nt spacers are sequences designed from the 16S rRNA molecule, 5S rRNA molecule, or 23S rRNA molecule (i.e., three nucleotides upstream and downstream of the selected 16S targeting sequence is used as the 3-nt spacers). In some embodiments the spaces are non-nucleotide chemical spacers. Non-nucleotide chemical spacers include, but are not limited to, hexanediol, hexa-ethyleneglycol, or triethylene glycol spacers.

Readout Sequences

In some embodiments, a targeting sequence is concatenated to at least one readout sequence depending on the unique n-bit binary code assigned to the taxon that the targeting sequence is specific for. Each readout sequence is substantially complementary to the sequence of a corresponding labeled readout probe.

In some embodiments, a readout sequence is at least 15 nucleotides long, at least 16 nucleotides long, at least 17 nucleotides long, at least 18 nucleotides long, at least 19 nucleotides long, at least 20 nucleotides long, at least 21 nucleotides long, at least 22 nucleotides long, at least 23 nucleotides long, at least 24 nucleotides long, at least 25 nucleotides long, at least 26 nucleotides long, at least 27 nucleotides long, at least 28 nucleotides long, at least 29 nucleotides long, or at least 30 nucleotides long. In some embodiments, candidate readout sequences are blasted against a nucleotide database to ensure that they are not substantially complementary to regions of 16S ribosomal sequences.

Forward and Reverse Primers

In some embodiments, a targeting sequence is concatenated to a set of sequences (forward primer and reverse primer sequences) that are substantially complementary to primers that can be used to amplify the encoding probe in a polymerase chain reaction (PCR). In some embodiments, the forward and reverse primers are designed to have predicted melting temperatures of between about 55° C. and about 65° C. As used herein, the term “about” refers to an approximately ±10% variation from a given value. In some embodiments, the predicted melting temperature of the forward and reverse primers are 55° C., 56° C., 57° C., 58° C., 59° C., 60° C., 61° C., 62° C., 63° C., 64° C. or 65° C. In some embodiments, the forward and reverse primers have a GC content of about 55%, 60%, 65% or 70%.

In some embodiments, the set of forward and reverse primers are designed such that the set of forward and reverse primers are not substantially complementary to the targeting sequence or readout sequences. In some embodiments, the set of forward and reverse primers are designed such that the set of forward and reverse primers are not substantially complementary to any sequences that are substantially complementary to the targeting sequence or readout sequences. In a specific embodiment, the set of forward primer and reverse primer sequences comprise the nucleotide sequence CGATGCGCCAATTCCGGTTC (SEQ ID NO: 1808) and the nucleotide sequence GTCTATTTTCTTATCCGACG (SEQ ID NO: 1809).

In some embodiments, the forward primer or the reverse primer is at least 15 nucleotides long, at least 16 nucleotides long, at least 17 nucleotides long, at least 18 nucleotides long, at least 19 nucleotides long, at least 20 nucleotides long, at least 21 nucleotides long, at least 22 nucleotides long, at least 23 nucleotides long, at least 24 nucleotides long, at least 25 nucleotides long, at least 26 nucleotides long, at least 27 nucleotides long, at least 28 nucleotides long, at least 29 nucleotides long, or at least 30 nucleotides long.

Decoding Probes

In some embodiments, the present disclosure utilizes a set of n number of decoding probes representing an n-bit coding scheme where n is an integer. In some embodiments, each probe in the set of decoding probes corresponds to a digit in the plurality of unique n-bit binary codes.

In some embodiments, each probe in the set of decoding probes is conjugated with a label that provides a detectable signal.

In some embodiments, each probe in a set of decoding probes is labeled different from other probes in the set, and each decoding probe is substantially complementary to a corresponding readout sequence selected from a set of n number of readout sequences.

In some embodiments, the detectable signal is a cyanine dye (e.g., Cy2, Cy3, Cy3B, Cy5, Cy5.5, Cy7, etc.), Alexa Fluor dye, Atto dye, photo switchable dye, photoactivatable dye, fluorescent dye, metal nanoparticle, semiconductor nanoparticle or “quantum dots”, fluorescent protein such as GFP (Green Fluorescent Protein), or photoactivatable fluorescent protein, such as PAGFP, PSCFP, PSCFP2, Dendra, Dendra2, EosFP, tdEos, mEos2, mEos3, PAmCherry, PAtagRFP, mMaple, mMaple2, and mMaple3.

In a specific embodiment, the detectable signal is a fluorophore. In some embodiments, the detectable signal is a fluorophore that emits light in infrared or near-infrared. In a specific embodiment, the fluorophore is selected from the group consisting of Alexa 405, Pacific Blue, Pacific Green, Alexa 488, Alexa 532, Alexa 546, Rhodamine Red X, Alexa 610, Alexa 647, and DyLight-510-LS, Hydroxycoumarin, methoxycoumarin, Cy2, FAM, Fluorescein FITC, Alexa 430, R-phycoerythrin (PE), Tamara, Cy3.5 581, Rox, Alexa fluor 568, Red 613, Texas Red, Alexa fluor 594, Alexa fluor 633, Alexa fluor 660, Alexa fluor 680, Cy5, Cy5.5, Cy7, Allophycocyanin, and ROX (carboxy-X-rhodamine). In some embodiments, the detectable signal is Alexa Fluor 350, Alexa Fluor 405, Alexa Fluor 430, Alexa Fluor 488, Alexa Fluor 514, Alexa Fluor 532, Alexa Fluor 546, Alexa Fluor 555, Alexa Fluor 561, Alexa Fluor 568, Alexa Fluor 594, Alexa Fluor 610, Alexa Fluor 633, Alexa Fluor 635, Alexa Fluor 647, Alexa Fluor 647-R-phycoerythrin, Alexa Fluor 660, Alexa Fluor 680, Alexa Fluor 680-allophycocyanin, Alexa Fluor 700, Alexa Fluor 750, Alexa Fluor 790, Alexa Fluor Plus 405, Alexa Fluor Plus 488, Alexa Fluor Plus 555, Alexa Fluor Plus 594, Alexa Fluor Plus 647, Alexa Fluor Plus 680, Alexa Fluor Plus 750, Alexa Fluor Plus 800, Pacific Blue, Pacific Green, Rhodamine Red X, DyLight 485-LS, DyLight-510-LS, DyLight 515-LS, DyLight 521-LS, Hydroxycoumarin, methoxycoumarin, Cy2, FAM, Fluorescein FITC, R-phycoerythrin (PE), Tamara, Cy3.5 581, ROX (carboxy-X-rhodamine), Red 613, Texas Red, Cy5, Cy5.5, Cy7, Allophycocyanin, ATTO 430LS, ATTO 490LS, ATTO 390, ATTO 425, Cyan 500 NHS-Ester, ATTO 465, ATTO 488, ATTO 495, ATTO Rho110, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 643, ATTO 647, ATTO 647N, ATTO 655, ATTO Oxa12, ATTO 665, ATTO 680, ATTO 700, ATTO 725, ATTO 740.

In some embodiments, a readout probe is at least 10 nucleotides long, at least 11 nucleotides long, at least 12 nucleotides long, at least 13 nucleotides long, at least 14 nucleotides long, at least 15 nucleotides long, at least 16 nucleotides long, at least 17 nucleotides long, at least 18 nucleotides long, at least 19 nucleotides long, at least 20 nucleotides long, at least 21 nucleotides long, at least 22 nucleotides long, at least 23 nucleotides long, at least 24 nucleotides long, at least 25 nucleotides long, at least 26 nucleotides long, at least 27 nucleotides long, at least 28 nucleotides long, at least 29 nucleotides long, or at least 30 nucleotides long.

Imaging

In some embodiments, the labels used in the present methods are imaged using a microscope. In some embodiments, the microscope is a confocal microscope. In some embodiments, the microscope is a fluorescence microscope. In some embodiments, the microscope is a light-sheet microscope. In some embodiments, the microscope is a super-resolution microscope.

Barcode Decoding

In some embodiments, a support vector machine is trained on reference data to predict the barcode of single cells in the synthetic communities and environmental samples. In a specific embodiment, the support vector machine is Support Vector Regression (SVR) from Python package. As used herein, the term “support-vector machine” (SVM) refers to a supervised learning model with associated learning algorithms that analyze data used for classification and regression analysis. Given a set of training examples, each marked as belonging to one or the other of two categories, an SVM training algorithm builds a model that assigns new examples to one category or the other, making it a non-probabilistic binary linear classifier. An SVM model is a representation of the examples as points in space, mapped so that the examples of the separate categories are divided by a clear gap that is as wide as possible. New examples are then mapped into that same space and predicted to belong to a category based on which side of the gap they fall.

In some embodiments, the reference spectra are obtained through a brute force approach involving the measurement of the spectra of all possible barcodes using barcoded test E. coli cells. In some embodiments, the n-bit binary encoding is a 10-bit binary encoding and tire reference spectra are obtained through measuring 1023 reference spectra.

In some embodiments the reference spectra are obtained by simulation of all possible spectra. In some embodiments, the simulated spectral data can be used as reference examples for the support vector machine. In some embodiments, the spectra corresponding to individual n-bit binary codes are simulated by adding together the measured spectra of each individual fluorophore (e.g., the reference spectrum for 0000010011 is generated by adding the spectra of R1, R2, and R5; or the reference spectrum for 1010010100 is generated by adding the spectra of R3, R5, R8 and R10). In some embodiments, the spectra corresponding to individual n-bit binary codes are simulated by adding the measured spectra of each individual fluorophore weighted by the relative contribution to the emission signal of each fluorophore. In some embodiments, the relative contribution of each fluorophore is calculated using a Forster Resonant Energy Transfer (FRET) model.

In one aspect, the disclosure is directed to a computer-readable storage device storing computer readable instructions, which when executed by a processor causes the processor to assign each taxon in a list of taxa of microorganisms a unique n-bit binary code selected from a plurality of unique n-bit binary codes, and design decoding and encoding probes suitable for use in such n-bit binary coding scheme.

The phrase “computer-readable storage device” refers to a computer readable storage device or a computer readable signal medium. A computer-readable storage device, may be, for example, a magnetic, optical, electronic, electromagnetic, infrared, or semiconductor system, apparatus, or device, or any suitable combination of the foregoing; however, the computer readable storage device is not limited to these examples except a computer readable storage device excludes computer readable signal medium Additional examples of the computer readable storage device can include: a portable computer diskette, a hard disk, a magnetic storage device, a portable compact disc read-only memory (CD-ROM), a random access memory' (RAM), a read-only memory (ROM), an erasable programmable read-only memory (EPROM or Flash memory), an optical storage device, or any appropriate combination of the foregoing; however, the computer readable storage device is also not limited to these examples.

Any tangible medium that can contain, or store, a program for use by or in connection with an instruction execution system, apparatus, or device could be a computer readable storage device.

A computer readable signal medium may include a propagated data signal with computer readable program code embodied therein, such as, but not limited to, in baseband or as part of a carrier wave. A propagated signal may take any of a plurality of forms, including, but not limited to, electro-magnetic, optical, or any suitable combination thereof. A computer readable signal medium may be any computer readable medium (exclusive of computer readable storage device) that can communicate, propagate, or transport a program for use by or in connection with a system, apparatus, or device. Program code embodied on a computer readable signal medium may be transmitted using any appropriate medium, including but not limited to wireless, wired, optical fiber cable, RF, etc., or any suitable combination of the foregoing. The term “memory” as used herein comprises program memory' and working memory. The program memory may have one or more programs or software modules. The working memory stores data or information used by the CPU in executing the functionality described herein.

The term “processor” may include a single core processor, a multi-core processor, multiple processors located in a single device, or multiple processors in wired or wireless communication with each other and distributed over a network of devices, the Internet, or the cloud. Accordingly, as used herein, functions, features or instructions performed or configured to be performed by a “processor”, may include the performance of the functions, features or instructions by a single core processor, may include performance of the functions, features or instructions collectively or collaboratively by multiple cores of a multi-core processor, or may include performance of the functions, features or instructions collectively or collaboratively by multiple processors, where each processor or core is not required to perform every function, feature or instruction individually. The processor may be a CPU (central processing unit). The processor may comprise other types of processors such as a GPU (graphical processing unit). In other aspects of the disclosure, instead of or in addition to a CPU executing instructions that are programmed in the program memory, the processor may be an ASIC (application-specific integrated circuit), analog circuit or other functional logic, such as a FPGA (field-programmable gate array), PAL (Phase Alternating Line) or PLA (programmable logic array).

The CPU is configured to execute programs (also described herein as modules or instructions) stored in a program memory to perform the functionality described herein. The memory may be, but not limited to, RAM (random access memory), ROM (read only memory) and persistent storage. The memory is any piece of hardware that is capable of storing information, such as, for example without limitation, data, programs, instructions, program code, and/or other suitable information, either on a temporary basis and/or a permanent basis.

In some embodiments, a computer-readable storage device comprises instructions for assigning each taxon in a list of taxa of microorganisms a unique n-bit binary code selected from a plurality of unique n-bit binary codes; designing a set of n number of decoding probes, wherein each decoding probe corresponds to a digit in the n-bit binary code, and where each decoding probe is substantially complementary to a readout sequence selected from a set of n number of readout sequences, and designing a set of encoding probes, where the set of encoding probes includes a plurality of subsets of encoding probes, wherein each encoding probe comprises a targeting sequence and one or more readout sequences, the encoding probes within each subset comprise a targeting sequence that is specific to a taxon in tire list of taxa of microorganisms and is different from a targeting sequence of the encoding probes of another subset, and the readout sequences in the encoding probes within a subset are selected from the set of n number of readout sequences based on the unique n-bit binary code assigned to the taxon which the targeting sequence of the subset is specific to.

In some embodiments, the computer-readable storage device comprises instructions for designing encoding probes of a subset, wherein the targeting sequence in the encoding probes of a subset is substantially complementary to a consensus 16S ribosomal sequence specific to a taxon. In some embodiments, the computer-readable storage device comprises instructions for designing encoding probes of a subset, wherein the targeting sequence in the encoding probes of a subset is substantially complementary to a consensus 5S ribosomal sequence specific to a taxon. In some embodiments, the computer-readable storage device comprises instructions for designing encoding probes of a subset, wherein the targeting sequence in the encoding probes of a subset is substantially complementary to a consensus 23S ribosomal sequence specific to a taxon. In some embodiments, the computer-readable storage device comprises instructions for designing encoding probes of a subset, wherein the targeting sequence in the encoding probes of a subset is substantially complementary to a consensus 16S-5S ribosomal sequence specific to a taxon. In some embodiments, the computer-readable storage device comprises instructions for designing encoding probes of a subset, wherein the targeting sequence in the encoding probes of a subset is substantially complementary to a consensus 16S-5S-23S ribosomal sequence specific to a taxon. In some embodiments, the computer-readable storage device comprises instructions for designing encoding probes of a subset, wherein the targeting sequence in the encoding probes of a subset is substantially complementary to a consensus 16S-23S ribosomal sequence specific to a taxon. In some embodiments, the targeting sequence is blasted against a nucleotide database to ensure that the target sequence is not substantially complementary to any sequence other than the consensus 16S ribosomal sequence to which the target sequence is specific.

In some embodiments, a set of encoding probes comprises subsets of encoding probes, wherein each subset targets a specific taxon. In some embodiments, a subset of encoding probes contains one unique targeting sequence specific to a taxon; that is, the encoding probes within a subset share a common targeting sequence specific to a taxon. In some embodiments, a subset of encoding probes contains multiple unique targeting sequences, each unique targeting sequence being specific to the same taxon as other targeting sequences within the same subset.

### Microbial Cell Growth

In some embodiments, the microbial cell in the sample is identified and characterized directly from the sample. In some embodiments, the microbial cell in the sample is identified and characterized after culturing. In some embodiments, the microbial cell in the sample is cultured for numerous cell divisions. A skilled artisan would readily recognize that the number of cell divisions depends on the species doubling time, which varies from species to species. In some embodiments, the microbial cell in the sample is cultured for one to numerous cell divisions. In some embodiments, the microbial cell in the sample is cultured for less than one division cycle. In some embodiments, the microbial cell in the sample is cultured for very few cell division cycles. In some embodiments, the microbial cell in the sample is cultured for about 1 to about 12 cell division cycles. In some embodiments, the microbial cell in the sample is cultured for about 1, about 2, about 3, about 4, about 5, about 6, about 7, about 8, about 9, about 10, about 11, or about 12 cell division cycles. In some embodiments, the microbial cell in the sample is cultured for about 1 minute to about 12 hours. In some embodiments, the microbial cell in the sample is cultured for about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 15 minutes, about 16 minutes, about 17 minutes, about 18 minutes, about 19 minutes, about 20 minutes, about 25 minutes, about 30 minutes, about 35 minutes, about 40 minutes, about 45 minutes, about 50 minutes, about 55 minutes, about 60 minutes, about 80 minutes, about 90 minutes, about 1 hour, about 1.5 hours, about 2 hours, about 2.5 hours, about 3 hours, about 3.5 hours, about 4 hours, about 4.5 hours, about 5 hours, about 5.5 hours, about 6 hours, about 6.5 hours, about 7 hours, about 7.5 hours, about 8 hours, about 8.5 hours, about 9 hours, about 9.5 hours, about 10 hours, about 10.5 hours, about 11 hours, about 11.5 hours, or about 12 hours.

### Antimicrobial Susceptibility Testing

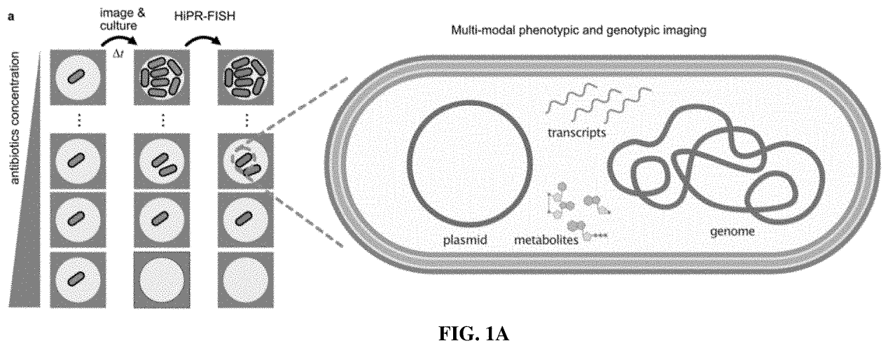

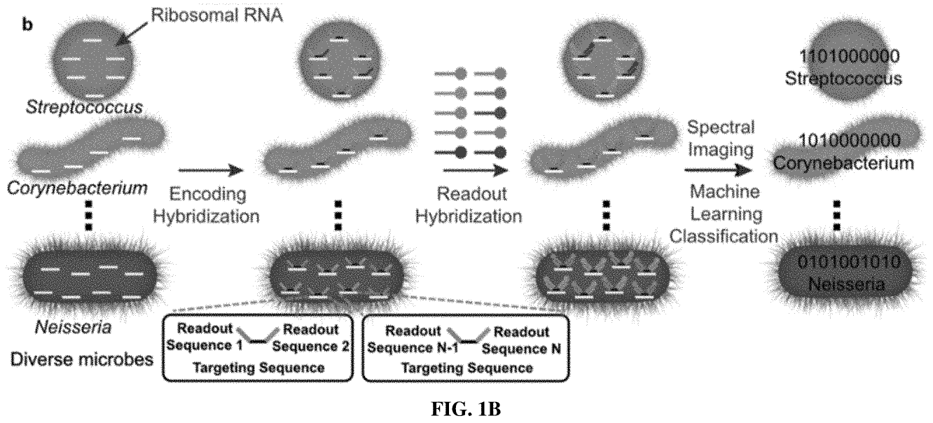

To enable rapid antimicrobial resistance profiling, the present methods combine fluorescence in situ hybridization to enable the first hybrid measurements of antimicrobial resistance (FIG. 1A) using both genotypic and phenotypic information. FIG. 1A shows a concept for rapid phenotypic profiling of antibiotic resistance followed by microbial identification using imaging. Microbes are cultured for a short amount of time (minutes) before fixation and imaging. Multimodal imaging using single-molecule FISH and metabolic labeling can provide phenotypic and genotypic information on cellular metabolism and antimicrobial resistance. This technique allows identification of microorganism species and assessment of microorganism growth and replication in the presence and absence of known concentrations of different antimicrobials in order to accurately determine antimicrobial susceptibility testing results in less time required than other methods known in the art.

One or more microbes in a specimen can be directly inoculated onto a device with patterned compartments. The testing can proceed with or without further culturing. In scenarios where the sample is not subjected to culturing, species identification FISH methods, such as HiPR-FISH, and single-molecule FISH to simultaneously image the species identity is combined with analysis regarding the presence or absence of one or more antimicrobial genes and metabolites, proteins, carbohydrates, and/or lipids in the same cells. This approach will enable a paired readout of microbial species identity and antimicrobial susceptibility. In situations where phenotypic readout of antimicrobial susceptibility is desired, the compartments will be filled with a culturing media containing an antimicrobial drug at a known concentration. An initial image will be taken to record the number of cells in each compartment of the device. The microbes are allowed to replicate for a defined period of time (minutes to a few hours). After the growth period, another image or measurement will be taken to record cellular state in each compartment of the device after the growth period and look for the presence of genes and metabolites, proteins, carbohydrates, and/or lipids that are known to confer antimicrobial resistance. The cellular state can potentially be read out in a few different ways. For example, cellular state can be measured simply by counting the number of cells in each compartment. Cell growth can also be measured by probing the metabolic product concentration in the solution such as dissolved CO₂ or measuring the amount of heat dissipation using calorimetry techniques. Cellular state can also be inferred by measuring the abundance of expressed metabolic genes or stress response genes using single-molecule fluorescence in situ hybridization. Cellular state may also be measured using a simple live/dead stain. After cellular state measurement, the identity of the cells will subsequently be read out using multiplexed fluorescence in situ hybridization (e.g. HiPR-FISH) (FIG. 1B). Binary labeling approach for highly multiplexed labeling of microbes for taxonomic identification. Microbes from different taxa are labeled with unique combinations of fluorophores. The combined spectra are measured using a microscope in spectral imaging mode. Measured spectra are classified using a custom machine learning algorithm. This test can be repeated in several different culture media to make the analysis as comprehensive as possible. Altogether, the present methods enable rapid measurement of pathogen identity, their associated minimally inhibitory concentration for antimicrobials, and potential future susceptibility to antimicrobials.

In some aspects, the present disclosure provides methods determining the susceptibility (or resistance) of the microbial cells in the sample to one or more antimicrobial agents. In some aspects, the present disclosure provides methods of identifying microbial cells in a sample in parallel with determination of the microbial cells in the sample susceptibility to one or more antimicrobial agents. As used herein, a microbial cell is “susceptible” to an antimicrobial when it is inhibited by the usually achievable concentration of the antimicrobial agent when the dosage recommended to treat the site of infection is used. Further, as used herein, a microbial cell is “resistant” to an antimicrobial when it is not inhibited by the usually achievable concentration of an antimicrobial agent with normal dosage schedules and/or that has a minimum inhibitory concentration that falls in the range in which specific microbial resistance mechanisms are likely.

In some embodiments, the microbial cells in a sample are exposed to different concentrations to determine the minimum inhibitory concentration of the antimicrobial agent. In some embodiments, the minimum inhibitory concentration (MIC) of the antimicrobial agent for the microbial cell in the sample is greater than the MIC of a typical microbial cell of the same strain. In some embodiments, the minimum inhibitory concentration (MIC) of the antimicrobial agent for the microbial cell in the sample is lower than the MIC of a typical microbial cell of the same strain.

In some embodiments, the microbial cells in the sample are exposed to one or more antimicrobial agents in a concentration range of about 2-fold to about 500-fold of the MIC of a typical microbial cell of the same strain. In some embodiments, the microbial cells in the sample are exposed to one or more antimicrobial agents in a concentration range of about 2-fold, about 3-fold, about 4-fold, about 5-fold, about 6-fold, about 7-fold, about 8-fold, about 9-fold, about 10-fold, about 11-fold, about 12-fold, about 13-fold, about 14-fold, about 15-fold, about 16-fold, about 17-fold, about 18-fold, about 19-fold, about 20-fold, about 25-fold, about 30-fold, about 35-fold, about 40-fold, about 45-fold, about 50-fold, about 60-fold, about 70-fold, about 80-fold, about 90-fold, about 100-fold, about 150-fold, about 200-fold, about 250-fold, about 300-fold, about 350-fold, about 400-fold, about 450-fold, or about 500-fold of the MIC of a typical microbial cell of the same strain.

Any appropriate antimicrobial agent effective against a microbial cell disclosed herein may be used in the methods of the present disclosure. In some embodiments, the one or more antimicrobial agents include, but are not limited to rifamycins, rifampicin, aminoglycosides, fluoroquinolones, penicillins, carbapenems, cephalosporins antibiotic, penicillinase-resistant penicillins, aminopenicillins, β-lactams, tetracyclines, sulfonamides, phenicols, trimethoprim, macrolides, fosfomycin, erythromycin, azithromycin, clarithromycin, dirithromycin, troleandomycin, synthetic drugs quinolones, sulfonamides, trimethoprim, sulfamethoxazole, streptomycin, glycopeptides, glycylcyclines, ketolides, lipopeptides, monobactams, nitroimidazoles, oxazolidinones, polymixins, benzilpenicilline, aminoglycosides, amikacin, arbekacin, gentamicin, kanamycin, neomycin, netilmicin, paromomycin, rhodostreptomycin, streptomycin, tobramycin, apramycin, amphotericin, nystatin, pimaricin, fluconazole, itraconazole, voriconazole, posaconazole, isavuconazole, ketoconazole, echinocandins, polyenes, allylamines, naftifine, terbinafine, morpholines, amorolfine, 5-fluorocytosine, atovaquone/proguanil, malarone, chloroquine, doxycycline, mefloquine, primaquine, meropenem, and tafenoquine.

In some embodiments, survival, growth or development of the microbial cell in a sample is determined by counting the number of cells observed. In some embodiments, survival, growth or development of the microbial cell in a sample is determined by counting the number of cells observed relative to unperturbed wells. In some embodiments, survival, growth or development of the microbial cell in a sample is determined by measuring cell metabolism. In some embodiments, growth or development of the microbial cell in a sample is determined by measuring cell metabolism at varying concentrations of one or more antimicrobial agents. In some embodiments, metabolic measurements include, but are not limited to, concentration of dissolved carbon dioxide, heat dissipation, oxygen consumption, expressed genes involved in cell homeostasis, stress response, division, and/or growth, and/or cell membrane integrity, and/or cell wall integrity, and/or S-layer integrity (live/dead stain).

### Inference of Potential Antimicrobial Resistance.

To enable prediction of antimicrobial resistance in the future, HiPR-FISH can be applied to not only measure the microbial identity via the rRNA sequences, but also measure the presence of antimicrobial genes, proteins, or metabolic products. To measure the presence of antimicrobial genes, panels of probes that are specific and only specific to a list of antimicrobial genes are designed. These probes are similarly encoded into binary barcodes by adding flanking sequences to the encoding sequences. These flanking sequences may be readout sequences or sequences for additional signal amplification. In the case where the flanking sequences are readout sequences, the specimen can be hybridized with readout probes and imaged on an imaging device. In the case where the flanking sequences are initiator sequences, the specimen is subjected to a round of signal amplification using amplifier probes. The amplifier probes may be conjugated with fluorophores. If the amplifier probes are already conjugated with fluorophores, the specimen can be imaged on an imaging device after amplification hybridization. If the amplifiers are not conjugated with fluorophores, the amplifier probes will contain a readout sequence. The amplified specimen is then hybridized with fluorescently labeled readout probes before being imaged on an imaging device. To measure the presence of antimicrobial proteins, antibodies conjugated with DNA readout sequences are engineered. The DNA barcoded antibodies will bind to proteins of interest, and the labeled specimen will be hybridized with fluorescently labeled readout probes before being imaged on an imaging device. To measure metabolic products such as sugars or lipids, DNA barcodes will be conjugated to molecules that bind specifically to the sugars or lipids of interest. The labeled specimen will then be hybridized with fluorescently labeled readout probes before being imaged on an imaging device. For measurement of proteins, sugars, and/or lipids, amplifier probes may also be used in a similar fashion as described for gene targets to increase signal and reduce the influence of noise. Examples of imaging devices include, but are not limited to, epifluorescent microscopes, confocal microscopes, multi-photon microscopes, and light-sheet microscopes.

Any number of genetic changes can affect the susceptibility of an organism to an antimicrobial agent or drug. For example, permeability changes in the bacterial cell wall can restrict antimicrobial access to target sites, changes in pumps can alter the efflux of the antimicrobial from the cell, proteins may enzymatically modify or degrade the antimicrobial agent, the cell may acquire an alternative metabolic pathway to that inhibited by the antimicrobial agent, the target of the antimicrobial agent may be modified, or the target enzyme may be overproduced.

In some embodiments, the present methods detect mutations that influence the development of antimicrobial resistance or susceptibility, such as nucleotide substitutions in the 23S rRNA gene that cause macrolide resistance, single nucleotide polymorphisms in ribosomal proteins such as L4 or L22, mutations within the rpsL gene, or frame shift mutation in ddl gene encoding a cytoplasm enzyme D-Ala-D-Ala ligase.

In some embodiments, the present methods can identify genetic changes in the microorganism compared to unmodified microorganisms of the same type. In some embodiments, the present methods identify deletions, duplications, single nucleotide polymorphisms (SNPs), frame-shift mutations, inversions, insertions, and/or substitutions associated with the development of susceptibility or resistance to a given antimicrobial agent. In some embodiments, the present methods identify mutations associated with increased drug resistance in genes including, but not limited to, genes encoding multidrug resistance proteins (e.g. PDR1, PDR3, PDR7, PDR9), ABC transporters (e.g. SNQ2, STE6, PDR5, PDR10, PDR11, YOR1), membrane associated transporters (GAS1, D4405), soluble proteins (e.g. G3PD), RNA polymerase, rpoB, gyrA, gyrB, 16S RNA, 23S rRNA, NADPH nitroreductase, sul2, strAB, tetAR, aac3-iid, aph, sph, cmy-2, floR, tetB, aadA, aac3-VIa, and sul1.

### Microorganisms

In some aspects, the present disclosure provides methods for identifying and characterizing an infectious microorganism such as a virus, bacterium, parasite, or fungus. The infectious microorganism can be a microorganism that causes infections in a human or an animal such as a species of livestock, poultry, and fish.

In some embodiments, the list of phyla of microorganisms include phyla Actinobacteria, Aquiflcae, Armatimonadetes, Bacteroidetes, Chlamydiae, Chlorobi, Chloroflexi, Chrysiogenetes, Cyanobacteria, Chrysiogenetes, Deferribacteres, Deinococcus-thermus, Dictyoglomi, Elusimicrobia, Fibrobacteres, Firmicutes, Fusobacteria, Gemmatimonadetes, Lentisphaerae, Nitrospirae, Planctomycetes, Proteobacteria, Spirochaetia, Synergistetes, Thermodesulfobacteria, Thermotogae, and Verrucomicrobia.

In some embodiments, the present disclosure provides methods for identifying and characterizing a virus including but not limited to, bacteriophage, RNA bacteriophage (e.g. MS2, AP205, PP7 and Qβ), Helicobacter pylori, infectious haematopoietic necrosis virus (IHNV), parvovirus, Herpes Simplex Virus, Hepatitis A virus, Hepatitis B virus, Hepatitis C virus, Measles virus, Mumps virus, Rubella virus, Human Immunodeficiency Virus (HIV), Influenza virus, Rhinovirus, Rotavirus A, Rotavirus B, Rotavirus C, Respiratory Syncytial Virus (RSV), Varicella zoster, Poliovirus, Norovirus, Zika Virus, Dengue Virus, Rabies Virus, Newcastle Disease Virus, and White Spot Syndrome Virus. In some embodiments, the methods identify and characterize a cell (e.g. human cell) infected with a virus of the disclosure.

In some embodiments, the present disclosure provides methods for identifying and characterizing a bacterium including but not limited to, Mycobacterium, Streptococcus, Staphylococcus, Shigella, Campylobacter, Salmonella, Clostridium, Corynebacterium, Pseudomonas, Neisseria, Listeria, Vibrio, Bordetella, E. coli (including pathogenic E. coli), Pseudomonas aeruginosa, Enterobacter cloacae, Mycobacterium tuberculosis, Staphylococcus aureus, Helicobacter pylori, and Legionella. In some embodiments, the present disclosure provides methods for identifying and characterizing a bacterium including, but not limited to, Acinetobacter baumannii, Citrobacter freundii, Citrobacter koseri, Enterobacter cloacae, Enterococcus faecalis, Enterococcus faecium, Escherichia coli, Klebsiella oxytoca, Klebsiella pneumoniae, Proteus mirabilis, Proteus vulgaris, Pseudomonas aeruginosa, Serratia marcescens, Staphylococcus aureus, Staphylococcus saprophyticus, Streptococcus agalactiae, or a combination thereof.

In some embodiments, the present disclosure provides methods for identifying and characterizing a parasite including but not limited to, Plasmodium (i.e. P. falciparum, P. malariae, P. ovale, P. knowlesi, and P. vivax), Trypanosoma, Toxoplasma, Giardia, and Leishmania, Cryptosporidium, helminthic parasites: Trichuris spp. (whipworms), Enterobius spp. (pinworms), Ascaris spp. (roundworms), Ancylostoma spp. and Necator spp. (hookworms), Strongyloides spp. (threadworms), Dracunculus spp. (Guinea worms), Onchocerca spp. and Wuchereria spp. (filarial worms), Taenia spp., Echinococcus spp., and Diphyllobothrium spp. (human and animal cestodes), Fasciola spp. (liver flukes) and Schistosoma spp. (blood flukes).

In some embodiments, the present disclosure provides methods for identifying and characterizing a fungus including but not limited to, Aspergillus, Candida, Blastomyces, Coccidioides, Cryptococcus, Pneumocystis, Mucor, Rhizopus, Rhizomucor, Fusarium, Scedosporium, and Histoplasma.

### Kits

Another aspect of the disclosure is directed to kits that allow practicing the methods of the present disclosure.

In some embodiments, the disclosure is directed to a kit which includes a list of taxa of microorganisms, wherein each taxon is assigned a unique n-bit binary code selected from a plurality of unique n-bit binary codes, wherein n is an integer greater than 1; a set of n number of decoding probes, wherein each decoding probe corresponds to a digit in the plurality of unique n-bit binary codes, is conjugated with a label that provides a detectable signal, wherein the labels on the decoding probes are different from each other, and is substantially complementary to a readout sequence selected from a set of n number of readout sequences; and instructions on how to design a set of encoding probes, wherein the set of encoding probes includes a plurality of subsets of encoding probes, wherein each encoding probe comprises a targeting sequence and one or more readout sequences, the encoding probes within each subset comprise a targeting sequence that is specific to a taxon in the list of taxa of microorganisms and is different from a targeting sequence of the encoding probes of another subset, and the readout sequences in the encoding probes within a subset are selected from the set of n number of readout sequences based on the unique n-bit binary code assigned to the taxon which the targeting sequence of the subset is specific to.

In some embodiments, the encoding probes within each subset comprise at least one targeting sequence that is specific to a taxon. In some embodiments, the encoding probes within each subset comprise at least two targeting sequences that are specific to the same taxon.

In some embodiments, the kit includes a device to practice the methods of the present disclosure. In some embodiments, the device is a multiwell platform. In some embodiments, the multiwell platform contains between 2 and 400 well, or 2 and 384 well, or 8 and 100 well. In some embodiments, the multiwell platform contains 2 wells, 3 wells, 4 wells, 5 wells, 6 wells, 7 wells, 8 wells, 9 wells, 10 wells, 12 wells, 24 wells, 25 wells, 30 wells, 48 wells, 50 wells, 75 wells, 96 wells, 100 wells, 150 wells, 200 wells, 250 wells, 300 wells, 350 wells, 384 wells, or 400 wells. In some embodiments, the wells contain drug-inoculated or drug-free agar, agarose, polyethylene glycol, or polyacrylamide. In some embodiments, the devices are a single or a double layer of silicon. In some embodiments, a plastic flow chamber is attached for HiPR-FISH processing and readout.

### Biological Samples

The methods disclosed herein can be performed directly in a biological sample, without the need to isolate and culture microorganisms. In some embodiments, the biological sample is a biological fluid or a tissue sample. In some embodiments, the biological sample includes, but is not limited to, bronchoalveolar lavage fluid (BAL), blood, serum, plasma, urine, cerebrospinal fluid, pleural fluid, synovial fluid, ocular fluid, peritoneal fluid, amniotic fluid, gastric fluid, lymph fluid, interstitial fluid, tissue homogenate, cell extracts, saliva, sputum, stool, physiological secretions, tears, mucus, sweat, milk, semen, seminal fluid, vaginal secretions, fluid from ulcers and other surface eruptions, blisters, and abscesses, and extracts of tissues associated with medical implants, and extracts of tissues including biopsies of normal, malignant, and suspect tissues or any other constituents of the body which may contain the microorganism of interest. In some embodiments, the sample is a human oral microbiome sample. In some embodiments, the sample is a whole organism.

In some embodiments, the sample is obtained from a patient diagnosed with, or suspected to be suffering from an infection, disease, or disorder. In some embodiments, the patient has been diagnosed with, or is suspected to be suffering from a bacterial, viral, fungal, or parasitic infection. In some embodiments, the infection includes, but is not limited to, tetanus, diphtheria, pertussis, pneumonia, meningitis, campylobacteriosis, mumps, measles, rubella, polio, flu, hepatitis, chickenpox, malaria, toxoplasmosis, giardiasis, or leishmaniasis.

In some embodiments, the patient has been diagnosed with, or is suspected to be suffering from an infection caused by a bacterium selected from the group consisting of: Mycobacterium, Streptococcus, Staphylococcus, Shigella, Campylobacter, Salmonella, Clostridium, Corynebacterium, Pseudomonas, Neisseria, Listeria, Vibrio, Bordetella, Helicobacter pylori, and Legionella.

In some embodiments, the patient has been diagnosed with, or is suspected to be suffering from an infection caused by a virus selected from the group consisting of: bacteriophage, RNA bacteriophage (e.g. MS2, AP205, PP7 and Qβ), Infectious Haematopoietic Necrosis Virus, Parvovirus, Herpes Simplex Virus, Hepatitis A virus, Hepatitis B virus, Hepatitis C virus, Measles virus, Mumps virus, Rubella virus, HIV, Influenza virus, Rhinovirus, Rotavirus A, Rotavirus B, Rotavirus C, Respiratory Syncytial Virus (RSV), Varicella zoster, and Poliovirus, Norovirus, Zika virus, Dengue Virus, Rabies Virus, Newcastle Disease Virus, and White Spot Syndrome Virus.

In some embodiments, the patient has been diagnosed with, or is suspected to be suffering from an infection caused by a parasite selected from the group consisting of: Plasmodium, Trypanosoma, Toxoplasma, Giardia, Leishmania, Cryptosporidium, helminthic parasites: Trichuris spp., Enterobius spp., Ascaris spp., Ancylostoma spp. and Necator spp., Strongyloides spp., Dracunculus spp., Onchocerca spp. and Wuchereria spp., Taenia spp., Echinococcus spp., and Diphyllobothrium spp., Fasciola spp., and Schistosoma spp.

### HiPR-Swap

Another aspect of the disclosure is directed to a method of analyzing a sample by performing multiple imaging rounds exchanging emissive readout probes which are referred to herein as HiPR-Swap.

HiPR-Swap is motivated by a need to target hundreds of thousands of rRNA, mRNA, and other molecules in the microbiomes and the host tissue in order to describe host-microbiome interactions. For example, to image on average 100 unique mRNAs in roughly 1000 taxa in the gut microbiome, along with all mammalian host transcripts would require us to be able to uniquely barcode ˜150,000 targets.

Several FISH-based methods use multiple rounds of imaging to achieve high multiplexity in their assays. Multiple rounds can be performed by: (1) photobleaching fluorescent probes before applying a next round of fluorescent probes; (2) applying DNAse to the specimen to degrade fluorescent probes before applying a next round of fluorescent probes; (3) adding photocleavable or chemically-cleavable linker molecules to the fluorescent probes, and performing the cleavage to remove fluorescence signal before applying a next round of fluorescent probes; (4) stripping probes using washes with high (>50%) formamide concentrations and/or low salt (≤2×SSC) and/or high temperatures (≥37° C.). These methods, however, are undesirable for a multitude of reasons, for example, they can be time consuming and have potential for photodamage. They can also be detrimental to sample integrity, are cost-prohibitive at scale, and possibly chemically incompatible. In addition, some can remove encoding probes necessary to conduct FISH-based methods. To overcome these deficiencies, the present disclosure uses DNA exchange as a method to quickly, specifically, carefully replace HiPR-FISH readout probes without disturbing encoding and/or amplifier probes. This method is referred to as HiPR-Swap.

High Phylogenetic Resolution microbiome mapping by Fluorescence in situ Hybridization (HiPR-FISH), is a versatile technology that uses binary encoding, spectral imaging, and machine learning based decoding to create micron-scale maps of the locations and identities of hundreds of microbial species in complex communities. See, for example, Shi, H. et al. “Highly multiplexed spatial mapping of microbial communities.” Nature vol. 588, 7839 (2020): 676-681 and PCT Patent Publication WO 2019/173555, filed Mar. 7, 2019. The contents of the aforementioned disclosures are each incorporated herein by reference in their entireties.

In the HiPR-Swap method, readout and encoding probes are designed such that the “landing pad” (the region on the encoding probe to which the readout probe binds) is shorter than or equal to in length to the readout probe. The landing pad being shorter than the readout probe creates a single-stranded overhang of the readout probe, as it extends past the end of the landing pad. The bigger the difference in length, the faster the exchange happens but there is also the risk of having a less stable readout probe being on the landing pad. Accordingly, there is a balance that needs to be struck to achieve a complete hybridization/exchange. In some instances, when the readout probe is of the same length as the landing pad, using a high concentration of exchange probes can result in a complete swap.

After a readout probe is bound, an exchange probe can be added to the specimen. The exchange probe can be constructed to be of equal length and a perfect reverse complement to the readout probe. In some instances, the exchange probe may contain locked nucleic acids to increase the stability of the exchange-readout pair. When added, the exchange probe seeds a hybridization to the exposed area of the readout probe. Over a short period of time the exchange probe completely hybridizes to the readout probe, thereby removing it from the encoding probe where it can be washed away. Importantly, orthogonal readout and exchange probes can be added simultaneously to reduce assay time.

Accordingly, a method for analyzing a sample can include:
contacting at least one encoding probe with the sample to produce a first complex, wherein each encoding probe comprises a targeting sequence, a first landing pad sequence, and a second landing pad sequence;
adding at least one first emissive readout probe to the first complex, wherein the first emissive readout probe comprises a label and a sequence complementary to the first landing pad sequence;
acquiring one or more emission spectra from the first emissive readout probe;
adding an exchange probe to the sample, wherein the exchange probe comprises a 100% complementary sequence to the first emissive readout probe sequence,
hybridizing the exchange probe to the first emissive readout probe to form a second complex;
removing the second complex from the sample,
adding at least one second emissive readout probe to the first complex, wherein the second emissive readout probe comprises a label and a sequence complementary to the second landing pad sequence;
acquiring one or more emission spectra from the second emissive readout probe;
repeating the aforementioned steps for at least one different encoding probe;
determining the spectra of “signal” (e.g., puncta, blobs) and assigning them to a species of interest; and
decoding the spectra into a single, targeted transcript through means of signal deconvolution, error correction, comparison to reference standards.

In some embodiments, more than one type of probe set (e.g., encoding probe, emissive readout probes, and exchange probes) may be introduced to a sample. For example, there may be from at least 2 to at least 1 billion distinguishable probe sets that are introduced to a sample. In some embodiments, at least 2, at least 5, at least 10, at least 25, at least 50, at least 75, at least 100, at least 300, at least 1,000, at least 3,000, at least 10,000, at least 30,000, at least 100,000, at least 500,000, or at least 1,000,000, at least 10,000,000, at least 50,000,000, at least 100,000,000, at least 500,000,000, or at least 1,000,000,000 distinguishable probe sets that are introduced to a sample. In some embodiments, the distinct probes are introduced simultaneously. In some embodiments, the distinct probes are introduced sequentially. In some embodiments, more than one type of probe set may be introduced to a sample over multiple rounds, with each round having multiple probe pools.

### Encoding Probe Hybridization

In the methods described herein for analyzing a sample, the method can include contacting at least one encoding probe with the sample to produce a first complex, wherein each encoding probe includes a targeting sequence, a first landing pad sequence, and a second landing pad sequence. This step may also be referred to as the “encoding probe hybridization” step. In here, at least one encoding probe is contacted with the sample to produce a first complex. The first complex can include the targeting sequence of the encoding probe hybridized to the nucleic acid target sequence.

In some embodiments, contacting the encoding probes with the sample is contacting the encoding probes with at least one nucleotide sequence of the sample. In some embodiments, contacting the encoding probes with the sample is hybridizing the encoding probe (e.g., via the targeting sequence present in the encoding probe) with a target sequence present in the sample.

In some embodiments, in order to contact encoding probes with the sample, the sample can be digested or lysed so as to allow the encoding probes (and other probes described herein) to contact with the target sequence.

In some embodiments, to contact the at least one encoding probe with the sample to produce a first complex, encoding buffer is added to the sample. In some embodiments, a pre-hybridization step can be performed prior to adding the encoding probe. In some embodiments, the encoding buffer can be added to the sample without the encoding probe. In some embodiments, the encoding buffer can be added to the sample about 30 minutes prior to adding the encoding probe.

In some embodiments, the encoding buffer can include a denaturing/deionizing agent, a salt buffer, a detergent, a polyanionic polymer, a blocking agent, acids, or combinations thereof. In some embodiments, the encoding buffer can include more than one type of agent, for example, the encoding buffer can include two or more polyanionic polymers and/or two or more blocking agents. In some embodiments, the encoding buffer can include a denaturing/deionizing agent, a salt buffer, a detergent, two polyanionic polymers, two blocking agents, and an acid.

In some embodiments, the encoding buffer can include a denaturing/deionizing agent. In some embodiments, the denaturing/deionizing agent can be formamide, ethylene carbonate, or urea. In some embodiments, the encoding buffer can include about 10% (v/v) to about 50% (v/v), about 15% (v/v) to about 45% (v/v), about 20% (v/v) to about 40% (v/v), about 25% (v/v) to about 35% (v/v), about 10% (v/v), 15% (v/v), 20% (v/v), 25% (v/v), or 30% (v/v) of a denaturing/deionizing agent (e.g., ethylene carbonate).

In some embodiments, the encoding buffer can include a salt buffer. In some embodiments, the salt buffer is saline sodium citrate (SSC), NaCl, or MgCl₂. In some embodiments, the encoding buffer can include about 2× to about 20×, about 5× to about 10×, or about 5× of a salt buffer (e.g., saline sodium citrate (SSC)).

In some embodiments, the encoding buffer can include at least one polyanionic polymer. In some embodiments, the encoding buffer can include one polyanionic polymer. In some embodiments, the encoding buffer can include two polyanionic polymers. In some embodiments, the polyanionic polymer can be dextran sulfate, heparin, or polyglutamic acid. In some embodiments, the encoding buffer can include about 2.5% (v/v) to about 25% (v/v), about 5% (v/v) to about 15% (v/v), about 7.5% (v/v) to about 12.5% (v/v), about 5% (v/v), or about 10% (v/v) of a polyanionic polymer (e.g., dextran sulfate). In some embodiments, the encoding buffer can include about 20 μg/mL to about 80 μg/mL, about 30 μg/mL to about 70 μg/mL, about 40 μg/mL to about 60 μg/mL, or about 50 μg/mL of a polyanionic polymer (e.g., heparin).

In some embodiments, the encoding buffer can include a detergent. In some embodiments, the detergent can be Tween 20, Tween 80, sodium dodecyl sulfate (SDS), Triton X-100, Triton X-114, NP-40, Brij-35, Brij-58. N-Dodecyl-beta-maltoside, Octyl-beta-glucoside, octylthioglucoside (OTG). In some embodiments, the encoding buffer can include about 0.01% (v/v) to about 1.0% (v/v), about 0.05% (v/v) to about 0.5% (v/v), or about 0.1% (v/v), or about 0.05% (v/v) of detergent (e.g., SDS).

In some embodiments, the encoding buffer can include an acid. In these embodiments, the acid lowers the pH of the buffer. In some embodiments, the acid can be citric acid. In some embodiments, the encoding buffer can include about 1 mM to about 30 mM, about 5 mM to about 15 mM, about 5 mM to about 10 mM, about 7 mM to about 10 mM, or about 9 mM of an acid (e.g., citric acid).

In some embodiments, the encoding buffer can include at least one blocking agent. In some embodiments, the encoding buffer can include one blocking agent. In some embodiments, the blocking agents can be Denhardt's solution, bovine serum albumin (BSA), salmon sperm DNA, Ficoll, polyvinyl pyrrolidone (PVP), E. coli tRNA, casein solution, or random hexamers. In some embodiments, the encoding buffer can include about 0.1× to about 10×, about 0.5× to about 5×, about 1× to about 2×, or about 1× of a blocking agent (e.g., Denhardt's solution).

In some embodiments, the encoding buffer can include ethylene carbonate, dextran sulfate, SSC, Denhardt's solution, and SDS. In some embodiments, the encoding buffer can include 10% ethylene carbonate, 10% dextran sulfate, 2×SSC, 5×Denhardt's solution, and 0.01% SDS.

### First Emissive Readout Probe Hybridization

Following the hybridization of the encoding probe with the target sequence to form a first complex, at least one first emissive readout probe is added to the first complex, wherein the first emissive readout probe comprises a label and a sequence complementary to the first landing pad sequence. In some embodiments, this step may be referred to as the “readout probe hybridization” step. In here, the emissive readout probes hybridize to their complementary sequences present in the first complex (e.g., first landing pad sequence).

In some embodiments, the encoding probe and the readout probe hybridization occur in the same step. In some embodiments, the readout probe hybridization is performed in the presence of the encoding buffer described above. In some embodiments, the encoding probe hybridization step, the readout probe hybridization step, and the readout step can occur sequentially or substantially in the same step.

In some embodiments, to hybridize the readout probes to the first complex, readout buffer is added to the sample. In some embodiments, to image the readout probes, a wash buffer is added to the sample.

In some embodiments, the wash buffer can include a salt buffer, a pH stabilizer, and a chelating agent.

In some embodiments, the readout probes are added so they achieve a final concentration of about 10 nM to about 20 μM, or about 10 nM to about 10 μM, or about 100 nM to about 1 μM, about 200 nM to about 500 nM, or about 200 nM, about 300 nM, about 400 nM, or about 500 nM for each readout probe. In some embodiments, the readout probes are added so they achieve a final concentration of about 400 nM.

In some embodiments, the wash buffer can include a salt buffer. In some embodiments, the salt buffer is saline sodium citrate (SSC), NaCl, or MgCl₂. In some embodiments, the wash buffer can include about 2× to about 20×, about 5× to about 10×, or about 5× of a salt buffer (e.g., saline sodium citrate (SSC)). In some embodiments, the wash buffer can include about 50 mM to about 500 mM, or about 100 mM to about 300 mM, or about 150 mM to about 250 mM, or about 215 mM or salt buffer (e.g., NaCl).

In some embodiments, the wash buffer can include a pH stabilizer. In some embodiments, the pH stabilizer can be at least one of tris-HCl, citric acid, SSC, 4-(2-hydroxyethyl)-1-piperazineethanesulfonic acid (HEPES), sucrose/EDTA/Tris-HCl (SET), potassium phosphate, tris(hydroxymethyl)methylamino]propanesulfonic acid (TAPS), NaOH, 3-(N-morpholino)propanesulfonic acid (MOPS), Tricine, Bicine, sodium pyrophosphate, piperazine-N,N′-bis(2-ethanesulfonic acid) (PIPES), SSPE. In some embodiments, the pH stabilizer can be tris-HCl. In some embodiments, the wash buffer can include about 5 mM to about 30 mM, about 10 mM to about 20 mM, about 10 mM, or about 20 mM of a pH stabilizer (e.g., tris-HCl).

In some embodiments, the wash buffer can include a chelating agent. In some embodiments, the chelating agent is at least one of EDTA, Ethylene glycol tetraacetic acid (EGTA), Salicylic acid, Triethanolamine (TEA), or Dimercaptopropanol. In some embodiments, the chelating agent is EDTA. In some embodiments, the wash buffer can include about 1 mM to about 10 mM, about 2 mM to about 5 mM, or about 5 mM of a chelating agent (e.g., EDTA).

In some embodiments, the wash buffer can include NaCl, tris-HCl, and EDTA. In some embodiments, the wash buffer can include 215 mM NaCl, 20 mM tris-HCl, and 5 mM EDTA.

### Exchange Probe Hybridization

After acquiring one or more emission spectra from the first emissive readout probe, an exchange probe is added so it removes the first emissive readout probe from the complex so it allows for another emissive readout probe and imaging step to occur. In some embodiments, the addition of the exchange probe and addition of the second emissive readout probe occur in the same step. In some embodiments, the addition of the exchange probe and addition of the second emissive readout probe occur sequentially.

In some embodiments, the exchange probes are added so they achieve a final concentration of about 10 nM to about 20 or about 10 nM to about 10 or about 100 nM to about 1 about 200 nM to about 500 nM, or about 200 nM, about 300 nM, about 400 nM, or about 500 nM for each exchange probe. In some embodiments, the exchange probes are added so they achieve a final concentration of about 400 nM.

In some embodiments, to contact the exchange probe with the first emissive readout probe to produce a second complex, exchange buffer is added to the sample. In some embodiments, the exchange buffer can include a denaturing/deionizing agent, a salt buffer, a detergent, a polyanionic polymer, a blocking agent, acids, or combinations thereof. In some embodiments, the exchange buffer can include more than one type of agent, for example, the encoding buffer can include two or more polyanionic polymers and/or two or more blocking agents. In some embodiments, the exchange buffer can include a denaturing/deionizing agent, a salt buffer, a detergent, two polyanionic polymers, two blocking agents, and an acid.

In some embodiments, the exchange buffer can include a denaturing/deionizing agent. In some embodiments, the denaturing/deionizing agent can be formamide, ethylene carbonate, or urea. In some embodiments, the exchange buffer can include about 10% (v/v) to about 50% (v/v), about 15% (v/v) to about 45% (v/v), about 20% (v/v) to about 40% (v/v), about 25% (v/v) to about 35% (v/v), about 10% (v/v), 15% (v/v), 20% (v/v), 25% (v/v), or 30% (v/v) of a denaturing/deionizing agent (e.g., ethylene carbonate).

In some embodiments, the exchange buffer can include a salt buffer. In some embodiments, the salt buffer is saline sodium citrate (SSC), NaCl, or MgCl₂. In some embodiments, the exchange buffer can include about 2× to about 20×, about 5× to about 10×, or about 5× of a salt buffer (e.g., saline sodium citrate (SSC)).

In some embodiments, the exchange buffer can include at least one polyanionic polymer. In some embodiments, the exchange buffer can include one polyanionic polymer. In some embodiments, the exchange buffer can include two polyanionic polymers. In some embodiments, the polyanionic polymer can be dextran sulfate, heparin, or polyglutamic acid. In some embodiments, the exchange buffer can include about 2.5% (v/v) to about 25% (v/v), about 5% (v/v) to about 15% (v/v), about 7.5% (v/v) to about 12.5% (v/v), about 5% (v/v), or about 10% (v/v) of a polyanionic polymer (e.g., dextran sulfate). In some embodiments, the exchange buffer can include about 20 μg/mL to about 80 μg/mL, about 30 μg/mL to about 70 μg/mL, about 40 μg/mL to about 60 μg/mL, or about 50 μg/mL of a polyanionic polymer (e.g., heparin).

In some embodiments, the exchange buffer can include a detergent. In some embodiments, the detergent can be Tween 20, Tween 80, sodium dodecyl sulfate (SDS), Triton X-100, Triton X-114, NP-40, Brij-35, Brij-58. N-Dodecyl-beta-maltoside, Octyl-beta-glucoside, octylthioglucoside (OTG). In some embodiments, the exchange buffer can include about 0.01% (v/v) to about 1.0% (v/v), about 0.05% (v/v) to about 0.5% (v/v), or about 0.1% (v/v), or about 0.05% (v/v) of detergent (e.g., SDS).

In some embodiments, the exchange buffer can include an acid. In these embodiments, the acid lowers the pH of the buffer. In some embodiments, the acid can be citric acid. In some embodiments, the exchange buffer can include about 1 mM to about 30 mM, about 5 mM to about 15 mM, about 5 mM to about 10 mM, about 7 mM to about 10 mM, or about 9 mM of an acid (e.g., citric acid).

In some embodiments, the exchange buffer can include at least one blocking agent. In some embodiments, the exchange buffer can include one blocking agent. In some embodiments, the blocking agents can be Denhardt's solution, bovine serum albumin (BSA), salmon sperm DNA, Ficoll, polyvinyl pyrrolidone (PVP), E. coli tRNA, casein solution, or random hexamers. In some embodiments, the exchange buffer can include about 0.1× to about 10×, about 0.5× to about 5×, about 1× to about 2×, or about 1× of a blocking agent (e.g., Denhardt's solution).

In some embodiments, the exchange buffer can include ethylene carbonate, dextran sulfate, SSC, Denhardt's solution, and SDS. In some embodiments, the exchange buffer can include 10% ethylene carbonate, 10% dextran sulfate, 2×SSC, 5×Denhardt's solution, and 0.01% SDS.

### Second Emissive Readout Probe Hybridization

Following the hybridization of the exchange probe to the first emissive readout probe, a second emissive readout probe is added. In some embodiments, this step may be referred to as the “second readout probe hybridization” step. In here, the second emissive readout probe hybridizes to its complementary sequences present in the first complex (e.g., second landing pad sequence).

In some embodiments, the second emissive readout probe hybridization is performed in the presence of the encoding buffer described above. In some embodiments, to image the second readout probes, a wash buffer is added to the sample. In some embodiments, the wash buffer is the wash buffer described above.

In some embodiments, the second emissive readout probes are added so they achieve a final concentration of about 10 nM to about 10 or about 100 nM to about 1 about 200 nM to about 500 nM, or about 200 nM, about 300 nM, about 400 nM, or about 500 nM for each readout probe. In some embodiments, the second emissive readout probes are added so they achieve a final concentration of about 400 nM.

In some embodiments, adding an exchange probe to the sample, hybridizing the exchange probe to the first emissive readout probe, and removing the second complex from the sample are performed in the same step. In some embodiments, adding an exchange probe to the sample, hybridizing the exchange probe to the first emissive readout probe, and removing the second complex from the sample are performed sequentially. In some embodiments, adding an exchange probe to the sample, hybridizing the exchange probe to the first emissive readout probe, removing the second complex from the sample, and adding the second emissive readout probe are performed in the same step. In some embodiments, adding an exchange probe to the sample, hybridizing the exchange probe to the first emissive readout probe, removing the second complex from the sample, and adding the second emissive readout probe are performed sequentially.

In some embodiments, hybridizing the exchange probe to the first or second emissive readout probe results in de-hybridization of the first or second emissive readout probe from the first or second landing pad sequence. In some embodiments, the step is achieved from about 30 seconds to about 1 hour. In some embodiments, the step is achieved within 30 seconds, 1 minute, 5 minutes, 10 minutes, 12 minutes, 15 minutes, 30 minutes, 45 minutes, or 1 hour. In some embodiments, the step is achieved within 1 hour. In some embodiments, the step is achieved overnight.

In another aspect, a method for analyzing a sample can include:
generating a set of probes, wherein each probe comprises:
(i) a targeting sequence;
(ii) a first landing pad sequence; and
(iii) a second landing pad sequence;
contacting the set of probes with the sample to permit hybridization of the probes to nucleotides present in the sample to produce a complex;
adding a first set of emissive readout probes to the complex, wherein each emissive readout probe comprises:
(i) a label, and
(ii) a sequence complementary to the first or second landing pad sequence;
acquiring one or more emission spectra from the first emissive readout probe;
adding a set of exchange probes to the sample, wherein each exchange probe comprises a 100% complementary sequence to the first emissive readout probe sequences,
hybridizing the exchange probes to the first emissive readout probes to form a second complex;
removing the second complex from the sample,
adding a second set of emissive readout probes to the complex, wherein each emissive readout probe comprises:
(i) a label, and
(ii) a sequence complementary to the first or second landing pad sequence;
acquiring one or more emission spectra from the second emissive readout probe;
determining the spectra of “signal” (e.g., puncta, blobs) and assigning them to a species of interest; and
decoding the spectra into a single, targeted transcript through means of signal deconvolution, error correction, comparison to reference standards.

Sample

In some embodiments, the sample is at least one of a cell, a cell suspension, a tissue biopsy, a tissue specimen, urine, stool, blood, serum, plasma, bone biopsies, bone marrow, respiratory specimens, sputum, induced sputum, tracheal aspirates, bronchoalveolar lavage fluid, sweat, saliva, tears, ocular fluid, cerebral spinal fluid, pericardial fluid, pleural fluid, peritoneal fluid, placenta, amnion, pus, nasal swabs, nasopharyngeal swabs, oropharyngeal swabs, ocular swabs, skin swabs, wound swabs, mucosal swabs, buccal swabs, vaginal swabs, vulvar swabs, nails, nail scrapings, hair follicles, corneal scrapings, gavage fluids, gargle fluids, abscess fluids, wastewater, or plant biopsies.

In some embodiments, the sample is a cell. In some embodiments, the cell is a bacterial cell. In some embodiments, the cell is a eukaryotic cell. In some embodiments the eukaryotic cell is a unicellular organism including protozoa, chromista, algae, or fungi. In some embodiments the eukaryotic cell is part of a multicellular organism from chromista, plantae, fungi, or animalia. In some embodiments the sample is a tissue composed of cells. In some embodiments the cell contains foreign DNA/RNA from viruses, plasmids, and bacteria.

In some embodiments, the sample can include a plurality of cells. In some embodiments, each cell in the plurality of cells can include a specific targeting sequence, which may or may not be the same from the other targeting sequences.

In some embodiments, the sample is a human oral microbiome sample. In some embodiments, the sample is a whole organism.

In some embodiments, the sample is obtained from a patient diagnosed with, or suspected to be suffering from an infection, disease, or disorder. In some embodiments, the patient has been diagnosed with, or is suspected to be suffering from a bacterial, viral, fungal, or parasitic infection. In some embodiments, the infection includes, but is not limited to, Acute Flaccid Myelitis, Anaplasmosis, Anthrax, Babesiosis, Botulism, Brucellosis, Campylobacteriosis, Carbapenem-resistant Infection (CRE/CRPA), Chancroid, Chickenpox, Chikungunya Virus Infection (Chikungunya), Chlamydia, Ciguatera (Harmful Algae Blooms (HABs)), Clostridium Difficile Infection, Clostridium Perfringens (Epsilon Toxin), Coccidioidomycosis fungal infection (Valley fever), COVID-19 (Coronavirus Disease 2019), Creutzfeldt-Jacob Disease, transmissible spongiform encephalopathy (CJD), Cryptosporidiosis (Crypto), Cyclosporiasis, Dengue, 1, 2, 3, 4 (Dengue Fever), Diphtheria, E. coli infection, Shiga toxin-producing (STEC), Eastern Equine Encephalitis (EEE), Ebola Hemorrhagic Fever (Ebola), Ehrlichiosis, Encephalitis, Arboviral or parainfectious, Enterovirus Infection, D68 (EV-D68), Enterovirus Infection, Non-Polio (Non-Polio Enterovirus), Giardiasis (Giardia), Glanders, Gonococcal Infection (Gonorrhea), Granuloma inguinale, Haemophilus Influenza disease, Type B (Hib or H-flu), Hantavirus Pulmonary Syndrome (HPS), Hemolytic Uremic Syndrome (HUS), Hepatitis (A, B, C, D, and/or E), Herpes Herpes Zoster, zoster VZV (Shingles), Histoplasmosis infection (Histoplasmosis), Human Immunodeficiency Virus/AIDS (HIV/AIDS), Human Papillomavirus (HPV), Influenza (Flu), Lead Poisoning, Legionellosis (Legionnaires Disease), Leishmaniasis, Leprosy (Hansens Disease), Leptospirosis, Listeriosis (Listeria), Lyme Disease, Lymphogranuloma venereum infection (LGV), Malaria, Measles, Melioidosis, Meningitis, Viral (Meningitis, viral), Meningococcal Disease, Bacterial (Meningitis, bacterial), Middle East Respiratory Syndrome Coronavirus (MERS-CoV), Mononucleosis, Multisystem Inflammatory Syndrome in Children (MIS-C), Mumps, Norovirus, Paralytic Shellfish Poisoning (Paralytic Shellfish Poisoning, Ciguatera), Pediculosis (Lice, Head and Body Lice), Pelvic Inflammatory Disease (PID), Pertussis (Whooping Cough), Plague; Bubonic, Septicemic, Pneumonic (Plague), Pneumococcal Disease (Pneumonia), Poliomyelitis (Polio), Powassan, Psittacosis (Parrot Fever), Phthiriasis (Crabs; Pubic Lice Infestation), Pustular Rash diseases (Small pox, monkeypox, cowpox), Q-Fever, Rabies, Ricin Poisoning, Rickettsiosis (Rocky Mountain Spotted Fever), Rubella, Salmonellosis gastroenteritis (Salmonella), Scabies Infestation (Scabies), Scombroid, Septic Shock (Sepsis), Severe Acute Respiratory Syndrome (SARS), Shigellosis gastroenteritis (Shigella), Smallpox, Staphylococcal Infection, Methicillin-resistant (MRSA), Staphylococcal Food Poisoning, Enterotoxin-B Poisoning (Staph Food Poisoning), Staphylococcal Infection, Vancomycin Intermediate (VISA), Staphylococcal Infection, Vancomycin Resistant (VRSA), Streptococcal Disease, Group A (invasive) (Strep A (invasive)), Streptococcal Disease, Group B (Strep-B), Streptococcal Toxic-Shock Syndrome, STSS, Toxic Shock (STSS, TSS), Syphilis, primary, secondary, early latent, late latent, congenital, Tetanus, Toxoplasmosis, Trichomoniasis (Trichomonas infection), Trichinosis Infection (Trichinosis), Tuberculosis (Latent) (LTBI), Tuberculosis (TB), Tularemia (Rabbit fever), Typhus, Typhoid Fever, Group D, Vaginosis, bacterial (Yeast Infection), Vaping-Associated Lung Injury (e-Cigarette Associated Lung Injury), Varicella (Chickenpox), Vibrio cholerae (Cholera), Vibriosis (Vibrio), Viral Hemorrhagic Fever (Ebola, Lassa, Marburg), West Nile Virus, Yellow Fever, Yersenia (Yersinia), or Zika Virus Infection (Zika).

In some embodiments, when the sample is obtained from a patient, the patient has been diagnosed with, or is suspected to be suffering from an infection caused by a bacterium selected from the group consisting of: Acinetobacter, Actinomyces, Aerococcus, Bacteroides, Bartonella, Brucella, Bordetella, Burkholderia, Campylobacter, Chlamydia, Citrobacter, Clostridium, Corynebacterium, Edwardsiella, Elizabethkingia, Enterobacter, Enterococcus, Escherichia, Fusobacterium, Haemophilus, Helicobacter, Klebsiella, Legionella, Leptospira, Listeria, Morganella, Mycobacterium, Mycoplasma, Neisseria, Pantoea, Prevotella, Proteus, Providencia, Pseudomonas, Raoultella, Salmonella, Serratia, Shigella, Staphylococcus, Stenotrophomonas, Streptococcus, Ureaplasma, and Vibrio.

In some embodiments, when the sample is obtained from a patient, the patient has been diagnosed with, or is suspected to be suffering from an infection caused by a virus selected from the group consisting of: bacteriophage, RNA bacteriophage (e.g., MS2, AP205, PP7 and Qβ), Infectious Haematopoietic Necrosis Virus, Parvovirus, Herpes Simplex Virus, Hepatitis A virus, Hepatitis B virus, Hepatitis C virus, Measles virus, Mumps virus, Rubella virus, HIV, Influenza virus, Rhinovirus, Rotavirus A, Rotavirus B, Rotavirus C, Respiratory Syncytial Virus (RSV), Varicella zoster, and Poliovirus, Norovirus, Zika virus, Dengue Virus, Rabies Virus, Newcastle Disease Virus, and White Spot Syndrome Virus.

In some embodiments, when the sample is obtained from a patient, the patient has been diagnosed with, or is suspected to be suffering from an infection caused by a parasite selected from the group consisting of: Plasmodium, Trypanosoma, Toxoplasma, Giardia, Leishmania, Cryptosporidium, helminthic parasites: Trichuris spp., Enterobius spp., Ascaris spp., Ancylostoma spp. and Necator spp., Strongyloides spp., Dracunculus spp., Onchocerca spp. and Wuchereria spp., Taenia spp., Echinococcus spp., and Diphyllobothrium spp., Fasciola spp., and Schistosoma spp.

Encoding Probes

Encoding probes are probes that bind directly to a target or targeting sequence and contain either 1 or 2 branches extending away from the hybridization site. The branches can either correspond to the readout sequences or first or second landing pad sequences. Encoding probes, for example, are designed to target bacterial ribosomal RNA (rRNA) and messenger RNA (mRNA) targets.

For example, rRNA-probes can contain (5′ to 3′):

a. Primer sequences to enrich probe pool.

b. A first landing pad sequence.

c. rRNA target complementary sequence.

d. A second landing pad sequence (different than b).

e. Primer sequences to enrich probe pool.

mRNA-probes contain (5′ to 3′):

a. Primer sequences to enrich probe pool.

b. A first landing pad sequence.

c. mRNA target complementary sequence.

d. A second landing pad sequence (different than b).

e. Primer sequences to enrich probe pool.

In some embodiments, each encoding probe can include a targeting sequence, a first landing pad sequence and a second landing pad sequence.

Primer Sequences

In some embodiments, the primer sequence can include about 10 to about 30, about 15 to about 25, about 18 to about 23, about 15, 16, 17, 18, 19, 20, 21, 22, or 23 nucleotides long.

Targeting Sequence

In some embodiments, the targeting sequence targets at least one of messenger RNA (mRNA), micro RNA (miRNA), long non coding RNA (lncRNA), ribosomal RNA (rRNA), small interfering RNA (siRNA), transfer RNA (tRNA), Crispr RNA (crRNA), trans-activating cirspr RNA (tracrRNA), mitochondria RNA, Intronic RNA, viral mRNA, viral genomic RNA, environmental RNA, double-stranded RNA (dsRNA), small nuclear RNA (snRNA), small nucleolar (snoRNA), piwi-interacting RNA (piRNA), genomic DNA, synthetic DNA, DNA, plasmid DNA, a plasmid, viral DNA, retroviral DNA, environmental DNA, extracellular DNA, a protein, a small molecule, or an antigenic target. In some embodiments, the target is mRNA. In some embodiments, the target is rRNA. In some embodiments, the target is mRNA and rRNA.

In some embodiments, the targeting sequence of the encoding probe is substantially complementary to a specific target sequence. By “substantially complementary” it is meant that the nucleic acid fragment is capable of hybridizing to at least one nucleic acid strand or duplex even if less than all nucleobases do not base pair with a counterpart nucleobase. In some embodiments, a “substantially complementary” nucleic add contains at least one sequence in which about 70%, about 71%, about 72%, about 73%, about 74%, about 75%, about 76%, about 77%, about 77%, 8%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range therein, of the nucleobase sequence is capable of basepairing with at least one single or double stranded nucleic acid molecule during hybridization.

In some embodiments, the targeting sequence is designed to have a predicted melting temperature of between about 55° C. and about 65° C. In some embodiments, the predicted melting temperature of the targeting sequence is 55° C., 56° C., 57° C., 58° C., 59° C., 60° C., 61° C., 62° C., 63° C., 64° C. or 65° C. In some embodiments, the targeting sequence can have a GC content of about 55%, 60%, 65% or 70%.

In some embodiments, the targeting sequence can include about 10 to about 35, about 15 to about 30, about 18 to about 30, about 15, 16, 17, 18, 19, 20, 21, 22, 23, 24, or 25 nucleotides long.

In some embodiments, the targeting sequence of an encoding probe is designed using publicly-available sequence data. In some embodiments, the targeting sequence of an encoding probe design is designed using custom catalogues of the target/sample. In some embodiments, the targeting sequence of an encoding probe is designed using a database that is relevant for a system. In a specific embodiment, the system is the gut microbiome. In some embodiments, the targeting sequence of an encoding probe is designed using a database that is relevant for a disease or infection.

Landing Pad Sequences

In some embodiments, the encoding probe can include a first landing pad sequence on the 5′ end and a second landing pad sequence on the 3′ end. In some embodiments, the first and second landing pad sequences have the same sequence.

In some embodiments, each landing pad sequence is about 10 to about 50, about 15 to about 50, about 15 to about 40, about 10 to about 30, about 15 to about 25, about 18 to about 23, about 15, 16, 17, 18, 19, 20, 21, 22, or 23 nucleotides long. In some embodiments, each landing pad sequence is substantially complementary to the first and/or second emissive readout sequences.

The encoding probes, and other probes described herein, may be introduced into the sample (e.g., cell) using any suitable method. In some cases, the sample may be sufficiently permeabilized such that the probes may be introduced into the sample by flowing a fluid containing the probes around the sample (e.g., cells). In some cases, the samples (e.g., cells) may be sufficiently permeabilized as part of a fixation process. In some embodiments, samples (e.g., cells) may be permeabilized by exposure to certain chemicals such as ethanol, methanol, Triton, or the like. In some embodiments, techniques such as electroporation or microinjection may be used to introduce the probes into a sample (e.g., cell).

Emissive Readout Probes

Emissive readouts probes are oligonucleotides bound with one of ten fluorescent dyes at the 5′- and/or 3′-end. In some embodiments, each emissive readout probe comprises a label and a sequence complementary to the first landing pad sequence.

In some embodiments, each emissive readout probe sequence is of the same length as the first or second landing pad sequence. In some embodiments, the emissive readout probe sequence is 0 nucleotides longer than the corresponding landing pad sequence.

In some embodiments, each emissive readout probe sequence is from at least 1 to at least 35 nucleotides longer than the corresponding landing pad sequence. In some embodiments, each emissive readout probe sequence is at least 1, 2, 3, 4, 5, 6, 7, 8, 9, 10, 15, 20, 25, 30, or 35 nucleotides longer than the corresponding landing pad sequence. In some embodiments, each emissive readout probe sequence is at least 1, 2, 3, 4, 5, 6, 7, 8, 9, or 10 nucleotides longer than the corresponding landing pad sequence. In some embodiments, each emissive readout probe sequence is at least 5 nucleotides longer than the corresponding landing pad sequence.

Readout probes can be designed as follows:

a. Are coupled to 1, 2, or more fluorescent dyes.

b. Are orthogonal to all biological sequences.

c. Are orthogonal to each other/each other's complementary sequences.

In some embodiments, the readout sequence is about 10 to about 50, about 15 to about 50, about 15 to about 45, about 15 to about 35, about 15 to about 30, about 18 to about 24, about 10, 11, 12, 13, 14, 15, 16, 17, 18, 19, 20, 21, 22, 23, or 24 nucleotides long.

In some embodiments, the emissive readout probe can include a label on the 5′ or 3′ end. In some embodiments, the emissive readout probe can include a label on the 5′ end and a label on the 3′ end. In some embodiments, the labels are the same. In some embodiments, the labels are different.

In some embodiments, the label is a fluorescent entity (fluorophore) or phosphorescent entity. In some embodiments, the label is a cyanine dye (e.g., Cy2, Cy3, Cy3B, Cy5, Cy5.5, Cy7, etc.), Alexa Fluor dye, Atto dye, photo switchable dye, photoactivatable dye, fluorescent dye, metal nanoparticle, semiconductor nanoparticle or “quantum dots”, fluorescent protein such as GFP (Green Fluorescent Protein), or photoactivatable fluorescent protein, such as PAGFP, PSCFP, PSCFP2, Dendra, Dendra2, EosFP, tdEos, mEos2, mEos3, PAmCherry, PAtagRFP, mMaple, mMaple2, and mMaple3.

In some embodiments, the label is Alexa Fluor 350, Alexa Fluor 405, Alexa Fluor 430, Alexa Fluor 488, Alexa Fluor 514, Alexa Fluor 532, Alexa Fluor 546, Alexa Fluor 555, Alexa Fluor 561, Alexa Fluor 568, Alexa Fluor 594, Alexa Fluor 610, Alexa Fluor 633, Alexa Fluor 635, Alexa Fluor 647, Alexa Fluor 647-R-phycoerythrin, Alexa Fluor 660, Alexa Fluor 680, Alexa Fluor 680-allophycocyanin, Alexa Fluor 700, Alexa Fluor 750, Alexa Fluor 790, Alexa Fluor Plus 405, Alexa Fluor Plus 488, Alexa Fluor Plus 555, Alexa Fluor Plus 594, Alexa Fluor Plus 647, Alexa Fluor Plus 680, Alexa Fluor Plus 750, Alexa Fluor Plus 800, Pacific Blue, Pacific Green, Rhodamine Red X, DyLight 485-LS, DyLight-510-LS, DyLight 515-LS, DyLight 521-LS, Hydroxycoumarin, methoxycoumarin, Cy2, FAM, Fluorescein FITC, R-phycoerythrin (PE), Tamara, Cy3.5 581, ROX (carboxy-X-rhodamine), Red 613, Texas Red, Cy5, Cy5.5, Cy7, Allophycocyanin, ATTO 430LS, ATTO 490LS, ATTO 390, ATTO 425, Cyan 500 NHS-Ester, ATTO 465, ATTO 488, ATTO 495, ATTO Rho110, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 643, ATTO 647, ATTO 647N, ATTO 655, ATTO Oxa12, ATTO 665, ATTO 680, ATTO 700, ATTO 725, ATTO 740.

In some embodiments, the label is imaged using widefield microscopy, point scanning confocal microscopy, spinning disk confocal microscopy, lattice lightsheet microscopy, or light field microscopy.

In some embodiments, the detection strategy used is channel, spectral, channel and fluorescence lifetime, or spectral and fluorescence lifetime.

In some embodiments, the labels used in the present methods are imaged using a microscope. In some embodiments, the microscope is a confocal microscope. In some embodiments, the microscope is a fluorescence microscope. In some embodiments, the microscope is a light-sheet microscope. In some embodiments, the microscope is a super-resolution microscope.

In some embodiments, the sample is on an analyzing platform, wherein the analyzing platform is a microscope slide, at least one chamber, at least one microfluidic device, at least one well, at least one plate, or at least one filter membrane.

Exchange Probes

Exchange probes are each about 10-50 or 15-50 nucleotide-long oligonucleotides. In some embodiments, each exchange probe comprises a 100% complementary sequence to a respective emissive readout probe sequence.

In some embodiments, the exchange sequence is about 10 to about 50, about 15 to about 50, about 15 to about 45, about 15 to about 35, about 15 to about 30, about 18 to about 24, about 10, 11, 12, 13, 14, 15, 16, 17, 18, 19, 20, 21, 22, 23, or 24 nucleotides long.

In some embodiments, the encoding probes contain locked nucleic acids to stabilize the exchange reaction.

In some embodiments, adding an exchange probe to the sample, hybridizing the exchange probe to the first emissive readout probe, and removing the second complex from the sample are performed in the same step. In some embodiments, adding an exchange probe to the sample, hybridizing the exchange probe to the first emissive readout probe, and removing the second complex from the sample are performed sequentially. In some embodiments, adding an exchange probe to the sample, hybridizing the exchange probe to the first emissive readout probe, removing the second complex from the sample, and adding the second emissive readout probe are performed in the same step. In some embodiments, adding an exchange probe to the sample, hybridizing the exchange probe to the first emissive readout probe, removing the second complex from the sample, and adding the second emissive readout probe are performed sequentially.

In some embodiments, hybridizing the exchange probe to the first or second emissive readout probe results in de-hybridization of the first or second emissive readout probe from the first or second landing pad sequence. In some embodiments, the step is achieved from about 30 seconds to about 1 hour. In some embodiments, the step is achieved within 30 seconds, 1 minute, 5 minutes, 10 minutes, 12 minutes, 15 minutes, 30 minutes, 45 minutes, or 1 hour. In some embodiments, the step is achieved within 1 hour. In some embodiments, the step is achieved overnight.

In another aspect, a method for analyzing a bacterial sample can include:
contacting at least one encoding probe with the sample to produce a first complex, wherein each encoding probe comprises a targeting sequence, a first landing pad sequence, and a second landing pad sequence;
adding at least one first emissive readout probe to the first complex, wherein the first emissive readout probe comprises a label and a sequence complementary to the first landing pad sequence;
detecting the first emissive readout probe with a confocal microscope;
adding an exchange probe to the sample, wherein the exchange probe comprises a 100% complementary sequence to the first emissive readout probe sequence,
hybridizing the exchange probe to the first emissive readout probe to form a second complex;
removing the second complex from the sample,
adding at least one second emissive readout probe to the first complex, wherein the second emissive readout probe comprises a label and a sequence complementary to the second landing pad sequence;
detecting the second emissive readout probe with a confocal microscope;
repeating the aforementioned steps for at least one different encoding probe;
determining the spectra of “signal” (e.g., puncta, blobs) and assigning them to a bacterium; and
decoding the spectra into a single, targeted transcript through means of signal deconvolution, error correction, comparison to reference standards.

In another aspect, a method for analyzing a bacterial sample, comprising:
generating a set of probes, wherein each probe comprises:
(i) a targeting sequence;
(ii) a first landing pad sequence; and
(iii) a second landing pad sequence;
contacting the set of probes with the sample to permit hybridization of the probes to nucleotides present in the sample to produce a complex;
adding a first set of emissive readout probes to the complex, wherein each emissive readout probe comprises:
(i) a label, and
(ii) a sequence complementary to the first or second landing pad sequence;
detecting the first set of emissive readout probes in the sample with a confocal microscope;
adding a set of exchange probes to the sample, wherein each exchange probe comprises a 100% complementary sequence to the first emissive readout probe sequences,
hybridizing the exchange probes to the first emissive readout probes to form a second complex;
removing the second complex from the sample,
adding a second set of emissive readout probes to the complex, wherein each emissive readout probe comprises:
(i) a label, and
(ii) a sequence complementary to the first or second landing pad sequence;
detecting the second set of emissive readout probes in the sample with a confocal microscope;
determining the spectra of “signal” (e.g., puncta, blobs) and assigning them to a bacterium; and
decoding the spectra into a single, targeted transcript through means of signal deconvolution, error correction, comparison to reference standards.

Constructs and Libraries

In another aspect, a construct can include:
a targeting sequence that is a region of interest on a nucleotide;
a first landing pad sequence;
a second landing pad sequence, wherein the second landing pad sequence is different from the first landing pad sequence;
a first emissive readout probe comprising a first label and a sequence complimentary to the first landing pad sequence;
an exchange probe comprising a 100% complementary sequence to the first emissive readout probe sequences; and
a second emissive readout probe comprising a second label and a sequence complimentary to the second landing pad sequence.

In another aspect, a library of constructs comprising a plurality of barcoded probes, wherein each barcoded probe can include:
a targeting sequence that is a region of interest on a nucleotide;
a first landing pad sequence;
a second landing pad sequence, wherein the second landing pad sequence is different from the first landing pad sequence;
a first emissive readout probe comprising a first label and a sequence complimentary to the first landing pad sequence;
an exchange probe comprising a 100% complementary sequence to the first emissive readout probe sequences; and
a second emissive readout probe comprising a second label and a sequence complimentary to the second landing pad sequence.

In some embodiments, the region of interest on a nucleotide is at least one of messenger RNA (mRNA), microRNA (miRNA), long non coding RNA (lncRNA), ribosomal RNA (rRNA), small interfering RNA (siRNA), transfer RNA (tRNA), Crispr RNA (crRNA), trans-activating CRISPR RNA (tracrRNA), mitochondrial RNA, intronic RNA, viral mRNA, viral genomic RNA, environmental RNA, double-stranded RNA (dsRNA), small nuclear RNA (snRNA), small nucleolar (snoRNA), PIWI-interacting RNA (piRNA), genomic DNA, synthetic DNA, DNA, plasmid DNA, a plasmid, viral DNA, retroviral DNA, environmental DNA, extracellular DNA, a protein, a small molecule, or an antigen.

In some embodiments, the region of interest on a nucleotide is mRNA.

In some embodiments, the region of interest on a nucleotide is rRNA.

In some embodiments, the region of interest on a nucleotide is mRNA and rRNA.

In some embodiments, the first and second landing pad sequences have the same sequence. In some embodiments, the first and second landing pad sequences have different sequences.

In some embodiments, the first and second landing pad sequences each are about 10 to about 50, about 10 to about 40, about 10 to about 30, about 15 to about 25, about 18 to about 23, about 15, 16, 17, 18, 19, 20, 21, 22, or 23 nucleotides long. In some embodiments, the first and second landing pad sequences each are substantially complementary to the first and/or second emissive readout sequences.

In some embodiments, the first and second emissive readout probes are each about 10 to about 50, about 10 to about 40, about 10 to about 30, about 15 to about 25, about 18 to about 23, about 15, 16, 17, 18, 19, 20, 21, 22, or 23 nucleotides long bound with one of ten fluorescent dyes at the 5′- and/or 3′-end. In some embodiments, the first and second emissive readout probes each comprise a label and a sequence complementary to the first or second landing pad sequence.

In some embodiments, the first and second emissive readout probes are each of the same length as the corresponding landing pad sequence. In some embodiments, the first and second emissive readout probes are each 0 nucleotides longer than the corresponding landing pad sequence. In some embodiments, the first and second emissive readout probes are each at least 2 to 50 nucleotides longer than the corresponding landing pad sequence. In some embodiments, the first and second emissive readout probes are each at least 1, 2, 3, 4, 5, 6, 7, 8, 9, 10, 15, 20, 25, 30, 35, 40, 45, or 50 nucleotides longer than the corresponding landing pad sequence. In some embodiments, the first and second emissive readout probes are each at least 1, 2, 3, 4, or 5 nucleotides longer than the corresponding landing pad sequence. In some embodiments, the first and second emissive readout probes are each at least 5 nucleotides longer than the corresponding landing pad sequence.

In some embodiments, the readout sequence of the first and second emissive readout probes are each about 15 to about 50, about 15 to about 45, about 15 to about 35, about 15 to about 30, about 18 to about 24, about 15, 16, 17, 18, 19, 20, 21, 22, 23, or 24 nucleotides long.

In some embodiments, the emissive readout probe can include a label on the 5′ or 3′ end. In some embodiments, the emissive readout probe can include a label on the 5′ end and a label on the 3′ end. In some embodiments, the emissive readout probe can contain internal labels which may be the same or different. In some embodiments, the labels are the same. In some embodiments, the labels are different.

In some embodiments, the label is a fluorescent entity (fluorophore) or phosphorescent entity. In some embodiments, the label is a cyanine dye (e.g., Cy2, Cy3, Cy3B, Cy5, Cy5.5, Cy7, etc.), Alexa Fluor dye, Atto dye, photo switchable dye, photoactivatable dye, fluorescent dye, metal nanoparticle, semiconductor nanoparticle or “quantum dots”, fluorescent protein such as GFP (Green Fluorescent Protein), or photoactivatable fluorescent protein, such as PAGFP, PSCFP, PSCFP2, Dendra, Dendra2, EosFP, tdEos, mEos2, mEos3, PAmCherry, PAtagRFP, mMaple, mMaple2, and mMaple3.

In some embodiments, the label is Alexa Fluor 350, Alexa Fluor 405, Alexa Fluor 430, Alexa Fluor 488, Alexa Fluor 514, Alexa Fluor 532, Alexa Fluor 546, Alexa Fluor 555, Alexa Fluor 561, Alexa Fluor 568, Alexa Fluor 594, Alexa Fluor 610, Alexa Fluor 633, Alexa Fluor 635, Alexa Fluor 647, Alexa Fluor 647-R-phycoerythrin, Alexa Fluor 660, Alexa Fluor 680, Alexa Fluor 680-allophycocyanin, Alexa Fluor 700, Alexa Fluor 750, Alexa Fluor 790, Alexa Fluor Plus 405, Alexa Fluor Plus 488, Alexa Fluor Plus 555, Alexa Fluor Plus 594, Alexa Fluor Plus 647, Alexa Fluor Plus 680, Alexa Fluor Plus 750, Alexa Fluor Plus 800, Pacific Blue, Pacific Green, Rhodamine Red X, DyLight 485-LS, DyLight-510-LS, DyLight 515-LS, DyLight 521-LS, Hydroxycoumarin, methoxycoumarin, Cy2, FAM, Fluorescein FITC, R-phycoerythrin (PE), Tamara, Cy3.5 581, Rox, Red 613, Texas Red, Cy5, Cy5.5, Cy7, Allophycocyanin, ATTO 430LS, ATTO 490LS, ATTO 390, ATTO 425, Cyan 500 NHS-Ester, ATTO 465, ATTO 488, ATTO 495, ATTO Rho110, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 643, ATTO 647, ATTO 647N, ATTO 655, ATTO Oxa12, ATTO 665, ATTO 680, ATTO 700, ATTO 725, ATTO 740.

### EMBODIMENTS

Embodiments of the present subject matter disclosed herein may be beneficial alone or in combination with one or more other embodiments. Without limiting the foregoing description, certain non-limiting embodiments of the disclosure, numbered I-1 to 11-37 are provided below. As will be apparent to those of skill in the art upon reading this disclosure, each of the individually numbered embodiments may be used or combined with any of the preceding or following individually numbered embodiments. This is intended to provide support for all such combinations of embodiments and is not limited to combinations of embodiments explicitly provided below.

### Embodiments of the Disclosure

Embodiment I-1: A method of characterizing a microbial cell from a biological sample, the method comprising a) directly inoculating the microbe onto a device; b) identifying the microbe; and c) detecting susceptibility to one or more antimicrobial agents.

Embodiment II-1: A method of characterizing a microbial cell from a biological sample, the method comprising a) directly inoculating the microbe onto a device; b) identifying the microbe; and c) detecting future susceptibility to one or more antimicrobial agents.

Embodiment II-2: The method of embodiments I-1 or II-1, wherein the sample is not subjected to culturing before the microbe is inoculated onto the device.

Embodiment II-3: The method of embodiments I-1 or II-1 to II-2, wherein the microbe in the sample is cultured for one to 12 cell divisions before it is inoculated onto the device.

Embodiment II-4: The method of embodiments I-1 or II-1 to II-3, wherein the microbe is identified by in situ hybridization.

Embodiment II-5: The method of embodiments I-1 or II-1 to II-4, wherein the microbe is identified by fluorescence in situ hybridization (FISH).

Embodiment II-6: The method of embodiments I-1 or II-1 to II-5, wherein the fluorescence in situ hybridization is high-phylogenetic-resolution fluorescence in situ hybridization (HiPR-FISH).

Embodiment II-7: The method of embodiments I-1 or II-1 to II-6, wherein the microbe is further characterized via live-cell imaging or dynamic calculation while in situ hybridization is performed.

Embodiment II-8: The method of embodiments I-1 or II-1 to II-7, wherein the microbe is identified by hybridization of a bar-coded probe a 16S ribosomal RNA sequence in the microbe, 5S ribosomal RNA sequence in the microbe, and/or 23S ribosomal RNA sequence in the microbe.

Embodiment II-9: The method of embodiments I-1 or II-1 to II-8, wherein the in situ hybridization is multiplexed.

Embodiment II-10: The method of embodiments I-1 or II-1 to II-9, wherein the susceptibility to one or more microbial agents is determined by measuring the minimum inhibitory concentration of the microbe when exposed to an antimicrobial agent.

Embodiment II-11: The method of embodiments I-1 or II-1 to II-10, wherein the susceptibility to one or more microbial agents is determined by measuring microbial cell metabolism when the microbe is exposed to an antimicrobial agent.

Embodiment II-12: The method of embodiments I-1 or II-1 to II-11, wherein microbial cell metabolism is measured by determining the concentration of dissolved carbon dioxide, oxygen consumption of microbes in the sample, expression of genes involved in cell division and/or growth, or expression of stress response genes.

Embodiment II-13: The method of embodiments I-1 or II-1 to II-12, wherein microbial cell susceptibility is determined by a live/dead stain.

Embodiment II-14: The method of embodiments I-1 or II-1 to II-13, wherein microbial cell susceptibility is determined by cell number.

Embodiment II-15: The method of embodiments I-1 or II-1 to II-14, wherein microbial cell susceptibility is determined by detecting the presence or absence of one or more antimicrobial genes in the microbial cell.

Embodiment II-16: The method of embodiments I-1 or II-1 to II-15, wherein microbial cell susceptibility is determined by detecting the presence or absence of one or more gene mutations associated with the development of antimicrobial resistance or susceptibility in the microbial cell.

Embodiment II-17: The method of embodiments I-1 or II-1 to II-16, wherein future microbial cell susceptibility is determined by detecting the presence or absence of one or more antimicrobial genes in the microbial cell.

Embodiment II-18: The method of embodiments I-1 or II-1 to II-17, wherein future microbial cell susceptibility is determined by detecting the presence or absence of one or more gene mutations associated with the development of antimicrobial resistance or susceptibility in the microbial cell.

Embodiment II-19: The method of embodiments I-1 or II-1 to II-18, wherein the one or more gene mutations associated with the development of antimicrobial resistance or susceptibility is selected from deletions, duplications, single nucleotide polymorphisms (SNPs), frame-shift mutations, inversions, insertions, and/or nucleotide substitutions.

Embodiment II-20: The method of embodiments I-1 or II-1 to II-19, wherein the one or more antimicrobial genes is selected from: genes encoding multidrug resistance proteins (e.g. PDR1, PDR3, PDR7, PDR9), ABC transporters (e.g. SNQ2, STE6, PDR5, PDR10, PDR11, YOR1), membrane associated transporters (GAS1, D4405), soluble proteins (e.g. G3PD), RNA polymerase, rpoB, gyrA, gyrB, 16S RNA, 23S rRNA, NADPH nitroreductase, sul2, strAB, tetAR, aac3-iid, aph, sph, cmy-2, floR, tetB; aadA, aac3-VIa, and sul1.

Embodiment II-21: The method of embodiments I-1 or II-1 to II-20, wherein the presence or absence of one or more antimicrobial genes, or the gene mutation associated with the development of antimicrobial resistance or susceptibility in the microbial cell is detected using in situ hybridization.

Embodiment II-22: The method of embodiments I-1 or II-1 to II-21, wherein the presence or absence of one or more antimicrobial genes, or the gene mutation associated with the development of antimicrobial resistance or susceptibility in the microbial cell is detected using fluorescence in situ hybridization (FISH).

Embodiment II-23: The method of embodiments I-1 or II-1 to II-22, wherein the fluorescence in situ hybridization is high-phylogenetic-resolution fluorescence in situ hybridization (HiPR-FISH).

Embodiment II-24: The method of embodiments I-1 or II-1 to II-23, wherein the identification of the microbial cell and the detection of susceptibility or future susceptibility to one or more antimicrobial agents occurs sequentially.

Embodiment II-25: The method of embodiments I-1 or II-1 to II-24, wherein the identification of the microbial cell and the detection of susceptibility or future susceptibility to one or more antimicrobial agents occurs simultaneously.

Embodiment II-26: The method of embodiments I-1 or II-1 to II-25, wherein the identification of the microbial cell and the detection of susceptibility or future susceptibility to one or more antimicrobial agents occurs in parallel.

Embodiment II-27: The method of embodiments I-1 or II-1 to II-26, wherein the biological sample is obtained from a patient.

Embodiment II-28: The method of embodiments I-1 or II-1 to II-27, wherein the biological sample is obtained from a patient diagnosed with or believed to be suffering from an infection or disorder.

Embodiment II-29: The method of embodiments I-1 or II-1 to II-28, wherein the disease or disorder is an infection.

Embodiment II-30: The method of embodiments I-1 or II-1 to II-29, wherein the infection is a bacterial, viral, fungal, or parasitic infections.

Embodiment II-31: The method of embodiments I-1 or II-1 to II-30, wherein the bacterial infection is selected from Mycobacterium, Streptococcus, Staphylococcus, Shigella, Campylobacter, Salmonella, Clostridium, Corynebacterium, Pseudomonas, Neisseria, Listeria, Vibrio, Bordetella, E. coli (including pathogenic E. coli), Pseudomonas aeruginosa, Enterobacter cloacae, Mycobacterium tuberculosis, Staphylococcus aureus, Helicobacter pylori, Legionella, Acinetobacter baumannii, Citrobacter freundii, Citrobacter koseri, Enterobacter cloacae, Enterococcus faecalis, Enterococcus faecium, Escherichia coli, Klebsiella oxytoca, Klebsiella pneumoniae, Proteus mirabilis, Proteus vulgaris, Pseudomonas aeruginosa, Serratia marcescens, Staphylococcus aureus, Staphylococcus saprophyticus, and Streptococcus agalactiae, or a combination thereof.

Embodiment II-32: The method of embodiments I-1 or II-1 to II-30, wherein the viral infection is selected from Helicobacter pylori, infectious haematopoietic necrosis virus (IHNV), Parvovirus B19, Herpes Simplex Virus, Varicella-zoster virus, Cytomegalovirus, Epstein-Barr virus, Hepatitis A virus, Hepatitis B virus, Hepatitis C virus, Hepatitis D virus, Hepatitis E virus, Measles virus, Mumps virus, Rubella virus, Human Immunodeficiency Virus (HIV), Influenza virus, Rhinovirus, Rotavirus A, Rotavirus B, Rotavirus C, Respiratory Syncytial Virus (RSV), Varicella zoster, Poliovirus, Norovirus, Zika Virus, Dengue Virus, Rabies Virus, Newcastle Disease Virus, and White Spot Syndrome Virus, or a combination thereof.

Embodiment II-33: The method of embodiments I-1 or II-1 to II-30, wherein the fungal infection is selected from Aspergillus, Candida, Pneumocystis, Blastomyces, Coccidioides, Cryptococcus, and Histoplasma, or a combination thereof.

Embodiment II-34: The method of embodiments I-1 or II-1 to II-30, wherein the parasitic infection is selected from Plasmodium (i.e. P. falciparum, P. malariae, P. ovale, P. knowlesi, and P. vivax), Trypanosoma, Toxoplasma, Giardia, and Leishmania, Cryptosporidium, helminthic parasites: Trichuris spp. (whipworms), Enterobius spp. (pinworms), Ascaris spp. (roundworms), Ancylostoma spp. and Necator spp. (hookworms), Strongyloides spp. (threadworms), Dracunculus spp. (Guinea worms), Onchocerca spp. and Wuchereria spp. (filarial worms), Taenia spp., Echinococcus spp., and Diphyllobothrium spp. (human and animal cestodes), Fasciola spp. (liver flukes) and Schistosoma spp. (blood flukes), or a combination thereof.

Embodiment II-35: The method of embodiments I-1 or II-1 to II-34, wherein the biological sample is selected from bronchoalveolar lavage fluid (BAL), blood, serum, plasma, urine, cerebrospinal fluid, pleural fluid, synovial fluid, ocular fluid, peritoneal fluid, amniotic fluid, gastric fluid, lymph fluid, interstitial fluid, tissue homogenate, cell extracts, saliva, sputum, stool, physiological secretions, tears, mucus, sweat, milk, semen, seminal fluid, vaginal secretions, fluid from ulcers and other surface eruptions, blisters, and abscesses, and extracts of tissues including biopsies of normal, malignant, and suspect tissues or any other constituents of the body which may contain the microorganism of interest.

Embodiment II-36: The method of embodiments I-1 or II-1 to II-34, wherein the biological sample is a human oral microbiome sample.

Embodiment II-37: The method of embodiments I-1 or II-1 to II-34, wherein the biological sample is a whole organism.

Embodiment III-1: A method for analyzing a sample, comprising:
contacting at least one encoding probe with the sample to produce a first complex, wherein each encoding probe comprises a targeting sequence, a first landing pad sequence, and a second landing pad sequence;
adding at least one first emissive readout probe to the first complex, wherein the first emissive readout probe comprises a label and a sequence complementary to the first landing pad sequence;
acquiring one or more emission spectra from the first emissive readout probe;
adding an exchange probe to the sample, wherein the exchange probe comprises a 100% complementary sequence to the first emissive readout probe sequence,
hybridizing the exchange probe to the first emissive readout probe to form a second complex;
removing the second complex from the sample,
adding at least one second emissive readout probe to the first complex, wherein the second emissive readout probe comprises a label and a sequence complementary to the second landing pad sequence;
acquiring one or more emission spectra from the second emissive readout probe;
repeating the aforementioned steps for at least one different encoding probe;
determining the spectra of “signal” (e.g., puncta, blobs) and assigning them to a species of interest; and
decoding the spectra into a single, targeted transcript through means of signal deconvolution, error correction, comparison to reference standards.

Embodiment IV-1: A method for analyzing a sample, comprising:
generating a set of probes, wherein each probe comprises:
(i) a targeting sequence;
(ii) a first landing pad sequence; and
(iii) a second landing pad sequence;
contacting the set of probes with the sample to permit hybridization of the probes to nucleotides present in the sample to produce a complex;
adding a first set of emissive readout probes to the complex, wherein each emissive readout probe comprises:
(i) a label, and
(ii) a sequence complementary to the first or second landing pad sequence;
acquiring one or more emission spectra from the first emissive readout probe;
adding a set of exchange probes to the sample, wherein each exchange probe comprises a 100% complementary sequence to the first emissive readout probe sequences,
hybridizing the exchange probes to the first emissive readout probes to form a second complex;
removing the second complex from the sample,
adding a second set of emissive readout probes to the complex, wherein each emissive readout probe comprises:
(i) a label, and
(ii) a sequence complementary to the first or second landing pad sequence;
acquiring one or more emission spectra from the second emissive readout probe;
determining the spectra of “signal” (e.g., puncta, blobs) and assigning them to a species of interest; and
decoding the spectra into a single, targeted transcript through means of signal deconvolution, error correction, comparison to reference standards.

Embodiment IV-2: The method of embodiments III-1 or IV-1, wherein the sample is at least one of a cell, a cell suspension, a tissue biopsy, a tissue specimen, urine, stool, blood, serum, plasma, bone biopsies, bone marrow, respiratory specimens, sputum, induced sputum, tracheal aspirates, bronchoalveolar lavage fluid, sweat, saliva, tears, ocular fluid, cerebral spinal fluid, pericardial fluid, pleural fluid, peritoneal fluid, placenta, amnion, pus, nasal swabs, nasopharyngeal swabs, oropharyngeal swabs, ocular swabs, skin swabs, wound swabs, mucosal swabs, buccal swabs, vaginal swabs, vulvar swabs, nails, nail scrapings, hair follicles, corneal scrapings, gavage fluids, gargle fluids, abscess fluids, wastewater, or plant biopsies.

Embodiment IV-3: The method of embodiment IV-2, wherein the sample is a cell.

Embodiment IV-4: The method of embodiment IV-3, wherein the cell is a bacterial or eukaryotic cell.

Embodiment IV-5: The method of embodiment IV-2, wherein the sample comprises a plurality of cells.

Embodiment IV-6: The method of embodiment IV-5, wherein each cell comprises a specific targeting sequence.

Embodiment IV-7: The method of Embodiments III-1 or IV-1, wherein the targeting sequence targets at least one of messenger RNA (mRNA), microRNA (miRNA), long non-coding RNA (lncRNA), ribosomal RNA (rRNA), small interfering RNA (siRNA), transfer RNA (tRNA), Crispr RNA (crRNA), trans-activating CRISPR RNA (tracrRNA), mitochondrial RNA, intronic RNA, viral mRNA, viral genomic RNA, environmental RNA, double-stranded RNA (dsRNA), small nuclear RNA (snRNA), small nucleolar (snoRNA), PIWI-interacting RNA (piRNA), genomic DNA, synthetic DNA, DNA, plasmid DNA, a plasmid, viral DNA, retroviral DNA, environmental DNA, extracellular DNA, a protein, a small molecule, or an antigenic target.

Embodiment IV-8: The method of embodiment IV-7, wherein the target is mRNA.

Embodiment IV-9: The method of embodiment IV-7, wherein the target is rRNA.

Embodiment IV-10: The method of embodiment IV-7, wherein the target is mRNA and rRNA.

Embodiment IV-11: The method of Embodiments III-1 or IV-1, wherein the at least one encoding probe comprises the first landing pad sequence on the 5′ end, and the second landing pad sequence on the 3′ end.

Embodiment IV-12: The method of Embodiments III-1 or IV-1, wherein the at least one encoding probe comprises the first landing pad sequence on the 3′ end, and the second landing pad sequence on the 5′ end.

Embodiment IV-13: The method of embodiment IV-12, wherein the first landing pad sequence and the second landing pad sequences have different sequences.

Embodiment IV-14: The method of Embodiments III-1 or IV-1, wherein the at least one first or second emissive readout probe comprises a label on the 5′ or 3′ end.

Embodiment IV-15: The method of Embodiments III-1 or IV-1, wherein the label is Alexa Fluor 350, Alexa Fluor 405, Alexa Fluor 430, Alexa Fluor 488, Alexa Fluor 514, Alexa Fluor 532, Alexa Fluor 546, Alexa Fluor 555, Alexa Fluor 561, Alexa Fluor 568, Alexa Fluor 594, Alexa Fluor 610, Alexa Fluor 633, Alexa Fluor 635, Alexa Fluor 647, Alexa Fluor 647-R-phycoerythrin, Alexa Fluor 660, Alexa Fluor 680, Alexa Fluor 680-allophycocyanin, Alexa Fluor 700, Alexa Fluor 750, Alexa Fluor 790, Alexa Fluor Plus 405, Alexa Fluor Plus 488, Alexa Fluor Plus 555, Alexa Fluor Plus 594, Alexa Fluor Plus 647, Alexa Fluor Plus 680, Alexa Fluor Plus 750, Alexa Fluor Plus 800, Pacific Blue, Pacific Green, Rhodamine Red X, DyLight 485-LS, DyLight-510-LS, DyLight 515-LS, DyLight 521-LS, Hydroxycoumarin, methoxycoumarin, Cy2, FAM, Fluorescein FITC, R-phycoerythrin (PE), Tamara, Cy3.5 581, Rox, Red 613, Texas Red, Cy5, Cy5.5, Cy7, Allophycocyanin, ATTO 430LS, ATTO 490LS, ATTO 390, ATTO 425, Cyan 500 NHS-Ester, ATTO 465, ATTO 488, ATTO 495, ATTO Rho110, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 643, ATTO 647, ATTO 647N, ATTO 655, ATTO Oxa12, ATTO 665, ATTO 680, ATTO 700, ATTO 725, ATTO 740.

Embodiment IV-16: The method of Embodiments III-1 or IV-1, wherein the one or more emission spectra of the first and/or second emissive readout probe is acquired via widefield microscopy, point scanning confocal microscopy, spinning disk confocal microscopy, lattice lightsheet microscopy, or light field microscopy.

Embodiment IV-17: The method of embodiment IV-17, wherein the detection strategy used is channel, spectral, channel and fluorescence lifetime, or spectral and fluorescence lifetime.

Embodiment IV-18: The method of Embodiments III-1 or IV-1, wherein the sample is on an analyzing platform, wherein the analyzing platform is a microscope slide, at least one chamber, at least one microfluidic device, at least one well, at least one plate, or at least one filter membrane.

Embodiment IV-19: The method of Embodiments III-1 or IV-1, wherein adding an exchange probe to the sample, hybridizing the exchange probe to the first emissive readout probe, and removing the second complex from the sample are performed in the same step.

Embodiment IV-20: The method of Embodiments III-1 or IV-1, wherein hybridizing the exchange probe to the first or second emissive readout probe results in de-hybridization of the first or second emissive readout probe from the first or second landing pad sequence.

Embodiment IV-21: The method of embodiments IV-19 or IV-20, wherein the step is achieved within 1 hour.

Embodiment IV-22: The method of embodiments IV-19 or IV-20, wherein the step is achieved overnight.

Embodiment IV-23: The method of any one of embodiments III-1, or IV-1 to IV-22, wherein the emissive readout probe sequence is at least 5 nucleotides longer than the first or second landing pad sequences.

Embodiment V-1: A construct comprising:
a targeting sequence that is a region of interest on a nucleotide;
a first landing pad sequence;
a second landing pad sequence, wherein the second landing pad sequence is different from the first landing pad sequence;
a first emissive readout probe comprising a first label and a sequence complimentary to the first landing pad sequence;
an exchange probe comprising a 100% complementary sequence to the first emissive readout probe sequences; and
a second emissive readout probe comprising a second label and a sequence complimentary to the second landing pad sequence.

Embodiment VI-1: A library of constructs comprising a plurality of barcoded probes, wherein each barcoded probe comprises:
a targeting sequence that is a region of interest on a nucleotide;
a first landing pad sequence;
a second landing pad sequence, wherein the second landing pad sequence is different from the first landing pad sequence;
a first emissive readout probe comprising a first label and a sequence complimentary to the first landing pad sequence;
an exchange probe comprising a 100% complementary sequence to the first emissive readout probe sequences; and
a second emissive readout probe comprising a second label and a sequence complimentary to the second landing pad sequence.

Embodiment VI-2: The construct of embodiments V-1 or VI-2, wherein the first emissive readout probe sequence is at least 5 nucleotides longer than the first landing pad sequence.

Embodiment VI-3: The construct of embodiments V-1 or VI-2, wherein the second emissive readout probe sequence is at least 5 nucleotides longer than the second landing pad sequence.

Embodiment VI-4: The construct of embodiments V-1 or VI-2, wherein the first landing pad sequence and the second landing pad sequences have different sequences.

Embodiment VI-5: The construct of embodiments V-1 or VI-2, wherein the first emissive readout probe comprises the first label on the 5′ or 3′ end.

Embodiment VI-6: The construct of embodiments V-1 or VI-2, wherein the second emissive readout probe comprises the second label on the 5′ or 3′ end.

Embodiment VI-7: The construct of embodiments V-1 or VI-2, wherein the first or second label is each Alexa Fluor 350, Alexa Fluor 405, Alexa Fluor 430, Alexa Fluor 488, Alexa Fluor 514, Alexa Fluor 532, Alexa Fluor 546, Alexa Fluor 555, Alexa Fluor 561, Alexa Fluor 568, Alexa Fluor 594, Alexa Fluor 610, Alexa Fluor 633, Alexa Fluor 635, Alexa Fluor 647, Alexa Fluor 647-R-phycoerythrin, Alexa Fluor 660, Alexa Fluor 680, Alexa Fluor 680-allophycocyanin, Alexa Fluor 700, Alexa Fluor 750, Alexa Fluor 790, Alexa Fluor Plus 405, Alexa Fluor Plus 488, Alexa Fluor Plus 555, Alexa Fluor Plus 594, Alexa Fluor Plus 647, Alexa Fluor Plus 680, Alexa Fluor Plus 750, Alexa Fluor Plus 800, Pacific Blue, Pacific Green, Rhodamine Red X, DyLight 485-LS, DyLight-510-LS, DyLight 515-LS, DyLight 521-LS, Hydroxycoumarin, methoxycoumarin, Cy2, FAM, Fluorescein FITC, R-phycoerythrin (PE), Tamara, Cy3.5 581, Rox, Red 613, Texas Red, Cy5, Cy5.5, Cy7, Allophycocyanin, ATTO 430LS, ATTO 490LS, ATTO 390, ATTO 425, Cyan 500 NHS-Ester, ATTO 465, ATTO 488, ATTO 495, ATTO Rho110, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 643, ATTO 647, ATTO 647N, ATTO 655, ATTO Oxa12, ATTO 665, ATTO 680, ATTO 700, ATTO 725, or ATTO 740.

Embodiment VII-1: A method for analyzing a bacterial sample, comprising:
contacting at least one encoding probe with the sample to produce a first complex, wherein each encoding probe comprises a targeting sequence, a first landing pad sequence, and a second landing pad sequence;
adding at least one first emissive readout probe to the first complex, wherein the first emissive readout probe comprises a label and a sequence complementary to the first landing pad sequence;
detecting the first emissive readout probe with a confocal microscope;
adding an exchange probe to the sample, wherein the exchange probe comprises a 100% complementary sequence to the first emissive readout probe sequence,
hybridizing the exchange probe to the first emissive readout probe to form a second complex;
removing the second complex from the sample,
adding at least one second emissive readout probe to the first complex, wherein the second emissive readout probe comprises a label and a sequence complementary to the second landing pad sequence;
detecting the second emissive readout probe with a confocal microscope;
repeating the aforementioned steps for at least one different encoding probe;
determining the spectra of “signal” (e.g., puncta, blobs) and assigning them to a bacterium; and
decoding the spectra into a single, targeted transcript through means of signal deconvolution, error correction, comparison to reference standards.

Embodiment VIII-1: A method for analyzing a bacterial sample, comprising:
generating a set of probes, wherein each probe comprises:
(i) a targeting sequence;
(ii) a first landing pad sequence; and
(iii) a second landing pad sequence;
contacting the set of probes with the sample to permit hybridization of the probes to nucleotides present in the sample to produce a complex;
adding a first set of emissive readout probes to the complex, wherein each emissive readout probe comprises:
(i) a label, and
(ii) a sequence complementary to the first or second landing pad sequence;
detecting the first set of emissive readout probes in the sample with a confocal microscope;
adding a set of exchange probes to the sample, wherein each exchange probe comprises a 100% complementary sequence to the first emissive readout probe sequences,
hybridizing the exchange probes to the first emissive readout probes to form a second complex;
removing the second complex from the sample,
adding a second set of emissive readout probes to the complex, wherein each emissive readout probe comprises:
(i) a label, and
(ii) a sequence complementary to the first or second landing pad sequence;
detecting the second set of emissive readout probes in the sample with a confocal microscope;
determining the spectra of “signal” (e.g., puncta, blobs) and assigning them to a bacterium; and
decoding the spectra into a single, targeted transcript through means of signal deconvolution, error correction, comparison to reference standards.

### EXAMPLES

### Abbreviations and Definitions

| Abbreviation | Definition |
|---|---|
| SSC | sodium chloride sodium citrate |
| SSCT | 2x SSC + 0.1% Tween 20 |
| SDS | Sodium dodecyl sulfate |
| EDTA | Ethylenediaminetetraacetic acid |
| Tris HCl | Tris Hydrochloride |
| | (tris(hydroxymethyl)aminomethane |
| | hydrochloride) |
| NaCl | Sodium chloride |
| PBS | Phosphate-buffered saline |
| RT | Room temperature |

### Example 1. Identification and Antimicrobial Susceptibility Characterization of Microbes

To enable parallel measurement of cellular state at different antibiotic concentrations, microbial cells are colocalized with a volume of antibiotic solution with a known concentration. This objective can potentially be achieved in several ways.

Solution 1: a buffer containing cells are applied to a plate with microfabricated wells (well size can be hundreds of microns to millimeters). Cells may be allowed to settle into individual wells by gravity or by centrifugation. After cell settlement, excess solutions are removed. Subsequently, a hydrated gel (agar, agarose, polyethylene glycol, or polyacrylamide, for example) loaded with an antimicrobial gradient can be applied over the top of the plate, allowing different wells to be exposed to different concentrations of antimicrobial compounds. Solution 2: a buffer containing cells are passed through a microfluidic device to convert the bulk solution into a solution of droplets, where each droplet may contain zero or more cells. The cell droplets are then merged with droplets of antimicrobial solutions using a second microfluidic device, allowing different cells to be exposed to antimicrobial solutions at different concentrations. The antimicrobial solution can be colored with food coloring, or other bacteria-compatible dyes, to allow them to be distinguished on an imaging device. Solution 3: a buffer containing cells are microencapsulated into semipermeable polymeric beads. The polymer beads containing microbial cells are then distributed into wells on a plate, where each well contains a known concentration of antimicrobial compounds.

### Example 2. Identification of Microbes in Patient Sample

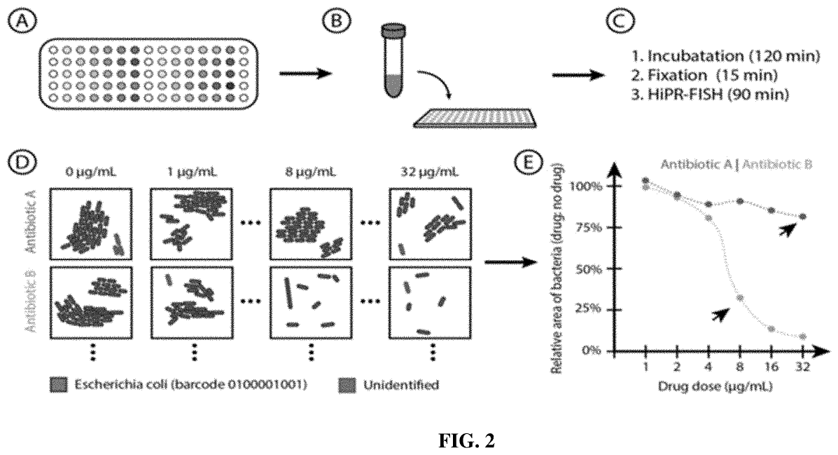

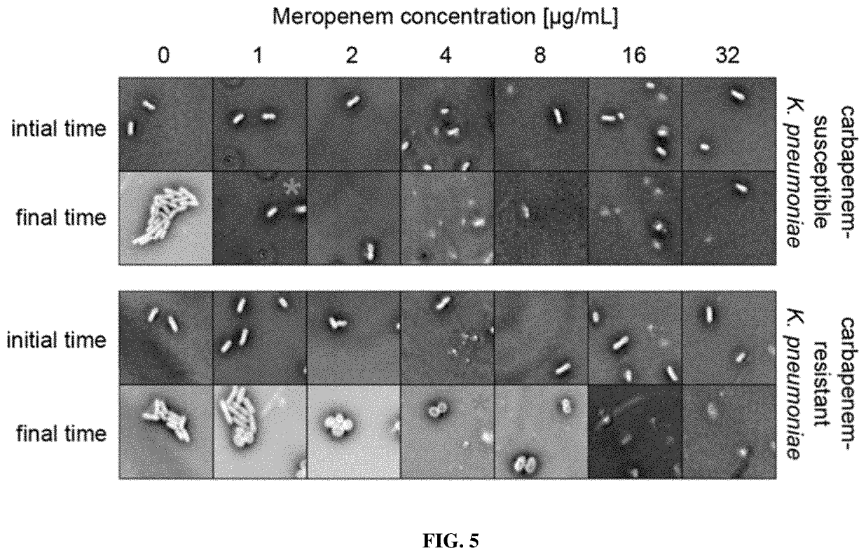

The methods of the disclosure were used to identify microbes and drug-resistance phenotype in patient urine samples. The experimental set up is shown in FIG. 2. A 75-well plate is created with 2-fold dilution series of ten different antibiotics, and the urine samples collected from the patients were deposited over each well. The plate was incubated for 2 hours, and fixed, and HiPR-FISH was performed. Samples were tested at time 0 and 2 hours of the incubation as shown in FIG. 5. After this process (about 4 hours total), spectral imaging was used to identify the microbial species in the patient sample. The detection panel used here detects and differentiates between the following bacteria:
Acinetobacter baumannii
Citrobacter freundii
Citrobacter koseri
Enterobacter cloacae
Enterococcus faecalis
Enterococcus faecium
Escherichia coli
Klebsiella oxytoca
Klebsiella pneumoniae
Proteus mirabilis
Proteus vulgaris
Pseudomonas aeruginosa
Serratia marcescens
Staphylococcus aureus
Staphylococcus saprophyticus
Streptococcus agalactiae

### Example 2.1

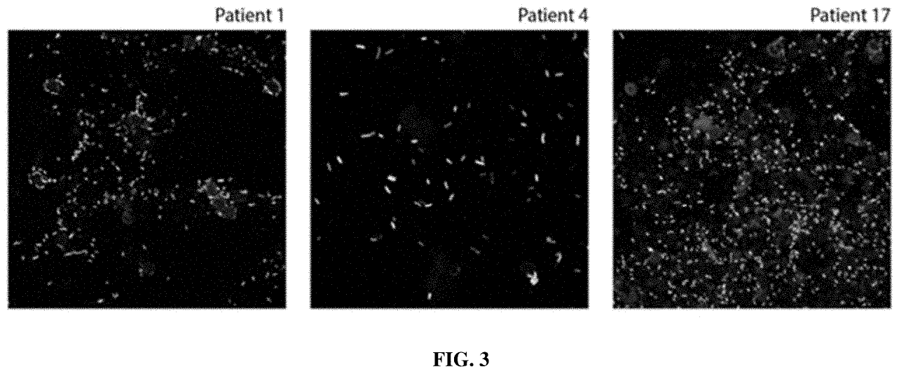

FIG. 3 shows the identification of E. coli in three different patient samples using the following methodology.

Specimens were stored in a mixture of urine supernatant and glycerol and frozen at −80° C. until time of processing. Specimens were thawed and deposited onto the device and incubated at 37° C. for one hour. The specimen was biologically fixed by depositing 2% formaldehyde onto the specimen and incubated for thirty minutes at room temperature. The specimens were washed using 1×PBS multiple times at room temperature. An encoding buffer (2×SSC, 10% dextran sulfate, 10% ethylene carbonate, 5×Denhardt's solution, 0.01% SDS) with probes designed for a panel of uropathogens (at roughly 200 nM per taxon) was deposited on cells and incubated for two hours at 37° C. A wash buffer (5 mM EDTA, 20 mM Tris HCl, 215 mM NaCl) was then deposited on specimens for fifteen minutes at 37° C. to remove unbound probes. A buffer containing readout probes (10 readout probes, each at 400 nM; buffer made up of 2×SSC, 10% dextran sulfate, 10% ethylene carbonate, 5×Denhardt's solution, 0.01% SDS) was incubated for 30 minutes at room temperature. A second round of wash buffer was deposited on specimens for fifteen minutes at 37° C. to remove unbound probe. The specimens were then suspended in 2×SSC and a coverslip was placed directly over the specimens for imaging on a confocal microscope.

### Example 2.2

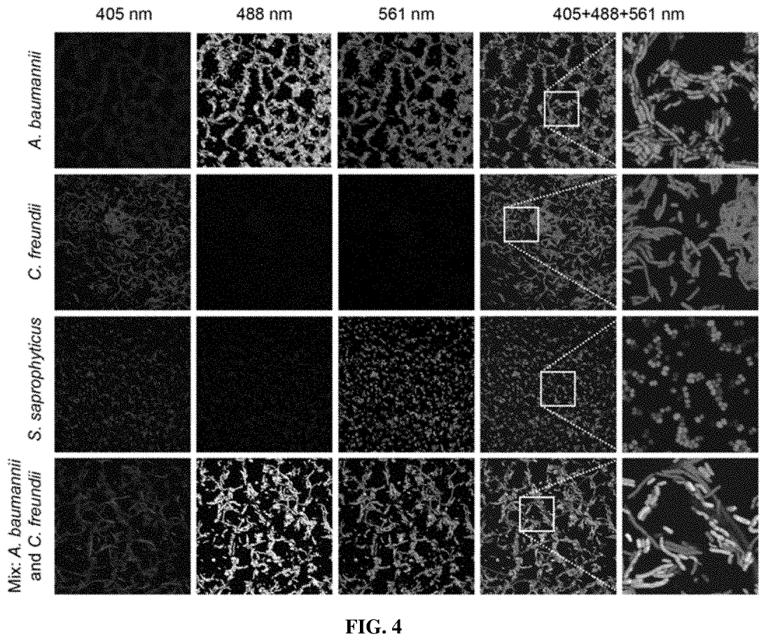

FIG. 4 shows the identification of different species including A. baumannii, C. freundii, S. saprophyticus, and a mixture of A. baumannii and C. freundii using different excitation wavelengths, using the following methodology.

Suspensions of individual monocultures were fixed by adding an equal volume of 2% formaldehyde, mixing, and incubating for 90 minutes at room temperature. Fixed cultures were then washed with 1×PBS and resuspended in 50% ethanol. Single taxa suspensions or mixed suspensions containing multiple taxa, were deposited onto glass microscope slides until 50% ethanol had evaporated. Lysosyme (10 mg/mL) was deposited onto each dry specimen to permeabilize the outer membrane and incubated for 30 minutes at 37° C., the slides were then washed with 1×PBS. An encoding probe hybridization buffer (2×SSC, 10% dextran sulfate, 10% ethylene carbonate, 5×Denhardt's solution, 0.01% SDS) with probes designed for a panel of uropathogens (at roughly 200 nM per taxon) was deposited on cells and incubated for two hours at 37° C. A wash buffer (5 mM EDTA, 20 mM Tris HCl, 215 mM NaCl) was then deposited on specimens for fifteen minutes at 37° C. to remove unbound probes. A buffer containing readout probes (10 readout probes, each at 400 nM; buffer made up of 2×SSC, 10% dextran sulfate, 10% ethylene carbonate, 5×Denhardt's solution, 0.01% SDS) was incubated for one hour at room temperature. A second round of wash buffer was deposited on specimens for fifteen minutes at 37° C. to remove unbound probe. The specimens were mounted with Prolong Glass and a coverslip was placed directly over the specimens for imaging on confocal microscope.

Table 1 shows the sequences of the readout probes used various Examples disclosed herein. Table 2 shows the sequences of the encoding probes used in Examples 2.1 and 2.2.

TABLE 1
Readout Probes

| SEQ | | |
|---|---|---|
| ID | | Sequence |
| NO: | Probe Name | (in 5′ to 3′ order) |
| 1 | Readout Probe 1 | /5Alex488N/TATCCTTCAATCCC |
| | | TCCACA |
| 2 | Readout Probe 2 | /5Alex546N/ACACTACCACCATT |
| | | TCCTAT |
| 3 | Readout Probe 3 | /56-ROXN/ACTCCACTACTACTCA |
| | | CTCT/3Rox_N/ |
| 4 | Readout Probe 4 | /5PacificGreenN/ACCCTCTA |
| | | ACTTCCATCACA |
| 5 | Readout Probe 5 | /5PacificBlueN/ACCACAACCC |
| | | ATTCCTTTCA |
| 6 | Readout Probe 6 | /5Atto610N/TTTACTCCCTACAC |
| | | CTCAA |
| 7 | Readout Probe 7 | /5Alex647N/ACCCTTTACAAACA |
| | | CACCCT |
| 8 | Readout Probe 8 | /5DyLight-510-LS/TCCTATTC |
| | | TCAACCTAACCT/3DyLight-510- |
| | | LS/ |
| 9 | Readout Probe 9 | /5Alex405N/TTCTCCCTCTATCA |
| | | ACTCTA |
| 10 | Readout Probe 10 | /5Alex532N/ACCCTTACTACTAC |
| | | ATCATC/3Alexa532N/ |

TABLE 2
Encoding Probes used in Examples 2.1 and 2.2

| SEQ ID | | |
|---|---|---|
| NO: | Probe Name | Sequence (in 5′ to 3′ order) |
| 11 | Encoding Probe 1 | TGTGGAGGGATTGAAGGATACACCTCCTTGCTAT |
| | | AGCCACCTTATGTGGAGGGATTGAAGGATA |
| 12 | Encoding Probe 2 | TGTGGAGGGATTGAAGGATAGGCAACATCAGAG |
| | | AAGCAAGCAAGTGTGGAGGGATTGAAGGATA |
| 13 | Encoding Probe 3 | TGTGGAGGGATTGAAGGATAAGCGACACAATGT |
| | | CTTCTCCCGTATGTGGAGGGATTGAAGGATA |
| 14 | Encoding Probe 4 | TGTGGAGGGATTGAAGGATATCTCAATGTCTTCT |
| | | CCCCATCAGTCTGTGGAGGGATTGAAGGATA |
| 15 | Encoding Probe 5 | TGTGGAGGGATTGAAGGATACATGGCACCTATTT |
| | | TCTATCTAGAGCGATGTGGAGGGATTGAAGGATA |
| 16 | Encoding Probe 6 | TGTGGAGGGATTGAAGGATACTGGAAGACACAA |
| | | TGTCTTCTCAGGTGTGGAGGGATTGAAGGATA |
| 17 | Encoding Probe 7 | TGTGGAGGGATTGAAGGATAGTCCAGCCTTAATG |
| | | AGTACCGCTATGTGGAGGGATTGAAGGATA |
| 18 | Encoding Probe 8 | TGTGGAGGGATTGAAGGATAGGATCGATTAAAA |
| | | CGATTATAGGTGGATGTGTGGAGGGATTGAAGG |
| | | ATA |
| 19 | Encoding Probe 9 | TGTGGAGGGATTGAAGGATAGGACGATTAAAAC |
| | | GATTATAGGTGGTTGTTGTGGAGGGATTGAAGGA |
| | | TA |
| 20 | Encoding Probe 10 | TGTGGAGGGATTGAAGGATAATTGACAGCAAGA |
| | | CCGTCTTTGTGTGTGGAGGGATTGAAGGATA |
| 21 | Encoding Probe 11 | TGTGGAGGGATTGAAGGATAGATATTGTCCAAAG |
| | | GACAATCCTGTTGTGGAGGGATTGAAGGATA |
| 22 | Encoding Probe 12 | TGTGGAGGGATTGAAGGATATTCACAATGTCTTC |
| | | TCCCCATGTGTGTGGAGGGATTGAAGGATA |
| 23 | Encoding Probe 13 | TGTGGAGGGATTGAAGGATAGGATCACCCATGTT |
| | | CTGACTCGGTTGTGGAGGGATTGAAGGATA |
| 24 | Encoding Probe 14 | TGTGGAGGGATTGAAGGATAATCCTCACGTTTCA |
| | | AAGGCTCGATTGTGGAGGGATTGAAGGATA |
| 25 | Encoding Probe 15 | TGTGGAGGGATTGAAGGATAAAGCGCTACCCTCA |
| | | GTTCATCCCGATGTGGAGGGATTGAAGGATA |
| 26 | Encoding Probe 16 | TGTGGAGGGATTGAAGGATAAAGCCTGACCAAG |
| | | GGTAGATCTGGTGTGGAGGGATTGAAGGATA |
| 27 | Encoding Probe 17 | TGTGGAGGGATTGAAGGATATTGCAACCTGACCA |
| | | AGGGTAGTAGTGTGGAGGGATTGAAGGATA |
| 28 | Encoding Probe 18 | TGTGGAGGGATTGAAGGATAGATATCAGAGAAG |
| | | CAAGCTTCAGCTGTGGAGGGATTGAAGGATA |
| 29 | Encoding Probe 19 | TGTGGAGGGATTGAAGGATAAGGTCAAGAGAGA |
| | | CAACATTTTCCTGTGTGGAGGGATTGAAGGATA |
| 30 | Encoding Probe 20 | TGTGGAGGGATTGAAGGATACTATTCGTCTAATG |
| | | TCGTCCTTTCATTGTGGAGGGATTGAAGGATA |
| 31 | Encoding Probe 21 | TGTGGAGGGATTGAAGGATATGACTAATGCAGC |
| | | GCGGATCCTAGTGTGGAGGGATTGAAGGATA |
| 32 | Encoding Probe 22 | TGTGGAGGGATTGAAGGATATATTGACAGCAAG |
| | | ACCGTCTTAGTTGTGGAGGGATTGAAGGATA |
| 33 | Encoding Probe 23 | TGTGGAGGGATTGAAGGATACAGCCGCTAACATC |
| | | AGAGAAGCTTCTGTGGAGGGATTGAAGGATA |
| 34 | Encoding Probe 24 | TGTGGAGGGATTGAAGGATACAGCTCCACATGTC |
| | | ACCATGCAAGTGTGGAGGGATTGAAGGATA |
| 35 | Encoding Probe 25 | TGTGGAGGGATTGAAGGATACAAAAAGCCAACA |
| | | CAGCTAGGCATTGTGGAGGGATTGAAGGATA |
| 36 | Encoding Probe 26 | ATAGGAAATGGTGGTAGTGTGATCAACAACGCAT |
| | | AAGCGTCGCACGATAGGAAATGGTGGTAGTGT |
| 37 | Encoding Probe 27 | ATAGGAAATGGTGGTAGTGTGGACCAACAACGC |
| | | ATAAGCGTCGCACGATAGGAAATGGTGGTAGTGT |
| 38 | Encoding Probe 28 | ATAGGAAATGGTGGTAGTGTGGACCAACAACGC |
| | | ATAAGCGTCGGACATAGGAAATGGTGGTAGTGT |
| 39 | Encoding Probe 29 | ATAGGAAATGGTGGTAGTGTAAGTCAGGAGACTT |
| | | TAAGTCTCACCCATAGGAAATGGTGGTAGTGT |
| 40 | Encoding Probe 30 | ATAGGAAATGGTGGTAGTGTTTGGGATTACGGGT |
| | | CTACGTTTCTATAGGAAATGGTGGTAGTGT |
| 41 | Encoding Probe 31 | ATAGGAAATGGTGGTAGTGTAGGCAGGAGACTTT |
| | | AAGTCTCAGCCTATAGGAAATGGTGGTAGTGT |
| 42 | Encoding Probe 32 | ATAGGAAATGGTGGTAGTGTGGAAGGAGACTTT |
| | | AAGTCTCAGGCTCATAGGAAATGGTGGTAGTGT |
| 43 | Encoding Probe 33 | ATAGGAAATGGTGGTAGTGTATGAACAACGCAT |
| | | AAGCGTCGCACGATAGGAAATGGTGGTAGTGT |
| 44 | Encoding Probe 34 | ATAGGAAATGGTGGTAGTGTGGACCAACAACGC |
| | | ATAAGCGTCCGAATAGGAAATGGTGGTAGTGT |
| 45 | Encoding Probe 35 | ATAGGAAATGGTGGTAGTGTGATCAACAACGCAT |
| | | AAGCGTCGGACATAGGAAATGGTGGTAGTGT |
| 46 | Encoding Probe 36 | ATAGGAAATGGTGGTAGTGTAGGCAGGAGACTTT |
| | | AAGTCTCACCCATAGGAAATGGTGGTAGTGT |
| 47 | Encoding Probe 37 | ATAGGAAATGGTGGTAGTGTGAAGGAGACTTTA |
| | | AGTCTCAGGCTCATAGGAAATGGTGGTAGTGT |
| 48 | Encoding Probe 38 | ATAGGAAATGGTGGTAGTGTAAGGAGACTTTAA |
| | | GTCTCAGGGTCTATAGGAAATGGTGGTAGTGT |
| 49 | Encoding Probe 39 | ATAGGAAATGGTGGTAGTGTTGTTCAGCGTTAAA |
| | | AGGTACCGCTAATAGGAAATGGTGGTAGTGT |
| 50 | Encoding Probe 40 | ATAGGAAATGGTGGTAGTGTTGGACAACGCATA |
| | | AGCGTCGCACGATAGGAAATGGTGGTAGTGT |
| 51 | Encoding Probe 41 | ATAGGAAATGGTGGTAGTGTGATCAACAACGCAT |
| | | AAGCGTCCGAATAGGAAATGGTGGTAGTGT |
| 52 | Encoding Probe 42 | ATAGGAAATGGTGGTAGTGTATGAACAACGCAT |
| | | AAGCGTCGGACATAGGAAATGGTGGTAGTGT |
| 53 | Encoding Probe 43 | ATAGGAAATGGTGGTAGTGTGGACCAACAACGC |
| | | ATAAGCGTGCGATAGGAAATGGTGGTAGTGT |
| 54 | Encoding Probe 44 | ATAGGAAATGGTGGTAGTGTTGTTCAGCGTTAAA |
| | | AGGTACCCCTATAGGAAATGGTGGTAGTGT |
| 55 | Encoding Probe 45 | ATAGGAAATGGTGGTAGTGTGTGCAGCGTTAAAA |
| | | GGTACCGCTAATAGGAAATGGTGGTAGTGT |
| 56 | Encoding Probe 46 | AGAGTGAGTAGTAGTGGAGTGTGCTCAGTGTTAA |
| | | AGTGCACCCCTAGAGTGAGTAGTAGTGGAGT |
| 57 | Encoding Probe 47 | AGAGTGAGTAGTAGTGGAGTCCGCTCTGCCAAGT |
| | | TCTGTGGTACAGAGTGAGTAGTAGTGGAGT |
| 58 | Encoding Probe 48 | AGAGTGAGTAGTAGTGGAGTGAGTGTTAAAGTG |
| | | CACCGGATTACGAGAGTGAGTAGTAGTGGAGT |
| 59 | Encoding Probe 49 | AGAGTGAGTAGTAGTGGAGTCATCAGCTAACGAT |
| | | AGTGTGACCTCAGAGTGAGTAGTAGTGGAGT |
| 60 | Encoding Probe 50 | AGAGTGAGTAGTAGTGGAGTTCTTTCTCCGCGAG |
| | | GATAACCGGTAGAGTGAGTAGTAGTGGAGT |
| 61 | Encoding Probe 51 | AGAGTGAGTAGTAGTGGAGTTTCCTTCTCCGCGA |
| | | GGATAACCCCTAGAGAGTGAGTAGTAGTGGAGT |
| 62 | Encoding Probe 52 | AGAGTGAGTAGTAGTGGAGTGTCCCATGGGTAA |
| | | ACCACTTCTGGAGAGTGAGTAGTAGTGGAGT |
| 63 | Encoding Probe 53 | AGAGTGAGTAGTAGTGGAGTAGTACGCCTCAGTG |
| | | TTAAAGTCGTAGAGTGAGTAGTAGTGGAGT |
| 64 | Encoding Probe 54 | AGAGTGAGTAGTAGTGGAGTGTGCTCAGTGTTAA |
| | | AGTGCACGCCAGAGTGAGTAGTAGTGGAGT |
| 65 | Encoding Probe 55 | AGAGTGAGTAGTAGTGGAGTCCGCAAGGCATCTC |
| | | TGCCAAGAAGAGAGTGAGTAGTAGTGGAGT |
| 66 | Encoding Probe 56 | AGAGTGAGTAGTAGTGGAGTAGTGTTAAAGTGC |
| | | ACCGGATTACGAGAGTGAGTAGTAGTGGAGT |
| 67 | Encoding Probe 57 | AGAGTGAGTAGTAGTGGAGTATAAGCTAACGAT |
| | | AGTGTGACCTCAGAGTGAGTAGTAGTGGAGT |
| 68 | Encoding Probe 58 | AGAGTGAGTAGTAGTGGAGTCTCTCTCCGCGAGG |
| | | ATAACCCGTAAGAGTGAGTAGTAGTGGAGT |
| 69 | Encoding Probe 59 | AGAGTGAGTAGTAGTGGAGTTCGCTCCGCGAGG |
| | | ATAACCCCTAGAGAGTGAGTAGTAGTGGAGT |
| 70 | Encoding Probe 60 | AGAGTGAGTAGTAGTGGAGTAGTTCCATGGGTAA |
| | | ACCACTTGTGAGAGTGAGTAGTAGTGGAGT |
| 71 | Encoding Probe 61 | AGAGTGAGTAGTAGTGGAGTGTACGCCTCAGTGT |
| | | TAAAGTGGTGAGAGTGAGTAGTAGTGGAGT |
| 72 | Encoding Probe 62 | AGAGTGAGTAGTAGTGGAGTTGCTCAGTGTTAAA |
| | | GTGCACCCCTAGAGTGAGTAGTAGTGGAGT |
| 73 | Encoding Probe 63 | AGAGTGAGTAGTAGTGGAGTGTACGCCTCAGTGT |
| | | TAAAGTGCTGGAGAGTGAGTAGTAGTGGAGT |
| 74 | Encoding Probe 64 | AGAGTGAGTAGTAGTGGAGTGAGTGTTAAAGTG |
| | | CACCGGATAACAGAGTGAGTAGTAGTGGAGT |
| 75 | Encoding Probe 65 | AGAGTGAGTAGTAGTGGAGTCGGAGTGTTAAAG |
| | | TGCACCGGATTTGGGAAGAGTGAGTAGTAGTGG |
| | | AGT |
| 76 | Encoding Probe 66 | AGAGTGAGTAGTAGTGGAGTCATCAGCTAACGAT |
| | | AGTGTGAGCTAGAGTGAGTAGTAGTGGAGT |
| 77 | Encoding Probe 67 | AGAGTGAGTAGTAGTGGAGTAGTACGCCTCAGTG |
| | | TTAAAGTGGTGAGAGTGAGTAGTAGTGGAGT |
| 78 | Encoding Probe 68 | AGAGTGAGTAGTAGTGGAGTAGTTCCATGGGTAA |
| | | ACCACTTCTGGAGAGTGAGTAGTAGTGGAGT |
| 79 | Encoding Probe 69 | AGAGTGAGTAGTAGTGGAGTCGATCCGCGAGGA |
| | | TAACCCCAAGTAGAGTGAGTAGTAGTGGAGT |
| 80 | Encoding Probe 70 | AGAGTGAGTAGTAGTGGAGTTTCCTTCTCCGCGA |
| | | GGATAACAGGAGAGTGAGTAGTAGTGGAGT |
| 81 | Encoding Probe 71 | TGTGATGGAAGTTAGAGGGTGAGGCTCAGTAGTT |
| | | TTGGATGCTCATGTGATGGAAGTTAGAGGGT |
| 82 | Encoding Probe 72 | TGTGATGGAAGTTAGAGGGTAGACGCGTCACTTA |
| | | CGTGACACGGCTGTGATGGAAGTTAGAGGGT |
| 83 | Encoding Probe 73 | TGTGATGGAAGTTAGAGGGTGTGGAGGTGCTGGT |
| | | AACTAAGCTGTGTGATGGAAGTTAGAGGGT |
| 84 | Encoding Probe 74 | TGTGATGGAAGTTAGAGGGTCTAGTTTTATGGGA |
| | | TTAGCTCCAGGATGTGATGGAAGTTAGAGGGT |
| 85 | Encoding Probe 75 | TGTGATGGAAGTTAGAGGGTGAGGAAAGTTCTCA |
| | | GCATGTCTTCTGTGATGGAAGTTAGAGGGT |
| 86 | Encoding Probe 76 | TGTGATGGAAGTTAGAGGGTACACCCATGCTCGG |
| | | CACTTCTCCCTGTGATGGAAGTTAGAGGGT |
| 87 | Encoding Probe 77 | TGTGATGGAAGTTAGAGGGTCGCGGTGTTTTTCA |
| | | CACCCATACATGTGATGGAAGTTAGAGGGT |
| 88 | Encoding Probe 78 | TGTGATGGAAGTTAGAGGGTTGGCCAGAGTGATA |
| | | CATGAGGGCGTGTGATGGAAGTTAGAGGGT |
| 89 | Encoding Probe 79 | TGTGATGGAAGTTAGAGGGTTGGCTATCTCCGAG |
| | | CTTGATTTCGTGTGATGGAAGTTAGAGGGT |
| 90 | Encoding Probe 80 | TGTGATGGAAGTTAGAGGGTGGCACACAGGAAA |
| | | TTCCACCAAGGTGTGATGGAAGTTAGAGGGT |
| 91 | Encoding Probe 81 | TGTGATGGAAGTTAGAGGGTAAGATCCAACTTGC |
| | | TGAACCAGGATGTGATGGAAGTTAGAGGGT |
| 92 | Encoding Probe 82 | TGTGATGGAAGTTAGAGGGTTGCGTCACCTAACA |
| | | AGTAGGCAGGTGTGATGGAAGTTAGAGGGT |
| 93 | Encoding Probe 83 | TGTGATGGAAGTTAGAGGGTCGTGTATTAACTTA |
| | | CTGCCCTTCGAGTGTGATGGAAGTTAGAGGGT |
| 94 | Encoding Probe 84 | TGTGATGGAAGTTAGAGGGTACAAGACAAAGTTT |
| | | CTCGTGCAGGTGTGATGGAAGTTAGAGGGT |
| 95 | Encoding Probe 85 | TGTGATGGAAGTTAGAGGGTAAACTTCAAAGATC |
| | | CTTTCGCCATTGTGATGGAAGTTAGAGGGT |
| 96 | Encoding Probe 86 | TGTGATGGAAGTTAGAGGGTGCACGCTAAAATCA |
| | | ATGAAGCTATTTGTGATGGAAGTTAGAGGGT |
| 97 | Encoding Probe 87 | TGTGATGGAAGTTAGAGGGTCGATCTGATAGCGT |
| | | GAGGTCCCTTTGTGATGGAAGTTAGAGGGT |
| 98 | Encoding Probe 88 | TGTGATGGAAGTTAGAGGGTATAATTCAGTACAA |
| | | GATACCTAGGAATTGTGATGGAAGTTAGAGGGT |
| 99 | Encoding Probe 89 | TGTGATGGAAGTTAGAGGGTAGGCGCTGAATCCA |
| | | GGAGCAACGATGTGATGGAAGTTAGAGGGT |
| 100 | Encoding Probe 90 | TGTGATGGAAGTTAGAGGGTCAAAACGCTCTATG |
| | | ATCGTCAATATGTGATGGAAGTTAGAGGGT |
| 101 | Encoding Probe 91 | TGTGATGGAAGTTAGAGGGTGCAGTGTTTTTCAC |
| | | ACCCATTGTGCATGTGATGGAAGTTAGAGGGT |
| 102 | Encoding Probe 92 | TGTGATGGAAGTTAGAGGGTCTGCGATCGGTTTT |
| | | ATGGGATATCTGTGATGGAAGTTAGAGGGT |
| 103 | Encoding Probe 93 | TGTGATGGAAGTTAGAGGGTGGATCGACGTGTCT |
| | | GTCTCGCTCATGTGATGGAAGTTAGAGGGT |
| 104 | Encoding Probe 94 | TGTGATGGAAGTTAGAGGGTGGTGCAGTAACCA |
| | | GAAGTACACCTTGTGATGGAAGTTAGAGGGT |
| 105 | Encoding Probe 95 | TGTGATGGAAGTTAGAGGGTAGTTCCAACTTGCT |
| | | GAACCACGATTGTGATGGAAGTTAGAGGGT |
| 106 | Encoding Probe 96 | TGAAAGGAATGGGTTGTGGTAAAGAGATTAGCTT |
| | | AGCCTCGGCTTGAAAGGAATGGGTTGTGGT |
| 107 | Encoding Probe 97 | TGAAAGGAATGGGTTGTGGTGTCCTCACGATCTG |
| | | CCTTCGAGCGTGAAAGGAATGGGTTGTGGT |
| 108 | Encoding Probe 98 | TGAAAGGAATGGGTTGTGGTTTAACCTAAAGGTG |
| | | TACTCCAGTCTGAAAGGAATGGGTTGTGGT |
| 109 | Encoding Probe 99 | TGAAAGGAATGGGTTGTGGTTCGTTGACTCCTCT |
| | | TCAGACTATGTGAAAGGAATGGGTTGTGGT |
| 110 | Encoding Probe 100 | TGAAAGGAATGGGTTGTGGTTGAGGCTGATCGTA |
| | | TGATCAGGTGTGAAAGGAATGGGTTGTGGT |
| 111 | Encoding Probe 101 | TGAAAGGAATGGGTTGTGGTTCTAGCTTAGCCTC |
| | | GCGACTTGCGTGAAAGGAATGGGTTGTGGT |
| 112 | Encoding Probe 102 | TGAAAGGAATGGGTTGTGGTACTTTTCCAAGTCA |
| | | TTCGACTATGACTGAAAGGAATGGGTTGTGGT |
| 113 | Encoding Probe 103 | TGAAAGGAATGGGTTGTGGTGATGGGTTTTTACC |
| | | CTCTTTGACACTTGAAAGGAATGGGTTGTGGT |
| 114 | Encoding Probe 104 | TGAAAGGAATGGGTTGTGGTTGATCCAAGTCATT |
| | | CGACTATCACTTGAAAGGAATGGGTTGTGGT |
| 115 | Encoding Probe 105 | TGAAAGGAATGGGTTGTGGTATGTCAAGGGATG |
| | | AACAGTTACAGATGAAAGGAATGGGTTGTGGT |
| 116 | Encoding Probe 106 | TGAAAGGAATGGGTTGTGGTGCAAGGGATGAAC |
| | | AGTTACTCTGTATGAAAGGAATGGGTTGTGGT |
| 117 | Encoding Probe 107 | TGAAAGGAATGGGTTGTGGTAGGCTAGGTGTCTT |
| | | CCACATTTGCATGAAAGGAATGGGTTGTGGT |
| 118 | Encoding Probe 108 | TGAAAGGAATGGGTTGTGGTAGTCGACTATCTGA |
| | | AAGAACTACCTATTGAAAGGAATGGGTTGTGGT |
| 119 | Encoding Probe 109 | TGAAAGGAATGGGTTGTGGTGTAGGTTTTGATTG |
| | | TTATACGGTATATCTGAAAGGAATGGGTTGTGGT |
| 120 | Encoding Probe 110 | TGAAAGGAATGGGTTGTGGTATTTCCTATTAAAG |
| | | ATGTTGGGTAGGTGAAAGGAATGGGTTGTGGT |
| 121 | Encoding Probe 111 | TGAAAGGAATGGGTTGTGGTCGTCTCCGGTGGAA |
| | | AAAGAAGGCATGAAAGGAATGGGTTGTGGT |
| 122 | Encoding Probe 112 | TGAAAGGAATGGGTTGTGGTCAAGCATGGTTACA |
| | | GGTGTATGGATGAAAGGAATGGGTTGTGGT |
| 123 | Encoding Probe 113 | TGAAAGGAATGGGTTGTGGTTATCCTAAAGGTGT |
| | | ACTCCACTCGTGAAAGGAATGGGTTGTGGT |
| 124 | Encoding Probe 114 | TGAAAGGAATGGGTTGTGGTGGACACAGCTTGTC |
| | | CTTAAGATTTTGAAAGGAATGGGTTGTGGT |
| 125 | Encoding Probe 115 | TGAAAGGAATGGGTTGTGGTTGTTCCGATCGTCT |
| | | GCATTCCAATTGAAAGGAATGGGTTGTGGT |
| 126 | Encoding Probe 116 | TGAAAGGAATGGGTTGTGGTTCGGTCCTTAAGAA |
| | | AAGAAGCATAACTGAAAGGAATGGGTTGTGGT |
| 127 | Encoding Probe 117 | TGAAAGGAATGGGTTGTGGTCGAGACAGACATTT |
| | | CCGATCGAGATGAAAGGAATGGGTTGTGGT |
| 128 | Encoding Probe 118 | TGAAAGGAATGGGTTGTGGTGACGCTGATCGTAT |
| | | GATCAGCTGGTGAAAGGAATGGGTTGTGGT |
| 129 | Encoding Probe 119 | TGAAAGGAATGGGTTGTGGTGCCCGGGCCGCTGT |
| | | TTTCTCAGATTGAAAGGAATGGGTTGTGGT |
| 130 | Encoding Probe 120 | TGAAAGGAATGGGTTGTGGTCTTTCGGTACTATT |
| | | ATTTCCCTCAGGTGAAAGGAATGGGTTGTGGT |
| 131 | Encoding Probe 121 | TTGGAGGTGTAGGGAGTAAACCCACGATTGTTGG |
| | | TAACCTGATCGTTGGAGGTGTAGGGAGTAAA |
| 132 | Encoding Probe 122 | TTGGAGGTGTAGGGAGTAAATGGAGCATTAAGT |
| | | GACCGGATAACTTGGAGGTGTAGGGAGTAAA |
| 133 | Encoding Probe 123 | TTGGAGGTGTAGGGAGTAAACTAGCCTAATCACT |
| | | CTGCCTAGTATTGGAGGTGTAGGGAGTAAA |
| 134 | Encoding Probe 124 | TTGGAGGTGTAGGGAGTAAAGGTGCAGTTACCAC |
| | | CAGTACGGCTTTTGGAGGTGTAGGGAGTAAA |
| 135 | Encoding Probe 125 | TTGGAGGTGTAGGGAGTAAAAGCGCTACAACGTT |
| | | TCACTTCACTTTGGAGGTGTAGGGAGTAAA |
| 136 | Encoding Probe 126 | TTGGAGGTGTAGGGAGTAAAGACGTCCACATTTC |
| | | ATAGTCTCCCTGGTTGGAGGTGTAGGGAGTAAA |
| 137 | Encoding Probe 127 | TTGGAGGTGTAGGGAGTAAAAAAGTCACTCAAG |
| | | GTGACAGGGTTCTTGGAGGTGTAGGGAGTAAA |
| 138 | Encoding Probe 128 | TTGGAGGTGTAGGGAGTAAAGAACCACCTTAGTG |
| | | GTTCGTCTAGTTGGAGGTGTAGGGAGTAAA |
| 139 | Encoding Probe 129 | TTGGAGGTGTAGGGAGTAAACAAGGGTACGATT |
| | | GTTGGTAAGGATTGGAGGTGTAGGGAGTAAA |
| 140 | Encoding Probe 130 | TTGGAGGTGTAGGGAGTAAAAAGTTAGGTCACTC |
| | | AAGGTGACAGGGTTCTTGGAGGTGTAGGGAGTA |
| | | AA |
| 141 | Encoding Probe 131 | TTGGAGGTGTAGGGAGTAAAGAAGGTCACTCAA |
| | | GGTGACAGGGAAGTGATTGGAGGTGTAGGGAGT |
| | | AAA |
| 142 | Encoding Probe 132 | TTGGAGGTGTAGGGAGTAAAGAAAGTTCCCGCC |
| | | ATCACGCGGACTTGGAGGTGTAGGGAGTAAA |
| 143 | Encoding Probe 133 | TTGGAGGTGTAGGGAGTAAAGTTGACTTCACTTA |
| | | CCGCCAGGCATTGGAGGTGTAGGGAGTAAA |
| 144 | Encoding Probe 134 | TTGGAGGTGTAGGGAGTAAAGAGGCGCCTGAGT |
| | | ATTCTCTAGGATTGGAGGTGTAGGGAGTAAA |
| 145 | Encoding Probe 135 | TTGGAGGTGTAGGGAGTAAATAACCTAATCACTC |
| | | TGCCTACAACGTTGGAGGTGTAGGGAGTAAA |
| 146 | Encoding Probe 136 | TTGGAGGTGTAGGGAGTAAACCTTGCCTAATCAC |
| | | TCTGCCTTGTTTGGAGGTGTAGGGAGTAAA |
| 147 | Encoding Probe 137 | TTGGAGGTGTAGGGAGTAAACCTTGCCTAATCAC |
| | | TCTGCCTACTACTTGGAGGTGTAGGGAGTAAA |
| 148 | Encoding Probe 138 | TTGGAGGTGTAGGGAGTAAAGGAATCGCAGTTA |
| | | CCACCAGTACCCCTTGGAGGTGTAGGGAGTAAA |
| 149 | Encoding Probe 139 | TTGGAGGTGTAGGGAGTAAACGAGGCTCCGTCCG |
| | | CAAGGGAGAATTGGAGGTGTAGGGAGTAAA |
| 150 | Encoding Probe 140 | TTGGAGGTGTAGGGAGTAAACGTGGACTTCACTT |
| | | ACCGCCAGGCATTGGAGGTGTAGGGAGTAAA |
| 151 | Encoding Probe 141 | TTGGAGGTGTAGGGAGTAAACCCACGATTGTTGG |
| | | TAACCTGTTCTTGGAGGTGTAGGGAGTAAA |
| 152 | Encoding Probe 142 | TTGGAGGTGTAGGGAGTAAAGTGCAGCATTAAGT |
| | | GACCGGAAAATTGGAGGTGTAGGGAGTAAA |
| 153 | Encoding Probe 143 | TTGGAGGTGTAGGGAGTAAATAACCTAATCACTC |
| | | TGCCTACTACTTGGAGGTGTAGGGAGTAAA |
| 154 | Encoding Probe 144 | TTGGAGGTGTAGGGAGTAAAGTACAGTTACCACC |
| | | AGTACGGGTTATTGGAGGTGTAGGGAGTAAA |
| 155 | Encoding Probe 145 | TTGGAGGTGTAGGGAGTAAATAGCGCTACAACGT |
| | | TTCACTTGACTTGGAGGTGTAGGGAGTAAA |
| 156 | Encoding Probe 146 | AGGGTGTGTTTGTAAAGGGTCGTACGCAAAGCGA |
| | | AACGCTTACCAGGGTGTGTTTGTAAAGGGT |
| 157 | Encoding Probe 147 | AGGGTGTGTTTGTAAAGGGTCCATTAACCTCACT |
| | | CCCTTCCAGGAGGGTGTGTTTGTAAAGGGT |
| 158 | Encoding Probe 148 | AGGGTGTGTTTGTAAAGGGTCAGTCATGCTGTCG |
| | | TTACGCATAAAAGGGTGTGTTTGTAAAGGGT |
| 159 | Encoding Probe 149 | AGGGTGTGTTTGTAAAGGGTGTACCGGCTGTAAC |
| | | GGTTCATTAGAGGGTGTGTTTGTAAAGGGT |
| 160 | Encoding Probe 150 | AGGGTGTGTTTGTAAAGGGTAAGGACCCTTAAAG |
| | | GGTCAGGCTCAGGGTGTGTTTGTAAAGGGT |
| 161 | Encoding Probe 151 | AGGGTGTGTTTGTAAAGGGTGAAGGACCCTTAAA |
| | | GGGTCAGCCTAGGGTGTGTTTGTAAAGGGT |
| 162 | Encoding Probe 152 | AGGGTGTGTTTGTAAAGGGTGTGCAGCGTTAGTA |
| | | ACGTTCCCCTAGGGTGTGTTTGTAAAGGGT |
| 163 | Encoding Probe 153 | AGGGTGTGTTTGTAAAGGGTTTCGCTTCACCTAC |
| | | CATCAGCCACAGGGTGTGTTTGTAAAGGGT |
| 164 | Encoding Probe 154 | AGGGTGTGTTTGTAAAGGGTGTCTTAGTAACGTT |
| | | CCGGATTTTGGAGGGTGTGTTTGTAAAGGGT |
| 165 | Encoding Probe 155 | AGGGTGTGTTTGTAAAGGGTGCAGTAACGTTCCG |
| | | GATTTACGACAGGGTGTGTTTGTAAAGGGT |
| 166 | Encoding Probe 156 | AGGGTGTGTTTGTAAAGGGTGCGTTTGCGCACCA |
| | | CGCAAAGGCTAGGGTGTGTTTGTAAAGGGT |
| 167 | Encoding Probe 157 | AGGGTGTGTTTGTAAAGGGTCAACCGTCCATCAT |
| | | GCTGTCGTATGAGGGTGTGTTTGTAAAGGGT |
| 168 | Encoding Probe 158 | AGGGTGTGTTTGTAAAGGGTCGACGTTACGCATT |
| | | TTGCGCAGGTAGGGTGTGTTTGTAAAGGGT |
| 169 | Encoding Probe 159 | AGGGTGTGTTTGTAAAGGGTAGTCCTCAGCGTTA |
| | | GTAACGTTCGCCAGGGTGTGTTTGTAAAGGGT |
| 170 | Encoding Probe 160 | AGGGTGTGTTTGTAAAGGGTAACTTCCCGACCGA |
| | | ATCGCTGCGTAGGGTGTGTTTGTAAAGGGT |
| 171 | Encoding Probe 161 | AGGGTGTGTTTGTAAAGGGTACTCTCCGTTAACC |
| | | GTCCATCTACAGGGTGTGTTTGTAAAGGGT |
| 172 | Encoding Probe 162 | AGGGTGTGTTTGTAAAGGGTGGCAACCGTCCATC |
| | | ATGCTGTGCAAGGGTGTGTTTGTAAAGGGT |
| 173 | Encoding Probe 163 | AGGGTGTGTTTGTAAAGGGTGGTCCAAAACGCTC |
| | | CACTGCCACTAGGGTGTGTTTGTAAAGGGT |
| 174 | Encoding Probe 164 | AGGGTGTGTTTGTAAAGGGTGTATGCTGTCGTTA |
| | | CGCATTTTCGCAGGGTGTGTTTGTAAAGGGT |
| 175 | Encoding Probe 165 | AGGGTGTGTTTGTAAAGGGTGCAACCGTCCATCA |
| | | TGCTGTCCAAAGGGTGTGTTTGTAAAGGGT |
| 176 | Encoding Probe 166 | AGGGTGTGTTTGTAAAGGGTCGAACTGCCTGATT |
| | | TTTGACGAACAGGGTGTGTTTGTAAAGGGT |
| 177 | Encoding Probe 167 | AGGGTGTGTTTGTAAAGGGTGCGCACGCAAAGC |
| | | GAAACGCTTTCCAAGGGTGTGTTTGTAAAGGGT |
| 178 | Encoding Probe 168 | AGGGTGTGTTTGTAAAGGGTCATGTCAATGAATA |
| | | AGGTTATTAACCAGTAGGGTGTGTTTGTAAAGGG |
| | | T |
| 179 | Encoding Probe 169 | AGGGTGTGTTTGTAAAGGGTCAGTCATGCTGTCG |
| | | TTACGCAAAAAGGGTGTGTTTGTAAAGGGT |
| 180 | Encoding Probe 170 | AGGGTGTGTTTGTAAAGGGTTGTGCCGGCTGTAA |
| | | CGGTTCAATAAGGGTGTGTTTGTAAAGGGT |
| 181 | Encoding Probe 171 | AGGTTAGGTTGAGAATAGGATGGGTCGCTTAAAG |
| | | CGACAGGATAAGGTTAGGTTGAGAATAGGA |
| 182 | Encoding Probe 172 | AGGTTAGGTTGAGAATAGGAGAAGTCCGTAGAC |
| | | ATTATGCGCATAGGTTAGGTTGAGAATAGGA |
| 183 | Encoding Probe 173 | AGGTTAGGTTGAGAATAGGATGCCCCCGACCCAG |
| | | TTTATGGCGGAGGTTAGGTTGAGAATAGGA |
| 184 | Encoding Probe 174 | AGGTTAGGTTGAGAATAGGAGAAGGTCGCTTAA |
| | | AGCGACAGGCTTAGGTTAGGTTGAGAATAGGA |
| 185 | Encoding Probe 175 | AGGTTAGGTTGAGAATAGGAAAGTTAGGTCGCTT |
| | | AAAGCGACTCCAGGTTAGGTTGAGAATAGGA |
| 186 | Encoding Probe 176 | AGGTTAGGTTGAGAATAGGAGCTCAGTTTATGGG |
| | | CCTAGGTATCAGGTTAGGTTGAGAATAGGA |
| 187 | Encoding Probe 177 | AGGTTAGGTTGAGAATAGGATCCGCTTAAAGCGA |
| | | CAGGGAACTGAGGTTAGGTTGAGAATAGGA |
| 188 | Encoding Probe 178 | AGGTTAGGTTGAGAATAGGAAAGTTAGGTCGCTT |
| | | AAAGCGACAGGGTTCAGGTTAGGTTGAGAATAG |
| | | GA |
| 189 | Encoding Probe 179 | AGGTTAGGTTGAGAATAGGAGGAAGGTCGCTTA |
| | | AAGCGACAGGGAACTGAGGTTAGGTTGAGAATA |
| | | GGA |
| 190 | Encoding Probe 180 | AGGTTAGGTTGAGAATAGGAGGAAGGTCGCTTA |
| | | AAGCGACACCCAGGTTAGGTTGAGAATAGGA |
| 191 | Encoding Probe 181 | AGGTTAGGTTGAGAATAGGAAAAGTTCCCACCAT |
| | | TACGTGCACCAGGTTAGGTTGAGAATAGGA |
| 192 | Encoding Probe 182 | AGGTTAGGTTGAGAATAGGACAATTTAGGTCGCT |
| | | TAAAGCGACAGGCTTAGGTTAGGTTGAGAATAG |
| | | GA |
| 193 | Encoding Probe 183 | AGGTTAGGTTGAGAATAGGACTGAGTTTATGGGC |
| | | CTAGGTTACTTAGGTTAGGTTGAGAATAGGA |
| 194 | Encoding Probe 184 | AGGTTAGGTTGAGAATAGGAGGCCCAGTTTATGG |
| | | GCCTAGGAATAGGTTAGGTTGAGAATAGGA |
| 195 | Encoding Probe 185 | AGGTTAGGTTGAGAATAGGAGAAGGTCGCTTAA |
| | | AGCGACAGCCTAGGTTAGGTTGAGAATAGGA |
| 196 | Encoding Probe 186 | AGGTTAGGTTGAGAATAGGACCACTTAAAGCGA |
| | | CAGGGAAGTGAAGGTTAGGTTGAGAATAGGA |
| 197 | Encoding Probe 187 | AGGTTAGGTTGAGAATAGGAACTTCCCACCATTA |
| | | CGTGCTGCGTAGGTTAGGTTGAGAATAGGA |
| 198 | Encoding Probe 188 | AGGTTAGGTTGAGAATAGGAGAAGGTCGCTTAA |
| | | AGCGACAGGGAAGTGAAGGTTAGGTTGAGAATA |
| | | GGA |
| 199 | Encoding Probe 189 | AGGTTAGGTTGAGAATAGGAAATTCGCTTAAAGC |
| | | GACAGGGTTCAGGTTAGGTTGAGAATAGGA |
| 200 | Encoding Probe 190 | AGGTTAGGTTGAGAATAGGAAACTTCCCACCATT |
| | | ACGTGCTCCGAGGTTAGGTTGAGAATAGGA |
| 201 | Encoding Probe 191 | AGGTTAGGTTGAGAATAGGAGGAACCCAGTTTAT |
| | | GGGCCTACCAAGGTTAGGTTGAGAATAGGA |
| 202 | Encoding Probe 192 | AGGTTAGGTTGAGAATAGGAAAAGTCGCTTAAA |
| | | GCGACAGGGTTCAGGTTAGGTTGAGAATAGGA |
| 203 | Encoding Probe 193 | AGGTTAGGTTGAGAATAGGACAGCGACCCAGTTT |
| | | ATGGGCCTAGCAAAGGTTAGGTTGAGAATAGGA |
| 204 | Encoding Probe 194 | AGGTTAGGTTGAGAATAGGACTGAGTTTATGGGC |
| | | CTAGGTTTCTAGGTTAGGTTGAGAATAGGA |
| 205 | Encoding Probe 195 | AGGTTAGGTTGAGAATAGGAGCTCAGTTTATGGG |
| | | CCTAGGTTACTTAGGTTAGGTTGAGAATAGGA |
| 206 | Encoding Probe 196 | TAGAGTTGATAGAGGGAGAACGAATGAGTAAAT |
| | | CACTTCACCTAGTATAGAGTTGATAGAGGGAGAA |
| 207 | Encoding Probe 197 | TAGAGTTGATAGAGGGAGAAGGATTCGCTTCATT |
| | | ACGCTATGTAAAGTAGAGTTGATAGAGGGAGAA |
| 208 | Encoding Probe 198 | TAGAGTTGATAGAGGGAGAATGTTCAGCGTTAAA |
| | | AAGGTACCGCTATAGAGTTGATAGAGGGAGAA |
| 209 | Encoding Probe 199 | TAGAGTTGATAGAGGGAGAACCAGCTTCATTACG |
| | | CTATGTATTGTGTAGAGTTGATAGAGGGAGAA |
| 210 | Encoding Probe 200 | TAGAGTTGATAGAGGGAGAATGTTCAGCGTTAAA |
| | | AAGGTACCCCTTAGAGTTGATAGAGGGAGAA |
| 211 | Encoding Probe 201 | TAGAGTTGATAGAGGGAGAAGCCCGCTTCATTAC |
| | | GCTATGTAAAGTAGAGTTGATAGAGGGAGAA |
| 212 | Encoding Probe 202 | TAGAGTTGATAGAGGGAGAAGCGCCCGGGTAAC |
| | | GGGTCCACGAATTAGAGTTGATAGAGGGAGAA |
| 213 | Encoding Probe 203 | TAGAGTTGATAGAGGGAGAAGTGCAGCGTTAAA |
| | | AAGGTACCGCTATAGAGTTGATAGAGGGAGAA |
| 214 | Encoding Probe 204 | TAGAGTTGATAGAGGGAGAAGGATTCGCTTCATT |
| | | ACGCTATGATATAGAGTTGATAGAGGGAGAA |
| 215 | Encoding Probe 205 | TAGAGTTGATAGAGGGAGAAGCGCCCGGGTAAC |
| | | GGGTCCACCAATAGAGTTGATAGAGGGAGAA |
| 216 | Encoding Probe 206 | TAGAGTTGATAGAGGGAGAATGGAGCGTTAAAA |
| | | AGGTACCGCTATAGAGTTGATAGAGGGAGAA |
| 217 | Encoding Probe 207 | TAGAGTTGATAGAGGGAGAAGGAGTTCGCTTCAT |
| | | TACGCTATGTAAAGTAGAGTTGATAGAGGGAGA |
| | | A |
| 218 | Encoding Probe 208 | TAGAGTTGATAGAGGGAGAAGGCTCGCTTCATTA |
| | | CGCTATGTATAGTTAGAGTTGATAGAGGGAGAA |
| 219 | Encoding Probe 209 | TAGAGTTGATAGAGGGAGAACGTCCGGGTAACG |
| | | GGTCCACGAATTAGAGTTGATAGAGGGAGAA |
| 220 | Encoding Probe 210 | TAGAGTTGATAGAGGGAGAAAATTCAATGTATCG |
| | | CTACACTTTGTTAGAGTTGATAGAGGGAGAA |
| 221 | Encoding Probe 211 | TAGAGTTGATAGAGGGAGAACGAATGAGTAAAT |
| | | CACTTCACCTTGTTAGAGTTGATAGAGGGAGAA |
| 222 | Encoding Probe 212 | TAGAGTTGATAGAGGGAGAAGTGCAGCGTTAAA |
| | | AAGGTACCCCTTAGAGTTGATAGAGGGAGAA |
| 223 | Encoding Probe 213 | TAGAGTTGATAGAGGGAGAAGGCTCGCTTCATTA |
| | | CGCTATGTTAATAGAGTTGATAGAGGGAGAA |
| 224 | Encoding Probe 214 | TAGAGTTGATAGAGGGAGAAGAGGCATACCTCA |
| | | CGATACACGAATAGAGTTGATAGAGGGAGAA |
| 225 | Encoding Probe 215 | TAGAGTTGATAGAGGGAGAAGGAGTTCGCTTCAT |
| | | TACGCTATGTTAATAGAGTTGATAGAGGGAGAA |
| 226 | Encoding Probe 216 | TAGAGTTGATAGAGGGAGAAGGATTCGCTTCATT |
| | | ACGCTATGTATTGTGTAGAGTTGATAGAGGGAGA |
| | | A |
| 227 | Encoding Probe 217 | TAGAGTTGATAGAGGGAGAAGGAGTTCGCTTCAT |
| | | TACGCTATCATTAGAGTTGATAGAGGGAGAA |
| 228 | Encoding Probe 218 | TAGAGTTGATAGAGGGAGAAGCCCGCTTCATTAC |
| | | GCTATGTATTGTGTAGAGTTGATAGAGGGAGAA |
| 229 | Encoding Probe 219 | TAGAGTTGATAGAGGGAGAAGGCTCGCTTCATTA |
| | | CGCTATGTATTGTGTAGAGTTGATAGAGGGAGAA |
| 230 | Encoding Probe 220 | TAGAGTTGATAGAGGGAGAAGGCTCGCTTCATTA |
| | | CGCTATGTAAAGTAGAGTTGATAGAGGGAGAA |
| 231 | Encoding Probe 221 | GATGATGTAGTAGTAAGGGTACCTCTTCGACTGG |
| | | TCTCAGCAGGGATGATGTAGTAGTAAGGGT |
| 232 | Encoding Probe 222 | GATGATGTAGTAGTAAGGGTTGCAATCGATGAGG |
| | | TTATTAACCTGTAGATGATGTAGTAGTAAGGGT |
| 233 | Encoding Probe 223 | GATGATGTAGTAGTAAGGGTCATCAGTCACACCC |
| | | GAAGGTGCTAGGGATGATGTAGTAGTAAGGGT |
| 234 | Encoding Probe 224 | GATGATGTAGTAGTAAGGGTGCAATCGATGAGGT |
| | | TATTAACCTGTAGATGATGTAGTAGTAAGGGT |
| 235 | Encoding Probe 225 | GATGATGTAGTAGTAAGGGTCATCAGTCACACCC |
| | | GAAGGTGCAGGGATGATGTAGTAGTAAGGGT |
| 236 | Encoding Probe 226 | GATGATGTAGTAGTAAGGGTATGAGTCACACCCG |
| | | AAGGTGCTAGGGATGATGTAGTAGTAAGGGT |
| 237 | Encoding Probe 227 | GATGATGTAGTAGTAAGGGTTCCCTTCACCTACA |
| | | CACCAGCGACGGATGATGTAGTAGTAAGGGT |
| 238 | Encoding Probe 228 | GATGATGTAGTAGTAAGGGTTCCCTTCACCTACA |
| | | CACCAGCCACGATGATGTAGTAGTAAGGGT |
| 239 | Encoding Probe 229 | GATGATGTAGTAGTAAGGGTTGACCGCAACCCCG |
| | | GTGAGGGCGGGATGATGTAGTAGTAAGGGT |
| 240 | Encoding Probe 230 | GATGATGTAGTAGTAAGGGTAGAGACTGGTCTCA |
| | | GCTCCACGGCGATGATGTAGTAGTAAGGGT |
| 241 | Encoding Probe 231 | GATGATGTAGTAGTAAGGGTATGAGTCACACCCG |
| | | AAGGTGCAGGGATGATGTAGTAGTAAGGGT |
| 242 | Encoding Probe 232 | GATGATGTAGTAGTAAGGGTTGCGTCACACCCGA |
| | | AGGTGCTAGGGATGATGTAGTAGTAAGGGT |
| 243 | Encoding Probe 233 | GATGATGTAGTAGTAAGGGTGTGCTCAGCCTTGA |
| | | TTATCCGCTAGATGATGTAGTAGTAAGGGT |
| 244 | Encoding Probe 234 | GATGATGTAGTAGTAAGGGTCCACGTCAATCGAT |
| | | GAGGTTAAATGATGATGTAGTAGTAAGGGT |
| 245 | Encoding Probe 235 | GATGATGTAGTAGTAAGGGTAATAACCTCATCGC |
| | | CTTCCTCAGGGATGATGTAGTAGTAAGGGT |
| 246 | Encoding Probe 236 | GATGATGTAGTAGTAAGGGTCCCACGTCAATCGA |
| | | TGAGGTTTAAGATGATGTAGTAGTAAGGGT |
| 247 | Encoding Probe 237 | GATGATGTAGTAGTAAGGGTCATCAGTCACACCC |
| | | GAAGGTGGAGGATGATGTAGTAGTAAGGGT |
| 248 | Encoding Probe 238 | GATGATGTAGTAGTAAGGGTCCCTTCACCTACAC |
| | | ACCAGCGACGGATGATGTAGTAGTAAGGGT |
| 249 | Encoding Probe 239 | ATAGGAAATGGTGGTAGTGTCTACCGACCGTGAT |
| | | TAGCTAAGGATGTGGAGGGATTGAAGGATA |
| 250 | Encoding Probe 240 | ATAGGAAATGGTGGTAGTGTCAACTGGAGCTTAG |
| | | AGGATTTTGGATGTGGAGGGATTGAAGGATA |
| 251 | Encoding Probe 241 | ATAGGAAATGGTGGTAGTGTCCCTTAAAGGCCCA |
| | | GGGAAGAGAGTGTGGAGGGATTGAAGGATA |
| 252 | Encoding Probe 242 | ATAGGAAATGGTGGTAGTGTAAGCGCTTATCTTT |
| | | TCCGCACAATTGTGGAGGGATTGAAGGATA |
| 253 | Encoding Probe 243 | ATAGGAAATGGTGGTAGTGTCCCTTCACCTACAT |
| | | GCCAGCGACGTGTGGAGGGATTGAAGGATA |
| 254 | Encoding Probe 244 | ATAGGAAATGGTGGTAGTGTCTGTGTCCTCACCC |
| | | CAGATTAACCTGTGGAGGGATTGAAGGATA |
| 255 | Encoding Probe 245 | ATAGGAAATGGTGGTAGTGTATGTTTAATGTTAC |
| | | CTGGAGCTATCTGTGGAGGGATTGAAGGATA |
| 256 | Encoding Probe 246 | ATAGGAAATGGTGGTAGTGTTCCATCAACTACTT |
| | | CTGCACCGATCTGTGGAGGGATTGAAGGATA |
| 257 | Encoding Probe 247 | ATAGGAAATGGTGGTAGTGTGCGCAGGGTTGATA |
| | | TGCAACCCCTTGTGGAGGGATTGAAGGATA |
| 258 | Encoding Probe 248 | ATAGGAAATGGTGGTAGTGTGATCAACAACGCTA |
| | | AGCGTCGGACTGTGGAGGGATTGAAGGATA |
| 259 | Encoding Probe 249 | ATAGGAAATGGTGGTAGTGTAGTTCCATCCGCGA |
| | | GGGACTTGTGTGTGGAGGGATTGAAGGATA |
| 260 | Encoding Probe 250 | ATAGGAAATGGTGGTAGTGTTAATGAACGTATTA |
| | | AGCTCACCTGGTGTGGAGGGATTGAAGGATA |
| 261 | Encoding Probe 251 | ATAGGAAATGGTGGTAGTGTGTCCATCAACTACT |
| | | TCTGCACCGATCTGTGGAGGGATTGAAGGATA |
| 262 | Encoding Probe 252 | ATAGGAAATGGTGGTAGTGTATACCCTTTGCTGC |
| | | GCGACTTAGGTGTGGAGGGATTGAAGGATA |
| 263 | Encoding Probe 253 | ATAGGAAATGGTGGTAGTGTCAGTACCTTGCAAC |
| | | TAATCGCGGTTGTGGAGGGATTGAAGGATA |
| 264 | Encoding Probe 254 | ATAGGAAATGGTGGTAGTGTCAGTTGATGAACGT |
| | | ATTAAGCTCAGGTTGTGGAGGGATTGAAGGATA |
| 265 | Encoding Probe 255 | ATAGGAAATGGTGGTAGTGTTATCAGACAGGATG |
| | | TCACGTGAGGTGTGGAGGGATTGAAGGATA |
| 266 | Encoding Probe 256 | ATAGGAAATGGTGGTAGTGTGATCATCGAACTCA |
| | | CGACCTGTCGTTGTGGAGGGATTGAAGGATA |
| 267 | Encoding Probe 257 | ATAGGAAATGGTGGTAGTGTTGTAGCCGATTCAG |
| | | GTTCTGGCGATGTGGAGGGATTGAAGGATA |
| 268 | Encoding Probe 258 | ATAGGAAATGGTGGTAGTGTATAATTCATGACAT |
| | | GATAATGTGTGCTTGTGGAGGGATTGAAGGATA |
| 269 | Encoding Probe 259 | ATAGGAAATGGTGGTAGTGTATAGGCAGTGTCCT |
| | | ACTCTCGGTATGTGGAGGGATTGAAGGATA |
| 270 | Encoding Probe 260 | ATAGGAAATGGTGGTAGTGTAATGGGCCGAGTTA |
| | | GAACATCTTTTGTGGAGGGATTGAAGGATA |
| 271 | Encoding Probe 261 | ATAGGAAATGGTGGTAGTGTAAAGCGCTTATCTT |
| | | TTCCGCAGAATGTGGAGGGATTGAAGGATA |
| 272 | Encoding Probe 262 | ATAGGAAATGGTGGTAGTGTAAAGGGCCTTAAA |
| | | GGCCCAGGCTTTGTGGAGGGATTGAAGGATA |
| 273 | Encoding Probe 263 | ATAGGAAATGGTGGTAGTGTTCCCCTTAAAGGCC |
| | | CAGGGAACTGTGTGGAGGGATTGAAGGATA |
| 274 | Encoding Probe 264 | AGAGTGAGTAGTAGTGGAGTCCCCGATTCCTGTG |
| | | TAACTGAAGGAATGTGGAGGGATTGAAGGATA |
| 275 | Encoding Probe 265 | AGAGTGAGTAGTAGTGGAGTGGACACGTATACA |
| | | AAGTATACATCCCGTTGTGGAGGGATTGAAGGAT |
| | | A |
| 276 | Encoding Probe 266 | AGAGTGAGTAGTAGTGGAGTACGGCAAGTAAGG |
| | | AAAAGGGTACGTGTGGAGGGATTGAAGGATA |
| 277 | Encoding Probe 267 | AGAGTGAGTAGTAGTGGAGTACGCACCTGTATCT |
| | | AGATTCCCGTTCTGTGGAGGGATTGAAGGATA |
| 278 | Encoding Probe 268 | AGAGTGAGTAGTAGTGGAGTACCGTCTGGATTGT |
| | | TTTCCTCTACTTGTGGAGGGATTGAAGGATA |
| 279 | Encoding Probe 269 | AGAGTGAGTAGTAGTGGAGTAGACGGATAGTAC |
| | | TCATAGGTATTGCCTGTGGAGGGATTGAAGGATA |
| 280 | Encoding Probe 270 | AGAGTGAGTAGTAGTGGAGTGAAAGTTCCCATCC |
| | | GAAATGCTGCGTTGTGGAGGGATTGAAGGATA |
| 281 | Encoding Probe 271 | AGAGTGAGTAGTAGTGGAGTCGAGCCACTAAAG |
| | | CCTCAAAGGAGGTGTGGAGGGATTGAAGGATA |
| 282 | Encoding Probe 272 | AGAGTGAGTAGTAGTGGAGTGAGCGTCAGTATTA |
| | | GGCCAGATGGGACTGTGGAGGGATTGAAGGATA |
| 283 | Encoding Probe 273 | AGAGTGAGTAGTAGTGGAGTTGGGAATTCTACCA |
| | | TCCTCTCCGTATGTGGAGGGATTGAAGGATA |
| 284 | Encoding Probe 274 | AGAGTGAGTAGTAGTGGAGTGGTCTCTCCCATAC |
| | | TCTAGCTGTGTGTGGAGGGATTGAAGGATA |
| 285 | Encoding Probe 275 | AGAGTGAGTAGTAGTGGAGTTCCGTTCACTCTTG |
| | | CTATGGTGCGTGTGGAGGGATTGAAGGATA |
| 286 | Encoding Probe 276 | AGAGTGAGTAGTAGTGGAGTGGATATTCAGACA |
| | | AGGTTTCACGTCGGTGTGGAGGGATTGAAGGATA |
| 287 | Encoding Probe 277 | AGAGTGAGTAGTAGTGGAGTAGAGTATTAACTA |
| | | AAGTAGCCTCCAGGTGTGGAGGGATTGAAGGAT |
| | | A |
| 288 | Encoding Probe 278 | AGAGTGAGTAGTAGTGGAGTGTATCAGACAAGG |
| | | TTTCACGTCGGTGTGGAGGGATTGAAGGATA |
| 289 | Encoding Probe 279 | AGAGTGAGTAGTAGTGGAGTTGATCATCATTATG |
| | | TGTGCCCAAATGTGGAGGGATTGAAGGATA |
| 290 | Encoding Probe 280 | AGAGTGAGTAGTAGTGGAGTAGATAAAACACAC |
| | | ATAACTTAATGGGAACTGTGGAGGGATTGAAGG |
| | | ATA |
| 291 | Encoding Probe 281 | AGAGTGAGTAGTAGTGGAGTGAAGCTCATCTATT |
| | | AGCGCAACCATGTGGAGGGATTGAAGGATA |
| 292 | Encoding Probe 282 | AGAGTGAGTAGTAGTGGAGTATAATTCATGTTGC |
| | | AATACCTACGAATGTGGAGGGATTGAAGGATA |
| 293 | Encoding Probe 283 | AGAGTGAGTAGTAGTGGAGTCAGCCGCTAGGTCC |
| | | GGTAGCAACGATGTGGAGGGATTGAAGGATA |
| 294 | Encoding Probe 284 | AGAGTGAGTAGTAGTGGAGTGTCGGTTCACTCTT |
| | | GCTATGGAGCTGTGGAGGGATTGAAGGATA |
| 295 | Encoding Probe 285 | AGAGTGAGTAGTAGTGGAGTAAAGATTAGCATC |
| | | ACATCGCTCACTGTGGAGGGATTGAAGGATA |
| 296 | Encoding Probe 286 | AGAGTGAGTAGTAGTGGAGTTAAGACTCGATTTC |
| | | TCTACGGGAGTGTGGAGGGATTGAAGGATA |
| 297 | Encoding Probe 287 | AGAGTGAGTAGTAGTGGAGTGGCCTCTTTGCAGT |
| | | TAGGCTAGGATTGTGGAGGGATTGAAGGATA |
| 298 | Encoding Probe 288 | AGAGTGAGTAGTAGTGGAGTTCTTCAGCATAGAG |
| | | TACCCCGCTATGTGGAGGGATTGAAGGATA |
| 299 | Encoding Probe 289 | AGAGTGAGTAGTAGTGGAGTAGGTCGTCTGGTTT |
| | | AGTTAGCGATATAGGAAATGGTGGTAGTGT |
| 300 | Encoding Probe 290 | AGAGTGAGTAGTAGTGGAGTGGAATCACTATATA |
| | | CTCTAGTACAGGTTAATAGGAAATGGTGGTAGTG |
| | | T |
| 301 | Encoding Probe 291 | AGAGTGAGTAGTAGTGGAGTCCGCCCGTTATCAT |
| | | AGGCTCCATGATAGGAAATGGTGGTAGTGT |
| 302 | Encoding Probe 292 | AGAGTGAGTAGTAGTGGAGTAGGACTGAGATTG |
| | | GCTTTAAGACTAATAGGAAATGGTGGTAGTGT |
| 303 | Encoding Probe 293 | AGAGTGAGTAGTAGTGGAGTAAAGGTCTACAAC |
| | | ATGATACTATGCGCATAGGAAATGGTGGTAGTGT |
| 304 | Encoding Probe 294 | AGAGTGAGTAGTAGTGGAGTAGGCCATGACACTT |
| | | TTGTGTCTAGATAGGAAATGGTGGTAGTGT |
| 305 | Encoding Probe 295 | AGAGTGAGTAGTAGTGGAGTTACACTTTTGTGTC |
| | | ATCCACACGAATAGGAAATGGTGGTAGTGT |
| 306 | Encoding Probe 296 | AGAGTGAGTAGTAGTGGAGTTGCCTCTTTGAATG |
| | | AATAGCTGCAAGATAGGAAATGGTGGTAGTGT |
| 307 | Encoding Probe 297 | AGAGTGAGTAGTAGTGGAGTACGCGAAGAGAAA |
| | | GCCTATCTCATCATAGGAAATGGTGGTAGTGT |
| 308 | Encoding Probe 298 | AGAGTGAGTAGTAGTGGAGTTTATCTGGTTTAGT |
| | | TAGCCTACACGATAGGAAATGGTGGTAGTGT |
| 309 | Encoding Probe 299 | AGAGTGAGTAGTAGTGGAGTCCTTTATCTGAGAT |
| | | TGGTAATCCGCCTATAGGAAATGGTGGTAGTGT |
| 310 | Encoding Probe 300 | AGAGTGAGTAGTAGTGGAGTGATTCCAAGAGAC |
| | | TTAACATCGACCGAATAGGAAATGGTGGTAGTGT |
| 311 | Encoding Probe 301 | AGAGTGAGTAGTAGTGGAGTGGTAGTCATCCAA |
| | | GCACTTTTGTTATAGGAAATGGTGGTAGTGT |
| 312 | Encoding Probe 302 | AGAGTGAGTAGTAGTGGAGTGGAAAGTCATCCA |
| | | AGCACTTTAGTATAGGAAATGGTGGTAGTGT |
| 313 | Encoding Probe 303 | AGAGTGAGTAGTAGTGGAGTAAAAAAGCGTACA |
| | | ATGGTTAAGGGTATAGGAAATGGTGGTAGTGT |
| 314 | Encoding Probe 304 | AGAGTGAGTAGTAGTGGAGTAAGGCGTTCTAGG |
| | | GCTTAACTAGAATAGGAAATGGTGGTAGTGT |
| 315 | Encoding Probe 305 | AGAGTGAGTAGTAGTGGAGTTAACGGGCTCGAA |
| | | CTTGTTGTTCCATAGGAAATGGTGGTAGTGT |
| 316 | Encoding Probe 306 | AGAGTGAGTAGTAGTGGAGTAATGTCACTTGGTA |
| | | GATTTTCCAGAGATAGGAAATGGTGGTAGTGT |
| 317 | Encoding Probe 307 | AGAGTGAGTAGTAGTGGAGTGGTCCTACCAACGT |
| | | TCTTCTCATTATAGGAAATGGTGGTAGTGT |
| 318 | Encoding Probe 308 | AGAGTGAGTAGTAGTGGAGTCTATGCTAAGGTTA |
| | | ATCTATCATTTTTTTATAGGAAATGGTGGTAGTGT |
| 319 | Encoding Probe 309 | AGAGTGAGTAGTAGTGGAGTGGACCAGGTAATT |
| | | CTTCTATAATGATATTATAGGAAATGGTGGTAGT |
| | | GT |
| 320 | Encoding Probe 310 | AGAGTGAGTAGTAGTGGAGTGTTCCGAAGTGTAA |
| | | ACACTTCCCAATAGGAAATGGTGGTAGTGT |
| 321 | Encoding Probe 311 | AGAGTGAGTAGTAGTGGAGTGTTCATCAGTCTAG |
| | | TGTAAACACGTTATAGGAAATGGTGGTAGTGT |
| 322 | Encoding Probe 312 | AGAGTGAGTAGTAGTGGAGTCTAGGATACTAGTC |
| | | ATTAACTAGTGCCAATAGGAAATGGTGGTAGTGT |
| 323 | Encoding Probe 313 | AGAGTGAGTAGTAGTGGAGTCGTTCATCAGTCTA |
| | | GTGTAAACACGTTATAGGAAATGGTGGTAGTGT |
| 324 | Encoding Probe 314 | TGTGATGGAAGTTAGAGGGTGTACTTGGACATGC |
| | | ACTTCCAATGCGTGTGGAGGGATTGAAGGATA |
| 325 | Encoding Probe 315 | TGTGATGGAAGTTAGAGGGTAGTCTTATGCCATG |
| | | CGGCATATTGTGTGGAGGGATTGAAGGATA |
| 326 | Encoding Probe 316 | TGTGATGGAAGTTAGAGGGTAGGCCACTACACCT |
| | | AATGGTGATCTGTGGAGGGATTGAAGGATA |
| 327 | Encoding Probe 317 | TGTGATGGAAGTTAGAGGGTGATCCTAATGGTGT |
| | | AGTCCACTCGTGTGGAGGGATTGAAGGATA |
| 328 | Encoding Probe 318 | TGTGATGGAAGTTAGAGGGTGATGTTCCGGTCTC |
| | | ATCGGCTGGATGTGGAGGGATTGAAGGATA |
| 329 | Encoding Probe 319 | TGTGATGGAAGTTAGAGGGTGGACACTCTTATGC |
| | | CATGCGGCTATTGTGGAGGGATTGAAGGATA |
| 330 | Encoding Probe 320 | TGTGATGGAAGTTAGAGGGTTCATTAATGCGTTT |
| | | GCTGCAGGTGTGTGGAGGGATTGAAGGATA |
| 331 | Encoding Probe 321 | TGTGATGGAAGTTAGAGGGTCCCTTTCACCCTCT |
| | | TTAGCGGTTATGTGGAGGGATTGAAGGATA |
| 332 | Encoding Probe 322 | TGTGATGGAAGTTAGAGGGTATGCTACATACTTA |
| | | TTCGCCCTTAATGTGGAGGGATTGAAGGATA |
| 333 | Encoding Probe 323 | TGTGATGGAAGTTAGAGGGTGTGCATCACTCATT |
| | | AACGAGCAAATGTGGAGGGATTGAAGGATA |
| 334 | Encoding Probe 324 | TGTGATGGAAGTTAGAGGGTCAAGGGACGTTCA |
| | | GTTACTAAACATGTGGAGGGATTGAAGGATA |
| 335 | Encoding Probe 325 | TGTGATGGAAGTTAGAGGGTTCCACGTTCAGTTA |
| | | CTAACGTGGATGTGGAGGGATTGAAGGATA |
| 336 | Encoding Probe 326 | TGTGATGGAAGTTAGAGGGTTAGCCTAGGTGTTG |
| | | TCAGCATAAGTGTGGAGGGATTGAAGGATA |
| 337 | Encoding Probe 327 | TGTGATGGAAGTTAGAGGGTTAGTCAACTATACT |
| | | AACAGACTACCTATTGTGGAGGGATTGAAGGATA |
| 338 | Encoding Probe 328 | TGTGATGGAAGTTAGAGGGTTCCACGTTCAGTTA |
| | | CTAACGTCGAATGTGGAGGGATTGAAGGATA |
| 339 | Encoding Probe 329 | TGTGATGGAAGTTAGAGGGTGGTCGGCATAAACT |
| | | GTTATGCCCATGTGGAGGGATTGAAGGATA |
| 340 | Encoding Probe 330 | TGTGATGGAAGTTAGAGGGTCGAGTATTCACTGA |
| | | AAAGTAATATCCATATGTGGAGGGATTGAAGGAT |
| | | A |
| 341 | Encoding Probe 331 | TGTGATGGAAGTTAGAGGGTGAGCTTTCCAATTG |
| | | AGTGCAACGTTGTGGAGGGATTGAAGGATA |
| 342 | Encoding Probe 332 | TGTGATGGAAGTTAGAGGGTCATGCATTTAACTC |
| | | TACTCAAGACTGTATGTGGAGGGATTGAAGGATA |
| 343 | Encoding Probe 333 | TGTGATGGAAGTTAGAGGGTCTTTCGCTACTATT |
| | | ATTTCGCTAGGTGTGGAGGGATTGAAGGATA |
| 344 | Encoding Probe 334 | TGTGATGGAAGTTAGAGGGTCCAGGGCAGTTGTT |
| | | TTCTCACATCTGTGGAGGGATTGAAGGATA |
| 345 | Encoding Probe 335 | TGTGATGGAAGTTAGAGGGTGACGCTGACCGAA |
| | | GTCAGCACAGGTGTGGAGGGATTGAAGGATA |
| 346 | Encoding Probe 336 | TGTGATGGAAGTTAGAGGGTGATACTAGCCTTCC |
| | | ACTTCCAAGGATGTGGAGGGATTGAAGGATA |
| 347 | Encoding Probe 337 | TGTGATGGAAGTTAGAGGGTACCCTTCAATTCTG |
| | | AGCTTCGGGCTGTGGAGGGATTGAAGGATA |
| 348 | Encoding Probe 338 | TGTGATGGAAGTTAGAGGGTCAGCTCCAACTATC |
| | | ACTAGCCTTGGTTGTGGAGGGATTGAAGGATA |
| 349 | Encoding Probe 339 | TGTGATGGAAGTTAGAGGGTCCTCAGTTAATGAT |
| | | AGTGTGTCGATTGATAGGAAATGGTGGTAGTGT |
| 350 | Encoding Probe 340 | TGTGATGGAAGTTAGAGGGTGGAGCCTTGGTTTT |
| | | CCGGATTACGATAGGAAATGGTGGTAGTGT |
| 351 | Encoding Probe 341 | TGTGATGGAAGTTAGAGGGTGTGTCTCATCTCTG |
| | | AAAACTTCCCACATAGGAAATGGTGGTAGTGT |
| 352 | Encoding Probe 342 | TGTGATGGAAGTTAGAGGGTGTCACCCCATTAAG |
| | | AGGCTCCGTGATAGGAAATGGTGGTAGTGT |
| 353 | Encoding Probe 343 | TGTGATGGAAGTTAGAGGGTCCACGTCAATGAGC |
| | | AAAGGTAAATATAGGAAATGGTGGTAGTGT |
| 354 | Encoding Probe 344 | TGTGATGGAAGTTAGAGGGTGTAAGCTCACAATA |
| | | TGTGCATAAAATAGGAAATGGTGGTAGTGT |
| 355 | Encoding Probe 345 | TGTGATGGAAGTTAGAGGGTGATACACACACTGA |
| | | TTCAGGCAGAATAGGAAATGGTGGTAGTGT |
| 356 | Encoding Probe 346 | TGTGATGGAAGTTAGAGGGTAGTCTTGGTTTTCC |
| | | GGATTTGGGAATAGGAAATGGTGGTAGTGT |
| 357 | Encoding Probe 347 | TGTGATGGAAGTTAGAGGGTACCTCAGTTAATGA |
| | | TAGTGTGTCGTTTATAGGAAATGGTGGTAGTGT |
| 358 | Encoding Probe 348 | TGTGATGGAAGTTAGAGGGTGAGCCTTGGTTTTC |
| | | CGGATTTCGGATAGGAAATGGTGGTAGTGT |
| 359 | Encoding Probe 349 | TGTGATGGAAGTTAGAGGGTGTATCATCTCTGAA |
| | | AACTTCCGACCATAGGAAATGGTGGTAGTGT |
| 360 | Encoding Probe 350 | TGTGATGGAAGTTAGAGGGTGTGCTCAGCCTTGG |
| | | TTTTCCGCTAATAGGAAATGGTGGTAGTGT |
| 361 | Encoding Probe 351 | TGTGATGGAAGTTAGAGGGTTGCGTCACCCCATT |
| | | AAGAGGCAGGATAGGAAATGGTGGTAGTGT |
| 362 | Encoding Probe 352 | TGTGATGGAAGTTAGAGGGTCATGTCAATGAGCA |
| | | AAGGTATTAAGAAATAGGAAATGGTGGTAGTGT |
| 363 | Encoding Probe 353 | TGTGATGGAAGTTAGAGGGTGTAAGCTCACAATA |
| | | TGTGCATTAAAATAGGAAATGGTGGTAGTGT |
| 364 | Encoding Probe 354 | TGTGATGGAAGTTAGAGGGTGAAACTAACACAC |
| | | ACACTGATTGTCATAGGAAATGGTGGTAGTGT |
| 365 | Encoding Probe 355 | TGTGATGGAAGTTAGAGGGTCTAAGTTAATGATA |
| | | GTGTGTCGATTGATAGGAAATGGTGGTAGTGT |
| 366 | Encoding Probe 356 | TGTGATGGAAGTTAGAGGGTGTGTCTCATCTCTG |
| | | AAAACTTCCGACCATAGGAAATGGTGGTAGTGT |
| 367 | Encoding Probe 357 | TGTGATGGAAGTTAGAGGGTAGGAAGGCACATT |
| | | CTCATCTCACTATAGGAAATGGTGGTAGTGT |
| 368 | Encoding Probe 358 | TGTGATGGAAGTTAGAGGGTCGTCACCCCATTAA |
| | | GAGGCTCGGTATAGGAAATGGTGGTAGTGT |
| 369 | Encoding Probe 359 | TGTGATGGAAGTTAGAGGGTGCGTCACCCCATTA |
| | | AGAGGCTAGGATAGGAAATGGTGGTAGTGT |
| 370 | Encoding Probe 360 | TGTGATGGAAGTTAGAGGGTCATGTCAATGAGCA |
| | | AAGGTATTATGAATAGGAAATGGTGGTAGTGT |
| 371 | Encoding Probe 361 | TGTGATGGAAGTTAGAGGGTTAGGCTCACAATAT |
| | | GTGCATTAAAATAGGAAATGGTGGTAGTGT |
| 372 | Encoding Probe 362 | TGTGATGGAAGTTAGAGGGTTGACACACACACTG |
| | | ATTCAGGGAGATAGGAAATGGTGGTAGTGT |
| 373 | Encoding Probe 363 | TGTGATGGAAGTTAGAGGGTCCTCAGTTAATGAT |
| | | AGTGTGTCGTTTATAGGAAATGGTGGTAGTGT |
| 374 | Encoding Probe 364 | TGTGATGGAAGTTAGAGGGTGTTGTGAACAAACT |
| | | TTCGACTACTCCAGAGTGAGTAGTAGTGGAGT |
| 375 | Encoding Probe 365 | TGTGATGGAAGTTAGAGGGTGGACGCTTAAAAC |
| | | GAATAATGGTGGATGAGAGTGAGTAGTAGTGGA |
| | | GT |
| 376 | Encoding Probe 366 | TGTGATGGAAGTTAGAGGGTGTACTTAAAACGAA |
| | | TAATGGTGGTAGTCAGAGTGAGTAGTAGTGGAGT |
| 377 | Encoding Probe 367 | TGTGATGGAAGTTAGAGGGTGGTGTCCTTACGGA |
| | | CAATCCAGTCAGAGTGAGTAGTAGTGGAGT |
| 378 | Encoding Probe 368 | TGTGATGGAAGTTAGAGGGTCAGACTCTTGCGGA |
| | | ACGTAAGAGGAGAGTGAGTAGTAGTGGAGT |
| 379 | Encoding Probe 369 | TGTGATGGAAGTTAGAGGGTTTGCTCGAGGAAAC |
| | | AATTTCCAGAAGAGTGAGTAGTAGTGGAGT |
| 380 | Encoding Probe 370 | TGTGATGGAAGTTAGAGGGTCGACTCCATAAATG |
| | | GTTACTCCACGCCAGAGTGAGTAGTAGTGGAGT |
| 381 | Encoding Probe 371 | TGTGATGGAAGTTAGAGGGTTAACCTAACACTCA |
| | | ATCTCACTGCTTCCTAGAGTGAGTAGTAGTGGAG |
| | | T |
| 382 | Encoding Probe 372 | TGTGATGGAAGTTAGAGGGTTTAGGTAACCCGAT |
| | | AAGGGCCGGAAGAGTGAGTAGTAGTGGAGT |
| 383 | Encoding Probe 373 | TGTGATGGAAGTTAGAGGGTCTCAGCTCCTTATC |
| | | TGTTCGCTGCTAGAGTGAGTAGTAGTGGAGT |
| 384 | Encoding Probe 374 | TGTGATGGAAGTTAGAGGGTCACCTCCTTGCCAT |
| | | TGTCACCAATAAGAGTGAGTAGTAGTGGAGT |
| 385 | Encoding Probe 375 | TGTGATGGAAGTTAGAGGGTGCTCGAGGAAACA |
| | | ATTTCCTCAGGAGAGTGAGTAGTAGTGGAGT |
| 386 | Encoding Probe 376 | TGTGATGGAAGTTAGAGGGTAATTACACGTTTGT |
| | | TCTTCCCATTAGAGTGAGTAGTAGTGGAGT |
| 387 | Encoding Probe 377 | TGTGATGGAAGTTAGAGGGTGTGAAGAGTGAAC |
| | | AAACTTTCGTGAAGAGTGAGTAGTAGTGGAGT |
| 388 | Encoding Probe 378 | TGTGATGGAAGTTAGAGGGTTACTCGTCTAGTCT |
| | | GTTCTTTTGTAAGAGAGAGTGAGTAGTAGTGGAG |
| | | T |
| 389 | Encoding Probe 379 | TGTGATGGAAGTTAGAGGGTAGGCGCTAACGTCA |
| | | AAGGAGCTTCAGAGTGAGTAGTAGTGGAGT |
| 390 | Encoding Probe 380 | TGTGATGGAAGTTAGAGGGTGATAGTGATAGCA |
| | | AAACCATCTTTCTGAAGAGTGAGTAGTAGTGGAG |
| | | T |
| 391 | Encoding Probe 381 | TGTGATGGAAGTTAGAGGGTTCGGCTCCTTATCT |
| | | GTTCGCTCCTGAGAGTGAGTAGTAGTGGAGT |
| 392 | Encoding Probe 382 | TGTGATGGAAGTTAGAGGGTCTCAGCTCCTTATC |
| | | TGTTCGCAGCAGAGTGAGTAGTAGTGGAGT |
| 393 | Encoding Probe 383 | TGTGATGGAAGTTAGAGGGTCACCTCCTTGCCAT |
| | | TGTCACCTTAAGAGTGAGTAGTAGTGGAGT |
| 394 | Encoding Probe 384 | TGTGATGGAAGTTAGAGGGTGGACACTCAATCTC |
| | | ACTGCTTCCTAGAGTGAGTAGTAGTGGAGT |
| 395 | Encoding Probe 385 | TGTGATGGAAGTTAGAGGGTGCCACCACAATTCT |
| | | AGCTAGAGCGAAGAGTGAGTAGTAGTGGAGT |
| 396 | Encoding Probe 386 | TGTGATGGAAGTTAGAGGGTCGGACACTCAATCT |
| | | CACTGCTACCAGAGTGAGTAGTAGTGGAGT |
| 397 | Encoding Probe 387 | TGTGATGGAAGTTAGAGGGTATCCTCACGTATCT |
| | | CAGGCTCCATGAGAGTGAGTAGTAGTGGAGT |
| 398 | Encoding Probe 388 | TGTGATGGAAGTTAGAGGGTAAGCAGCTGCACAT |
| | | ATCGCTAACGAGAGTGAGTAGTAGTGGAGT |

### Example 2.3

FIG. 5 shows the ability of the HiPR-FISH to differentiate drug-resistant from drug-susceptible microbes in a sample. The following methodology was employed. Carbapenem-resistant or -susceptible Pseudomonas aeruginosa were cultured in liquid tryptic soy broth for several passages. An 8-well device was constructed where each well was filled with 25 μL of tryptic soy agar at 42° C. with various concentrations of meropenem and allowed to dry to create a growth pad for bacteria. For both resistant and susceptible cultures, 1 μL of culture suspension was deposited at the center of each pad and the liquid was allowed to dry/absorb. A #1 coverslip was used to seal the bottom of the device. The device was placed on a custom built stage that enabled temperature regulation on a Nikon TiE widefield epifluorescence microscope. The custom stage contained a chamber that used two Peltier units to keep the bottom of the stage at 37° C. for incubation and the top at 42° C. to prevent condensation. A 40×phase contrast objective was used to continuously image a single field of view in each well of the device every 30 seconds for two hours. At the conclusion of the experiment, 2% formaldehyde was added to each well to fix colonies for down stream assays.

### Example 3. Identification of Fungi

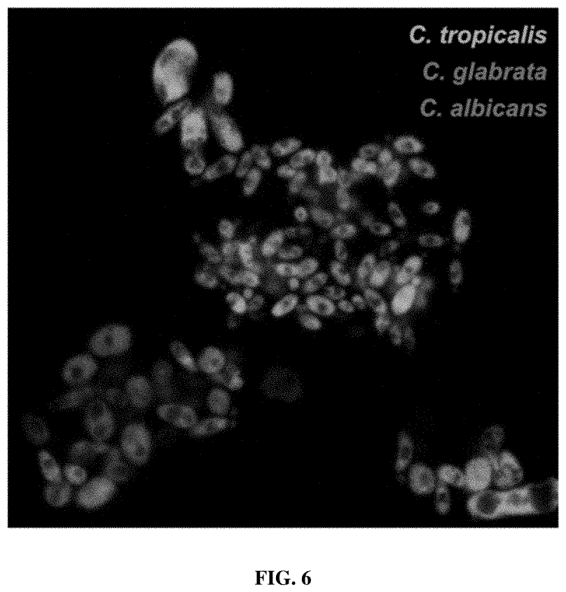

FIG. 6 shows the identification of different fungi species including C. tropicalis, C. glabrata, and C. albicans, using the following methodology.

Suspensions of individual monocultures were fixed by adding an equal volume of 2% formaldehyde, mixing, and incubating for 90 minutes at room temperature. Fixed cultures were then washed with 1×PBS and resuspended in 50% ethanol. Suspensions were deposited onto glass microscope slides until 50% ethanol had evaporated. Zymolysae (5 U per mL in a buffer with 1.2 M sorbitol and 0.1 M potassium phosphate buffer, pH 7.5) was deposited onto each dry specimen to permeabilize the outer membrane and incubated for 90 minutes at 30° C., the slides were then washed with 1×PBS. An encoding probe hybridization buffer (2×SSC, 10% dextran sulfate, 10% ethylene carbonate, 5×Denhardt's solution, 0.01% SDS) with probes designed for the fungal species (at roughly 200 nM) was deposited on cells and incubated for two hours at 37° C. A wash buffer (5 mM EDTA, 20 mM Tris HCl, 215 mM NaCl) was then deposited on specimens for fifteen minutes at 37° C. to remove unbound probes. A buffer containing readout probes (10 readout probes, each at 400 nM; buffer made up of 2×SSC, 10% dextran sulfate, 10% ethylene carbonate, 5×Denhardt's solution, 0.01% SDS) was incubated for one hour at room temperature. A second round of wash buffer was deposited on specimens for fifteen minutes at 37° C. to remove unbound probes. The specimens were mounted with Prolong Glass and a coverslip was placed directly over the specimens for imaging on a confocal microscope.

Table 3 shows the sequences of the encoding probes used in this example. The readout probes are shown in Table 1.

TABLE 3
Encoding probes used in Example 3

| SEQ ID | | |
|---|---|---|
| NO: | Probe Name | Sequence |
| 399 | Encoding Probe 389 | AGGGTGTGTTTGTAAAGGGTACTTCCCCGTGGTTG |
| | | AGTCAAAAAT |
| 400 | Encoding Probe 390 | TGTGGAGGGATTGAAGGATAACTTCCCCGTGGTT |
| | | GAGTCAAAAAT |
| 401 | Encoding Probe 391 | AGGGTGTGTTTGTAAAGGGTACCCCAGACTTGGC |
| | | CTTCCAATTGTAGG |
| 402 | Encoding Probe 392 | TGTGGAGGGATTGAAGGATAACCCCAGACTTGGC |
| | | CTTCCAATTGTAGG |
| 403 | Encoding Probe 393 | AGGGTGTGTTTGTAAAGGGTGAGTTCCAGAATGA |
| | | GGTTGCCAGG |
| 404 | Encoding Probe 394 | TGTGGAGGGATTGAAGGATAGAGTTCCAGAATGA |
| | | GGTTGCCAGG |
| 405 | Encoding Probe 395 | AGGGTGTGTTTGTAAAGGGTAGAGTTCCAGAATG |
| | | AGGTTGCCAGG |
| 406 | Encoding Probe 396 | TGTGGAGGGATTGAAGGATAAGAGTTCCAGAATG |
| | | AGGTTGCCAGG |
| 407 | Encoding Probe 397 | AGGGTGTGTTTGTAAAGGGTAGGGTTCGCCATAA |
| | | ATGGCTACCGTC |
| 408 | Encoding Probe 398 | TGTGGAGGGATTGAAGGATAAGGGTTCGCCATAA |
| | | ATGGCTACCGTC |
| 409 | Encoding Probe 399 | AGGGTGTGTTTGTAAAGGGTTGACATCGACTTGG |
| | | AGTCGATTCA |
| 410 | Encoding Probe 400 | TGTGGAGGGATTGAAGGATATGACATCGACTTGG |
| | | AGTCGATTCA |
| 411 | Encoding Probe 401 | AGGGTGTGTTTGTAAAGGGTCGTTGACTACTGGC |
| | | AGGATCAACCACTA |
| 412 | Encoding Probe 402 | TGTGGAGGGATTGAAGGATACGTTGACTACTGGC |
| | | AGGATCAACCACTA |
| 413 | Encoding Probe 403 | AGGGTGTGTTTGTAAAGGGTACTTCCCCGTGGTTG |
| | | AGTCAATAA |
| 414 | Encoding Probe 404 | TGTGGAGGGATTGAAGGATAACTTCCCCGTGGTT |
| | | GAGTCAATAA |
| 415 | Encoding Probe 405 | AGGGTGTGTTTGTAAAGGGTGGATTCGCCATAAA |
| | | TGGCTACCGTC |
| 416 | Encoding Probe 406 | TGTGGAGGGATTGAAGGATAGGATTCGCCATAAA |
| | | TGGCTACCGTC |
| 417 | Encoding Probe 407 | AGGGTGTGTTTGTAAAGGGTGTAACTTGGAGTCG |
| | | ATAGTCCCAGA |
| 418 | Encoding Probe 408 | TGTGGAGGGATTGAAGGATAGTAACTTGGAGTCG |
| | | ATAGTCCCAGA |
| 419 | Encoding Probe 409 | AGGGTGTGTTTGTAAAGGGTTCGATGACTACTGG |
| | | CAGGATCAACCACTA |
| 420 | Encoding Probe 410 | TGTGGAGGGATTGAAGGATATCGATGACTACTGG |
| | | CAGGATCAACCACTA |
| 421 | Encoding Probe 411 | AGGGTGTGTTTGTAAAGGGTAGTACCTCCCCTGA |
| | | ATCGGGATTCCC |
| 422 | Encoding Probe 412 | TGTGGAGGGATTGAAGGATAAGTACCTCCCCTGA |
| | | ATCGGGATTCCC |
| 423 | Encoding Probe 413 | AGAGTGAGTAGTAGTGGAGTAACTTGCTTTTCTTC |
| | | CTCTAATGACCTTC |
| 424 | Encoding Probe 414 | TTGGAGGTGTAGGGAGTAAAAACTTGCTTTTCTTC |
| | | CTCTAATGACCTTC |
| 425 | Encoding Probe 415 | AGAGTGAGTAGTAGTGGAGTACGTGCTTTTCTTCC |
| | | TCTAATGACCATCA |
| 426 | Encoding Probe 416 | TTGGAGGTGTAGGGAGTAAAACGTGCTTTTCTTCC |
| | | TCTAATGACCATCA |
| 427 | Encoding Probe 417 | AGAGTGAGTAGTAGTGGAGTCGAGCTTTTCTTCCT |
| | | CTAATGACCAACAA |
| 428 | Encoding Probe 418 | TTGGAGGTGTAGGGAGTAAACGAGCTTTTCTTCCT |
| | | CTAATGACCAACAA |
| 429 | Encoding Probe 419 | AGAGTGAGTAGTAGTGGAGTTGTCATGGCTAATC |
| | | TAGCGGGTTA |
| 430 | Encoding Probe 420 | TTGGAGGTGTAGGGAGTAAATGTCATGGCTAATC |
| | | TAGCGGGTTA |
| 431 | Encoding Probe 421 | AGAGTGAGTAGTAGTGGAGTCTGGCATGGCTAAT |
| | | CTAGCGGCTA |
| 432 | Encoding Probe 422 | TTGGAGGTGTAGGGAGTAAACTGGCATGGCTAAT |
| | | CTAGCGGCTA |
| 433 | Encoding Probe 423 | AGAGTGAGTAGTAGTGGAGTGGATTCGCCAAAAG |
| | | GCTAGCCAGTTC |
| 434 | Encoding Probe 424 | TTGGAGGTGTAGGGAGTAAAGGATTCGCCAAAAG |
| | | GCTAGCCAGTTC |
| 435 | Encoding Probe 425 | AGAGTGAGTAGTAGTGGAGTCTGGCATGGCTAAT |
| | | CTAGCGGGAATA |
| 436 | Encoding Probe 426 | TTGGAGGTGTAGGGAGTAAACTGGCATGGCTAAT |
| | | CTAGCGGGAATA |
| 437 | Encoding Probe 427 | AGAGTGAGTAGTAGTGGAGTACCCGCCAAAAGGC |
| | | TAGCCAGAACCT |
| 438 | Encoding Probe 428 | TTGGAGGTGTAGGGAGTAAAACCCGCCAAAAGGC |
| | | TAGCCAGAACCT |
| 439 | Encoding Probe 429 | AGAGTGAGTAGTAGTGGAGTTCTTGCATGGCTAA |
| | | TCTAGCGGGAGTT |
| 440 | Encoding Probe 430 | TTGGAGGTGTAGGGAGTAAATCTTGCATGGCTAA |
| | | TCTAGCGGGAGTT |
| 441 | Encoding Probe 431 | AGAGTGAGTAGTAGTGGAGTCTGGCATGGCTAAT |
| | | CTAGCGGGTTA |
| 442 | Encoding Probe 432 | TTGGAGGTGTAGGGAGTAAACTGGCATGGCTAAT |
| | | CTAGCGGGTTA |
| 443 | Encoding Probe 433 | AGAGTGAGTAGTAGTGGAGTTGTCATGGCTAATC |
| | | TAGCGGGAATA |
| 444 | Encoding Probe 434 | TTGGAGGTGTAGGGAGTAAATGTCATGGCTAATC |
| | | TAGCGGGAATA |
| 445 | Encoding Probe 435 | AGAGTGAGTAGTAGTGGAGTAGGGTTCGCCAAAA |
| | | GGCTAGCGTC |
| 446 | Encoding Probe 436 | TTGGAGGTGTAGGGAGTAAAAGGGTTCGCCAAAA |
| | | GGCTAGCGTC |
| 447 | Encoding Probe 437 | TGTGGAGGGATTGAAGGATATATTCTCTTCCAAG |
| | | AGGTCGAGATTTATT |
| 448 | Encoding Probe 438 | TTGGAGGTGTAGGGAGTAAATATTCTCTTCCAAG |
| | | AGGTCGAGATTTATT |
| 449 | Encoding Probe 439 | TGTGGAGGGATTGAAGGATAGAGATTACCGCGGG |
| | | CTGCTGGGTG |
| 450 | Encoding Probe 440 | TTGGAGGTGTAGGGAGTAAAGAGATTACCGCGGG |
| | | CTGCTGGGTG |
| 451 | Encoding Probe 441 | TGTGGAGGGATTGAAGGATAGTCTCTCCGCTCTG |
| | | AAGTGGAGTCCGG |
| 452 | Encoding Probe 442 | TTGGAGGTGTAGGGAGTAAAGTCTCTCCGCTCTG |
| | | AAGTGGAGTCCGG |
| 453 | Encoding Probe 443 | TGTGGAGGGATTGAAGGATAAAAGTACACGAAAA |
| | | AATCGGACCGGAGT |
| 454 | Encoding Probe 444 | TTGGAGGTGTAGGGAGTAAAAAAGTACACGAAAA |
| | | AATCGGACCGGAGT |
| 455 | Encoding Probe 445 | TGTGGAGGGATTGAAGGATAGTACAGTACACGAA |
| | | AAAATCGGACCGCGG |
| 456 | Encoding Probe 446 | TTGGAGGTGTAGGGAGTAAAGTACAGTACACGAA |
| | | AAAATCGGACCGCGG |
| 457 | Encoding Probe 447 | TGTGGAGGGATTGAAGGATAGTGCCTCCCTGTGT |
| | | CAGGATTCCC |
| 458 | Encoding Probe 448 | TTGGAGGTGTAGGGAGTAAAGTGCCTCCCTGTGT |
| | | CAGGATTCCC |
| 459 | Encoding Probe 449 | TGTGGAGGGATTGAAGGATAGTGCCTCCCTGTGT |
| | | CAGGATTGCCA |
| 460 | Encoding Probe 450 | TTGGAGGTGTAGGGAGTAAAGTGCCTCCCTGTGT |
| | | CAGGATTGCCA |
| 461 | Encoding Probe 451 | TGTGGAGGGATTGAAGGATACATGTGCCGAGTGG |
| | | GTCACTAATTT |
| 462 | Encoding Probe 452 | TTGGAGGTGTAGGGAGTAAACATGTGCCGAGTGG |
| | | GTCACTAATTT |
| 463 | Encoding Probe 453 | TGTGGAGGGATTGAAGGATACTCGGTCACTAAAA |
| | | AAACACCACCCGTAG |
| 464 | Encoding Probe 454 | TTGGAGGTGTAGGGAGTAAACTCGGTCACTAAAA |
| | | AAACACCACCCGTAG |
| 465 | Encoding Probe 455 | TGTGGAGGGATTGAAGGATAAGAGCCAAGGTTAG |
| | | ACTCGCTGCGA |
| 466 | Encoding Probe 456 | TTGGAGGTGTAGGGAGTAAAAGAGCCAAGGTTAG |
| | | ACTCGCTGCGA |
| 467 | Encoding Probe 457 | TGTGGAGGGATTGAAGGATACTGGCATGGCTTAA |
| | | TTTTTAGACAAAATG |
| 468 | Encoding Probe 458 | TTGGAGGTGTAGGGAGTAAACTGGCATGGCTTAA |
| | | TTTTTAGACAAAATG |
| 469 | Encoding Probe 459 | TGTGGAGGGATTGAAGGATATTAATCTCTTCCAA |
| | | GAGGTCGAGATTAAT |
| 470 | Encoding Probe 460 | TTGGAGGTGTAGGGAGTAAATTAATCTCTTCCAA |
| | | GAGGTCGAGATTAAT |

### Example 4. HiPR-FAST One Pot

Escherichia coli (E. coli) cells were cultured at 30° C. for several passages prior to the start of the experiment. At experiment passage, cultured E. coli were grown in suspension at 30° C. ambient temperature for ninety minutes. Then, their vessel was sealed and placed in a water bath at 46° C. for five minutes. Immediately following the heat shock, the vessel was placed on ice for one minute before a volume of 2% formaldehyde (equal to the volume of the suspension) was added to the suspension and mixed for suspension. Fixing cells were incubated at room temperature for one hour. After one hour, fixed cells were washed with 1×PBS and resuspended in 50% ethanol. A small volume (0.75 μL) was deposited on a glass slide and allowed to dry. The deposition was then rehydrated with 10 mg/ml lysozyme and placed at 37° C. for 15 minutes to encourage permeabilization. Cells were then washed with 1×PBS for ten minutes at room temperature. A hybridization buffer (2×SSC, 10% dextran sulfate, 10% ethylene carbonate, 5×Denhardt's solution, 0.01% SDS) containing rRNA (1 μM per species) and mRNA (1 μM per gene) was added to cells and the slide was placed at 37° C. for one hour. Immediately following hybridization, cells were incubated in wash buffer (5 mM EDTA, 20 mM Tris HCl, 215 mM NaCl) for 15 minutes at 48° C. Finally, the wash buffer was removed and the cells were mounted with Prolong Glass under a #1 coverslip for imaging.

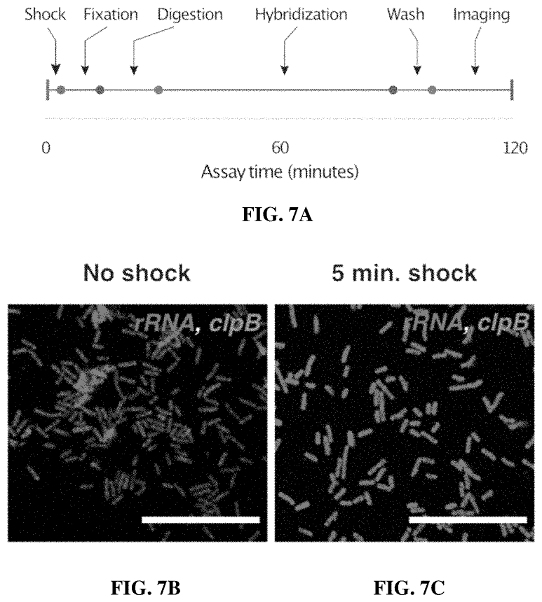

FIGS. 7A-7C shows gene expression measurement enable rapid detection of stress response in HiPR-FISH. Table 4 shows the sequences of the encoding probes used in this example. The readout probes are shown in Table 1.

TABLE 4
Encoding Probes used in Example 4

| SEQ | | | |
|---|---|---|---|
| ID | Tar- | Probe | |
| NO: | get | Name | Sequence (in 5′ to 3′ order) |
| 471 | rRNA | Encoding | AGGGTGTGTTTGTAAAGGGTCCTCAGTTAAT |
| | | Probe 461 | GATAGTGTGTCGATTG |
| 472 | rRNA | Encoding | ATAGGAAATGGTGGTAGTGTGGAGCCTTGG |
| | | Probe 462 | TTTTCCGGATTACG |
| 473 | rRNA | Encoding | AGGGTGTGTTTGTAAAGGGTGTGTCTCATCT |
| | | Probe 463 | CTGAAAACTTCCCAC |
| 474 | rRNA | Encoding | ATAGGAAATGGTGGTAGTGTGTCACCCCATT |
| | | Probe 464 | AAGAGGCTCCGTG |
| 475 | rRNA | Encoding | AGGGTGTGTTTGTAAAGGGTCCACGTCAATG |
| | | Probe 465 | AGCAAAGGTAAAT |
| 476 | rRNA | Encoding | ATAGGAAATGGTGGTAGTGTGTAAGCTCAC |
| | | Probe 466 | AATATGTGCATAAA |
| 477 | rRNA | Encoding | AGGGTGTGTTTGTAAAGGGTGATACACACA |
| | | Probe 467 | CTGATTCAGGCAGA |
| 478 | rRNA | Encoding | ATAGGAAATGGTGGTAGTGTAGTCTTGGTTT |
| | | Probe 468 | TCCGGATTTGGGA |
| 479 | rRNA | Encoding | AGGGTGTGTTTGTAAAGGGTACCTCAGTTAA |
| | | Probe 469 | TGATAGTGTGTCGTTT |
| 480 | rRNA | Encoding | ATAGGAAATGGTGGTAGTGTGAGCCTTGGTT |
| | | Probe 470 | TTCCGGATTTCGG |
| 481 | rRNA | Encoding | AGGGTGTGTTTGTAAAGGGTGTATCATCTCT |
| | | Probe 471 | GAAAACTTCCGACC |
| 482 | rRNA | Encoding | ATAGGAAATGGTGGTAGTGTGTGCTCAGCCT |
| | | Probe 472 | TGGTTTTCCGCTA |
| 483 | rRNA | Encoding | AGGGTGTGTTTGTAAAGGGTTGCGTCACCCC |
| | | Probe 473 | ATTAAGAGGCAGG |
| 484 | rRNA | Encoding | ATAGGAAATGGTGGTAGTGTCATGTCAATG |
| | | Probe 474 | AGCAAAGGTATTAAGAA |
| 485 | rRNA | Encoding | AGGGTGTGTTTGTAAAGGGTGTAAGCTCAC |
| | | Probe 475 | AATATGTGCATTAAA |
| 486 | rRNA | Encoding | ATAGGAAATGGTGGTAGTGTGAAACTAACA |
| | | Probe 476 | CACACACTGATTGTC |
| 487 | rRNA | Encoding | AGGGTGTGTTTGTAAAGGGTCTAAGTTAATG |
| | | Probe 477 | ATAGTGTGTCGATTG |
| 488 | rRNA | Encoding | ATAGGAAATGGTGGTAGTGTGTGTCTCATCT |
| | | Probe 478 | CTGAAAACTTCCGACC |
| 489 | rRNA | Encoding | AGGGTGTGTTTGTAAAGGGTAGGAAGGCAC |
| | | Probe 479 | ATTCTCATCTCACT |
| 490 | rRNA | Encoding | ATAGGAAATGGTGGTAGTGTCGTCACCCCAT |
| | | Probe 480 | TAAGAGGCTCGGT |
| 491 | rRNA | Encoding | AGGGTGTGTTTGTAAAGGGTGCGTCACCCCA |
| | | Probe 481 | TTAAGAGGCTAGG |
| 492 | rRNA | Encoding | ATAGGAAATGGTGGTAGTGTCATGTCAATG |
| | | Probe 482 | AGCAAAGGTATTATGA |
| 493 | rRNA | Encoding | AGGGTGTGTTTGTAAAGGGTTAGGCTCACA |
| | | Probe 483 | ATATGTGCATTAAA |
| 494 | rRNA | Encoding | ATAGGAAATGGTGGTAGTGTTGACACACAC |
| | | Probe 484 | ACTGATTCAGGGAG |
| 495 | rRNA | Encoding | AGGGTGTGTTTGTAAAGGGTCCTCAGTTAAT |
| | | Probe 485 | GATAGTGTGTCGTTT |
| 496 | mRNA | Encoding | CGTCGGAGTGGGTTCAGTCTATCATCGCCAG |
| | | Probe 486 | CGCCTTACAAAGCTCT |
| 497 | mRNA | Encoding | GATGATGTAGTAGTAAGGGTCGGTTCGAGC |
| | | Probe 487 | TGCGTTGCGGCTTCCA |
| 498 | mRNA | Encoding | GATGATGTAGTAGTAAGGGTAAGACCACGC |
| | | Probe 488 | GCCAGTGCAGGTTTCA |
| 499 | mRNA | Encoding | GATGATGTAGTAGTAAGGGTGCACGCTGCT |
| | | Probe 489 | GCAACAATTGCCGGGT |
| 500 | mRNA | Encoding | GATGATGTAGTAGTAAGGGTGTCTACGCGG |
| | | Probe 490 | CGGCCTTTCAACCCTT |
| 501 | mRNA | Encoding | GATGATGTAGTAGTAAGGGTTTAACGCTGC |
| | | Probe 491 | GCCAGACCTTCAACGA |
| 502 | mRNA | Encoding | GATGATGTAGTAGTAAGGGTCAGAAGCGTC |
| | | Probe 492 | GTGGCACCTACGCAGT |
| 503 | mRNA | Encoding | GATGATGTAGTAGTAAGGGTCAGACCCACA |
| | | Probe 493 | CGGCGAGCCTGTTCAA |
| 504 | mRNA | Encoding | GATGATGTAGTAGTAAGGGTCGCACGACGC |
| | | Probe 494 | ACCGCTTCGGTCAGAT |
| 505 | mRNA | Encoding | GATGATGTAGTAGTAAGGGTAAAATCCGGC |
| | | Probe 495 | AGCTGACGGTCAGCAA |
| 506 | mRNA | Encoding | GATGATGTAGTAGTAAGGGTAAATCGCGCT |
| | | Probe 496 | CGCTTTCCATCATGCG |
| 507 | mRNA | Encoding | GATGATGTAGTAGTAAGGGTGAATACCCGC |
| | | Probe 497 | GCCGACCATGGTATGT |
| 508 | mRNA | Encoding | GATGATGTAGTAGTAAGGGTCTATGGGCAT |
| | | Probe 498 | CGGCAAGAGCAAGCTG |
| 509 | mRNA | Encoding | GATGATGTAGTAGTAAGGGTACGTTTCAGG |
| | | Probe 499 | ATGTCGGCCAGCGTGC |
| 510 | mRNA | Encoding | GATGATGTAGTAGTAAGGGTAGCATACGGA |
| | | Probe 500 | CCATCGCCTCGTCGCT |
| 511 | mRNA | Encoding | GATGATGTAGTAGTAAGGGTGCATGCAGCA |
| | | Probe 501 | CCTGAATGGTACGGCG |
| 512 | mRNA | Encoding | GATGATGTAGTAGTAAGGGTTTATTGCACAT |
| | | Probe 502 | GGTGGTGCAGCTCGT |
| 513 | mRNA | Encoding | GATGATGTAGTAGTAAGGGTCTTTTGGTTGT |
| | | Probe 503 | CGTGCCCGAGTGCAA |
| 514 | mRNA | Encoding | GATGATGTAGTAGTAAGGGTAACAGTAATG |
| | | Probe 504 | TTGGCGGTGGTCGCCC |
| 515 | mRNA | Encoding | GATGATGTAGTAGTAAGGGTGGTATCGGGC |
| | | Probe 505 | GATTTGGATCCGCCAG |
| 516 | mRNA | Encoding | GATGATGTAGTAGTAAGGGTTCATTGCCTTG |
| | | Probe 506 | TTCGGCTCGTTCGGT |
| 517 | mRNA | Encoding | GATGATGTAGTAGTAAGGGTTTGGCGCACC |
| | | Probe 507 | AGATCCTGTGATGGCT |
| 518 | mRNA | Encoding | GATGATGTAGTAGTAAGGGTTACGCTTACCA |
| | | Probe 508 | CACCGAGCACCAGCT |
| 519 | mRNA | Encoding | GATGATGTAGTAGTAAGGGTCTACGTTCACG |
| | | Probe 509 | CTTTCACCTCCACGC |
| 520 | mRNA | Encoding | GATGATGTAGTAGTAAGGGTGTGCCGTTGG |
| | | Probe 510 | GCCGAGGAACAGGAAT |
| 521 | mRNA | Encoding | GATGATGTAGTAGTAAGGGTCCGGCACCAA |
| | | Probe 511 | CCAGACGAGACACCGA |
| 522 | mRNA | Encoding | GATGATGTAGTAGTAAGGGTGACGACGGCG |
| | | Probe 512 | ACGATCCGGTCTTCAT |
| 523 | mRNA | Encoding | GATGATGTAGTAGTAAGGGTATTCGCGATG |
| | | Probe 513 | GTGCAGTTCTTGCTCC |
| 524 | mRNA | Encoding | GATGATGTAGTAGTAAGGGTGACGAAACGA |
| | | Probe 514 | CGTTCCAGCGCAGCAT |

### Example 5. HiPR Swap

DNA exchange was used as a method to quickly, specifically, carefully replace the HiPR-FISH readout probes without disturbing encoding and/or amplifier probes. This method is referred to as HiPR-Swap.

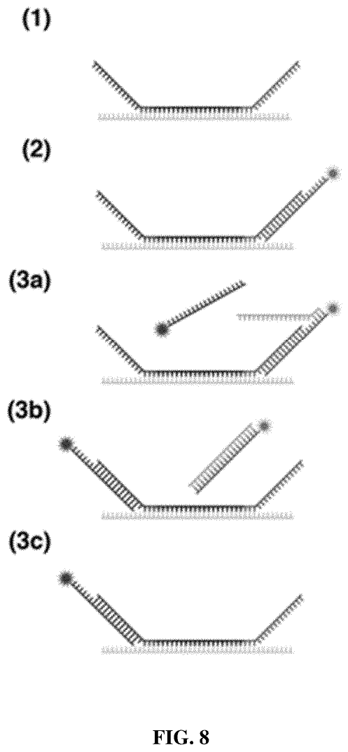

In the HiPR-Swap method, readout and encoding probes are designed such that the “landing pad” (the region on the encoding probe to which the readout probe binds) is complementary to the readout probe. In some instances, the landing pad sequence is shorter than the readout probe. This would create a single-stranded overhang of the readout probe, as it extends past the end of the landing pad (see FIG. 8, line (2)).

After a readout probe is bound, an exchange probe can be added to the specimen. The exchange probe is constructed to be of equal length and a perfect reverse complement to the readout probe. When added, the exchange probe seeds a hybridization to the exposed area of the readout probe (see FIG. 8, line 3a). Over a short period of time the exchange probe completely hybridizes to the readout probe, thereby removing it from the encoding probe where it can be washed away. Importantly, orthogonal (non-interacting at room temperature to 42° C.) readout and exchange probes can be added simultaneously to reduce assay time (see FIG. 8, lines 3b and 3c.)

In theory, there is no limit to the number of times the assay can be performed. The maximum number of probes needed is the number of fluorescent probes observable in a single round (for example, 10) multiplied by the number of rounds. For example, if 4 rounds are performed, this will require 40 unique probes each bound with one of 10 fluorescent dyes. This would allow the target multiplexity to be (2\hat{ }(10)−1)\hat{ }4=1,095,222,947,841 targets.

Advantages

Thermodynamics models can be applied to understand the extent to which probe swapping is likely to succeed. For example, the Boltzmann factor can be naively implemented to illustrate the improved likelihood of the readout-exchange probe duplex over the readout-encoding probe duplex (false assumption that the system is at equilibrium).

The probability of being in a state is given by the distribution:

$$P ( {ε}_{n} ) = \frac{1}{Z} {e}^{- {ε}_{n} / {k}_{B} T} .$$

Knowing this, one can find the Boltzmann factor as the ratio of probabilities (P(readout-exchange)/P(readout-encoding)).

The Boltzmann factor for various combinations of the readout probes was determined, where the overhang can be 1 to 5 nt from the 3′ or 5′ end (yellow or blue, respectively) and found that the likelihood of being in the readout-exchange state increases dramatically as the length of the overhang increases; the Boltzmann factor can exceed 10,000×.

HiPR-Swap, in combination with other technologies, will create a FISH-based assay with the highest multiplexity yet achieved. Its application to spectral barcoding and classification, to the study of microbiomes and bacteria, and its use to profile rRNA and mRNA (and potentially other analytes) make this method an improvement over the prior art.

### Example 6. HiPR-Swap

An experiment was performed where three species of bacteria (Escherichia coli, Pseudomonas aeruginosa, and Klebsiella pneumoniae) were encoded with 18-24 encoding probes, with 15-nt landing pads. Each species was encoded such that they were hybridized with a single, unique bit (or dye).

The experiment was performed to (1) show the addition of exchange probes removes readout probes (and thereby fluorescence signal) and (2) following the exchange, new readout probes can be re-hybridized to the specimens without the addition of new encoding probes.

The procedure was as follows: Cells were adhered to a coverslip via evaporation. Each species of bacteria was separated from the others using a gasket. The cells were then digested with lysozyme at 37° C. for 30 minutes and washed with 1×PBS at room temperature for 15 minutes. The encoding probe hybridization and readout probe hybridization were performed in a single step. The hybridization buffer was prepared separately for each species (10% ethylene carbonate, 10% dextran sulfate, 2×SSC, 5×Denhardt's solution, 0.01% SDS, 400 nM of the readout probe, 2 uM per taxa of the encoding probes). The hybridization buffer was then added to the cells at 37° C. for 2 hours. The wash buffer (215 mM NaCl, 20 mM Tris-HCl (pH 8.0), and 5 mM EDTA) was then added to the cells at 30° C. for 15 minutes. The cells were imaged in the wash buffer. The cells were removed from the scope. The exchange buffer was then added to the cells at 37° C. and left overnight. The exchange buffer was prepared separately for each species (10% ethylene carbonate, 10% dextran sulfate, 2×SSC, 5×Denhardt's solution, 0.01% SDS, and 2 uM of the exchange probe). The wash buffer was added to the cells at 30° C. for 15 minutes. The cells were imaged in the wash buffer. The readout buffer (prepared separately for each species: 10% ethylene carbonate, 10% dextran sulfate, 2×SSC, 5×Denhardt's solution, 0.01% SDS, and 400 nM of the readout probe) was added to the cells and incubated at 37° C. for 2 hours. The wash buffer was added to the cells at 30° C. for 15 minutes. The cells were imaged in the wash buffer. The cells were removed from the scope and stored at 4° C.

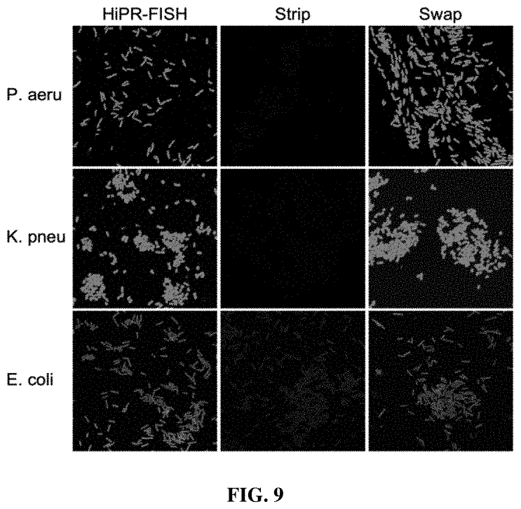

As evidenced by FIG. 9, the results of the experiment showed that each species was properly encoded after the first round (“HiPR-FISH”). Further, each species had their fluorescence signal, and thus readout probes, removed with exchange probes (“Strip”), and the original signal was fully recovered (with correct encoding), with addition of readout probes after the Strip (“Swap”).

The encoding, readout, and exchange probes used in this example are shown in Table 5 below.

TABLE 5
Encoding, readout, and exchange probes used in Example 6.

| SEQ ID NO: | Probe Name | Sequence |
|---|---|---|
| 525 | Encoding Probe 515 | TGGAAGTTAGAGGGTCCTCAGTTAATGATAGT |
| | | GTGTCGATTG |
| 526 | Encoding Probe 516 | TGGAAGTTAGAGGGTGGAGCCTTGGTTTTCCG |
| | | GATTACG |
| 527 | Encoding Probe 517 | TGGAAGTTAGAGGGTGTGTCTCATCTCTGAAA |
| | | ACTTCCCAC |
| 528 | Encoding Probe 518 | TGGAAGTTAGAGGGTGTCACCCCATTAAGAG |
| | | GCTCCGTG |
| 529 | Encoding Probe 519 | TGGAAGTTAGAGGGTCCACGTCAATGAGCAA |
| | | AGGTAAAT |
| 530 | Encoding Probe 520 | TGGAAGTTAGAGGGTGTAAGCTCACAATATGT |
| | | GCATAAA |
| 531 | Encoding Probe 521 | TGGAAGTTAGAGGGTGATACACACACTGATTC |
| | | AGGCAGA |
| 532 | Encoding Probe 522 | TGGAAGTTAGAGGGTAGTCTTGGTTTTCCGGA |
| | | TTTGGGA |
| 533 | Encoding Probe 523 | TGGAAGTTAGAGGGTACCTCAGTTAATGATAG |
| | | TGTGTCGTTT |
| 534 | Encoding Probe 524 | TGGAAGTTAGAGGGTGAGCCTTGGTTTTCCGG |
| | | ATTTCGG |
| 535 | Encoding Probe 525 | TGGAAGTTAGAGGGTGTATCATCTCTGAAAAC |
| | | TTCCGACC |
| 536 | Encoding Probe 526 | TGGAAGTTAGAGGGTGTGCTCAGCCTTGGTTT |
| | | TCCGCTA |
| 537 | Encoding Probe 527 | TGGAAGTTAGAGGGTTGCGTCACCCCATTAAG |
| | | AGGCAGG |
| 538 | Encoding Probe 528 | TGGAAGTTAGAGGGTCATGTCAATGAGCAAA |
| | | GGTATTAAGAA |
| 539 | Encoding Probe 529 | TGGAAGTTAGAGGGTGTAAGCTCACAATATGT |
| | | GCATTAAA |
| 540 | Encoding Probe 530 | TGGAAGTTAGAGGGTGAAACTAACACACACA |
| | | CTGATTGTC |
| 541 | Encoding Probe 531 | TGGAAGTTAGAGGGTCTAAGTTAATGATAGTG |
| | | TGTCGATTG |
| 542 | Encoding Probe 532 | TGGAAGTTAGAGGGTGTGTCTCATCTCTGAAA |
| | | ACTTCCGACC |
| 543 | Encoding Probe 533 | TGGAAGTTAGAGGGTAGGAAGGCACATTCTC |
| | | ATCTCACT |
| 544 | Encoding Probe 534 | TGGAAGTTAGAGGGTCGTCACCCCATTAAGA |
| | | GGCTCGGT |
| 545 | Encoding Probe 535 | TGGAAGTTAGAGGGTGCGTCACCCCATTAAG |
| | | AGGCTAGG |
| 546 | Encoding Probe 536 | TGGAAGTTAGAGGGTCATGTCAATGAGCAAA |
| | | GGTATTATGA |
| 547 | Encoding Probe 537 | TGGAAGTTAGAGGGTTAGGCTCACAATATGTG |
| | | CATTAAA |
| 548 | Encoding Probe 538 | TGGAAGTTAGAGGGTTGACACACACACTGATT |
| | | CAGGGAG |
| 549 | Encoding Probe 539 | AGGTTGAGAATAGGAGAGGCTCAGTAGTTTT |
| | | GGATGCTCA |
| 550 | Encoding Probe 540 | AGGTTGAGAATAGGAAGACGCGTCACTTACG |
| | | TGACACGGC |
| 551 | Encoding Probe 541 | AGGTTGAGAATAGGAGTGGAGGTGCTGGTAA |
| | | CTAAGCTG |
| 552 | Encoding Probe 542 | AGGTTGAGAATAGGACTAGTTTTATGGGATTA |
| | | GCTCCAGGA |
| 553 | Encoding Probe 543 | AGGTTGAGAATAGGAGAGGAAAGTTCTCAGC |
| | | ATGTCTTC |
| 554 | Encoding Probe 544 | AGGTTGAGAATAGGAACACCCATGCTCGGCA |
| | | CTTCTCCC |
| 555 | Encoding Probe 545 | AGGTTGAGAATAGGACGCGGTGTTTTTCACAC |
| | | CCATACA |
| 556 | Encoding Probe 546 | AGGTTGAGAATAGGATGGCCAGAGTGATACA |
| | | TGAGGGCG |
| 557 | Encoding Probe 547 | AGGTTGAGAATAGGATGGCTATCTCCGAGCTT |
| | | GATTTCG |
| 558 | Encoding Probe 548 | AGGTTGAGAATAGGAGGCACACAGGAAATTC |
| | | CACCAAGG |
| 559 | Encoding Probe 549 | AGGTTGAGAATAGGAAAGATCCAACTTGCTG |
| | | AACCAGGA |
| 560 | Encoding Probe 550 | AGGTTGAGAATAGGATGCGTCACCTAACAAG |
| | | TAGGCAGG |
| 561 | Encoding Probe 551 | AGGTTGAGAATAGGACGTGTATTAACTTACTG |
| | | CCCTTCGAG |
| 562 | Encoding Probe 552 | AGGTTGAGAATAGGAACAAGACAAAGTTTCT |
| | | CGTGCAGG |
| 563 | Encoding Probe 553 | AGGTTGAGAATAGGAAAACTTCAAAGATCCT |
| | | TTCGCCAT |
| 564 | Encoding Probe 554 | AGGTTGAGAATAGGAGCACGCTAAAATCAAT |
| | | GAAGCTATT |
| 565 | Encoding Probe 555 | AGGTTGAGAATAGGACGATCTGATAGCGTGA |
| | | GGTCCCTT |
| 566 | Encoding Probe 556 | AGGTTGAGAATAGGAATAATTCAGTACAAGA |
| | | TACCTAGGAAT |
| 567 | Encoding Probe 557 | AGGTTGAGAATAGGAAGGCGCTGAATCCAGG |
| | | AGCAACGA |
| 568 | Encoding Probe 558 | AGGTTGAGAATAGGACAAAACGCTCTATGAT |
| | | CGTCAATA |
| 569 | Encoding Probe 559 | AGGTTGAGAATAGGAGCAGTGTTTTTCACACC |
| | | CATTGTGCA |
| 570 | Encoding Probe 560 | AGGTTGAGAATAGGACTGCGATCGGTTTTATG |
| | | GGATATC |
| 571 | Encoding Probe 561 | AGGTTGAGAATAGGAGGATCGACGTGTCTGT |
| | | CTCGCTCA |
| 572 | Encoding Probe 562 | AGGTTGAGAATAGGAGGTGCAGTAACCAGAA |
| | | GTACACCT |
| 573 | Encoding Probe 563 | GGTGTAGGGAGTAAAACCTCTTCGACTGGTCT |
| | | CAGCAGG |
| 574 | Encoding Probe 564 | GGTGTAGGGAGTAAATGCAATCGATGAGGTT |
| | | ATTAACCTGTA |
| 575 | Encoding Probe 565 | GGTGTAGGGAGTAAACATCAGTCACACCCGA |
| | | AGGTGCTAGG |
| 576 | Encoding Probe 566 | GGTGTAGGGAGTAAAGCAATCGATGAGGTTA |
| | | TTAACCTGTA |
| 577 | Encoding Probe 567 | GGTGTAGGGAGTAAACATCAGTCACACCCGA |
| | | AGGTGCAGG |
| 578 | Encoding Probe 568 | GGTGTAGGGAGTAAAATGAGTCACACCCGAA |
| | | GGTGCTAGG |
| 579 | Encoding Probe 569 | GGTGTAGGGAGTAAATCCCTTCACCTACACAC |
| | | CAGCGACG |
| 580 | Encoding Probe 570 | GGTGTAGGGAGTAAATCCCTTCACCTACACAC |
| | | CAGCCAC |
| 581 | Encoding Probe 571 | GGTGTAGGGAGTAAATGACCGCAACCCCGGT |
| | | GAGGGCGG |
| 582 | Encoding Probe 572 | GGTGTAGGGAGTAAAAGAGACTGGTCTCAGC |
| | | TCCACGGC |
| 583 | Encoding Probe 573 | GGTGTAGGGAGTAAAATGAGTCACACCCGAA |
| | | GGTGCAGG |
| 584 | Encoding Probe 574 | GGTGTAGGGAGTAAATGCGTCACACCCGAAG |
| | | GTGCTAGG |
| 585 | Encoding Probe 575 | GGTGTAGGGAGTAAAGTGCTCAGCCTTGATTA |
| | | TCCGCTA |
| 586 | Encoding Probe 576 | GGTGTAGGGAGTAAACCACGTCAATCGATGA |
| | | GGTTAAAT |
| 587 | Encoding Probe 577 | GGTGTAGGGAGTAAAAATAACCTCATCGCCTT |
| | | CCTCAGG |
| 588 | Encoding Probe 578 | GGTGTAGGGAGTAAACCCACGTCAATCGATG |
| | | AGGTTTAA |
| 589 | Encoding Probe 579 | GGTGTAGGGAGTAAACATCAGTCACACCCGA |
| | | AGGTGGAG |
| 590 | Encoding Probe 580 | GGTGTAGGGAGTAAACCCTTCACCTACACACC |
| | | AGCGACG |
| 4 | Readout Probe 4 | /5PacificGreenN/ACCCTCTAACTTCCATCACA |
| 6 | Readout Probe 6 | /5Atto610N/TTTACTCCCTACACCTCCAA |
| 8 | Readout Probe 8 | /5DyLight-510-LS/ |
| | | TCCTATTCTCAACCTAACCT/3DyLight-510-LS/ |
| 591 | Exchange Probe 1 | TGTGATGGAAGTTAGAGGGT |
| 592 | Exchange Probe 2 | TTGGAGGTGTAGGGAGTAAA |
| 593 | Exchange Probe 3 | AGGTTAGGTTGAGAATAGGA |

### Example 7. Timescale Determination the Exchange Reaction in HiPR-Swap

The experiment was continued after 5 days in the same samples as described in Example 6. To determine the timescale of the exchange reaction, the reaction was performed for 1 hour.

The experiment was performed to show that the stripping of readout probes can be achieved within 1 hour, as opposed to a longer period of time, such as over 12 hours.

The procedure was as follows. The cells were removed from the 4° C. refrigerator after 5 days and imaged in the wash buffer. The cells were removed from the scope and the exchange buffer was added to the cells at 37° C. for 1 hour. The wash buffer was then added to the cells at 30° C. for 15 minutes and the cells were imaged in the wash buffer. The encoding, readout, and exchange probes used in this example are shown in Table 5.

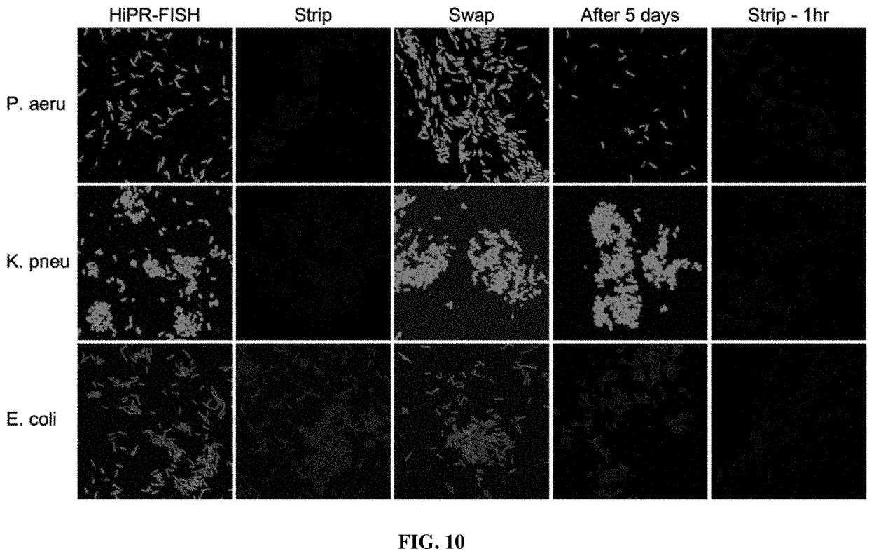

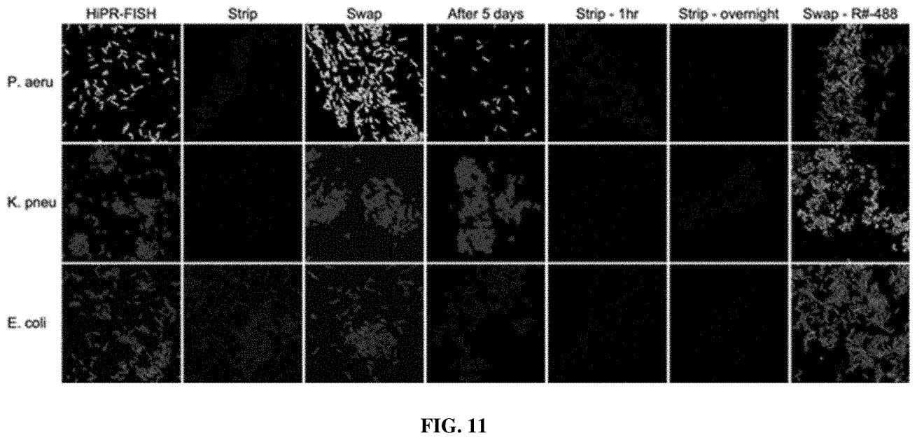

As can be seen in FIG. 10, the experiment showed that the fluorescence signal from P. aeruginosa and K. pneumoniae did not degrade significantly after 5 days. The fluorescence signal from E. coli had degraded significantly due to rapid photobleaching and instability of the Atto-390 dye in the wash buffer (“After 5 days”). Each species had most of their readout probes removed within a span of 1 hour (“Strip—1 hr”). There is a small fluorescence signal left after 1 hour. Therefore, the whole exchange reaction can be completed within 1.5-2 hours or less.

### Example 8. Recovery Of Signal With Different Readout Probes in HiPR-Swap

This experiment was performed to show the sequential repeatability of the HiPR-Swap method and continues from Example 7.

After stripping the readout probes for 1 hour, the stripping reaction was continued overnight to remove the remaining readout probes. Following this, each species was encoded with the readout probes that correspond to their respective readout pads but tagged with the same dye (Alexa-488).

The procedure was as follows. The exchange buffer was added to the cells at 37° C. and left overnight. The wash buffer was then added to the cells at 30° C. for 15 minutes and the cells were imaged in the wash buffer. The cells were removed from the scope. A readout buffer was prepared separately for each species containing one of the following probes: R4-488, R6-488, R8-488. The readout buffer was then added to the cells and incubated at 37° C. for 2 hours. The wash buffer was added to the cells at 30° C. for 15 minutes and the cells were imaged in the wash buffer.

As shown in FIG. 11, the experiment showed that the fluorescent signal was completely removed from each species (“Strip-overnight”) and the fluorescence signal was recovered with the encoded color (green, not shown) after adding the readout probes (“Swap—R #-488”).

Overall, these results demonstrate the full two cycles of HiPR-swap assay with robust removal and re-hybridization of the readout probes.

The R4-488, R6-488, R8-488 probes are shown in Table 6 below.

TABLE 6
488 Readout Probes.

| SEQ ID | | |
|---|---|---|
| NO: | Probe Name | Sequence (in 5′ to 3′ order) |
| 594 | R4-488 | /5Alex488N/ACCCTCTAACTTCCATCACA |
| 595 | R6-488 | /5Alex488N/TTTACTCCCTACACCTCCAA |
| 596 | R8-488 | /5Alex488N/TCCTATTCTCAACCTAACCT |

### Example 9. Single-Step Probe Exchange and New Readout Addition

As shown in Examples 6-7, the readout probes can be removed (stripped) and replaced (swapped) in two subsequent steps. As long as the second round of readout probes differs from the first set that is being removed with exchange probes, the strip and swap can be performed in a single step.

Single-step HiPR-Swap and two-step HiPR-Swap was performed on a single slide with neighboring wells. In both wells, a mixture of E. coli and P. aeruginosa cells was adhered to the surface.

Round 1: In the first round for both wells, the taxa encoding probes for both species (including EUB which will serve as a tool to segment cells for analysis) were added and readout probes only for E. coli. The encoding and readout hybridization reactions were performed in a single step. Both wells were imaged following the first round of encoding and readout.

Round 2: In round two of the single-step well, the readout probes from E. coli were stripped and swapped with the readout probes for P. aeruginosa. For the two-step well, only the readout probes were stripped from E. coli. Both wells were imaged following this hybridization step.

Round 3: In round three of the single-step well, the readout probes from P. aeruginosa were stripped and swapped with the readout probes for E. coli. For the two-step well, only the readout probes were stripped from P. aeruginosa. Both wells were imaged following this hybridization step.

The experiment was conducted as follows mixtures of cells were adhered to a coverslip via evaporation. The cells were digested with lysozyme at 37° C. for 30 minutes. The cells were washed with 1×PBS at room temperature for 15 minutes.

Round 1: The encoding probe hybridization and readout probe hybridization were performed in a single step. The hybridization buffer was prepared as follows for both the wells: 10% ethylene carbonate, 10% dextran sulfate, 2×SSC, 5×Denhardt's solution, 0.01% SDS, 2 uM per taxa of the encoding probes, 400 nM of the Eubacterium probe, and 400 nM of the readout probe for E. coli. The hybridization buffer was added to the cells at 37° C. for 2 hours. The wash buffer (215 mM NaCl, 20 mM Tris-HCl (pH 8.0); and 5 mM EDTA) was added to the cells at 30° C. for 15 minutes. All wells were filled in excess with 2×SSC. A glass coverslip was placed on top of the wells to minimize evaporation. The cells were imaged under 2×SSC. Then, the cells were removed from the scope. The cells were washed with wash buffer for 1 min at RT. The cells were stored overnight in the wash buffer at 4° C.

Round 2: The exchange buffers were prepared separately for each well. Well: Single Step: 10% ethylene carbonate, 10% dextran sulfate, 2×SSC, 5×Denhardt's solution, 0.01% SDS, 6 uM of the exchange probe for E. coli, and 400 nM of the readout probes for P. aeruginosa. Well: Two Step: 10% ethylene carbonate, 10% dextran sulfate, 2×SSC, 5×Denhardt's solution, 0.01% SDS, and 6 uM of the exchange probe for E. coli.

The exchange buffers were added to the cells at 37° C. for 2 hours. The wash buffer was added to the cells at 30° C. for 15 minutes. All wells were filled in excess with 2×SSC. A glass coverslip was placed on top of the wells to minimize evaporation. The cells were imaged under 2×SSC. The cells were removed from the scope. The cells were washed with wash buffer for 1 min at RT.

Round 3: The exchange buffers were prepared separately for each well. Well: Single Step: 10% ethylene carbonate, 10% dextran sulfate, 2×SSC, 5×Denhardt's solution, 0.01% SDS, 6 uM of the exchange probe for P. aeruginosa, and 400 nM of the readout probes for E. coli. Well: Two Step: 10% ethylene carbonate, 10% dextran sulfate, 2×SSC, 5×Denhardt's solution, 0.01% SDS, and 400 nM of the readout probes probe for P. aeruginosa. The exchange buffers were added to the cells at 37° C. for 2 hours. The wash buffer was added to the cells at 30° C. for 15 minutes. All wells were filled in excess with 2×SSC. A glass coverslip was placed on top of the wells to minimize evaporation. The cells were imaged under 2×SSC. The encoding, readout, and exchange probes used in this example are shown in Table 5.

The single-step strip and swap reaction works equally well as the two-step reaction. This enables us to perform multiple rounds of HiPR-Swap (for example, at least 3 rounds for 30 bit barcode) in less than 12 hours.

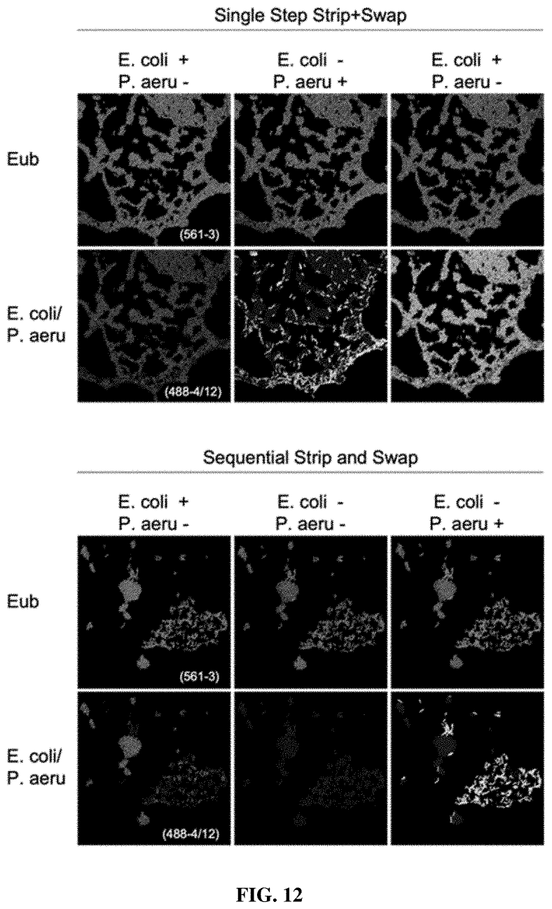

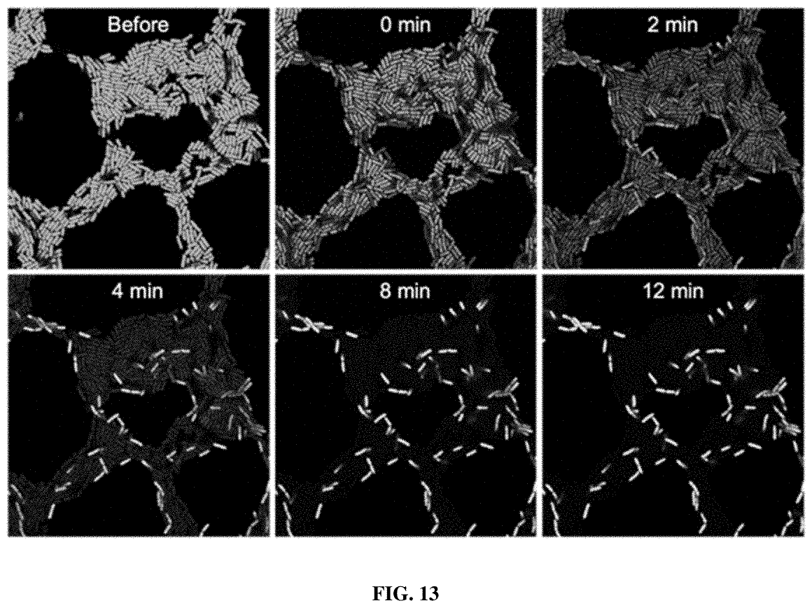

As shown in FIG. 12, with single-step condition, successful demonstration of HiPR-Swap was shown up to 3 rounds.

In single step condition, E. coli in round 1 is dimmer than the E. coli in round 3. This is likely because of the inefficient binding of readout probes to the readout pads in the first round of encoding/readout, where single step encoding and readout was used to perform HiPR-FISH. An addition of pre-hybridization incubation step before encoding/readout step can improve the binding efficiency of readout probes in round 1.

### Example 10. Realtime Measurement of HiPR-Swap Using Single-Step Strip and Swap Reaction

The single step strip and swap reaction was shown to work equally well as the two-step reaction. In this example, the single-step reaction was used to measure the stripping and swapping of the probes in real time.

Single-step HiPR-Swap was performed with a mixture of E. coli and P. aeruginosa cells.

Round 1: In the first round, the taxa encoding probes were added for both species and readout probes only for E. coli. The encoding and readout hybridization reactions were performed in a single step. The cells were imaged after this hybridization step.

Round 2: In the second round, the cells were placed under the microscope. the readout probes were stripped from E. coli and swapped with the readout probes for P. aeruginosa. Images were acquired while the stripping and swapping reaction was undergoing.

The following was performed in this example. Mixture of cells were adhered to a coverslip via evaporation. The cells were digested with lysozyme at 37° C. for 30 minutes and washed with 1×PBS at room temperature for 15 minutes. The pre-hybridization buffer (10% ethylene carbonate, 10% dextran sulfate, 2×SSC, 5×Denhardt's solution, 0.01% SDS) was added to the cells at 37° C. for 30 mins.

Round 1: The encoding probe hybridization and readout probe hybridization were performed in a single step. The hybridization buffer (both wells; 10% ethylene carbonate, 10% dextran sulfate, 2×SSC, 5×Denhardt's solution, 0.01% SDS, 2 μM per taxa of the encoding probes, and 400 nM of the readout probe for E. coli) was added to the cells at 37° C. for 2 hours. The wash buffer (215 mM NaCl, 20 mM Tris-HCl (pH 8.0); and 5 mM EDTA) was added to the cells at 30° C. for 15 minutes. The cells were placed on the microscope and imaged under the wash buffer before acquiring the timelapse.

Round 2: The wash buffer was removed and the well was filled with the exchange buffer (10% ethylene carbonate, 10% dextran sulfate, 2×SSC, 5×Denhardt's solution, 0.01% SDS, 50 nM of the exchange probe for E. coli, and 25 nM of the readout probes for P. aeruginosa) under the microscope. The timelapse was started and images were acquired at a 15 seconds interval. The encoding, readout, and exchange probes used in this example are shown in Table 5.

As shown in FIG. 13, the real time stripping and swapping of the readout probes in the mixture of two species was demonstrated.

To capture the kinetics of this reaction, the reaction was purposefully slowed down dramatically by using a very low concentration of the exchange probes (50 nM) and the readout probes (25 nM). At higher concentrations, such as 2 uM for exchange probes and 400 nM for readout probes, in here the strip and swap reactions can be completed within a few minutes.

Notably, with the addition of the pre-hybridization step, the binding efficiency of the readout probes in the first round improved dramatically, as evident from the intensity of the “before” image in timelapse.

### Example 11. E. coli Encoding with 30-Bit Barcode, and Measured in 3 Rounds

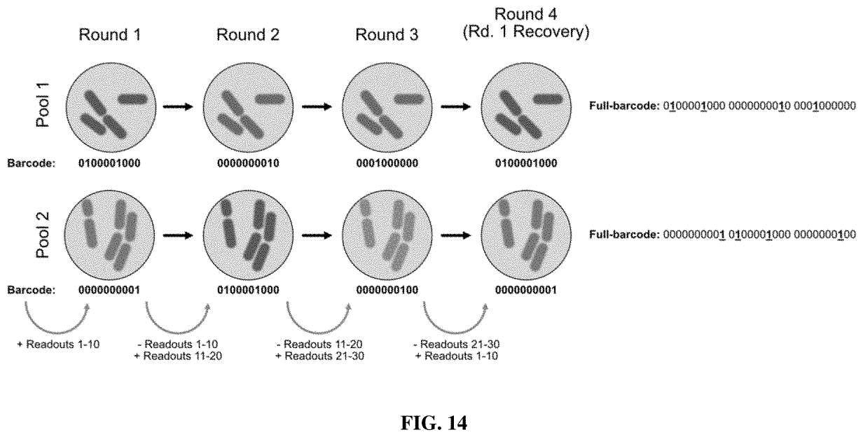

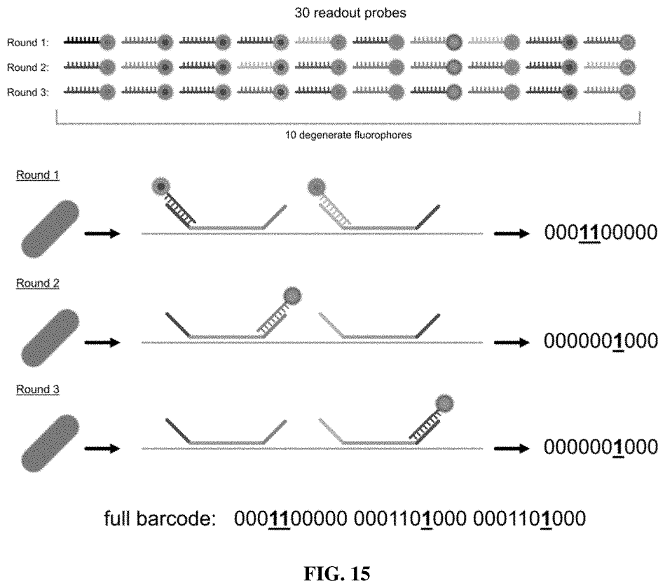

To show the full potential of using HiPR-Swap towards increasing the multiplexity of HiPR-FISH related assays, including HIPR-FAST and HIPR-cycle, the ability to identify over 1 billion taxa (or other targets; 1023\hat{ }3) in about 12 hours was shown. FIG. 14 shows a schematic for this example and FIG. 15 shows an overview of HiPR-Swap as in this example.

This example was performed with E. coli bacteria bound to the coverslips in three wells. The bacteria in each well was encoded with a unique 30-bit barcode (e.g. 0110001000-0100100111-1101001000). The 30-bit experiment was performed in three rounds using HIPR-Swap, with each round containing up to 10-bits. A fourth round was added for error correction by going back to the same readouts as round 1.

Round 1: In the first round, the taxa encoding probes for bacteria were added in each well and incubated overnight. The first set of readout probes were added in each well. The cells were imaged after this hybridization step.

Round 2: In the second round, the first set of exchange probes to strip readout probes of round 1 was added, and second set of readout probes in each well. The cells were imaged after this hybridization step.

Round 3: In the third round, the second set of exchange probes to strip readout probes of round 2 was added, and third set of readout probes in each well. The cells were imaged after this hybridization step.

Round 4: In the fourth round, the third set of exchange probes to strip readout probes of round 3 were added, and first set of readout probes in each well. This was done to go back to the same sets of readout probes as used in round 1. The cells were imaged after this hybridization step.

The single step HiPR-Swap protocol was utilized as follows: cells were adhered to a coverslip via evaporation. The cells were digested with lysozyme at 37° C. for 30 minutes. The cells were washed with 1×PBS at room temperature for 15 minutes.

Round 1: The encoding buffer was prepared separately for each well as follows: 10% ethylene carbonate, 10% dextran sulfate, 2×SSC, 5×Denhardt's solution, 0.01% SDS, 2 μM each of encoding probes combination (C #, where #=readout probe #) as described below—
Well 1: C11+C13+C16+C18
Well 2: C12+C15+C21
Well 3: C13+C17+C19
   A combination of encoding probes, C #, encompasses 24 encoding probes (as shown in Table 7 below) each concatenated to readout landing pads sequences corresponding to a specific readout probe number (#). For example, Combination 11 (C11) corresponds to 24 encoding probes each concatenated to landing pad sequence 11: TTAATATGGGTAGTTGGG (SEQ ID NO.: 1810). The landing pad sequence is partially complementary to the sequence of Readout Probe 11 (SEQ ID NO.: 597).

The encoding buffer was added to the cells at 37° C. and incubated overnight. The wash buffer was prepared as 215 mM NaCl, 20 mM Tris-HCl (pH 8.0), 5 mM EDTA. The wash buffer was added to the cells at 42° C. for 15 minutes. The readout buffer was prepared as follows 10% ethylene carbonate, 10% dextran sulfate, 2×SSC, 5×Denhardt's solution, 0.01% SDS, and 400 nM each of readout probe 11, 12, 13. The readout buffer was added to the cells at 37° C. for 1 hour. The wash buffer was added to the cells at 42° C. for 15 minutes. All wells were filled in excess with 2×SSCT. A glass coverslip was placed on top of the wells to minimize evaporation. The cells were imaged under 2×SSCT. The cells were removed from the scope. The cells were washed with 2×SSC for 1 min at RT.

Round 2: The exchange buffer for round 2 was prepared as follows 10% ethylene carbonate, 10% dextran sulfate, 2×SSC, 5×Denhardt's solution, 0.01% SDS, 10 uM each of exchange probe 5, 8, and 10, 400 nM each of readout probe 14-17. The exchange buffer was added to the cells at 37° C. for 1 hour. The wash buffer was added to the cells at 42° C. for 15 minutes. All wells were filled in excess with 2×SSCT. A glass coverslip was placed on top of the wells to minimize evaporation. The cells were imaged under 2×SSCT. The cells were removed from the scope. The cells were washed with 2×SSC for 1 min at RT.

Round 3: The exchange buffer for round 3 was prepared as follows: 10% ethylene carbonate, 10% dextran sulfate, 2×SSC, 5×Denhardt's solution, 0.01% SDS, 10 uM each of exchange probe 14, 15, 17, 18, 400 nM each of readout probe 18-21. The exchange buffer was added to the cells at 37° C. for 1 hour. The wash buffer was added to the cells at 42° C. for 15 minutes. All wells were filled in excess with 2×SSCT. A glass coverslip was placed on top of the wells to minimize evaporation. The cells were imaged under 2×SSCT. The cells were removed from the scope. The cells were washed with 2×SSC for 1 min at RT.

Round 4: The exchange buffer for round 4 was prepared as follows: 10% ethylene carbonate, 10% dextran sulfate, 2×SSC, 5×Denhardt's solution, 0.01% SDS, 10 uM each of exchange probe 24, 25, 28, 30, 400 nM each of readout probe 11, 12, and 13. The exchange buffer was added to the cells at 37° C. for 1 hour. The wash buffer was added to the cells at 42° C. for 15 minutes. All wells were filled in excess with 2×SSCT. A glass coverslip was placed on top of the wells to minimize evaporation. The cells were imaged under 2×SSCT.

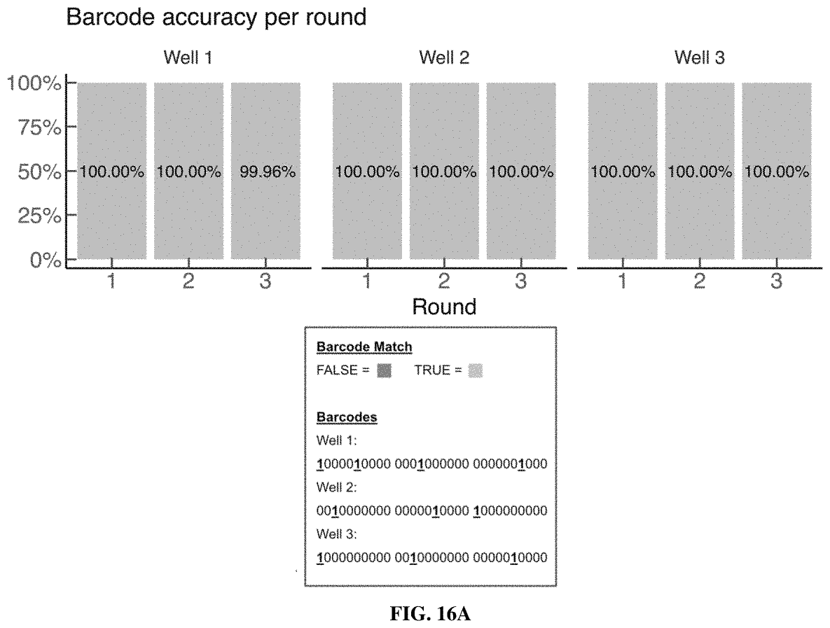

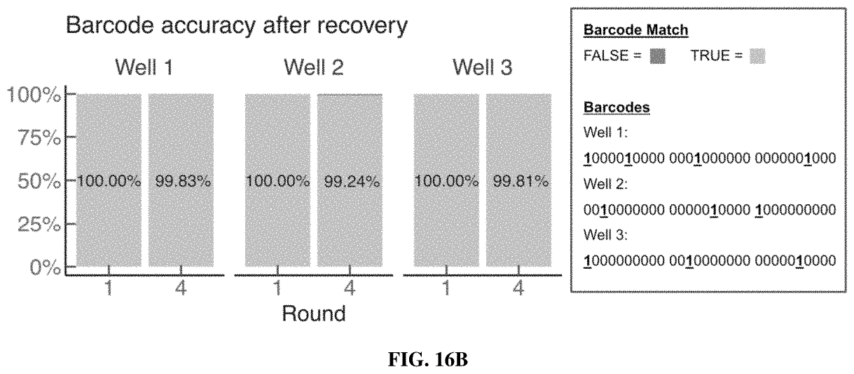

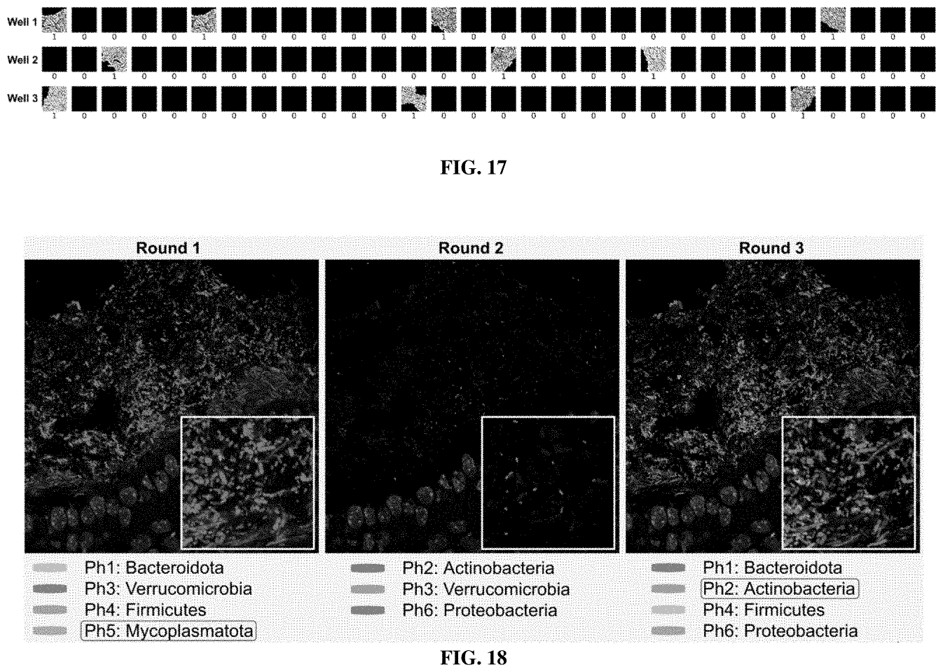

FIGS. 16A-16B shows a summary of classification accuracy for this example. As shown in FIG. 17, bacteria fluorescence matches the expected barcode. In each well a mask for the most abundant barcode applied to the maximum spectral projection. Fluorescent bacteria only appear in channels corresponding to the “1” bit.

Microscopy

As indicated above, in each round, imaging using confocal microscopy (Zeiss i880 confocal microscope) with emission collected was collected on a spectral detector between roughly the excitation wavelength and 693 nm in 8.9 nm bins. A Plan-Apochromat 63×/1.4 Oil DIC M27 was used and collected data as 2000×2000 pixel images (134.95 μm×134.95 μm). The laser settings for the example were as shown in Table 6 below.

TABLE 6
Laser settings

| | | | | Pixel | | | | | | |
|---|---|---|---|---|---|---|---|---|---|---|
| Laser | | Pinhole | Laser | Dwell | | | | | | |
| Excitation | | Size | Power | Time | Bit | Scanning | Scanning | Master | Digital | Digital |
| (nm) | Laser | (nm) | (%) | (μsec) | Depth | Direction | Repeats | Gain | Offset | Gain |
| 488 | Argon | 56.0 | 0.5 | 2.1 | 16-bit | Bidirectional | 4 | 800 | 0 | 1 |
| 514 | Argon | 58.0 | 1.5 | 2.1 | 16-bit | Bidirectional | 4 | 800 | 0 | 1 |
| 561 | DPSS | 60.0 | 0.125 | 2.1 | 16-bit | Bidirectional | 4 | 800 | 0 | 1 |
| | 561-10 | | | | | | | | | |
| 633 | HeNe633 | 64.0 | 1.5 | 2.1 | 16-bit | Bidirectional | 4 | 800 | 0 | 1 |

The encoding, readout, and exchange probes used in this example are shown in table 7 below.

TABLE 7
Encoding, readout, and exchange probes used in Example 11

| SEQ ID NO: | Probe Name | Sequence (in 5′ to 3′ order) |
|---|---|---|
| 597 | Readout Probe 11 | /5Alex532/CCCAACTACCCATATTAACACACCC |
| 598 | Readout Probe 12 | /56-ROXN/CCCTTCTCACTAAATTCCAACACCC |
| 599 | Readout Probe 13 | /5Alex647N/CACCCTCATATCTATTACCCTCCCA |
| 600 | Readout Probe 14 | /5Alex488N/TCCCTCCTTACTATTACACTCACCC |
| 601 | Readout Probe 15 | /5Alex532/CATCCCTCCTTATTATCCTCATCCC |
| 602 | Readout Probe 16 | /5Alex546N/CCCTTCTACTACTTCCATACATCCC |
| 603 | Readout Probe 17 | /56-ROXN/CCCTTCTAATCCTATACACTCACCC |
| 604 | Readout Probe 18 | /5Alex488N/CCCATCTCTCTAATTCTACTCCACC |
| 605 | Readout Probe 19 | /5Alex532/TCCCTCCTCTTAATACATCCTCCTC |
| 606 | Readout Probe 20 | /56-ROXN/CATCCCTACTTACTTATCCTCCACC |
| 607 | Readout Probe 21 | /5Alex647N/CCCTTCTCCATAACTATACCCTTCC |
| 608 | Exchange Probe 5 | GGGTGTGTTAATATGGGTAGTTGGG |
| 609 | Exchange Probe 8 | GGGTGTTGGAATTTAGTGAGAAGGG |
| 610 | Exchange Probe 10 | TGGGAGGGTAATAGATATGAGGGTG |
| 611 | Exchange Probe 14 | GGGTGAGTGTAATAGTAAGGAGGGA |
| 612 | Exchange Probe 15 | GGGATGAGGATAATAAGGAGGGATG |
| 613 | Exchange Probe 17 | GGGATGTATGGAAGTAGTAGAAGGG |
| 614 | Exchange Probe 18 | GGGTGAGTGTATAGGATTAGAAGGG |
| 615 | Exchange Probe 24 | GGTGGAGTAGAATTAGAGAGATGGG |
| 616 | Exchange Probe 25 | GAGGAGGATGTATTAAGAGGAGGGA |
| 617 | Exchange Probe 28 | GGTGGAGGATAAGTAAGTAGGGATG |
| 618 | Exchange Probe 30 | GGAAGGGTATAGTTATGGAGAAGGG |
| 619 | Encoding Probe 581 | *CCTCAGTTAATGATAGTGTGTCGATTG |
| 620 | Encoding Probe 582 | *GGAGCCTTGGTTTTCCGGATTACG |
| 621 | Encoding Probe 583 | *GTGTCTCATCTCTGAAAACTTCCCAC |
| 622 | Encoding Probe 584 | *GTCACCCCATTAAGAGGCTCCGTG |
| 623 | Encoding Probe 585 | *CCACGTCAATGAGCAAAGGTAAAT |
| 624 | Encoding Probe 586 | *GTAAGCTCACAATATGTGCATAAA |
| 625 | Encoding Probe 587 | *GATACACACACTGATTCAGGCAGA |
| 626 | Encoding Probe 588 | *AGTCTTGGTTTTCCGGATTTGGGA |
| 627 | Encoding Probe 589 | *ACCTCAGTTAATGATAGTGTGTCGTTT |
| 628 | Encoding Probe 590 | *GAGCCTTGGTTTTCCGGATTTCGG |
| 629 | Encoding Probe 591 | *GTATCATCTCTGAAAACTTCCGACC |
| 630 | Encoding Probe 592 | *GTGCTCAGCCTTGGTTTTCCGCTA |
| 631 | Encoding Probe 593 | *TGCGTCACCCCATTAAGAGGCAGG |
| 632 | Encoding Probe 594 | *CATGTCAATGAGCAAAGGTATTAAGAA |
| 633 | Encoding Probe 595 | *GTAAGCTCACAATATGTGCATTAAA |
| 634 | Encoding Probe 596 | *GAAACTAACACACACACTGATTGTC |
| 635 | Encoding Probe 597 | *CTAAGTTAATGATAGTGTGTCGATTG |
| 636 | Encoding Probe 598 | *GTGTCTCATCTCTGAAAACTTCCGACC |
| 637 | Encoding Probe 599 | *AGGAAGGCACATTCTCATCTCACT |
| 638 | Encoding Probe 600 | *CGTCACCCCATTAAGAGGCTCGGT |
| 639 | Encoding Probe 601 | *GCGTCACCCCATTAAGAGGCTAGG |
| 640 | Encoding Probe 602 | *CATGTCAATGAGCAAAGGTATTATGA |
| 641 | Encoding Probe 603 | *TAGGCTCACAATATGTGCATTAAA |
| 642 | Encoding Probe 604 | *TGACACACACACTGATTCAGGGAG |
| 1810 | Landing Pad 11 | TTAATATGGGTAGTTGGG- |
| 1811 | Landing Pad 12 | GGAATTTAGTGAGAAGGG- |
| 1812 | Landing Pad 13 | GTAATAGATATGAGGGTG- |
| 1813 | Landing Pad 14 | TGTAATAGTAAGGAGGGA- |
| 1814 | Landing Pad 15 | GGATAATAAGGAGGGATG- |
| 1815 | Landing Pad 16 | ATGGAAGTAGTAGAAGGG- |
| 1816 | Landing Pad 17 | TGTATAGGATTAGAAGGG- |
| 1817 | Landing Pad 18 | TAGAATTAGAGAGATGGG- |
| 1818 | Landing Pad 19 | ATGTATTAAGAGGAGGGA- |
| 1819 | Landing Pad 20 | GATAAGTAAGTAGGGATG- |
| 1820 | Landing Pad 21 | TATAGTTATGGAGAAGGG- |

The asterisk (*) represents the concatenated landing pad sequence for each combination (C#). For instance, in C11, each of the encoding probes 581-604 has the sequence of landing pad 11 appended to its 3′ end. For example, Encoding Probe 581 when present in C11, would have a sequence of TTAATATGGGTAGTTGGGCCTCAGTTAATGATAGTGTGTCGATTG (SEQ ID NO: 1821) corresponding to Landing Pad 11 + Encoding Probe 581 as shown in the table. For C12, each of the encoding probes 581-604 has the sequence of landing pad 12 appended to its 3′ end, and so on. The dash (-) on each landing pad sequence represents the point of attachment to the encoding probe.

### Example 12. Phylum-Level Swap in Tissue Samples

To examine the ability to perform HiPR-Swap on a tissue specimen (colon of a healthy mouse) probes were designed to perform a simple taxon identification experiment, barcoding the six most abundant bacteria phyla with either one or two readout probes, such that each readout probe was only present in one of three imaging rounds. As shown in FIG. 18, we identified each phylum targeted (Bacteroidota, Verrucomicrobia, Actinobacteria, Firmicutes, Mycoplasmatota, and Proteobacteria). The two most abundant taxa were confirmed to be Firmicutes and Bacteroidota, as expected. Taxonomy of each segmented microbe was classified the across each round of imaging, finding that it was possible to accurately identify over 90% of fluorescently labeled bacteria in each image, and deriving similar abundance measurements for taxa labeled in different rounds (e.g. roughly 50% of bacteria identified as Bacteroidota in both rounds 1 and 3).

Phylum-level swap protocol: OCT (optimal cutting temperature)-embedded formalin-fixed tissue was sectioned at 10-micron thickness onto circular glass coverslips made for Bioptechs FCS2 flow cell. The tissue was covered with 2% formaldehyde for two hours at room temperature to fix the sample. The sample was washed by removing the buffer and replacing it with 1×PBS for 5 minutes (this was repeated two more times). The fixed tissue specimen was stored in 70% ethanol at 4° C. overnight. The following buffers were prepared:
Encoding buffer: Encoding probes (204 per taxa); 2×sodium chloride sodium citrate (SSC), 5×Denhardt's solution, 10% dextran sulfate, 10% ethylene carbonate, and 0.01% sodium dodecyl sulfate (SDS)
Round 1 readout buffer: 400 nM each of readout probes: 11+13+9; 2×sodium chloride sodium citrate (SSC), 5×Denhardt's solution, 10% dextran sulfate, 10% ethylene carbonate, and 0.01% sodium dodecyl sulfate (SDS)
Round 2 readout buffer: 10 uM each of exchange probe: 5+10, 400 nM each of readout probes 14+16+17, 2×sodium chloride sodium citrate (SSC), 5×Denhardt's solution, 10% dextran sulfate, 10% ethylene carbonate, and 0.01% sodium dodecyl sulfate (SDS)
Round 3 readout buffer: 10 uM each of exchange probe: 14+17+18, 400 nM each of readout probes: 19-21, 2×sodium chloride sodium citrate (SSC), 5×Denhardt's solution, 10% dextran sulfate, 10% ethylene carbonate, 0.01% sodium dodecyl sulfate (SDS)
Wash buffer: 215 mM NaCl, 20 mM Tris-HCl (pH 7.5), 5 mM EDTA

Place 10 mg/mL lysozyme to completely cover the specimen and incubate for 30 minutes at 37° C. in a humidified chamber. Wash the specimen with 1×PBS for 15 minutes at room temperature. Dry the specimen by submerging it in 100% ethanol and allowing it to air dry. Place the coverslip on the FCS2 flow cell (Bioptechs) and assemble. Place the flow cell assembly on the microscope stage (Zeiss i880 confocal). Connect the flow cell input port to the Aria Automated Perfusion System (Fluigent). Calibrate the Aria Automated Perfusion System using DI water. Load encoding, readout buffers, wash buffer, 1×PBS buffer, 5×SSC+DAPI buffer (40 ng/mL DAPI in 5×SSC), and 2×SSC buffer into Aria Automated Perfusion System at the desired reservoir locations. Execute the following sequence on the Aria:
a. Incubate the specimen with 1×PBS at room temperature for 15 minutes.
b. Incubate the specimen in the Encoding buffer at 37° C. for 2 hours.
c. Incubate the specimen in the Wash buffer at 42° C. for 15 minutes.
d. Incubate the specimen in the Round 1 readout buffer at 37° C. for 1 hour.
e. Incubate the specimen in the Wash buffer at 42° C. for 15 minutes.
f. Incubate the specimen in 5×SSC+DAPI at room temperature for 15 minutes.
g. Flush the specimen with 2×SSC for imaging.
h. Perform image acquisition, exciting the specimen with 633 nm, 561 nm, 514 nm, 488 nm, and 405 nm lines and collect spectra.
i. Incubate the specimen in the Round 2 readout buffer at 37° C. for 1 hour.
j. Repeat steps e-h.
k. Incubate the specimen in the Round 3 readout buffer at 37° C. for 1 hour.
l. Repeat steps e-h.

Table 8 shows the encoding, readout, and exchange probe sequences used in this example.

TABLE 8
Encoding, readout, and exchange probe sequences used in Example 12

| SEQ ID | | |
|---|---|---|
| NO: | Probe Name | Sequence (in 5′ to 3′ order) |
| 597 | Readout Probe 11 | /5Alex532/CCCAACTACCCATATTAACACACCC |
| 9 | Readout Probe 9 | /5Alex405N/TTCTCCCTCTATCAACTCTA |
| 599 | Readout Probe 13 | /5Alex647N/CACCCTCATATCTATTACCCTCCCA |
| 600 | Readout Probe 14 | /5Alex488N/TCCCTCCTTACTATTACACTCACCC |
| 602 | Readout Probe 16 | /5Alex546N/CCCTTCTACTACTTCCATACATCCC |
| 603 | Readout Probe 17 | /56-ROXN/CCCTTCTAATCCTATACACTCACCC |
| 605 | Readout Probe 19 | /5Alex532/TCCCTCCTCTTAATACATCCTCCTC |
| 606 | Readout Probe 20 | /56-ROXN/CATCCCTACTTACTTATCCTCCACC |
| 607 | Readout Probe 21 | /5Alex647N/CCCTTCTCCATAACTATACCCTTCC |
| 608 | Exchange Probe 5 | GGGTGTGTTAATATGGGTAGTTGGG |
| 610 | Exchange Probe 10 | TGGGAGGGTAATAGATATGAGGGTG |
| 611 | Exchange Probe 14 | GGGTGAGTGTAATAGTAAGGAGGGA |
| 613 | Exchange Probe 17 | GGGATGTATGGAAGTAGTAGAAGGG |
| 614 | Exchange Probe 18 | GGGTGAGTGTATAGGATTAGAAGGG |
| 616 | Exchange Probe 25 | GAGGAGGATGTATTAAGAGGAGGGA |
| 617 | Exchange Probe 28 | GGTGGAGGATAAGTAAGTAGGGATG |
| 618 | Exchange Probe 30 | GGAAGGGTATAGTTATGGAGAAGGG |
| 643 | Encoding Probe 605 | TTAATATGGGTAGTTGGGTGGATGCCCCTCGACT |
| | | TGCATGACA |
| 644 | Encoding Probe 606 | TTAATATGGGTAGTTGGGACAGGGACCTTCCTCT |
| | | CAGAAAGG |
| 645 | Encoding Probe 607 | TTAATATGGGTAGTTGGGCGTGAGTTAGCCGAT |
| | | GCTTTTAGA |
| 646 | Encoding Probe 608 | TTAATATGGGTAGTTGGGCATCTGCCTTCGCAAT |
| | | CGGAGAAG |
| 647 | Encoding Probe 609 | TTAATATGGGTAGTTGGGTCCCCTCGCGTATCAT |
| | | CGAATATT |
| 648 | Encoding Probe 610 | TTAATATGGGTAGTTGGGCCCTGCGCTCGTTATG |
| | | GCACTATT |
| 649 | Encoding Probe 611 | TTAATATGGGTAGTTGGGTGTACTGATGCGCGAT |
| | | TACTAGGCT |
| 650 | Encoding Probe 612 | TTAATATGGGTAGTTGGGTGGCGGCTTCCATGGC |
| | | TTGACCCC |
| 651 | Encoding Probe 613 | TTAATATGGGTAGTTGGGTGCTTGCATGTGTTAA |
| | | GCCTGTGCG |
| 652 | Encoding Probe 614 | TTAATATGGGTAGTTGGGCCCACCTTCCTCTCAG |
| | | AACCCGAT |
| 653 | Encoding Probe 615 | GATAAGTAAGTAGGGATGGGATGCCCCTCGACT |
| | | TGCATGACA |
| 654 | Encoding Probe 616 | GATAAGTAAGTAGGGATGACAGGGACCTTCCTC |
| | | TCAGAAAGG |
| 655 | Encoding Probe 617 | GATAAGTAAGTAGGGATGCGTGAGTTAGCCGAT |
| | | GCTTTTAGA |
| 656 | Encoding Probe 618 | GATAAGTAAGTAGGGATGCATCTGCCTTCGCAA |
| | | TCGGAGAAG |
| 657 | Encoding Probe 619 | GATAAGTAAGTAGGGATGTCCCCTCGCGTATCA |
| | | TCGAATATT |
| 658 | Encoding Probe 620 | GATAAGTAAGTAGGGATGCCCTGCGCTCGTTAT |
| | | GGCACTATT |
| 659 | Encoding Probe 621 | GATAAGTAAGTAGGGATGGTACTGATGCGCGAT |
| | | TACTAGGCT |
| 660 | Encoding Probe 622 | GATAAGTAAGTAGGGATGTGGCGGCTTCCATGG |
| | | CTTGACCCC |
| 661 | Encoding Probe 623 | GATAAGTAAGTAGGGATGGCTTGCATGTGTTAA |
| | | GCCTGTGCG |
| 662 | Encoding Probe 624 | GATAAGTAAGTAGGGATGCCCACCTTCCTCTCA |
| | | GAACCCGAT |
| 663 | Encoding Probe 625 | ATGGAAGTAGTAGAAGGGTCCCAGGTTGGTCAC |
| | | GTGTTAGAG |
| 664 | Encoding Probe 626 | ATGGAAGTAGTAGAAGGGTGGACCTACTACCTA |
| | | ATGGGCCCGC |
| 665 | Encoding Probe 627 | ATGGAAGTAGTAGAAGGGTGAACTGCTGAAAGC |
| | | GGTTTACTTG |
| 666 | Encoding Probe 628 | ATGGAAGTAGTAGAAGGGAAGGATATCTGCGCA |
| | | TTCCACGCG |
| 667 | Encoding Probe 629 | ATGGAAGTAGTAGAAGGGTAATTTGAGTTTTAG |
| | | CCTTGCCCG |
| 668 | Encoding Probe 630 | ATGGAAGTAGTAGAAGGGTGAATGCTGGCAACA |
| | | CGGGACTCC |
| 669 | Encoding Probe 631 | ATGGAAGTAGTAGAAGGGCCGCGGGTGCAGAC |
| | | GACTCGGCAC |
| 670 | Encoding Probe 632 | ATGGAAGTAGTAGAAGGGTGGAACCCTCCACAC |
| | | CTTCGACCGG |
| 671 | Encoding Probe 633 | ATGGAAGTAGTAGAAGGGCCCAGGTTGGTCACG |
| | | TGTTACAGT |
| 672 | Encoding Probe 634 | ATGGAAGTAGTAGAAGGGTGGACTACTACCTAA |
| | | TGGGCCGGCT |
| 673 | Encoding Probe 635 | TAGAATTAGAGAGATGGGTCCCAGGTTGGTCAC |
| | | GTGTTAGAG |
| 674 | Encoding Probe 636 | TAGAATTAGAGAGATGGGTGGACCTACTACCTA |
| | | ATGGGCCCGC |
| 675 | Encoding Probe 637 | TAGAATTAGAGAGATGGGTGAACTGCTGAAAGC |
| | | GGTTTACTTG |
| 676 | Encoding Probe 638 | TAGAATTAGAGAGATGGGAAGGATATCTGCGCA |
| | | TTCCACGCG |
| 677 | Encoding Probe 639 | TAGAATTAGAGAGATGGGTAATTTGAGTTTTAG |
| | | CCTTGCCCG |
| 678 | Encoding Probe 640 | TAGAATTAGAGAGATGGGTGAATGCTGGCAACA |
| | | CGGGACTCC |
| 679 | Encoding Probe 641 | TAGAATTAGAGAGATGGGCCGCGGGTGCAGACG |
| | | ACTCGGCAC |
| 680 | Encoding Probe 642 | TAGAATTAGAGAGATGGGTGGAACCCTCCACAC |
| | | CTTCGACCGG |
| 681 | Encoding Probe 643 | TAGAATTAGAGAGATGGGCCCAGGTTGGTCACG |
| | | TGTTACAGT |
| 682 | Encoding Probe 644 | TAGAATTAGAGAGATGGGTGGACTACTACCTAA |
| | | TGGGCCGGCT |
| 683 | Encoding Probe 645 | GGAATTTAGTGAGAAGGGTTATTCGATACTATG |
| | | CGGTATTTTA |
| 684 | Encoding Probe 646 | GGAATTTAGTGAGAAGGGCAACCCCATTGTGAA |
| | | TGATTCTGCT |
| 685 | Encoding Probe 647 | GGAATTTAGTGAGAAGGGTGGACCATTACTCTA |
| | | GTCTCGCTCA |
| 686 | Encoding Probe 648 | GGAATTTAGTGAGAAGGGCACCCTCTCGATATC |
| | | TACGCAAAA |
| 687 | Encoding Probe 649 | GGAATTTAGTGAGAAGGGTGCGCCGAAGAGTCG |
| | | CATGCTTAGT |
| 688 | Encoding Probe 650 | GGAATTTAGTGAGAAGGGTGGAGGCATAAGGGC |
| | | CATACTGTGG |
| 689 | Encoding Probe 651 | GGAATTTAGTGAGAAGGGCGCTGGCTTCAGATA |
| | | CTTCGGCAC |
| 690 | Encoding Probe 652 | GGAATTTAGTGAGAAGGGTGGTTACCAGTCTCA |
| | | CCTTAGGTGG |
| 691 | Encoding Probe 653 | GGAATTTAGTGAGAAGGGTTATTCGATACTATG |
| | | CGGTATTATAG |
| 692 | Encoding Probe 654 | GGAATTTAGTGAGAAGGGAAACCCATTGTGAAT |
| | | GATTCTCCTG |
| 693 | Encoding Probe 655 | TGTAATAGTAAGGAGGGATTATTCGATACTATG |
| | | CGGTATTTTA |
| 694 | Encoding Probe 656 | TGTAATAGTAAGGAGGGACAACCCCATTGTGAA |
| | | TGATTCTGCT |
| 695 | Encoding Probe 657 | TGTAATAGTAAGGAGGGAGGACCATTACTCTAG |
| | | TCTCGCTCA |
| 696 | Encoding Probe 658 | TGTAATAGTAAGGAGGGACACCCTCTCGATATC |
| | | TACGCAAAA |
| 697 | Encoding Probe 659 | TGTAATAGTAAGGAGGGAGCGCCGAAGAGTCGC |
| | | ATGCTTAGT |
| 698 | Encoding Probe 660 | TGTAATAGTAAGGAGGGAGGAGGCATAAGGGC |
| | | CATACTGTGG |
| 699 | Encoding Probe 661 | TGTAATAGTAAGGAGGGACGCTGGCTTCAGATA |
| | | CTTCGGCAC |
| 700 | Encoding Probe 662 | TGTAATAGTAAGGAGGGAGGTTACCAGTCTCAC |
| | | CTTAGGTGG |
| 701 | Encoding Probe 663 | TGTAATAGTAAGGAGGGATTATTCGATACTATG |
| | | CGGTATTATAG |
| 702 | Encoding Probe 664 | TGTAATAGTAAGGAGGGAAAACCCATTGTGAAT |
| | | GATTCTCCTG |
| 703 | Encoding Probe 665 | GTAATAGATATGAGGGTGTAGCGCGTTACTCAC |
| | | CCGTCCCGG |
| 704 | Encoding Probe 666 | GTAATAGATATGAGGGTGAACGAAGATTCCCTA |
| | | CTGCTGGGA |
| 705 | Encoding Probe 667 | GTAATAGATATGAGGGTGGCGGCCACCTACGTA |
| | | TTACCGGCC |
| 706 | Encoding Probe 668 | GTAATAGATATGAGGGTGAAGCGCTACACTAGG |
| | | AATTCCCGA |
| 707 | Encoding Probe 669 | GTAATAGATATGAGGGTGGCGTGCTTCGAATTA |
| | | AACCACTAC |
| 708 | Encoding Probe 670 | GTAATAGATATGAGGGTGAACGGACTTAACCCA |
| | | ACATCTGTG |
| 709 | Encoding Probe 671 | GTAATAGATATGAGGGTGGGTCATTGTAGCACG |
| | | TGTGTACGG |
| 710 | Encoding Probe 672 | GTAATAGATATGAGGGTGTGTCTCTCATGGTGTG |
| | | ACGGGACC |
| 711 | Encoding Probe 673 | GTAATAGATATGAGGGTGACGACGCGTTACTCA |
| | | CCCGTCGCG |
| 712 | Encoding Probe 674 | GTAATAGATATGAGGGTGCAACCCTCTCAGGTC |
| | | GGCTACCGT |
| 713 | Encoding Probe 675 | ATGTATTAAGAGGAGGGATAGCGCGTTACTCAC |
| | | CCGTCCCGG |
| 714 | Encoding Probe 676 | ATGTATTAAGAGGAGGGAAACGAAGATTCCCTA |
| | | CTGCTGGGA |
| 715 | Encoding Probe 677 | ATGTATTAAGAGGAGGGAGCGGCCACCTACGTA |
| | | TTACCGGCC |
| 716 | Encoding Probe 678 | ATGTATTAAGAGGAGGGAAAGCGCTACACTAGG |
| | | AATTCCCGA |
| 717 | Encoding Probe 679 | ATGTATTAAGAGGAGGGAGCGTGCTTCGAATTA |
| | | AACCACTAC |
| 718 | Encoding Probe 680 | ATGTATTAAGAGGAGGGAAACGGACTTAACCCA |
| | | ACATCTGTG |
| 719 | Encoding Probe 681 | ATGTATTAAGAGGAGGGAGGTCATTGTAGCACG |
| | | TGTGTACGG |
| 720 | Encoding Probe 682 | ATGTATTAAGAGGAGGGATGTCTCTCATGGTGT |
| | | GACGGGACC |
| 721 | Encoding Probe 683 | ATGTATTAAGAGGAGGGAACGACGCGTTACTCA |
| | | CCCGTCGCG |
| 722 | Encoding Probe 684 | ATGTATTAAGAGGAGGGACAACCCTCTCAGGTC |
| | | GGCTACCGT |
| 723 | Encoding Probe 685 | GGTAATTGAGTAGAAGGGTGTCCCCTCCTTAAG |
| | | CAGATCTGAG |
| 724 | Encoding Probe 686 | GGTAATTGAGTAGAAGGGTGTACGAATAACTTC |
| | | TTCGTTCAGCG |
| 725 | Encoding Probe 687 | GGTAATTGAGTAGAAGGGTCTTACTATGCCATCT |
| | | ACGCAAGG |
| 726 | Encoding Probe 688 | GGTAATTGAGTAGAAGGGTGACCTCTGTCATAC |
| | | TCTAGCTAAC |
| 727 | Encoding Probe 689 | GGTAATTGAGTAGAAGGGTGTGCCTGTATGCAC |
| | | GCTATCCAGG |
| 728 | Encoding Probe 690 | GGTAATTGAGTAGAAGGGCGAGGTCATATAGGG |
| | | CATGATGTAA |
| 729 | Encoding Probe 691 | GGTAATTGAGTAGAAGGGTGCTCCATCTTCATG |
| | | AAGTCGACAA |
| 730 | Encoding Probe 692 | GGTAATTGAGTAGAAGGGTGCTCCCTCTTTCGTT |
| | | AGGCCTGG |
| 731 | Encoding Probe 693 | GGTAATTGAGTAGAAGGGCGACCTCGTCTTAAG |
| | | GGTAGGAAT |
| 732 | Encoding Probe 694 | GGTAATTGAGTAGAAGGGTGTACGAATAACTTC |
| | | TTCGTTCTGC |
| 733 | Encoding Probe 695 | TTGTAAAATAGGGAGGGAGTCCCCTCCTTAAGC |
| | | AGATCTGAG |
| 734 | Encoding Probe 696 | TTGTAAAATAGGGAGGGAGTACGAATAACTTCT |
| | | TCGTTCAGCG |
| 735 | Encoding Probe 697 | TTGTAAAATAGGGAGGGATCTTACTATGCCATCT |
| | | ACGCAAGG |
| 736 | Encoding Probe 698 | TTGTAAAATAGGGAGGGATGACCTCTGTCATAC |
| | | TCTAGCTAAC |
| 737 | Encoding Probe 699 | TTGTAAAATAGGGAGGGAGTGCCTGTATGCACG |
| | | CTATCCAGG |
| 738 | Encoding Probe 700 | TTGTAAAATAGGGAGGGACGAGGTCATATAGGG |
| | | CATGATGTAA |
| 739 | Encoding Probe 701 | TTGTAAAATAGGGAGGGAGCTCCATCTTCATGA |
| | | AGTCGACAA |
| 740 | Encoding Probe 702 | TTGTAAAATAGGGAGGGATGCTCCCTCTTTCGTT |
| | | AGGCCTGG |
| 741 | Encoding Probe 703 | TTGTAAAATAGGGAGGGACGACCTCGTCTTAAG |
| | | GGTAGGAAT |
| 742 | Encoding Probe 704 | TTGTAAAATAGGGAGGGATGTACGAATAACTTC |
| | | TTCGTTCTGC |
| 743 | Encoding Probe 705 | TGTATAGGATTAGAAGGGACACCCACGAGCGGA |
| | | CACGTTGGC |
| 744 | Encoding Probe 706 | TGTATAGGATTAGAAGGGTGTGGGAATAGCTGG |
| | | ATCAGGCAAC |
| 745 | Encoding Probe 707 | TGTATAGGATTAGAAGGGCAACCGGTGCTTATT |
| | | CTTAGAGATG |
| 746 | Encoding Probe 708 | TGTATAGGATTAGAAGGGTGTGCCCTCTGACAC |
| | | ACTCTAGAGC |
| 747 | Encoding Probe 709 | TGTATAGGATTAGAAGGGAATAGAGCTTCCTGA |
| | | CATGTCTTC |
| 748 | Encoding Probe 710 | TGTATAGGATTAGAAGGGTGGATCGTAGCAACT |
| | | AGTGACATCC |
| 749 | Encoding Probe 711 | TGTATAGGATTAGAAGGGTGACATCCGGACTAC |
| | | GATCGGTAAA |
| 750 | Encoding Probe 712 | TGTATAGGATTAGAAGGGCCAGGCTAACGACTT |
| | | CTGGTATTG |
| 751 | Encoding Probe 713 | TGTATAGGATTAGAAGGGAACCCCCACGAGCGG |
| | | ACACGTAGG |
| 752 | Encoding Probe 714 | TGTATAGGATTAGAAGGGTGCGAATAGCTGGAT |
| | | CAGGCTTCGC |
| 753 | Encoding Probe 715 | TATAGTTATGGAGAAGGGACACCCACGAGCGGA |
| | | CACGTTGGC |
| 754 | Encoding Probe 716 | TATAGTTATGGAGAAGGGTGTGGGAATAGCTGG |
| | | ATCAGGCAAC |
| 755 | Encoding Probe 717 | TATAGTTATGGAGAAGGGCAACCGGTGCTTATT |
| | | CTTAGAGATG |
| 756 | Encoding Probe 718 | TATAGTTATGGAGAAGGGTGTGCCCTCTGACAC |
| | | ACTCTAGAGC |
| 757 | Encoding Probe 719 | TATAGTTATGGAGAAGGGAATAGAGCTTCCTGA |
| | | CATGTCTTC |
| 758 | Encoding Probe 720 | TATAGTTATGGAGAAGGGTGGATCGTAGCAACT |
| | | AGTGACATCC |
| 759 | Encoding Probe 721 | TATAGTTATGGAGAAGGGTGACATCCGGACTAC |
| | | GATCGGTAAA |
| 760 | Encoding Probe 722 | TATAGTTATGGAGAAGGGCCAGGCTAACGACTT |
| | | CTGGTATTG |
| 761 | Encoding Probe 723 | TATAGTTATGGAGAAGGGAACCCCCACGAGCGG |
| | | ACACGTAGG |
| 762 | Encoding Probe 724 | TATAGTTATGGAGAAGGGTGCGAATAGCTGGAT |
| | | CAGGCTTCGC |

### Example 13. Species-Level Swap in Tissue Samples

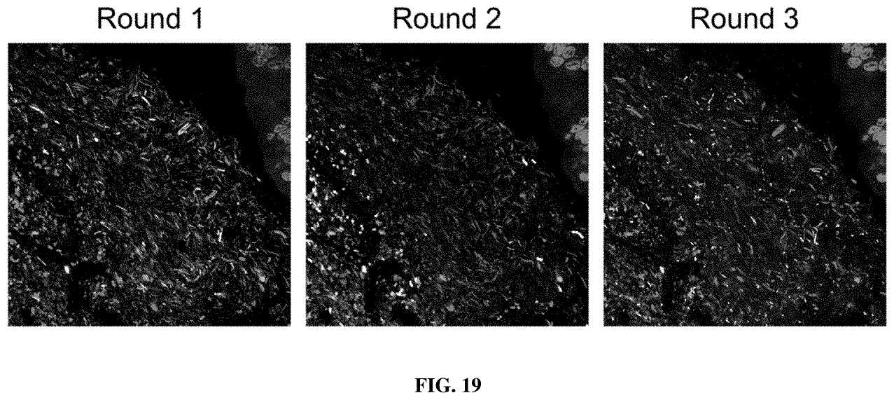

To extend the ability to perform HiPR-Swap at the phylum level on a tissue specimen (colon of a healthy mouse) to the species level probes to perform a simple taxon identification experiment were designed, barcoding the sixty-five most abundant species in healthy mouse stool (measured internally by PacBio 16S long-read sequencing). As shown in FIG. 19, we were able to identified several dozen species. Signal exchange correctly matched expectations for taxa present in the encoding panel.

Species-level swap protocol: OCT-embedded formalin-fixed tissue was sectioned at 10-micron thickness onto circular glass coverslips made for Bioptechs FCS2 flow cell. The tissue was covered with 2% formaldehyde for two hours at room temperature to fix the sample. The sample was washed by removing the buffer and replacing it with 1×PBS for 5 minutes (this was repeated two more times). The fixed tissue specimen was stored in 70% ethanol at 4° C. overnight. The following buffers were prepared:
Encoding buffer: Encoding probes (100 nM of each encoding probe in complex pool, 5 nM of blocking probes in the complex probe pool+EUB at 1 μM); 2× sodium chloride sodium citrate (SSC); 5×Denhardt's solution; 10% dextran sulfate; 10% ethylene carbonate; and 0.01% sodium dodecyl sulfate (SDS)
Round 1 readout buffer: 400 nM each of readout probes: 21, 11, 22, 12, 13, and 9; 2× sodium chloride sodium citrate (SSC); 5×Denhardt's solution; 10% dextran sulfate; 10% ethylene carbonate; and 0.01% sodium dodecyl sulfate (SDS)
Round 2 readout buffer: 10 μM each of exchange probe: 4-5, 7-8, and 10; 400 nM each of readout probes: 14, 24, 16, 17, and 23; 2× sodium chloride sodium citrate (SSC); 5×Denhardt's solution; 10% dextran sulfate; 10% ethylene carbonate; and 0.01% sodium dodecyl sulfate (SDS)
Round 3 readout buffer: 10 μM each of exchange probe: 14, 21, 17-18, and 20; 400 nM each of readout probes: 18, 19, 25, 20, and 21; 2× sodium chloride sodium citrate (SSC); 5×Denhardt's solution; 10% dextran sulfate; 10% ethylene carbonate; 0.01% sodium dodecyl sulfate (SDS)
Wash buffer: 215 mM NaCl; 20 mM Tris-HCl (pH 7.5); and 5 mM EDTA

Place 10 mg/mL lysozyme to completely cover the specimen and incubate for 30 minutes at 37° C. in a humidified chamber. Wash the specimen with 1×PBS for 15 minutes at room temperature. Dry the specimen by submerging it in 100% ethanol and allowing it to air dry. Place the coverslip on the FCS2 flow cell (Bioptechs) and assemble. Place the flow cell assembly on the microscope stage (Zeiss i880 confocal). Connect the flow cell input port to the Aria Automated Perfusion System (Fluigent). Calibrate the Aria Automated Perfusion System using DI water. Load encoding, readout buffers, wash buffer, 1×PBS buffer, 5×SSC+DAPI buffer (40 ng/mL DAPI in 5×SSC), and 2×SSC buffer into Aria Automated Perfusion System at the desired reservoir locations. Execute the following sequence on the Aria:
a. Incubate the specimen with 1×PBS at room temperature for 15 minutes.
b. Incubate the specimen in the Encoding buffer at 37° C. for 2 hours.
c. Incubate the specimen in the Wash buffer at 42° C. for 15 minutes.
d. Incubate the specimen in the Round 1 readout buffer at 37° C. for 1 hour.
e. Incubate the specimen in the Wash buffer at 42° C. for 15 minutes.
f. Incubate the specimen in 5×SSC+DAPI at room temperature for 15 minutes.
g. Flush the specimen with 2×SSC for imaging.
h. Perform image acquisition, exciting the specimen with 633 nm, 561 nm, 514 nm, 488 nm, and 405 nm lines and collect spectra.
i. Incubate the specimen in the Round 2 readout buffer at 37° C. for 1 hour.
j. Repeat steps e-h.
k. Incubate the specimen in the Round 3 readout buffer at 37° C. for 1 hour.
l. Repeat steps e-h.

Table 9 shows the encoding, readout, and exchange probe sequences used in this example.

TABLE 9
Encoding, readout, and exchange probe sequences used in Example 13

| SEQ ID | | |
|---|---|---|
| NO: | Probe Name | Sequence (in 5′ to 3′ order) |
| 763 | Readout Probe | /5Alex488N/CCCTTCTACTCAATTACCTCATCCC |
| | 21 | |
| 597 | Readout Probe | /5Alex532/CCCAACTACCCATATTAACACACCC |
| | 11 | |
| 764 | Readout Probe | /5Alex546N/CCCATCTCACTATCTTATCACCCAC |
| | 22 | |
| 598 | Readout Probe | /56-ROXN/CCCTTCTCACTAAATTCCAACACCC |
| | 12 | |
| 9 | Readout Probe | /5Alex405N/TTCTCCCTCTATCAACTCTA |
| | 9 | |
| 599 | Readout Probe | /5Alex647N/CACCCTCATATCTATTACCCTCCCA |
| | 13 | |
| 600 | Readout Probe | /5Alex488N/TCCCTCCTTACTATTACACTCACCC |
| | 14 | |
| 602 | Readout Probe | /5Alex546N/CCCTTCTACTACTTCCATACATCCC |
| | 16 | |
| 603 | Readout Probe | /56-ROXN/CCCTTCTAATCCTATACACTCACCC |
| | 17 | |
| 765 | Readout Probe | /5Alex647N/CCCACTCAACATATCATTCCACCAC |
| | 23 | |
| 766 | Readout Probe | /5Alex532/CCCAACTATACTCTATCCTCCATCC |
| | 24 | |
| 604 | Readout Probe | /5Alex488N/CCCATCTCTCTAATTCTACTCCACC |
| | 18 | |
| 605 | Readout Probe | /5Alex532/TCCCTCCTCTTAATACATCCTCCTC |
| | 19 | |
| 767 | Readout Probe | /5Alex546N/CCTCACCCTAATAATACTCCAACCC |
| | 25 | |
| 606 | Readout Probe | /56-ROXN/CATCCCTACTTACTTATCCTCCACC |
| | 20 | |
| 607 | Readout Probe | /5Alex647N/CCCTTCTCCATAACTATACCCTTCC |
| | 21 | |
| 768 | Exchange | GGGATGAGGTAATTGAGTAGAAGGG |
| | Probe 4 | |
| 608 | Exchange | GGGTGTGTTAATATGGGTAGTTGGG |
| | Probe 5 | |
| 769 | Exchange | GTGGGTGATAAGATAGTGAGATGGG |
| | Probe 7 | |
| 609 | Exchange | GGGTGTTGGAATTTAGTGAGAAGGG |
| | Probe 8 | |
| 610 | Exchange | TGGGAGGGTAATAGATATGAGGGTG |
| | Probe 10 | |
| 611 | Exchange | GGGTGAGTGTAATAGTAAGGAGGGA |
| | Probe 14 | |
| 613 | Exchange | GGGATGTATGGAAGTAGTAGAAGGG |
| | Probe 17 | |
| 614 | Exchange | GGGTGAGTGTATAGGATTAGAAGGG |
| | Probe 18 | |
| 770 | Exchange | GTGGTGGAATGATATGTTGAGTGGG |
| | Probe 20 | |
| 771 | Exchange | GGATGGAGGATAGAGTATAGTTGGG |
| | Probe 21 | |
| 772 | Encoding | GTAATAGATATGAGGGTGCGGAGCGTCAGTAGGGC |
| | Probe 725 | GCCGCATTGGGAGGGTAATAGATAT |
| 773 | Encoding | GTAATAGATATGAGGGTGCGCACGCGGTATTAGACG |
| | Probe 726 | GAATTTGAATGGGAGGGTAATAGATAT |
| 774 | Encoding | GTAATAGATATGAGGGTGGACCCCCCGCTGCCCCTC |
| | Probe 727 | GACAACTGGGAGGGTAATAGATAT |
| 775 | Encoding | GTAATAGATATGAGGGTGCGCACGCGGTATTAGACG |
| | Probe 728 | GAATTAGATGGGAGGGTAATAGATAT |
| 776 | Encoding | GTAATAGATATGAGGGTGAATCCGCCGACTAGCTAA |
| | Probe 729 | TGCCGGTGGGAGGGTAATAGATAT |
| 777 | Encoding | GTAATAGATATGAGGGTGCGTCTTGCTCCCCGGCAA |
| | Probe 730 | AAGTCCTGGGAGGGTAATAGATAT |
| 778 | Encoding | GTAATAGATATGAGGGTGGACCCCCCGCTGCCCCTC |
| | Probe 731 | GACTACGTGGGAGGGTAATAGATAT |
| 779 | Encoding | GTAATAGATATGAGGGTGATGCGCCGACTAGCTAAT |
| | Probe 732 | GCGGGCTGGGAGGGTAATAGATAT |
| 780 | Encoding | ATGTATTAAGAGGAGGGACGGAGCGTCAGTAGGGC |
| | Probe 733 | GCCGCATGAGGAGGATGTATTAAGA |
| 781 | Encoding | ATGTATTAAGAGGAGGGACGCACGCGGTATTAGACG |
| | Probe 734 | GAATTTGAAGAGGAGGATGTATTAAGA |
| 782 | Encoding | ATGTATTAAGAGGAGGGAGACCCCCCGCTGCCCCTC |
| | Probe 735 | GACAACGAGGAGGATGTATTAAGA |
| 783 | Encoding | ATGTATTAAGAGGAGGGACGCACGCGGTATTAGACG |
| | Probe 736 | GAATTAGAGAGGAGGATGTATTAAGA |
| 784 | Encoding | ATGTATTAAGAGGAGGGAAATCCGCCGACTAGCTAA |
| | Probe 737 | TGCCGGGAGGAGGATGTATTAAGA |
| 785 | Encoding | ATGTATTAAGAGGAGGGACGTCTTGCTCCCCGGCAA |
| | Probe 738 | AAGTCCGAGGAGGATGTATTAAGA |
| 786 | Encoding | ATGTATTAAGAGGAGGGAGACCCCCCGCTGCCCCTC |
| | Probe 739 | GACTACGGAGGAGGATGTATTAAGA |
| 787 | Encoding | ATGTATTAAGAGGAGGGAATGCGCCGACTAGCTAAT |
| | Probe 740 | GCGGGCGAGGAGGATGTATTAAGA |
| 788 | Encoding | TGTAATAGTAAGGAGGGAGGCGTTAAGCCCCGGCAT |
| | Probe 741 | TTCTGAGGGTGAGTGTAATAGTAA |
| 789 | Encoding | TGTAATAGTAAGGAGGGACCACCCAACACCTAGTAA |
| | Probe 742 | TCATGCAGGGTGAGTGTAATAGTAA |
| 790 | Encoding | TGTAATAGTAAGGAGGGACTTGAAAGTGACTTTGCT |
| | Probe 743 | CACAGCGGGTGAGTGTAATAGTAA |
| 791 | Encoding | TGTAATAGTAAGGAGGGAGCAGTAGCCCTGATCATA |
| | Probe 744 | AGGCCGTGGGTGAGTGTAATAGTAA |
| 792 | Encoding | TGTAATAGTAAGGAGGGACCTCATCGTATACCACCA |
| | Probe 745 | GAGTAAAGGGTGAGTGTAATAGTAA |
| 793 | Encoding | TGTAATAGTAAGGAGGGAAGATGCACTCTAGCTGCA |
| | Probe 746 | CAGAAAGGGTGAGTGTAATAGTAA |
| 794 | Encoding | TGTAATAGTAAGGAGGGAGAGCTGCACTCTAGCTGC |
| | Probe 747 | ACACAAGGGTGAGTGTAATAGTAA |
| 795 | Encoding | TGTAATAGTAAGGAGGGACCTCATCGTATACCACCA |
| | Probe 748 | GAGAAAGGGTGAGTGTAATAGTAA |
| 796 | Encoding | TATAGTTATGGAGAAGGGTGGCGTTAAGCCCCGGCA |
| | Probe 749 | TTTCTGAGGAAGGGTATAGTTATGG |
| 797 | Encoding | TATAGTTATGGAGAAGGGCCACCCAACACCTAGTAA |
| | Probe 750 | TCATGCAGGAAGGGTATAGTTATGG |
| 798 | Encoding | TATAGTTATGGAGAAGGGCTTGAAAGTGACTTTGCT |
| | Probe 751 | CACAGCGGAAGGGTATAGTTATGG |
| 799 | Encoding | TATAGTTATGGAGAAGGGTGCAGTAGCCCTGATCAT |
| | Probe 752 | AAGGCCGGGAAGGGTATAGTTATGG |
| 800 | Encoding | TATAGTTATGGAGAAGGGCCTCATCGTATACCACCA |
| | Probe 753 | GAGTAAAGGAAGGGTATAGTTATGG |
| 801 | Encoding | TATAGTTATGGAGAAGGGAGATGCACTCTAGCTGCA |
| | Probe 754 | CAGAAAGGAAGGGTATAGTTATGG |
| 802 | Encoding | TATAGTTATGGAGAAGGGTGAGCTGCACTCTAGCTG |
| | Probe 755 | CACACAAGGAAGGGTATAGTTATGG |
| 803 | Encoding | TATAGTTATGGAGAAGGGCCTCATCGTATACCACCA |
| | Probe 756 | GAGAAAGGAAGGGTATAGTTATGG |
| 804 | Encoding | TTAATATGGGTAGTTGGGTGGAGAAAGGCAGGTTCC |
| | Probe 757 | TCACCGCGGGTGTGTTAATATGGGT |
| 805 | Encoding | TTAATATGGGTAGTTGGGTTCGTACCGTCTTCTGCTC |
| | Probe 758 | TTAGGTGGGTGTGTTAATATGGGT |
| 806 | Encoding | TTAATATGGGTAGTTGGGTCCCCGTCTTCTGCTCTTC |
| | Probe 759 | CCGGAGGGTGTGTTAATATGGGT |
| 807 | Encoding | TTAATATGGGTAGTTGGGTGGAGAAAGGCAGGTTCC |
| | Probe 760 | TCACGGCAGGGTGTGTTAATATGGGT |
| 808 | Encoding | TTAATATGGGTAGTTGGGATTCACATAATCCACCGC |
| | Probe 761 | TTGACGTGGGTGTGTTAATATGGGT |
| 809 | Encoding | TTAATATGGGTAGTTGGGCCCTCAGTCCCCGCACAC |
| | Probe 762 | CTACATGGGTGTGTTAATATGGGT |
| 810 | Encoding | TTAATATGGGTAGTTGGGTCTAACAGTTTCAAATGC |
| | Probe 763 | AGTTGTGTGGGTGTGTTAATATGGGT |
| 811 | Encoding | TTAATATGGGTAGTTGGGTGGAGGATTTCACATCTG |
| | Probe 764 | ACTTGTAAGTGGGTGTGTTAATATGGGT |
| 812 | Encoding | AATGATATGTTGAGTGGGTGGAGAAAGGCAGGTTCC |
| | Probe 765 | TCACCGCGTGGTGGAATGATATGTT |
| 813 | Encoding | AATGATATGTTGAGTGGGTTCGTACCGTCTTCTGCTC |
| | Probe 766 | TTAGGGTGGTGGAATGATATGTT |
| 814 | Encoding | AATGATATGTTGAGTGGGTCCCCGTCTTCTGCTCTTC |
| | Probe 767 | CCGGAGTGGTGGAATGATATGTT |
| 815 | Encoding | AATGATATGTTGAGTGGGTGGAGAAAGGCAGGTTCC |
| | Probe 768 | TCACGGCAGTGGTGGAATGATATGTT |
| 816 | Encoding | AATGATATGTTGAGTGGGATTCACATAATCCACCGC |
| | Probe 769 | TTGACGGTGGTGGAATGATATGTT |
| 817 | Encoding | AATGATATGTTGAGTGGGCCCTCAGTCCCCGCACAC |
| | Probe 770 | CTACATGTGGTGGAATGATATGTT |
| 818 | Encoding | AATGATATGTTGAGTGGGTCTAACAGTTTCAAATGC |
| | Probe 771 | AGTTGTGGTGGTGGAATGATATGTT |
| 819 | Encoding | AATGATATGTTGAGTGGGTGGAGGATTTCACATCTG |
| | Probe 772 | ACTTGTAAGGTGGTGGAATGATATGTT |
| 820 | Encoding | TGTATAGGATTAGAAGGGTGAGAGAACCCCTAGACA |
| | Probe 773 | TCGTGCGTGGGTGAGTGTATAGGATT |
| 821 | Encoding | TGTATAGGATTAGAAGGGACGCAGCGTCAGTTGGGC |
| | Probe 774 | GCCGCATGGGTGAGTGTATAGGATT |
| 822 | Encoding | TGTATAGGATTAGAAGGGATGAACGCTTTCGCTGTG |
| | Probe 775 | CCAAGGTGGGTGAGTGTATAGGATT |
| 823 | Encoding | TGTATAGGATTAGAAGGGTGGCAGTCTCGACAGAGT |
| | Probe 776 | CCTCTCGTGGGTGAGTGTATAGGATT |
| 824 | Encoding | TGTATAGGATTAGAAGGGACGGTGTTAGGCCTGTCG |
| | Probe 777 | CTACGCGGGTGAGTGTATAGGATT |
| 825 | Encoding | TGTATAGGATTAGAAGGGCGTTGTTAGGCCTGTCGC |
| | Probe 778 | TAGGCAGGGTGAGTGTATAGGATT |
| 826 | Encoding | TGTATAGGATTAGAAGGGCGGCTAGCTAATGTCACG |
| | Probe 779 | CATCGGTGGGTGAGTGTATAGGATT |
| 827 | Encoding | TGTATAGGATTAGAAGGGATGCTCGCCCACTCAAGA |
| | Probe 780 | CCGAGTGGGTGAGTGTATAGGATT |
| 828 | Encoding | TAGAATTAGAGAGATGGGTGAGAGAACCCCTAGAC |
| | Probe 781 | ATCGTGCGGGTGGAGTAGAATTAGAG |
| 829 | Encoding | TAGAATTAGAGAGATGGGACGCAGCGTCAGTTGGGC |
| | Probe 782 | GCCGCATGGTGGAGTAGAATTAGAG |
| 830 | Encoding | TAGAATTAGAGAGATGGGATGAACGCTTTCGCTGTG |
| | Probe 783 | CCAAGGTGGTGGAGTAGAATTAGAG |
| 831 | Encoding | TAGAATTAGAGAGATGGGTGGCAGTCTCGACAGAGT |
| | Probe 784 | CCTCTCGGGTGGAGTAGAATTAGAG |
| 832 | Encoding | TAGAATTAGAGAGATGGGACGGTGTTAGGCCTGTCG |
| | Probe 785 | CTACGCGGTGGAGTAGAATTAGAG |
| 833 | Encoding | TAGAATTAGAGAGATGGGCGTTGTTAGGCCTGTCGC |
| | Probe 786 | TAGGCAGGTGGAGTAGAATTAGAG |
| 834 | Encoding | TAGAATTAGAGAGATGGGCGGCTAGCTAATGTCACG |
| | Probe 787 | CATCGGTGGTGGAGTAGAATTAGAG |
| 835 | Encoding | TAGAATTAGAGAGATGGGATGCTCGCCCACTCAAGA |
| | Probe 788 | CCGAGTGGTGGAGTAGAATTAGAG |
| 836 | Encoding | GGAATTTAGTGAGAAGGGACGTGAAACTATACCATC |
| | Probe 789 | GGGTTAAGGGTGTTGGAATTTAGTG |
| 837 | Encoding | GGAATTTAGTGAGAAGGGTAGGCGGTGAAACTATAC |
| | Probe 790 | CATGCCGGGTGTTGGAATTTAGTG |
| 838 | Encoding | GGAATTTAGTGAGAAGGGTGTAAAAATGGTATGCAT |
| | Probe 791 | ACCAAAGAAGGGTGTTGGAATTTAGTG |
| 839 | Encoding | GGAATTTAGTGAGAAGGGTTTGGTATGCATACCAAA |
| | Probe 792 | CTTTAAAGTGGGTGTTGGAATTTAGTG |
| 840 | Encoding | GGAATTTAGTGAGAAGGGTACGTGAAACTATACCAT |
| | Probe 793 | CGGGATAGGGTGTTGGAATTTAGTG |
| 841 | Encoding | GGAATTTAGTGAGAAGGGTGTATGGTATGCATACCA |
| | Probe 794 | AACTAATGGGTGTTGGAATTTAGTG |
| 842 | Encoding | GGAATTTAGTGAGAAGGGCGCGAAACTATACCATCG |
| | Probe 795 | GGTAAATGGGTGTTGGAATTTAGTG |
| 843 | Encoding | GGAATTTAGTGAGAAGGGCATTACAAAATGGTATGC |
| | Probe 796 | ATACCTTTGGGTGTTGGAATTTAGTG |
| 844 | Encoding | GGATAGAGTATAGTTGGGACGTGAAACTATACCATC |
| | Probe 797 | GGGTTAAGGATGGAGGATAGAGTAT |
| 845 | Encoding | GGATAGAGTATAGTTGGGTAGGCGGTGAAACTATAC |
| | Probe 798 | CATGCCGGATGGAGGATAGAGTAT |
| 846 | Encoding | GGATAGAGTATAGTTGGGTGTAAAAATGGTATGCAT |
| | Probe 799 | ACCAAAGAAGGATGGAGGATAGAGTAT |
| 847 | Encoding | GGATAGAGTATAGTTGGGTTTGGTATGCATACCAAA |
| | Probe 800 | CTTTAAAGGGATGGAGGATAGAGTAT |
| 848 | Encoding | GGATAGAGTATAGTTGGGTACGTGAAACTATACCAT |
| | Probe 801 | CGGGATAGGATGGAGGATAGAGTAT |
| 849 | Encoding | GGATAGAGTATAGTTGGGTGTATGGTATGCATACCA |
| | Probe 802 | AACTAATGGATGGAGGATAGAGTAT |
| 850 | Encoding | GGATAGAGTATAGTTGGGCGCGAAACTATACCATCG |
| | Probe 803 | GGTAAATGGATGGAGGATAGAGTAT |
| 851 | Encoding | GGATAGAGTATAGTTGGGCATTACAAAATGGTATGC |
| | Probe 804 | ATACCTTTGGATGGAGGATAGAGTAT |
| 852 | Encoding | GGTAATTGAGTAGAAGGGAATGGGTATTAGTACCAA |
| | Probe 805 | TTTCTCTCAGGGATGAGGTAATTGAGT |
| 853 | Encoding | GGTAATTGAGTAGAAGGGCGTCCTTCGCAGGGTAGC |
| | Probe 806 | TGCGGAGGGATGAGGTAATTGAGT |
| 854 | Encoding | GGTAATTGAGTAGAAGGGCGGGAAGGGAAACGCTC |
| | Probe 807 | TTTCTTCGGGATGAGGTAATTGAGT |
| 855 | Encoding | GGTAATTGAGTAGAAGGGTGCGACCGCAACTATTCT |
| | Probe 808 | CTAGAGGTGGGATGAGGTAATTGAGT |
| 856 | Encoding | GGTAATTGAGTAGAAGGGTGGAACATTTCACCTCTA |
| | Probe 809 | ACTTATCATTGTGGGATGAGGTAATTGAGT |
| 857 | Encoding | GGTAATTGAGTAGAAGGGCGGTCCTTATTCGTACGA |
| | Probe 810 | TACTTAGTGGGATGAGGTAATTGAGT |
| 858 | Encoding | GGTAATTGAGTAGAAGGGTGTCCCCCTATGTATCGT |
| | Probe 811 | CGCCAACGGGATGAGGTAATTGAGT |
| 859 | Encoding | GGTAATTGAGTAGAAGGGAATGGGTATTAGTACCAA |
| | Probe 812 | TTTCTCACACGGGATGAGGTAATTGAGT |
| 860 | Encoding | AGTATTATTAGGGTGAGGAATGGGTATTAGTACCAA |
| | Probe 813 | TTTCTCTCAGGGTTGGAGTATTATTAG |
| 861 | Encoding | AGTATTATTAGGGTGAGGCGTCCTTCGCAGGGTAGC |
| | Probe 814 | TGCGGAGGGTTGGAGTATTATTAG |
| 862 | Encoding | AGTATTATTAGGGTGAGGCGGGAAGGGAAACGCTCT |
| | Probe 815 | TTCTTCGGGTTGGAGTATTATTAG |
| 863 | Encoding | AGTATTATTAGGGTGAGGGCGACCGCAACTATTCTC |
| | Probe 816 | TAGAGGTGGGTTGGAGTATTATTAG |
| 864 | Encoding | AGTATTATTAGGGTGAGGTGGAACATTTCACCTCTA |
| | Probe 817 | ACTTATCATTGTGGGTTGGAGTATTATTAG |
| 865 | Encoding | AGTATTATTAGGGTGAGGCGGTCCTTATTCGTACGA |
| | Probe 818 | TACTTAGTGGGTTGGAGTATTATTAG |
| 866 | Encoding | AGTATTATTAGGGTGAGGGTCCCCCTATGTATCGTC |
| | Probe 819 | GCCAACGGGTTGGAGTATTATTAG |
| 867 | Encoding | AGTATTATTAGGGTGAGGAATGGGTATTAGTACCAA |
| | Probe 820 | TTTCTCACACGGGTTGGAGTATTATTAG |
| 868 | Encoding | GGAATTTAGTGAGAAGGGAGGCACTCGAATGCCAC |
| | Probe 821 | ATGATTACTGGGTGTTGGAATTTAGTG |
| 869 | Encoding | GGAATTTAGTGAGAAGGGTGACCTCAAGTTACACAG |
| | Probe 822 | TTTCCTCTGGGTGTTGGAATTTAGTG |
| 870 | Encoding | GGAATTTAGTGAGAAGGGAGGCACTCGAATGCCAC |
| | Probe 823 | ATGATAACGGGTGTTGGAATTTAGTG |
| 871 | Encoding | GGAATTTAGTGAGAAGGGTGGACCGCCACTCGAATG |
| | Probe 824 | CCACAACTGGGTGTTGGAATTTAGTG |
| 872 | Encoding | GGAATTTAGTGAGAAGGGTGAGCTGCACTCAAGTTA |
| | Probe 825 | CACACAAGGGTGTTGGAATTTAGTG |
| 873 | Encoding | GGAATTTAGTGAGAAGGGAGGCACTCGAATGCCAC |
| | Probe 826 | ATGAAAAGGGTGTTGGAATTTAGTG |
| 874 | Encoding | GGAATTTAGTGAGAAGGGTGGACCGCCACTCGAATG |
| | Probe 827 | CCACTACGGGTGTTGGAATTTAGTG |
| 875 | Encoding | GGAATTTAGTGAGAAGGGTGGCCGCCACTCGAATGC |
| | Probe 828 | CACAACTGGGTGTTGGAATTTAGTG |
| 876 | Encoding | AATGATATGTTGAGTGGGAGGCACTCGAATGCCACA |
| | Probe 829 | TGATTACTGTGGTGGAATGATATGTT |
| 877 | Encoding | AATGATATGTTGAGTGGGTGACCTCAAGTTACACAG |
| | Probe 830 | TTTCCTCTGTGGTGGAATGATATGTT |
| 878 | Encoding | AATGATATGTTGAGTGGGAGGCACTCGAATGCCACA |
| | Probe 831 | TGATAACGTGGTGGAATGATATGTT |
| 879 | Encoding | AATGATATGTTGAGTGGGTGGACCGCCACTCGAATG |
| | Probe 832 | CCACAACTGTGGTGGAATGATATGTT |
| 880 | Encoding | AATGATATGTTGAGTGGGTGAGCTGCACTCAAGTTA |
| | Probe 833 | CACACAAGTGGTGGAATGATATGTT |
| 881 | Encoding | AATGATATGTTGAGTGGGAGGCACTCGAATGCCACA |
| | Probe 834 | TGAAAAGTGGTGGAATGATATGTT |
| 882 | Encoding | AATGATATGTTGAGTGGGTGGACCGCCACTCGAATG |
| | Probe 835 | CCACTACGTGGTGGAATGATATGTT |
| 883 | Encoding | AATGATATGTTGAGTGGGTGGCCGCCACTCGAATGC |
| | Probe 836 | CACAACTGTGGTGGAATGATATGTT |
| 884 | Encoding | ATAAGATAGTGAGATGGGAGAGACACTCTAGCAAA |
| | Probe 837 | ACAGTAAGGTGGGTGATAAGATAGTG |
| 885 | Encoding | ATAAGATAGTGAGATGGGTGGAGTTTTTCACACACT |
| | Probe 838 | GCCTACGTGGGTGATAAGATAGTG |
| 886 | Encoding | ATAAGATAGTGAGATGGGCCGCGTTACCGGCCCGCC |
| | Probe 839 | AGGGCCTGTGGGTGATAAGATAGTG |
| 887 | Encoding | ATAAGATAGTGAGATGGGTAGAAAACTTCATCTTAA |
| | Probe 840 | TCGCTAGCGTGGGTGATAAGATAGTG |
| 888 | Encoding | ATAAGATAGTGAGATGGGTGAGACACTCTAGCAAA |
| | Probe 841 | ACAGTAAGGTGGGTGATAAGATAGTG |
| 889 | Encoding | ATAAGATAGTGAGATGGGAGGAAACTTCATCTTAAT |
| | Probe 842 | CGCTTGCAGTGGGTGATAAGATAGTG |
| 890 | Encoding | ATAAGATAGTGAGATGGGCGGGTTACCGGCCCGCCA |
| | Probe 843 | GGGCCTGTGGGTGATAAGATAGTG |
| 891 | Encoding | ATAAGATAGTGAGATGGGTGCCTTTTTCACACACTG |
| | Probe 844 | CCATCGCGTGGGTGATAAGATAGTG |
| 892 | Encoding | AATGATATGTTGAGTGGGAGAGACACTCTAGCAAAA |
| | Probe 845 | CAGTAAGGTGGTGGAATGATATGTT |
| 893 | Encoding | AATGATATGTTGAGTGGGTGGAGTTTTTCACACACT |
| | Probe 846 | GCCTACGTGGTGGAATGATATGTT |
| 894 | Encoding | AATGATATGTTGAGTGGGCCGCGTTACCGGCCCGCC |
| | Probe 847 | AGGGCCTGTGGTGGAATGATATGTT |
| 895 | Encoding | AATGATATGTTGAGTGGGTAGAAAACTTCATCTTAA |
| | Probe 848 | TCGCTAGCGTGGTGGAATGATATGTT |
| 896 | Encoding | AATGATATGTTGAGTGGGTGAGACACTCTAGCAAAA |
| | Probe 849 | CAGTAAGGTGGTGGAATGATATGTT |
| 897 | Encoding | AATGATATGTTGAGTGGGAGGAAACTTCATCTTAAT |
| | Probe 850 | CGCTTGCAGTGGTGGAATGATATGTT |
| 898 | Encoding | AATGATATGTTGAGTGGGCGGGTTACCGGCCCGCCA |
| | Probe 851 | GGGCCTGTGGTGGAATGATATGTT |
| 899 | Encoding | AATGATATGTTGAGTGGGTGCCTTTTTCACACACTGC |
| | Probe 852 | CATCGCGTGGTGGAATGATATGTT |
| 900 | Encoding | GGTAATTGAGTAGAAGGGAGTCGGTACCTGCAAACA |
| | Probe 853 | TCCACAGCAGGGATGAGGTAATTGAGT |
| 901 | Encoding | GGTAATTGAGTAGAAGGGTGGACCCGAAAGATAGG |
| | Probe 854 | CCATGGACGGGATGAGGTAATTGAGT |
| 902 | Encoding | GGTAATTGAGTAGAAGGGCGAGCCGCCGACTGTATA |
| | Probe 855 | TCGGGCGGGATGAGGTAATTGAGT |
| 903 | Encoding | GGTAATTGAGTAGAAGGGTGACGATGACTTTAAGGA |
| | Probe 856 | TTGGACGCTGGGATGAGGTAATTGAGT |
| 904 | Encoding | GGTAATTGAGTAGAAGGGATCCCAATCACCGGTTTC |
| | Probe 857 | ACCGATGGGATGAGGTAATTGAGT |
| 905 | Encoding | GGTAATTGAGTAGAAGGGAACCCACAAAATTTCACG |
| | Probe 858 | GCAGCGTGGGATGAGGTAATTGAGT |
| 906 | Encoding | GGTAATTGAGTAGAAGGGAATGATAAATCTTTGCTC |
| | Probe 859 | CGACAGTCGGGATGAGGTAATTGAGT |
| 907 | Encoding | GGTAATTGAGTAGAAGGGTGGCTCTGGATCTTTCCT |
| | Probe 860 | CTGGTTGGGATGAGGTAATTGAGT |
| 908 | Encoding | AATGATATGTTGAGTGGGAGTCGGTACCTGCAAACA |
| | Probe 861 | TCCACAGCAGTGGTGGAATGATATGTT |
| 909 | Encoding | AATGATATGTTGAGTGGGTGGACCCGAAAGATAGGC |
| | Probe 862 | CATGGACGTGGTGGAATGATATGTT |
| 910 | Encoding | AATGATATGTTGAGTGGGCGAGCCGCCGACTGTATA |
| | Probe 863 | TCGGGCGTGGTGGAATGATATGTT |
| 911 | Encoding | AATGATATGTTGAGTGGGTGACGATGACTTTAAGGA |
| | Probe 864 | TTGGACGCTGTGGTGGAATGATATGTT |
| 912 | Encoding | AATGATATGTTGAGTGGGATCCCAATCACCGGTTTC |
| | Probe 865 | ACCGATGTGGTGGAATGATATGTT |
| 913 | Encoding | AATGATATGTTGAGTGGGAACCCACAAAATTTCACG |
| | Probe 866 | GCAGCGGTGGTGGAATGATATGTT |
| 914 | Encoding | AATGATATGTTGAGTGGGAATGATAAATCTTTGCTC |
| | Probe 867 | CGACAGTCGTGGTGGAATGATATGTT |
| 915 | Encoding | AATGATATGTTGAGTGGGTGGCTCTGGATCTTTCCTC |
| | Probe 868 | TGGTTGTGGTGGAATGATATGTT |
| 916 | Encoding | GTAATAGATATGAGGGTGAGTTGGTACATACAAAAT |
| | Probe 869 | GGTATACAATGTGGGAGGGTAATAGATAT |
| 917 | Encoding | GTAATAGATATGAGGGTGATGAATGGTATACATACC |
| | Probe 870 | AAACTTTAAAGTGGGAGGGTAATAGATAT |
| 918 | Encoding | GTAATAGATATGAGGGTGTGTATGGTATACATACCA |
| | Probe 871 | AACTTTATAGGTGGGAGGGTAATAGATAT |
| 919 | Encoding | GTAATAGATATGAGGGTGGTAGGTACATACAAAATG |
| | Probe 872 | GTATACAATGTGGGAGGGTAATAGATAT |
| 920 | Encoding | GTAATAGATATGAGGGTGAGTTGGTACATACAAAAT |
| | Probe 873 | GGTATACTATTGGGAGGGTAATAGATAT |
| 921 | Encoding | GTAATAGATATGAGGGTGGTTTGGTATACATACCAA |
| | Probe 874 | ACTTTATTAGGTGGGAGGGTAATAGATAT |
| 922 | Encoding | GTAATAGATATGAGGGTGCCAACATACCAAACTTTA |
| | Probe 875 | TTCCCATAATTTGGGAGGGTAATAGATAT |
| 923 | Encoding | TGTATAGGATTAGAAGGGAGTTGGTACATACAAAAT |
| | Probe 876 | GGTATACAATGTGGGTGAGTGTATAGGATT |
| 924 | Encoding | TGTATAGGATTAGAAGGGATGAATGGTATACATACC |
| | Probe 877 | AAACTTTAAAGTGGGTGAGTGTATAGGATT |
| 925 | Encoding | TGTATAGGATTAGAAGGGTGTATGGTATACATACCA |
| | Probe 878 | AACTTTATAGGTGGGTGAGTGTATAGGATT |
| 926 | Encoding | TGTATAGGATTAGAAGGGTGTAGGTACATACAAAAT |
| | Probe 879 | GGTATACAATGTGGGTGAGTGTATAGGATT |
| 927 | Encoding | TGTATAGGATTAGAAGGGAGTTGGTACATACAAAAT |
| | Probe 880 | GGTATACTATGGGTGAGTGTATAGGATT |
| 928 | Encoding | TGTATAGGATTAGAAGGGTGTTTGGTATACATACCA |
| | Probe 881 | AACTTTATTAGGTGGGTGAGTGTATAGGATT |
| 929 | Encoding | TGTATAGGATTAGAAGGGCCAACATACCAAACTTTA |
| | Probe 882 | TTCCCATAATTGGGTGAGTGTATAGGATT |
| 930 | Encoding | ATAAGATAGTGAGATGGGTGCGTCTCCACTATTGCT |
| | Probe 883 | AGCGCTTGTGGGTGATAAGATAGTG |
| 931 | Encoding | ATAAGATAGTGAGATGGGTGGACTCAACTGTACTCA |
| | Probe 884 | AGGACGCGTCGTGGGTGATAAGATAGTG |
| 932 | Encoding | ATAAGATAGTGAGATGGGCAGTTGCAGTTTAGTGAG |
| | Probe 885 | CTGGGAGTGGGTGATAAGATAGTG |
| 933 | Encoding | ATAAGATAGTGAGATGGGTGGAATCCATCGAAGACT |
| | Probe 886 | AGGTCCCGTGGGTGATAAGATAGTG |
| 934 | Encoding | ATAAGATAGTGAGATGGGTGGATTCATAAAGTACAT |
| | Probe 887 | ACAAAAAGGGTGGTGGGTGATAAGATAGTG |
| 935 | Encoding | ATAAGATAGTGAGATGGGAGCATCTCCACTATTGCT |
| | Probe 888 | AGCGCTTGTGGGTGATAAGATAGTG |
| 936 | Encoding | ATAAGATAGTGAGATGGGTGGCACAGCGGTGATTGC |
| | Probe 889 | TCAGACGTGGGTGATAAGATAGTG |
| 937 | Encoding | ATAAGATAGTGAGATGGGACCATTGGCATCCACTTG |
| | Probe 890 | CGTCCAGTGGGTGATAAGATAGTG |
| 938 | Encoding | TGTATAGGATTAGAAGGGTGCGTCTCCACTATTGCT |
| | Probe 891 | AGCGCTTGGGTGAGTGTATAGGATT |
| 939 | Encoding | TGTATAGGATTAGAAGGGTGGACTCAACTGTACTCA |
| | Probe 892 | AGGACGCGTCGGGTGAGTGTATAGGATT |
| 940 | Encoding | TGTATAGGATTAGAAGGGCAGTTGCAGTTTAGTGAG |
| | Probe 893 | CTGGGAGGGTGAGTGTATAGGATT |
| 941 | Encoding | TGTATAGGATTAGAAGGGTGGAATCCATCGAAGACT |
| | Probe 894 | AGGTCCCGGGTGAGTGTATAGGATT |
| 942 | Encoding | TGTATAGGATTAGAAGGGTGGATTCATAAAGTACAT |
| | Probe 895 | ACAAAAAGGGTGTGGGTGAGTGTATAGGATT |
| 943 | Encoding | TGTATAGGATTAGAAGGGAGCATCTCCACTATTGCT |
| | Probe 896 | AGCGCTTGGGTGAGTGTATAGGATT |
| 944 | Encoding | TGTATAGGATTAGAAGGGTGGCACAGCGGTGATTGC |
| | Probe 897 | TCAGACGGGTGAGTGTATAGGATT |
| 945 | Encoding | TGTATAGGATTAGAAGGGACCATTGGCATCCACTTG |
| | Probe 898 | CGTCCAGGGTGAGTGTATAGGATT |
| 946 | Encoding | TTAATATGGGTAGTTGGGTGTGTCACTTGGACGAAT |
| | Probe 899 | CCTCGTAGTGGGTGTGTTAATATGGGT |
| 947 | Encoding | TTAATATGGGTAGTTGGGACGCCCAGCTGTATCATG |
| | Probe 900 | CGGTTAAGGGTGTGTTAATATGGGT |
| 948 | Encoding | TTAATATGGGTAGTTGGGCGCTTTGCCTCTCTTTGTT |
| | Probe 901 | GGAGCGGGTGTGTTAATATGGGT |
| 949 | Encoding | TTAATATGGGTAGTTGGGACGCCCAGCTGTATCATG |
| | Probe 902 | CGGTAAATGGGTGTGTTAATATGGGT |
| 950 | Encoding | TTAATATGGGTAGTTGGGAGTTGGACGAATCCTCGA |
| | Probe 903 | TCCTTAGGGTGTGTTAATATGGGT |
| 951 | Encoding | TTAATATGGGTAGTTGGGTGGACTTCACTTGGACGA |
| | Probe 904 | ATCCTGCTGGGTGTGTTAATATGGGT |
| 952 | Encoding | TTAATATGGGTAGTTGGGACGCCCAGCTGTATCATG |
| | Probe 905 | CGGATAGGGTGTGTTAATATGGGT |
| 953 | Encoding | TTAATATGGGTAGTTGGGTGCACTTTGCCTCTCTTTG |
| | Probe 906 | TTGGAGCGGGTGTGTTAATATGGGT |
| 954 | Encoding | TGTATAGGATTAGAAGGGTGTGTCACTTGGACGAAT |
| | Probe 907 | CCTCGTAGTGGGTGAGTGTATAGGATT |
| 955 | Encoding | TGTATAGGATTAGAAGGGACGCCCAGCTGTATCATG |
| | Probe 908 | CGGTTAAGGGTGAGTGTATAGGATT |
| 956 | Encoding | TGTATAGGATTAGAAGGGCGCTTTGCCTCTCTTTGTT |
| | Probe 909 | GGAGCGGGTGAGTGTATAGGATT |
| 957 | Encoding | TGTATAGGATTAGAAGGGACGCCCAGCTGTATCATG |
| | Probe 910 | CGGTAAATGGGTGAGTGTATAGGATT |
| 958 | Encoding | TGTATAGGATTAGAAGGGAGTTGGACGAATCCTCGA |
| | Probe 911 | TCCTTAGGGTGAGTGTATAGGATT |
| 959 | Encoding | TGTATAGGATTAGAAGGGTGGACTTCACTTGGACGA |
| | Probe 912 | ATCCTGCTGGGTGAGTGTATAGGATT |
| 960 | Encoding | TGTATAGGATTAGAAGGGACGCCCAGCTGTATCATG |
| | Probe 913 | CGGATAGGGTGAGTGTATAGGATT |
| 961 | Encoding | TGTATAGGATTAGAAGGGTGCACTTTGCCTCTCTTTG |
| | Probe 914 | TTGGAGCGGGTGAGTGTATAGGATT |
| 962 | Encoding | GGTAATTGAGTAGAAGGGTCGTGACTTTCTAAGTAA |
| | Probe 915 | TTACCGAGTGGGATGAGGTAATTGAGT |
| 963 | Encoding | GGTAATTGAGTAGAAGGGTGTTTCTGATGCAATTCT |
| | Probe 916 | CCGGAACGGGATGAGGTAATTGAGT |
| 964 | Encoding | GGTAATTGAGTAGAAGGGCTTGCTTTAAGAGATCCG |
| | Probe 917 | CTTCGGTGGGATGAGGTAATTGAGT |
| 965 | Encoding | GGTAATTGAGTAGAAGGGTCTTGCGTCTAGTGTTGT |
| | Probe 918 | TATCGCCGGGATGAGGTAATTGAGT |
| 966 | Encoding | GGTAATTGAGTAGAAGGGCTTATCTTTCAAACTCTA |
| | Probe 919 | GACATGGCAGGGATGAGGTAATTGAGT |
| 967 | Encoding | GGTAATTGAGTAGAAGGGTCTGCTGACTCCTATAAA |
| | Probe 920 | GGTTTAGTGGGATGAGGTAATTGAGT |
| 968 | Encoding | GGTAATTGAGTAGAAGGGTGGATCTCTTAGGTTTGC |
| | Probe 921 | ACTGCTAGGGATGAGGTAATTGAGT |
| 969 | Encoding | GGTAATTGAGTAGAAGGGTCCGAAACCTCCCAACAC |
| | Probe 922 | TTACGTGGGATGAGGTAATTGAGT |
| 970 | Encoding | TGTATAGGATTAGAAGGGTCGTGACTTTCTAAGTAA |
| | Probe 923 | TTACCGAGTGGGTGAGTGTATAGGATT |
| 971 | Encoding | TGTATAGGATTAGAAGGGTGTTTCTGATGCAATTCTC |
| | Probe 924 | CGGAACGGGTGAGTGTATAGGATT |
| 972 | Encoding | TGTATAGGATTAGAAGGGCTTGCTTTAAGAGATCCG |
| | Probe 925 | CTTCGGTGGGTGAGTGTATAGGATT |
| 973 | Encoding | TGTATAGGATTAGAAGGGTCTTGCGTCTAGTGTTGTT |
| | Probe 926 | ATCGCCGGGTGAGTGTATAGGATT |
| 974 | Encoding | TGTATAGGATTAGAAGGGCTTATCTTTCAAACTCTA |
| | Probe 927 | GACATGGCAGGGTGAGTGTATAGGATT |
| 975 | Encoding | TGTATAGGATTAGAAGGGTCTGCTGACTCCTATAAA |
| | Probe 928 | GGTTTAGTGGGTGAGTGTATAGGATT |
| 976 | Encoding | TGTATAGGATTAGAAGGGTGGATCTCTTAGGTTTGC |
| | Probe 929 | ACTGCTAGGGTGAGTGTATAGGATT |
| 977 | Encoding | TGTATAGGATTAGAAGGGTCCGAAACCTCCCAACAC |
| | Probe 930 | TTACGTGGGTGAGTGTATAGGATT |
| 978 | Encoding | GTAATAGATATGAGGGTGGCCACCCTTGGGTCCCCG |
| | Probe 931 | ACACCATCTGGGAGGGTAATAGATAT |
| 979 | Encoding | GTAATAGATATGAGGGTGCTCCCTTGGGTCCCCGAC |
| | Probe 932 | ACCATCTGGGAGGGTAATAGATAT |
| 980 | Encoding | GTAATAGATATGAGGGTGCCTCCCTTGGGTCCCCGA |
| | Probe 933 | CACGATTGGGAGGGTAATAGATAT |
| 981 | Encoding | GTAATAGATATGAGGGTGCCTCCCTTGGGTCCCCGA |
| | Probe 934 | CACCATCTGGGAGGGTAATAGATAT |
| 982 | Encoding | GTAATAGATATGAGGGTGGCCACCCTTGGGTCCCCG |
| | Probe 935 | ACACGATTGGGAGGGTAATAGATAT |
| 983 | Encoding | GTAATAGATATGAGGGTGAAAGGGTTTGCTTACCGT |
| | Probe 936 | CACGCCTGGGAGGGTAATAGATAT |
| 984 | Encoding | GTAATAGATATGAGGGTGGCCACCCTTGGGTCCCCG |
| | Probe 937 | ACAGGATGGGAGGGTAATAGATAT |
| 985 | Encoding | ATGGAAGTAGTAGAAGGGTGCCACCCTTGGGTCCCC |
| | Probe 938 | GACACCATCGGGATGTATGGAAGTAGT |
| 986 | Encoding | ATGGAAGTAGTAGAAGGGCTCCCTTGGGTCCCCGAC |
| | Probe 939 | ACCATCGGGATGTATGGAAGTAGT |
| 987 | Encoding | ATGGAAGTAGTAGAAGGGCCTCCCTTGGGTCCCCGA |
| | Probe 940 | CACGATGGGATGTATGGAAGTAGT |
| 988 | Encoding | ATGGAAGTAGTAGAAGGGCCTCCCTTGGGTCCCCGA |
| | Probe 941 | CACCATCGGGATGTATGGAAGTAGT |
| 989 | Encoding | ATGGAAGTAGTAGAAGGGTGCCACCCTTGGGTCCCC |
| | Probe 942 | GACACGATGGGATGTATGGAAGTAGT |
| 990 | Encoding | ATGGAAGTAGTAGAAGGGAAAGGGTTTGCTTACCGT |
| | Probe 943 | CACGCCGGGATGTATGGAAGTAGT |
| 991 | Encoding | ATGGAAGTAGTAGAAGGGTGCCACCCTTGGGTCCCC |
| | Probe 944 | GACAGGAGGGATGTATGGAAGTAGT |
| 992 | Encoding | GGAATTTAGTGAGAAGGGAGTTCAGACCTAAGCAAC |
| | Probe 945 | CGCGACGGGTGTTGGAATTTAGTG |
| 993 | Encoding | GGAATTTAGTGAGAAGGGATCGTGACTTTCTGGTTG |
| | Probe 946 | GATACGCAGGGTGTTGGAATTTAGTG |
| 994 | Encoding | GGAATTTAGTGAGAAGGGTCGCAGTTGCAGACCAGA |
| | Probe 947 | CAGGGCGGGTGTTGGAATTTAGTG |
| 995 | Encoding | GGAATTTAGTGAGAAGGGTGGACATAAAGGTTAGG |
| | Probe 948 | CCACCCTGTGGGTGTTGGAATTTAGTG |
| 996 | Encoding | GGAATTTAGTGAGAAGGGCTTGAACGCCTTATCTCT |
| | Probe 949 | AAGGAATGGGTGTTGGAATTTAGTG |
| 997 | Encoding | GGAATTTAGTGAGAAGGGTCAGGCCAGTGCGTACGA |
| | Probe 950 | CTTCGTGGGTGTTGGAATTTAGTG |
| 998 | Encoding | GGAATTTAGTGAGAAGGGTTATGGCAACTAGTAACA |
| | Probe 951 | AGGCAAGGGTGTTGGAATTTAGTG |
| 999 | Encoding | GGAATTTAGTGAGAAGGGTTCATCTTTCAAACAAAA |
| | Probe 952 | GCCATGACCGGGTGTTGGAATTTAGTG |
| 1000 | Encoding | ATGGAAGTAGTAGAAGGGAGTTCAGACCTAAGCAA |
| | Probe 953 | CCGCGACGGGATGTATGGAAGTAGT |
| 1001 | Encoding | ATGGAAGTAGTAGAAGGGATCGTGACTTTCTGGTTG |
| | Probe 954 | GATACGCAGGGATGTATGGAAGTAGT |
| 1002 | Encoding | ATGGAAGTAGTAGAAGGGTCGCAGTTGCAGACCAG |
| | Probe 955 | ACAGGGCGGGATGTATGGAAGTAGT |
| 1003 | Encoding | ATGGAAGTAGTAGAAGGGTGGACATAAAGGTTAGG |
| | Probe 956 | CCACCCTGTGGGATGTATGGAAGTAGT |
| 1004 | Encoding | ATGGAAGTAGTAGAAGGGCTTGAACGCCTTATCTCT |
| | Probe 957 | AAGGAATGGGATGTATGGAAGTAGT |
| 1005 | Encoding | ATGGAAGTAGTAGAAGGGTCAGGCCAGTGCGTACG |
| | Probe 958 | ACTTCGTGGGATGTATGGAAGTAGT |
| 1006 | Encoding | ATGGAAGTAGTAGAAGGGTTATGGCAACTAGTAACA |
| | Probe 959 | AGGCAAGGGATGTATGGAAGTAGT |
| 1007 | Encoding | ATGGAAGTAGTAGAAGGGTTCATCTTTCAAACAAAA |
| | Probe 960 | GCCATGACCGGGATGTATGGAAGTAGT |
| 1008 | Encoding | TTAATATGGGTAGTTGGGTAATTCCTTTCCCAGCAAG |
| | Probe 961 | CTCCAGGGTGTGTTAATATGGGT |
| 1009 | Encoding | TTAATATGGGTAGTTGGGTGAACCAGCAAGCTGGTC |
| | Probe 962 | CATTGTAGGGTGTGTTAATATGGGT |
| 1010 | Encoding | TTAATATGGGTAGTTGGGTGTTCTTGGTAAGGTTCTC |
| | Probe 963 | CGCCAAGGGTGTGTTAATATGGGT |
| 1011 | Encoding | TTAATATGGGTAGTTGGGTGCCCCATCCCATAGCGA |
| | Probe 964 | TAAAAGAGGGTGTGTTAATATGGGT |
| 1012 | Encoding | TTAATATGGGTAGTTGGGAAGATCTGACTTGCCCTG |
| | Probe 965 | CCAGGAGGGTGTGTTAATATGGGT |
| 1013 | Encoding | TTAATATGGGTAGTTGGGTGCTGGACTCCCCGGCTA |
| | Probe 966 | AGGGCGGTGGGTGTGTTAATATGGGT |
| 1014 | Encoding | TTAATATGGGTAGTTGGGTGGAGGATTATCTCCGGC |
| | Probe 967 | AGTCAGGTGGGTGTGTTAATATGGGT |
| 1015 | Encoding | TTAATATGGGTAGTTGGGTAGAACTGGGACGGTTTT |
| | Probe 968 | TTTCACGGGTGTGTTAATATGGGT |
| 1016 | Encoding | ATGGAAGTAGTAGAAGGGTAATTCCTTTCCCAGCAA |
| | Probe 969 | GCTCCAGGGATGTATGGAAGTAGT |
| 1017 | Encoding | ATGGAAGTAGTAGAAGGGTGAACCAGCAAGCTGGT |
| | Probe 970 | CCATTGTAGGGATGTATGGAAGTAGT |
| 1018 | Encoding | ATGGAAGTAGTAGAAGGGTGTTCTTGGTAAGGTTCT |
| | Probe 971 | CCGCCAAGGGATGTATGGAAGTAGT |
| 1019 | Encoding | ATGGAAGTAGTAGAAGGGTGCCCCATCCCATAGCGA |
| | Probe 972 | TAAAAGAGGGATGTATGGAAGTAGT |
| 1020 | Encoding | ATGGAAGTAGTAGAAGGGAAGATCTGACTTGCCCTG |
| | Probe 973 | CCAGGAGGGATGTATGGAAGTAGT |
| 1021 | Encoding | ATGGAAGTAGTAGAAGGGTGCTGGACTCCCCGGCTA |
| | Probe 974 | AGGGCGGTGGGATGTATGGAAGTAGT |
| 1022 | Encoding | ATGGAAGTAGTAGAAGGGTGGAGGATTATCTCCGGC |
| | Probe 975 | AGTCAGGTGGGATGTATGGAAGTAGT |
| 1023 | Encoding | ATGGAAGTAGTAGAAGGGTAGAACTGGGACGGTTTT |
| | Probe 976 | TTTCACGGGATGTATGGAAGTAGT |
| 1024 | Encoding | GGTAATTGAGTAGAAGGGAAACATCAGACTTAAAA |
| | Probe 977 | GACCGGGAGGGATGAGGTAATTGAGT |
| 1025 | Encoding | GGTAATTGAGTAGAAGGGTGACTCCAAAAGGTTACC |
| | Probe 978 | CCACGCCGGGATGAGGTAATTGAGT |
| 1026 | Encoding | GGTAATTGAGTAGAAGGGCCGTAAGAGATTTGCTAA |
| | Probe 979 | ACCTCGGCCGGGATGAGGTAATTGAGT |
| 1027 | Encoding | GGTAATTGAGTAGAAGGGTGTTACTCGGTGATAAAG |
| | Probe 980 | AAGTTAGCGGGATGAGGTAATTGAGT |
| 1028 | Encoding | GGTAATTGAGTAGAAGGGTGGACTCTAGGATTGTCA |
| | Probe 981 | AAAGATGTGTTGGGATGAGGTAATTGAGT |
| 1029 | Encoding | GGTAATTGAGTAGAAGGGTTGTCCAACACTTAGCAT |
| | Probe 982 | TCAAGCGGGATGAGGTAATTGAGT |
| 1030 | Encoding | GGTAATTGAGTAGAAGGGCGCCCCATCCAAAAGCG |
| | Probe 983 | GTAGGTAGGGATGAGGTAATTGAGT |
| 1031 | Encoding | GGTAATTGAGTAGAAGGGACCAGATACCGTCGAAA |
| | Probe 984 | CGTGAACAGAATGGGATGAGGTAATTGAGT |
| 1032 | Encoding | ATGGAAGTAGTAGAAGGGAAACATCAGACTTAAAA |
| | Probe 985 | GACCGGGAGGGATGTATGGAAGTAGT |
| 1033 | Encoding | ATGGAAGTAGTAGAAGGGTGACTCCAAAAGGTTACC |
| | Probe 986 | CCACGCCGGGATGTATGGAAGTAGT |
| 1034 | Encoding | ATGGAAGTAGTAGAAGGGCCGTAAGAGATTTGCTAA |
| | Probe 987 | ACCTCGGCCGGGATGTATGGAAGTAGT |
| 1035 | Encoding | ATGGAAGTAGTAGAAGGGTGTTACTCGGTGATAAAG |
| | Probe 988 | AAGTTAGCGGGATGTATGGAAGTAGT |
| 1036 | Encoding | ATGGAAGTAGTAGAAGGGTGGACTCTAGGATTGTCA |
| | Probe 989 | AAAGATGTGTTGGGATGTATGGAAGTAGT |
| 1037 | Encoding | ATGGAAGTAGTAGAAGGGTTGTCCAACACTTAGCAT |
| | Probe 990 | TCAAGCGGGATGTATGGAAGTAGT |
| 1038 | Encoding | ATGGAAGTAGTAGAAGGGCGCCCCATCCAAAAGCG |
| | Probe 991 | GTAGGTAGGGATGTATGGAAGTAGT |
| 1039 | Encoding | ATGGAAGTAGTAGAAGGGACCAGATACCGTCGAAA |
| | Probe 992 | CGTGAACAGAATGGGATGTATGGAAGTAGT |
| 1040 | Encoding | GTAATAGATATGAGGGTGGTGGTCTATATGTCCCGA |
| | Probe 993 | AGGTTCTGGGAGGGTAATAGATAT |
| 1041 | Encoding | GTAATAGATATGAGGGTGGATTTAATATTGGCAACC |
| | Probe 994 | GGAGTATGGGAGGGTAATAGATAT |
| 1042 | Encoding | GTAATAGATATGAGGGTGGCTTACCGTCATTCTTCAT |
| | Probe 995 | CCGAGTGGGAGGGTAATAGATAT |
| 1043 | Encoding | GTAATAGATATGAGGGTGGATTGTTATCCCGATGAC |
| | Probe 996 | AGACCGTGGGAGGGTAATAGATAT |
| 1044 | Encoding | GTAATAGATATGAGGGTGGACCAGTAACCTTTTTAC |
| | Probe 997 | CCCATACTGGGAGGGTAATAGATAT |
| 1045 | Encoding | GTAATAGATATGAGGGTGCAATACCCCCTTCGTCTA |
| | Probe 998 | GTAAGGTGGGAGGGTAATAGATAT |
| 1046 | Encoding | GTAATAGATATGAGGGTGGGTCATCGGTTTTACCTT |
| | Probe 999 | CGGGCCTGGGAGGGTAATAGATAT |
| 1047 | Encoding | GTAATAGATATGAGGGTGCGACCAGTTTTATGTGCA |
| | Probe 1000 | ATTCCCGCTGGGAGGGTAATAGATAT |
| 1048 | Encoding | GGATAGAGTATAGTTGGGTGTGGTCTATATGTCCCG |
| | Probe 1001 | AAGGTTCGGATGGAGGATAGAGTAT |
| 1049 | Encoding | GGATAGAGTATAGTTGGGTGATTTAATATTGGCAAC |
| | Probe 1002 | CGGAGTAGGATGGAGGATAGAGTAT |
| 1050 | Encoding | GGATAGAGTATAGTTGGGTGCTTACCGTCATTCTTCA |
| | Probe 1003 | TCCGAGGGATGGAGGATAGAGTAT |
| 1051 | Encoding | GGATAGAGTATAGTTGGGTGATTGTTATCCCGATGA |
| | Probe 1004 | CAGACCGGGATGGAGGATAGAGTAT |
| 1052 | Encoding | GGATAGAGTATAGTTGGGTGACCAGTAACCTTTTTA |
| | Probe 1005 | CCCCATACGGATGGAGGATAGAGTAT |
| 1053 | Encoding | GGATAGAGTATAGTTGGGCAATACCCCCTTCGTCTA |
| | Probe 1006 | GTAAGGTGGATGGAGGATAGAGTAT |
| 1054 | Encoding | GGATAGAGTATAGTTGGGTGGTCATCGGTTTTACCTT |
| | Probe 1007 | CGGGCCGGATGGAGGATAGAGTAT |
| 1055 | Encoding | GGATAGAGTATAGTTGGGCGACCAGTTTTATGTGCA |
| | Probe 1008 | ATTCCCGCGGATGGAGGATAGAGTAT |
| 1056 | Encoding | ATAAGATAGTGAGATGGGAGTCGCGACCCTTCCTCC |
| | Probe 1009 | CGATCCGTGGGTGATAAGATAGTG |
| 1057 | Encoding | ATAAGATAGTGAGATGGGTGACAGAAGTTTACGTAC |
| | Probe 1010 | CGAAAATGGTGGGTGATAAGATAGTG |
| 1058 | Encoding | ATAAGATAGTGAGATGGGTGTAGGCCAAGAGGAAT |
| | Probe 1011 | CATGCCCAGTGGGTGATAAGATAGTG |
| 1059 | Encoding | ATAAGATAGTGAGATGGGTGGTCGGCCAAGAGGAA |
| | Probe 1012 | TCATGCCCAGTGGGTGATAAGATAGTG |
| 1060 | Encoding | ATAAGATAGTGAGATGGGTGAGTCGCGACCCTTCCT |
| | Probe 1013 | CCCGTTCGTGGGTGATAAGATAGTG |
| 1061 | Encoding | ATAAGATAGTGAGATGGGACGATAGAAGTTTACGTA |
| | Probe 1014 | CCGAATATGTGGGTGATAAGATAGTG |
| 1062 | Encoding | ATAAGATAGTGAGATGGGAGCTGCCGGGCAGATGTC |
| | Probe 1015 | AAGCTGGTGGGTGATAAGATAGTG |
| 1063 | Encoding | ATAAGATAGTGAGATGGGTGAGCTGCCGGGCAGAT |
| | Probe 1016 | GTCAACCTGTGGGTGATAAGATAGTG |
| 1064 | Encoding | GGATAGAGTATAGTTGGGAGTCGCGACCCTTCCTCC |
| | Probe 1017 | CGATCCGGATGGAGGATAGAGTAT |
| 1065 | Encoding | GGATAGAGTATAGTTGGGTGACAGAAGTTTACGTAC |
| | Probe 1018 | CGAAAATGGGATGGAGGATAGAGTAT |
| 1066 | Encoding | GGATAGAGTATAGTTGGGTGTAGGCCAAGAGGAATC |
| | Probe 1019 | ATGCCCAGGATGGAGGATAGAGTAT |
| 1067 | Encoding | GGATAGAGTATAGTTGGGTGGTCGGCCAAGAGGAAT |
| | Probe 1020 | CATGCCCAGGATGGAGGATAGAGTAT |
| 1068 | Encoding | GGATAGAGTATAGTTGGGTGAGTCGCGACCCTTCCT |
| | Probe 1021 | CCCGTTCGGATGGAGGATAGAGTAT |
| 1069 | Encoding | GGATAGAGTATAGTTGGGACGATAGAAGTTTACGTA |
| | Probe 1022 | CCGAATATGGATGGAGGATAGAGTAT |
| 1070 | Encoding | GGATAGAGTATAGTTGGGAGCTGCCGGGCAGATGTC |
| | Probe 1023 | AAGCTGGGATGGAGGATAGAGTAT |
| 1071 | Encoding | GGATAGAGTATAGTTGGGTGAGCTGCCGGGCAGATG |
| | Probe 1024 | TCAACCTGGATGGAGGATAGAGTAT |
| 1072 | Encoding | GGTAATTGAGTAGAAGGGCGTGGAGGGTCCATACCC |
| | Probe 1025 | TCCCTGTGGGATGAGGTAATTGAGT |
| 1073 | Encoding | GGTAATTGAGTAGAAGGGCCGCGGAGGGTCCATACC |
| | Probe 1026 | CTCCGTGTGGGATGAGGTAATTGAGT |
| 1074 | Encoding | GGTAATTGAGTAGAAGGGTGCCCCGGAGGGTCCATA |
| | Probe 1027 | CCCTCGCTGGGATGAGGTAATTGAGT |
| 1075 | Encoding | GGTAATTGAGTAGAAGGGTGCCCCGGAGGGTCCATA |
| | Probe 1028 | CCCTCCCTGTGGGATGAGGTAATTGAGT |
| 1076 | Encoding | GGTAATTGAGTAGAAGGGCCGCGGAGGGTCCATACC |
| | Probe 1029 | CTCGCTGGGATGAGGTAATTGAGT |
| 1077 | Encoding | GGTAATTGAGTAGAAGGGCGTGGAGGGTCCATACCC |
| | Probe 1030 | TCCGTGTGGGATGAGGTAATTGAGT |
| 1078 | Encoding | GGTAATTGAGTAGAAGGGTGTGGAGGGTCCATACCC |
| | Probe 1031 | TCCGTGTGGGATGAGGTAATTGAGT |
| 1079 | Encoding | GGTAATTGAGTAGAAGGGCCGCGGAGGGTCCATACC |
| | Probe 1032 | CTCCGTGTGGGATGAGGTAATTGAGT |
| 1080 | Encoding | GGATAGAGTATAGTTGGGCGTGGAGGGTCCATACCC |
| | Probe 1033 | TCCCTGGGATGGAGGATAGAGTAT |
| 1081 | Encoding | GGATAGAGTATAGTTGGGCCGCGGAGGGTCCATACC |
| | Probe 1034 | CTCCGTGTGGATGGAGGATAGAGTAT |
| 1082 | Encoding | GGATAGAGTATAGTTGGGTGCCCCGGAGGGTCCATA |
| | Probe 1035 | CCCTCGCTGGATGGAGGATAGAGTAT |
| 1083 | Encoding | GGATAGAGTATAGTTGGGTGCCCCGGAGGGTCCATA |
| | Probe 1036 | CCCTCCCTGGGATGGAGGATAGAGTAT |
| 1084 | Encoding | GGATAGAGTATAGTTGGGCCGCGGAGGGTCCATACC |
| | Probe 1037 | CTCGCTGGATGGAGGATAGAGTAT |
| 1085 | Encoding | GGATAGAGTATAGTTGGGCGTGGAGGGTCCATACCC |
| | Probe 1038 | TCCGTGTGGATGGAGGATAGAGTAT |
| 1086 | Encoding | GGATAGAGTATAGTTGGGTGTGGAGGGTCCATACCC |
| | Probe 1039 | TCCGTGTGGATGGAGGATAGAGTAT |
| 1087 | Encoding | GGATAGAGTATAGTTGGGCCGCGGAGGGTCCATACC |
| | Probe 1040 | CTCCCTGGGATGGAGGATAGAGTAT |
| 1088 | Encoding | GTAATAGATATGAGGGTGGAGCGGCACTCTAGAAA |
| | Probe 1041 | AACAGAAATGGGAGGGTAATAGATAT |
| 1089 | Encoding | GTAATAGATATGAGGGTGAATTTTGGGATTTGCTAG |
| | Probe 1042 | GCAAGCTGGGAGGGTAATAGATAT |
| 1090 | Encoding | GTAATAGATATGAGGGTGGAGCGGCACTCTAGAAA |
| | Probe 1043 | AACACAATGGGAGGGTAATAGATAT |
| 1091 | Encoding | GTAATAGATATGAGGGTGAGTCCGAAGAGATCATCT |
| | Probe 1044 | TAAATGGAATGGGAGGGTAATAGATAT |
| 1092 | Encoding | GTAATAGATATGAGGGTGCAATTTTGGGATTTGCTA |
| | Probe 1045 | GGCTAGTGGGAGGGTAATAGATAT |
| 1093 | Encoding | GTAATAGATATGAGGGTGGAGCGGCACTCTAGAAA |
| | Probe 1046 | AACAGTAAGTGGGAGGGTAATAGATAT |
| 1094 | Encoding | GTAATAGATATGAGGGTGGCGAGTCATATAAGACTC |
| | Probe 1047 | AATCCGTTCTGGGAGGGTAATAGATAT |
| 1095 | Encoding | GTAATAGATATGAGGGTGCGAGTCATATAAGACTCA |
| | Probe 1048 | ATCCGTTCTGGGAGGGTAATAGATAT |
| 1096 | Encoding | TGTAATAGTAAGGAGGGAGAGCGGCACTCTAGAAA |
| | Probe 1049 | AACAGAAAGGGTGAGTGTAATAGTAA |
| 1097 | Encoding | TGTAATAGTAAGGAGGGAAATTTTGGGATTTGCTAG |
| | Probe 1050 | GCAAGCGGGTGAGTGTAATAGTAA |
| 1098 | Encoding | TGTAATAGTAAGGAGGGAGAGCGGCACTCTAGAAA |
| | Probe 1051 | AACACAAGGGTGAGTGTAATAGTAA |
| 1099 | Encoding | TGTAATAGTAAGGAGGGAAGTCCGAAGAGATCATCT |
| | Probe 1052 | TAAATGGAAGGGTGAGTGTAATAGTAA |
| 1100 | Encoding | TGTAATAGTAAGGAGGGACAATTTTGGGATTTGCTA |
| | Probe 1053 | GGCTAGTGGGTGAGTGTAATAGTAA |
| 1101 | Encoding | TGTAATAGTAAGGAGGGAGAGCGGCACTCTAGAAA |
| | Probe 1054 | AACAGTAAGTGGGTGAGTGTAATAGTAA |
| 1102 | Encoding | TGTAATAGTAAGGAGGGAGCGAGTCATATAAGACTC |
| | Probe 1055 | AATCCGTTCGGGTGAGTGTAATAGTAA |
| 1103 | Encoding | TGTAATAGTAAGGAGGGACGAGTCATATAAGACTCA |
| | Probe 1056 | ATCCGTTCGGGTGAGTGTAATAGTAA |
| 1104 | Encoding | GGAATTTAGTGAGAAGGGTGTCGCGGGCTCATCTTA |
| | Probe 1057 | TACTTGGTGGGTGTTGGAATTTAGTG |
| 1105 | Encoding | GGAATTTAGTGAGAAGGGTTTTCCTCAAAATCGCTT |
| | Probe 1058 | CGCAGCGGGTGTTGGAATTTAGTG |
| 1106 | Encoding | GGAATTTAGTGAGAAGGGATTCCCTGCCTTTCACTTC |
| | Probe 1059 | AGTGAGGGTGTTGGAATTTAGTG |
| 1107 | Encoding | GGAATTTAGTGAGAAGGGAACCCAGATTACTCCTTT |
| | Probe 1060 | GCCAGGTGGGTGTTGGAATTTAGTG |
| 1108 | Encoding | GGAATTTAGTGAGAAGGGCCAGGGAGATGTCAAGA |
| | Probe 1061 | CTTGCATGGGTGTTGGAATTTAGTG |
| 1109 | Encoding | GGAATTTAGTGAGAAGGGCGTTTCCAAAGCAGTTCA |
| | Probe 1062 | GGGCAAGGGTGTTGGAATTTAGTG |
| 1110 | Encoding | GGAATTTAGTGAGAAGGGTTTTTCCTCAAAATCGCT |
| | Probe 1063 | TCGGAGTGGGTGTTGGAATTTAGTG |
| 1111 | Encoding | GGAATTTAGTGAGAAGGGAGTCGCGGGCTCATCTTA |
| | Probe 1064 | TACTTGGTGGGTGTTGGAATTTAGTG |
| 1112 | Encoding | TGTAATAGTAAGGAGGGAGTCGCGGGCTCATCTTAT |
| | Probe 1065 | ACTTGGTGGGTGAGTGTAATAGTAA |
| 1113 | Encoding | TGTAATAGTAAGGAGGGATTTTCCTCAAAATCGCTT |
| | Probe 1066 | CGCAGCGGGTGAGTGTAATAGTAA |
| 1114 | Encoding | TGTAATAGTAAGGAGGGAATTCCCTGCCTTTCACTTC |
| | Probe 1067 | AGTGAGGGTGAGTGTAATAGTAA |
| 1115 | Encoding | TGTAATAGTAAGGAGGGAAACCCAGATTACTCCTTT |
| | Probe 1068 | GCCAGGTGGGTGAGTGTAATAGTAA |
| 1116 | Encoding | TGTAATAGTAAGGAGGGACCAGGGAGATGTCAAGA |
| | Probe 1069 | CTTGCATGGGTGAGTGTAATAGTAA |
| 1117 | Encoding | TGTAATAGTAAGGAGGGACGTTTCCAAAGCAGTTCA |
| | Probe 1070 | GGGCAAGGGTGAGTGTAATAGTAA |
| 1118 | Encoding | TGTAATAGTAAGGAGGGATTTTTCCTCAAAATCGCT |
| | Probe 1071 | TCGGAGTGGGTGAGTGTAATAGTAA |
| 1119 | Encoding | TGTAATAGTAAGGAGGGAAGTCGCGGGCTCATCTTA |
| | Probe 1072 | TACTTGGTGGGTGAGTGTAATAGTAA |
| 1120 | Encoding | ATAAGATAGTGAGATGGGTGTAGAAAACTTCCGTAC |
| | Probe 1073 | TAAGACAGGGTGGGTGATAAGATAGTG |
| 1121 | Encoding | ATAAGATAGTGAGATGGGTAAATGGAAATATCATGC |
| | Probe 1074 | GGTTAGGTGGGTGATAAGATAGTG |
| 1122 | Encoding | ATAAGATAGTGAGATGGGAACGGAAATATCATGCG |
| | Probe 1075 | GTATCAGGGTGGGTGATAAGATAGTG |
| 1123 | Encoding | ATAAGATAGTGAGATGGGTTGCCGTACTAAGACCCC |
| | Probe 1076 | GTTGCTGTGGGTGATAAGATAGTG |
| 1124 | Encoding | ATAAGATAGTGAGATGGGAAACTTCTGACTTGCATG |
| | Probe 1077 | GCCCGGGTGGGTGATAAGATAGTG |
| 1125 | Encoding | ATAAGATAGTGAGATGGGAGGAAACTTCCGTACTAA |
| | Probe 1078 | GACCGGCGTGGGTGATAAGATAGTG |
| 1126 | Encoding | ATAAGATAGTGAGATGGGTGGTCGAAAACTTCCGTA |
| | Probe 1079 | CTAAGTGGGTGGGTGATAAGATAGTG |
| 1127 | Encoding | ATAAGATAGTGAGATGGGATAGATGGAAATATCATG |
| | Probe 1080 | CGGATAGTGGGTGATAAGATAGTG |
| 1128 | Encoding | TGTAATAGTAAGGAGGGAGTAGAAAACTTCCGTACT |
| | Probe 1081 | AAGACAGGTGGGTGAGTGTAATAGTAA |
| 1129 | Encoding | TGTAATAGTAAGGAGGGATAAATGGAAATATCATGC |
| | Probe 1082 | GGTTAGTGGGTGAGTGTAATAGTAA |
| 1130 | Encoding | TGTAATAGTAAGGAGGGAAACGGAAATATCATGCG |
| | Probe 1083 | GTATCAGGTGGGTGAGTGTAATAGTAA |
| 1131 | Encoding | TGTAATAGTAAGGAGGGATTGCCGTACTAAGACCCC |
| | Probe 1084 | GTTGCTGGGTGAGTGTAATAGTAA |
| 1132 | Encoding | TGTAATAGTAAGGAGGGAAAACTTCTGACTTGCATG |
| | Probe 1085 | GCCCGGTGGGTGAGTGTAATAGTAA |
| 1133 | Encoding | TGTAATAGTAAGGAGGGAAGGAAACTTCCGTACTAA |
| | Probe 1086 | GACCGGCGGGTGAGTGTAATAGTAA |
| 1134 | Encoding | TGTAATAGTAAGGAGGGAGGTCGAAAACTTCCGTAC |
| | Probe 1087 | TAAGTGGTGGGTGAGTGTAATAGTAA |
| 1135 | Encoding | TGTAATAGTAAGGAGGGAATAGATGGAAATATCATG |
| | Probe 1088 | CGGATAGGGTGAGTGTAATAGTAA |
| 1136 | Encoding | TTAATATGGGTAGTTGGGTCGTGCGACTCAGCTGCA |
| | Probe 1089 | TTATCGCGGGTGTGTTAATATGGGT |
| 1137 | Encoding | TTAATATGGGTAGTTGGGCTCATGCGACTCAGCTGC |
| | Probe 1090 | ATTTACGGGTGTGTTAATATGGGT |
| 1138 | Encoding | TTAATATGGGTAGTTGGGTGGTCGACTCAGCTGCAT |
| | Probe 1091 | TATGCCCAGGGTGTGTTAATATGGGT |
| 1139 | Encoding | TTAATATGGGTAGTTGGGTGTAGACTCAGCTGCATT |
| | Probe 1092 | ATGCCCAGGGTGTGTTAATATGGGT |
| 1140 | Encoding | TTAATATGGGTAGTTGGGTGGTCGACTCAGCTGCAT |
| | Probe 1093 | TATGGCCGGGTGTGTTAATATGGGT |
| 1141 | Encoding | TTAATATGGGTAGTTGGGTCGTGCGACTCAGCTGCA |
| | Probe 1094 | TTAACGTGGGTGTGTTAATATGGGT |
| 1142 | Encoding | TTAATATGGGTAGTTGGGCGGGCGACTCAGCTGCAT |
| | Probe 1095 | TATCGCGGGTGTGTTAATATGGGT |
| 1143 | Encoding | TTAATATGGGTAGTTGGGCGGGCGACTCAGCTGCAT |
| | Probe 1096 | TATGGCCGGGTGTGTTAATATGGGT |
| 1144 | Encoding | TGTAATAGTAAGGAGGGATCGTGCGACTCAGCTGCA |
| | Probe 1097 | TTATCGCGGGTGAGTGTAATAGTAA |
| 1145 | Encoding | TGTAATAGTAAGGAGGGACTCATGCGACTCAGCTGC |
| | Probe 1098 | ATTTACGGGTGAGTGTAATAGTAA |
| 1146 | Encoding | TGTAATAGTAAGGAGGGAGGTCGACTCAGCTGCATT |
| | Probe 1099 | ATGCCCAGGGTGAGTGTAATAGTAA |
| 1147 | Encoding | TGTAATAGTAAGGAGGGAGTAGACTCAGCTGCATTA |
| | Probe 1100 | TGCCCAGGGTGAGTGTAATAGTAA |
| 1148 | Encoding | TGTAATAGTAAGGAGGGAGGTCGACTCAGCTGCATT |
| | Probe 1101 | ATGGCCGGGTGAGTGTAATAGTAA |
| 1149 | Encoding | TGTAATAGTAAGGAGGGATCGTGCGACTCAGCTGCA |
| | Probe 1102 | TTAACGTGGGTGAGTGTAATAGTAA |
| 1150 | Encoding | TGTAATAGTAAGGAGGGACGGGCGACTCAGCTGCAT |
| | Probe 1103 | TATCGCGGGTGAGTGTAATAGTAA |
| 1151 | Encoding | TGTAATAGTAAGGAGGGACGGGCGACTCAGCTGCAT |
| | Probe 1104 | TATGGCCGGGTGAGTGTAATAGTAA |
| 1152 | Encoding | GTAATAGATATGAGGGTGGTCCCATCCATATCCACA |
| | Probe 1105 | GCTCAGTGGGAGGGTAATAGATAT |
| 1153 | Encoding | GTAATAGATATGAGGGTGGACGCACTGAATTCTCTC |
| | Probe 1106 | CAAGTGTGGGAGGGTAATAGATAT |
| 1154 | Encoding | GTAATAGATATGAGGGTGAAATCTTACAACAGAGCT |
| | Probe 1107 | TTACGATGGCTGGGAGGGTAATAGATAT |
| 1155 | Encoding | GTAATAGATATGAGGGTGGCTGCTTTTACTTCAGAC |
| | Probe 1108 | TTATACAAGGCTGGGAGGGTAATAGATAT |
| 1156 | Encoding | GTAATAGATATGAGGGTGGTCAGCTGTGAAATGTAC |
| | Probe 1109 | TCCCAATGGGAGGGTAATAGATAT |
| 1157 | Encoding | GTAATAGATATGAGGGTGAGAAGGGCCTTTATTGCC |
| | Probe 1110 | ATGAGTTGGGAGGGTAATAGATAT |
| 1158 | Encoding | GTAATAGATATGAGGGTGAAAGTTCCGCTTACAATC |
| | Probe 1111 | TCTTCGATGGGAGGGTAATAGATAT |
| 1159 | Encoding | GTAATAGATATGAGGGTGCTGCTCACTCCCGTAGGT |
| | Probe 1112 | TGTGCGTGTGGGAGGGTAATAGATAT |
| 1160 | Encoding | TATAGTTATGGAGAAGGGTGTCCCATCCATATCCAC |
| | Probe 1113 | AGCTCAGGGAAGGGTATAGTTATGG |
| 1161 | Encoding | TATAGTTATGGAGAAGGGTGACGCACTGAATTCTCT |
| | Probe 1114 | CCAAGTGGGAAGGGTATAGTTATGG |
| 1162 | Encoding | TATAGTTATGGAGAAGGGAAATCTTACAACAGAGCT |
| | Probe 1115 | TTACGATGGCGGAAGGGTATAGTTATGG |
| 1163 | Encoding | TATAGTTATGGAGAAGGGTGCTGCTTTTACTTCAGA |
| | Probe 1116 | CTTATACAAGGCGGAAGGGTATAGTTATGG |
| 1164 | Encoding | TATAGTTATGGAGAAGGGTGTCAGCTGTGAAATGTA |
| | Probe 1117 | CTCCCAAGGAAGGGTATAGTTATGG |
| 1165 | Encoding | TATAGTTATGGAGAAGGGAGAAGGGCCTTTATTGCC |
| | Probe 1118 | ATGAGTGGAAGGGTATAGTTATGG |
| 1166 | Encoding | TATAGTTATGGAGAAGGGAAAGTTCCGCTTACAATC |
| | Probe 1119 | TCTTCGAGGAAGGGTATAGTTATGG |
| 1167 | Encoding | TATAGTTATGGAGAAGGGCTGCTCACTCCCGTAGGT |
| | Probe 1120 | TGTGCGTGGGAAGGGTATAGTTATGG |
| 1168 | Encoding | GGAATTTAGTGAGAAGGGAGAGTCAGGTACTGTCAC |
| | Probe 1121 | TTTCAAGTGGGTGTTGGAATTTAGTG |
| 1169 | Encoding | GGAATTTAGTGAGAAGGGTGAATCAGGTACTGTCAC |
| | Probe 1122 | TTTCAAGTGGGTGTTGGAATTTAGTG |
| 1170 | Encoding | GGAATTTAGTGAGAAGGGAATCAGGTACTGTCACTT |
| | Probe 1123 | TCTTAGGTGGGTGTTGGAATTTAGTG |
| 1171 | Encoding | GGAATTTAGTGAGAAGGGCCCTCTTAGTCAGGTACT |
| | Probe 1124 | GTCACAAAGGGTGTTGGAATTTAGTG |
| 1172 | Encoding | GGAATTTAGTGAGAAGGGAGAGTCAGGTACTGTCAC |
| | Probe 1125 | TTTCTAGGTGGGTGTTGGAATTTAGTG |
| 1173 | Encoding | GGAATTTAGTGAGAAGGGAGAGTCAGGTACTGTCAC |
| | Probe 1126 | TTTGAAGGGTGTTGGAATTTAGTG |
| 1174 | Encoding | GGAATTTAGTGAGAAGGGATCAGGTACTGTCACTTT |
| | Probe 1127 | CTTCGGAGGGTGTTGGAATTTAGTG |
| 1175 | Encoding | GGAATTTAGTGAGAAGGGTGAATCAGGTACTGTCAC |
| | Probe 1128 | TTTCTAGGTGGGTGTTGGAATTTAGTG |
| 1176 | Encoding | TATAGTTATGGAGAAGGGAGAGTCAGGTACTGTCAC |
| | Probe 1129 | TTTCAAGGGAAGGGTATAGTTATGG |
| 1177 | Encoding | TATAGTTATGGAGAAGGGTGAATCAGGTACTGTCAC |
| | Probe 1130 | TTTCAAGGGAAGGGTATAGTTATGG |
| 1178 | Encoding | TATAGTTATGGAGAAGGGAATCAGGTACTGTCACTT |
| | Probe 1131 | TCTTAGGTGGAAGGGTATAGTTATGG |
| 1179 | Encoding | TATAGTTATGGAGAAGGGCCCTCTTAGTCAGGTACT |
| | Probe 1132 | GTCACAAAGGAAGGGTATAGTTATGG |
| 1180 | Encoding | TATAGTTATGGAGAAGGGAGAGTCAGGTACTGTCAC |
| | Probe 1133 | TTTCTAGGTGGAAGGGTATAGTTATGG |
| 1181 | Encoding | TATAGTTATGGAGAAGGGAGAGTCAGGTACTGTCAC |
| | Probe 1134 | TTTGAAGGAAGGGTATAGTTATGG |
| 1182 | Encoding | TATAGTTATGGAGAAGGGATCAGGTACTGTCACTTT |
| | Probe 1135 | CTTCGGAGGAAGGGTATAGTTATGG |
| 1183 | Encoding | TATAGTTATGGAGAAGGGTGAATCAGGTACTGTCAC |
| | Probe 1136 | TTTCTAGGTGGAAGGGTATAGTTATGG |
| 1184 | Encoding | ATAAGATAGTGAGATGGGATCAGTTCGTTATGCAAT |
| | Probe 1137 | CCTGTCGTGGGTGATAAGATAGTG |
| 1185 | Encoding | ATAAGATAGTGAGATGGGCGAGGCACCGAGGATTC |
| | Probe 1138 | CTCCCGCTGTGGGTGATAAGATAGTG |
| 1186 | Encoding | ATAAGATAGTGAGATGGGTACGGCCCATCTTTTACC |
| | Probe 1139 | GAATATTAGTGGGTGATAAGATAGTG |
| 1187 | Encoding | ATAAGATAGTGAGATGGGCGACAATTATTTTCGCTC |
| | Probe 1140 | GACTTCGTGTGGGTGATAAGATAGTG |
| 1188 | Encoding | ATAAGATAGTGAGATGGGTGACACTGGGTTTTTGTG |
| | Probe 1141 | CTTTCGAGTGGGTGATAAGATAGTG |
| 1189 | Encoding | ATAAGATAGTGAGATGGGCTAGGGCCCATCTTTTAC |
| | Probe 1142 | CGAATATTAGTGGGTGATAAGATAGTG |
| 1190 | Encoding | ATAAGATAGTGAGATGGGCGAACTTTGTTTCCAGCC |
| | Probe 1143 | ATTCATGTGGGTGATAAGATAGTG |
| 1191 | Encoding | ATAAGATAGTGAGATGGGCGACAATTATTTTCGCTC |
| | Probe 1144 | GACTACGGTGGGTGATAAGATAGTG |
| 1192 | Encoding | TATAGTTATGGAGAAGGGATCAGTTCGTTATGCAAT |
| | Probe 1145 | CCTGTCGGAAGGGTATAGTTATGG |
| 1193 | Encoding | TATAGTTATGGAGAAGGGCGAGGCACCGAGGATTCC |
| | Probe 1146 | TCCCGCTGGAAGGGTATAGTTATGG |
| 1194 | Encoding | TATAGTTATGGAGAAGGGTACGGCCCATCTTTTACC |
| | Probe 1147 | GAATATTAGGAAGGGTATAGTTATGG |
| 1195 | Encoding | TATAGTTATGGAGAAGGGCGACAATTATTTTCGCTC |
| | Probe 1148 | GACTTCGTGGAAGGGTATAGTTATGG |
| 1196 | Encoding | TATAGTTATGGAGAAGGGTGACACTGGGTTTTTGTG |
| | Probe 1149 | CTTTCGAGGAAGGGTATAGTTATGG |
| 1197 | Encoding | TATAGTTATGGAGAAGGGCTAGGGCCCATCTTTTAC |
| | Probe 1150 | CGAATATTAGGAAGGGTATAGTTATGG |
| 1198 | Encoding | TATAGTTATGGAGAAGGGCGAACTTTGTTTCCAGCC |
| | Probe 1151 | ATTCATGGAAGGGTATAGTTATGG |
| 1199 | Encoding | TATAGTTATGGAGAAGGGCGACAATTATTTTCGCTC |
| | Probe 1152 | GACTACGGGAAGGGTATAGTTATGG |
| 1200 | Encoding | TTAATATGGGTAGTTGGGTGTGGGAATTCCGATCTC |
| | Probe 1153 | CCCTTGGTGGGTGTGTTAATATGGGT |
| 1201 | Encoding | TTAATATGGGTAGTTGGGTGTGGGAATTCCGATCTC |
| | Probe 1154 | CCCTAGGCGGGTGTGTTAATATGGGT |
| 1202 | Encoding | TTAATATGGGTAGTTGGGATGCGCCTACTACCTAAT |
| | Probe 1155 | GGGCGCGTGGGTGTGTTAATATGGGT |
| 1203 | Encoding | TTAATATGGGTAGTTGGGTGTGGGAATTCCGATCTC |
| | Probe 1156 | CCCATGTGGGTGTGTTAATATGGGT |
| 1204 | Encoding | TTAATATGGGTAGTTGGGATACGCCTACTACCTAAT |
| | Probe 1157 | GGGAGCGGGTGTGTTAATATGGGT |
| 1205 | Encoding | TTAATATGGGTAGTTGGGTGTGGGAATTCCGATCTC |
| | Probe 1158 | CCCTTGGTGGGTGTGTTAATATGGGT |
| 1206 | Encoding | TTAATATGGGTAGTTGGGTGGGCCTACTACCTAATG |
| | Probe 1159 | GGCCCGCGGGTGTGTTAATATGGGT |
| 1207 | Encoding | TTAATATGGGTAGTTGGGTGGGCCTACTACCTAATG |
| | Probe 1160 | GGCGCGTGGGTGTGTTAATATGGGT |
| 1208 | Encoding | TATAGTTATGGAGAAGGGTGTGGGAATTCCGATCTC |
| | Probe 1161 | CCCTTGGTGGAAGGGTATAGTTATGG |
| 1209 | Encoding | TATAGTTATGGAGAAGGGTGTGGGAATTCCGATCTC |
| | Probe 1162 | CCCTAGGCGGAAGGGTATAGTTATGG |
| 1210 | Encoding | TATAGTTATGGAGAAGGGATGCGCCTACTACCTAAT |
| | Probe 1163 | GGGCGCGGGAAGGGTATAGTTATGG |
| 1211 | Encoding | TATAGTTATGGAGAAGGGTGTGGGAATTCCGATCTC |
| | Probe 1164 | CCCATGGGAAGGGTATAGTTATGG |
| 1212 | Encoding | TATAGTTATGGAGAAGGGATACGCCTACTACCTAAT |
| | Probe 1165 | GGGAGCGGAAGGGTATAGTTATGG |
| 1213 | Encoding | TATAGTTATGGAGAAGGGTGTGGGAATTCCGATCTC |
| | Probe 1166 | CCCTTGGTGGAAGGGTATAGTTATGG |
| 1214 | Encoding | TATAGTTATGGAGAAGGGTGGGCCTACTACCTAATG |
| | Probe 1167 | GGCCCGCGGAAGGGTATAGTTATGG |
| 1215 | Encoding | TATAGTTATGGAGAAGGGTGGGCCTACTACCTAATG |
| | Probe 1168 | GGCGCGGGAAGGGTATAGTTATGG |
| 1216 | Encoding | GGTAATTGAGTAGAAGGGATACACCCTAATTACCAG |
| | Probe 1169 | TCCATGTGGGATGAGGTAATTGAGT |
| 1217 | Encoding | GGTAATTGAGTAGAAGGGTGTCGCGAGCTCATCTTT |
| | Probe 1170 | GGACCTAGGGATGAGGTAATTGAGT |
| 1218 | Encoding | GGTAATTGAGTAGAAGGGCAAGTCCCCGATTAAAGA |
| | Probe 1171 | TCTTATGTGGGATGAGGTAATTGAGT |
| 1219 | Encoding | GGTAATTGAGTAGAAGGGATTCCCCAGATTTCACTT |
| | Probe 1172 | CTGTGAGGGATGAGGTAATTGAGT |
| 1220 | Encoding | GGTAATTGAGTAGAAGGGCCTGGGTCAATACCTCCC |
| | Probe 1173 | ACAGGAGGGATGAGGTAATTGAGT |
| 1221 | Encoding | GGTAATTGAGTAGAAGGGTGGATGTATCAACTAACC |
| | Probe 1174 | GTAAGGCAAGGGATGAGGTAATTGAGT |
| 1222 | Encoding | GGTAATTGAGTAGAAGGGAAATTCCCTCTGTATGAC |
| | Probe 1175 | TGCGTAGGGATGAGGTAATTGAGT |
| 1223 | Encoding | GGTAATTGAGTAGAAGGGTGCCGTTATCCCCCATCC |
| | Probe 1176 | AAAGCGTGGGATGAGGTAATTGAGT |
| 1224 | Encoding | TATAGTTATGGAGAAGGGATACACCCTAATTACCAG |
| | Probe 1177 | TCCATGGGAAGGGTATAGTTATGG |
| 1225 | Encoding | TATAGTTATGGAGAAGGGTGTCGCGAGCTCATCTTT |
| | Probe 1178 | GGACCTAGGAAGGGTATAGTTATGG |
| 1226 | Encoding | TATAGTTATGGAGAAGGGCAAGTCCCCGATTAAAGA |
| | Probe 1179 | TCTTATGGGAAGGGTATAGTTATGG |
| 1227 | Encoding | TATAGTTATGGAGAAGGGATTCCCCAGATTTCACTT |
| | Probe 1180 | CTGTGAGGAAGGGTATAGTTATGG |
| 1228 | Encoding | TATAGTTATGGAGAAGGGCCTGGGTCAATACCTCCC |
| | Probe 1181 | ACAGGAGGAAGGGTATAGTTATGG |
| 1229 | Encoding | TATAGTTATGGAGAAGGGTGGATGTATCAACTAACC |
| | Probe 1182 | GTAAGGCAAGGAAGGGTATAGTTATGG |
| 1230 | Encoding | TATAGTTATGGAGAAGGGAAATTCCCTCTGTATGAC |
| | Probe 1183 | TGCGTAGGAAGGGTATAGTTATGG |
| 1231 | Encoding | TATAGTTATGGAGAAGGGTGCCGTTATCCCCCATCC |
| | Probe 1184 | AAAGCGTGGAAGGGTATAGTTATGG |
| 1232 | Encoding | GTAATAGATATGAGGGTGCGACTCTTTACAGTTGGC |
| | Probe 1185 | TCAGTCTGGGAGGGTAATAGATAT |
| 1233 | Encoding | GTAATAGATATGAGGGTGAAGATCACTGTGTTGCTT |
| | Probe 1186 | CCCAGATGGGAGGGTAATAGATAT |
| 1234 | Encoding | GTAATAGATATGAGGGTGTAGGCGATAAAATTAGTA |
| | Probe 1187 | TATGCGCATTGGGAGGGTAATAGATAT |
| 1235 | Encoding | GTAATAGATATGAGGGTGGAAAAAGTAAACTTTCGA |
| | Probe 1188 | TTAAGTTCCAATGGGAGGGTAATAGATAT |
| 1236 | Encoding | GTAATAGATATGAGGGTGACGCCTCTTTACAGTTGG |
| | Probe 1189 | CTCTGTTGGGAGGGTAATAGATAT |
| 1237 | Encoding | GTAATAGATATGAGGGTGGAACATCACTGTGTTGCT |
| | Probe 1190 | TCCGAGTGGGAGGGTAATAGATAT |
| 1238 | Encoding | GTAATAGATATGAGGGTGATATGCGATAAAATTAGT |
| | Probe 1191 | ATATGCGCATTGGGAGGGTAATAGATAT |
| 1239 | Encoding | GTAATAGATATGAGGGTGGTTGTAAACTTTCGATTA |
| | Probe 1192 | AGTTCGAAGTGGGAGGGTAATAGATAT |
| 1240 | Encoding | GATAAGTAAGTAGGGATGCGACTCTTTACAGTTGGC |
| | Probe 1193 | TCAGTCGGTGGAGGATAAGTAAGT |
| 1241 | Encoding | GATAAGTAAGTAGGGATGAAGATCACTGTGTTGCTT |
| | Probe 1194 | CCCAGAGGTGGAGGATAAGTAAGT |
| 1242 | Encoding | GATAAGTAAGTAGGGATGTAGGCGATAAAATTAGTA |
| | Probe 1195 | TATGCGCATGGTGGAGGATAAGTAAGT |
| 1243 | Encoding | GATAAGTAAGTAGGGATGGAAAAAGTAAACTTTCG |
| | Probe 1196 | ATTAAGTTCCAAGGTGGAGGATAAGTAAGT |
| 1244 | Encoding | GATAAGTAAGTAGGGATGACGCCTCTTTACAGTTGG |
| | Probe 1197 | CTCTGTGGTGGAGGATAAGTAAGT |
| 1245 | Encoding | GATAAGTAAGTAGGGATGGAACATCACTGTGTTGCT |
| | Probe 1198 | TCCGAGGGTGGAGGATAAGTAAGT |
| 1246 | Encoding | GATAAGTAAGTAGGGATGATATGCGATAAAATTAGT |
| | Probe 1199 | ATATGCGCATGGTGGAGGATAAGTAAGT |
| 1247 | Encoding | GATAAGTAAGTAGGGATGGTTGTAAACTTTCGATTA |
| | Probe 1200 | AGTTCGAAGGGTGGAGGATAAGTAAGT |
| 1248 | Encoding | GGAATTTAGTGAGAAGGGTCCGATGTCAAGGACTGG |
| | Probe 1201 | TAAGCAAGGGTGTTGGAATTTAGTG |
| 1249 | Encoding | GGAATTTAGTGAGAAGGGTGCCTCGCCTCACTCTGT |
| | Probe 1202 | TGGCTGGTGGGTGTTGGAATTTAGTG |
| 1250 | Encoding | GGAATTTAGTGAGAAGGGTGTCGGATGTCAAGGACT |
| | Probe 1203 | GGTATCCGGGTGTTGGAATTTAGTG |
| 1251 | Encoding | GGAATTTAGTGAGAAGGGCGGGCAGGCTTATGCGGT |
| | Probe 1204 | ATTTCGTGGGTGTTGGAATTTAGTG |
| 1252 | Encoding | GGAATTTAGTGAGAAGGGACGTCTTCCCTCCGGAGA |
| | Probe 1205 | GTTCCGAGCGGGTGTTGGAATTTAGTG |
| 1253 | Encoding | GGAATTTAGTGAGAAGGGAGACCTCCGGAGAGTTCC |
| | Probe 1206 | GTCCCGTGGGTGTTGGAATTTAGTG |
| 1254 | Encoding | GGAATTTAGTGAGAAGGGTGGAGTTATCGAGCCTGC |
| | Probe 1207 | CTTGCTGGGTGTTGGAATTTAGTG |
| 1255 | Encoding | GGAATTTAGTGAGAAGGGTGGACAGGCTTATGCGGT |
| | Probe 1208 | ATTACGTGGGTGTTGGAATTTAGTG |
| 1256 | Encoding | GATAAGTAAGTAGGGATGTCCGATGTCAAGGACTGG |
| | Probe 1209 | TAAGCAAGGTGGAGGATAAGTAAGT |
| 1257 | Encoding | GATAAGTAAGTAGGGATGGCCTCGCCTCACTCTGTT |
| | Probe 1210 | GGCTGGTGGTGGAGGATAAGTAAGT |
| 1258 | Encoding | GATAAGTAAGTAGGGATGGTCGGATGTCAAGGACTG |
| | Probe 1211 | GTATCCGGTGGAGGATAAGTAAGT |
| 1259 | Encoding | GATAAGTAAGTAGGGATGCGGGCAGGCTTATGCGGT |
| | Probe 1212 | ATTTCGGGTGGAGGATAAGTAAGT |
| 1260 | Encoding | GATAAGTAAGTAGGGATGACGTCTTCCCTCCGGAGA |
| | Probe 1213 | GTTCCGAGCGGTGGAGGATAAGTAAGT |
| 1261 | Encoding | GATAAGTAAGTAGGGATGAGACCTCCGGAGAGTTCC |
| | Probe 1214 | GTCCCGGGTGGAGGATAAGTAAGT |
| 1262 | Encoding | GATAAGTAAGTAGGGATGTGGAGTTATCGAGCCTGC |
| | Probe 1215 | CTTGCTGGTGGAGGATAAGTAAGT |
| 1263 | Encoding | GATAAGTAAGTAGGGATGGGACAGGCTTATGCGGTA |
| | Probe 1216 | TTACGTGGTGGAGGATAAGTAAGT |
| 1264 | Encoding | ATAAGATAGTGAGATGGGTGGCTCAGTTTTTACCCC |
| | Probe 1217 | TGTTGGGTGGGTGATAAGATAGTG |
| 1265 | Encoding | ATAAGATAGTGAGATGGGCTGCTCCCTCCTGGTTAG |
| | Probe 1218 | GTTCCCGTGGGTGATAAGATAGTG |
| 1266 | Encoding | ATAAGATAGTGAGATGGGTGACGTGGTCGCTTCTCT |
| | Probe 1219 | TTGAAAGTGGGTGATAAGATAGTG |
| 1267 | Encoding | ATAAGATAGTGAGATGGGTGTACCTCAGTTTTTACC |
| | Probe 1220 | CCTGATGGTGGGTGATAAGATAGTG |
| 1268 | Encoding | ATAAGATAGTGAGATGGGCTGCTCCCTCCTGGTTAG |
| | Probe 1221 | GTTGCCAGTGGGTGATAAGATAGTG |
| 1269 | Encoding | ATAAGATAGTGAGATGGGTGTGCCTCAGTTTTTACC |
| | Probe 1222 | CCTGATGGTGGGTGATAAGATAGTG |
| 1270 | Encoding | ATAAGATAGTGAGATGGGTGTACCTCAGTTTTTACC |
| | Probe 1223 | CCTCATGTGGGTGATAAGATAGTG |
| 1271 | Encoding | ATAAGATAGTGAGATGGGTGGACTGGTTAGGTTGGG |
| | Probe 1224 | TCACGCCGTGGGTGATAAGATAGTG |
| 1272 | Encoding | GATAAGTAAGTAGGGATGTGGCTCAGTTTTTACCCC |
| | Probe 1225 | TGTTGGTGGTGGAGGATAAGTAAGT |
| 1273 | Encoding | GATAAGTAAGTAGGGATGCTGCTCCCTCCTGGTTAG |
| | Probe 1226 | GTTCCCGGTGGAGGATAAGTAAGT |
| 1274 | Encoding | GATAAGTAAGTAGGGATGTGACGTGGTCGCTTCTCT |
| | Probe 1227 | TTGAAAGGTGGAGGATAAGTAAGT |
| 1275 | Encoding | GATAAGTAAGTAGGGATGTGTACCTCAGTTTTTACC |
| | Probe 1228 | CCTGATGGGTGGAGGATAAGTAAGT |
| 1276 | Encoding | GATAAGTAAGTAGGGATGCTGCTCCCTCCTGGTTAG |
| | Probe 1229 | GTTGCCAGGTGGAGGATAAGTAAGT |
| 1277 | Encoding | GATAAGTAAGTAGGGATGGTGCCTCAGTTTTTACCC |
| | Probe 1230 | CTGATGGGTGGAGGATAAGTAAGT |
| 1278 | Encoding | GATAAGTAAGTAGGGATGTGTACCTCAGTTTTTACC |
| | Probe 1231 | CCTCATGGTGGAGGATAAGTAAGT |
| 1279 | Encoding | GATAAGTAAGTAGGGATGGGACTGGTTAGGTTGGGT |
| | Probe 1232 | CACGCCGGTGGAGGATAAGTAAGT |
| 1280 | Encoding | TTAATATGGGTAGTTGGGTCTGTCGAAAACACGGTG |
| | Probe 1233 | AAGAGGTGGGTGTGTTAATATGGGT |
| 1281 | Encoding | TTAATATGGGTAGTTGGGCAGAGTCTGGATGATCAT |
| | Probe 1234 | CCTGAGTGGGTGTGTTAATATGGGT |
| 1282 | Encoding | TTAATATGGGTAGTTGGGTATTCTCGCTTATAAAAGC |
| | Probe 1235 | AGTAATGGGTGTGTTAATATGGGT |
| 1283 | Encoding | TTAATATGGGTAGTTGGGTCAAGCTAATAGTCTGAA |
| | Probe 1236 | TGGTTGTCGTGGGTGTGTTAATATGGGT |
| 1284 | Encoding | TTAATATGGGTAGTTGGGCAGTACCCAAAACTGCTA |
| | Probe 1237 | GTATCGTAGGGTGTGTTAATATGGGT |
| 1285 | Encoding | TTAATATGGGTAGTTGGGATGGACCAGGAAACGTAT |
| | Probe 1238 | TCAGGCGGGTGTGTTAATATGGGT |
| 1286 | Encoding | TTAATATGGGTAGTTGGGCCCGTCCTACCAGAAAAA |
| | Probe 1239 | TCCAAGACGGGTGTGTTAATATGGGT |
| 1287 | Encoding | TTAATATGGGTAGTTGGGTCTGTCGAAAACACGGTG |
| | Probe 1240 | AAGCGGAGGGTGTGTTAATATGGGT |
| 1288 | Encoding | GATAAGTAAGTAGGGATGTCTGTCGAAAACACGGTG |
| | Probe 1241 | AAGAGGTGGTGGAGGATAAGTAAGT |
| 1289 | Encoding | GATAAGTAAGTAGGGATGCAGAGTCTGGATGATCAT |
| | Probe 1242 | CCTGAGGGTGGAGGATAAGTAAGT |
| 1290 | Encoding | GATAAGTAAGTAGGGATGTATTCTCGCTTATAAAAG |
| | Probe 1243 | CAGTAATGGTGGAGGATAAGTAAGT |
| 1291 | Encoding | GATAAGTAAGTAGGGATGTCAAGCTAATAGTCTGAA |
| | Probe 1244 | TGGTTGTCGGGTGGAGGATAAGTAAGT |
| 1292 | Encoding | GATAAGTAAGTAGGGATGCAGTACCCAAAACTGCTA |
| | Probe 1245 | GTATCGTAGGTGGAGGATAAGTAAGT |
| 1293 | Encoding | GATAAGTAAGTAGGGATGATGGACCAGGAAACGTA |
| | Probe 1246 | TTCAGGCGGTGGAGGATAAGTAAGT |
| 1294 | Encoding | GATAAGTAAGTAGGGATGCCCGTCCTACCAGAAAAA |
| | Probe 1247 | TCCAAGACGGTGGAGGATAAGTAAGT |
| 1295 | Encoding | GATAAGTAAGTAGGGATGTCTGTCGAAAACACGGTG |
| | Probe 1248 | AAGCGGAGGTGGAGGATAAGTAAGT |
| 1296 | Encoding | GGTAATTGAGTAGAAGGGTGAGCGTCAGTACACCGT |
| | Probe 1249 | CCAGGTCGGGATGAGGTAATTGAGT |
| 1297 | Encoding | GGTAATTGAGTAGAAGGGACGATGCTGCCGGCAGG |
| | Probe 1250 | ATGTGTTGGGATGAGGTAATTGAGT |
| 1298 | Encoding | GGTAATTGAGTAGAAGGGTGTGCAGTCATCGGATCT |
| | Probe 1251 | GCCTAGCGGGATGAGGTAATTGAGT |
| 1299 | Encoding | GGTAATTGAGTAGAAGGGAGGTCCGAAAAAATTCC |
| | Probe 1252 | GCCCCGGAGGGATGAGGTAATTGAGT |
| 1300 | Encoding | GGTAATTGAGTAGAAGGGTGGACCGAAAAAATTCC |
| | Probe 1253 | GCCCCGGAGGGATGAGGTAATTGAGT |
| 1301 | Encoding | GGTAATTGAGTAGAAGGGTGGTCCGCACCGCATGCG |
| | Probe 1254 | CTTTGGCGGGATGAGGTAATTGAGT |
| 1302 | Encoding | GGTAATTGAGTAGAAGGGCGTGCATCCCTCTGTTAA |
| | Probe 1255 | CGCGTAGGGATGAGGTAATTGAGT |
| 1303 | Encoding | GGTAATTGAGTAGAAGGGTATGAAGTACTCCATCGC |
| | Probe 1256 | TCAGCGTGGGATGAGGTAATTGAGT |
| 1304 | Encoding | GATAAGTAAGTAGGGATGGAGCGTCAGTACACCGTC |
| | Probe 1257 | CAGGTCGGTGGAGGATAAGTAAGT |
| 1305 | Encoding | GATAAGTAAGTAGGGATGACGATGCTGCCGGCAGG |
| | Probe 1258 | ATGTGTTGGTGGAGGATAAGTAAGT |
| 1306 | Encoding | GATAAGTAAGTAGGGATGGTGCAGTCATCGGATCTG |
| | Probe 1259 | CCTAGCGGTGGAGGATAAGTAAGT |
| 1307 | Encoding | GATAAGTAAGTAGGGATGAGGTCCGAAAAAATTCC |
| | Probe 1260 | GCCCCGGAGGTGGAGGATAAGTAAGT |
| 1308 | Encoding | GATAAGTAAGTAGGGATGGGACCGAAAAAATTCCG |
| | Probe 1261 | CCCCGGAGGTGGAGGATAAGTAAGT |
| 1309 | Encoding | GATAAGTAAGTAGGGATGGGTCCGCACCGCATGCGC |
| | Probe 1262 | TTTGGCGGTGGAGGATAAGTAAGT |
| 1310 | Encoding | GATAAGTAAGTAGGGATGCGTGCATCCCTCTGTTAA |
| | Probe 1263 | CGCGTAGGTGGAGGATAAGTAAGT |
| 1311 | Encoding | GATAAGTAAGTAGGGATGTATGAAGTACTCCATCGC |
| | Probe 1264 | TCAGCGGGTGGAGGATAAGTAAGT |
| 1312 | Encoding | GTAATAGATATGAGGGTGGGTCGGCAGCGCAGGATT |
| | Probe 1265 | ATGGCCTGGGAGGGTAATAGATAT |
| 1313 | Encoding | GTAATAGATATGAGGGTGTACGCAGCGCAGGATTAT |
| | Probe 1266 | GCGCATTGGGAGGGTAATAGATAT |
| 1314 | Encoding | GTAATAGATATGAGGGTGACGCAGCGCAGGATTATG |
| | Probe 1267 | CGGATATGGGAGGGTAATAGATAT |
| 1315 | Encoding | GTAATAGATATGAGGGTGGGTCGGCAGCGCAGGATT |
| | Probe 1268 | ATGCCCATGGGAGGGTAATAGATAT |
| 1316 | Encoding | GTAATAGATATGAGGGTGGTAGGCAGCGCAGGATTA |
| | Probe 1269 | TGCGGATATGGGAGGGTAATAGATAT |
| 1317 | Encoding | GTAATAGATATGAGGGTGGTAGGCAGCGCAGGATTA |
| | Probe 1270 | TGCGCATTGGGAGGGTAATAGATAT |
| 1318 | Encoding | GTAATAGATATGAGGGTGGTAGGCAGCGCAGGATTA |
| | Probe 1271 | TGCCCATGGGAGGGTAATAGATAT |
| 1319 | Encoding | GTAATAGATATGAGGGTGTACGCAGCGCAGGATTAT |
| | Probe 1272 | GCGGATATGGGAGGGTAATAGATAT |
| 1320 | Encoding | AGTATTATTAGGGTGAGGTGGTCGGCAGCGCAGGAT |
| | Probe 1273 | TATGGCCGGGTTGGAGTATTATTAG |
| 1321 | Encoding | AGTATTATTAGGGTGAGGTACGCAGCGCAGGATTAT |
| | Probe 1274 | GCGCATGGGTTGGAGTATTATTAG |
| 1322 | Encoding | AGTATTATTAGGGTGAGGACGCAGCGCAGGATTATG |
| | Probe 1275 | CGGATAGGGTTGGAGTATTATTAG |
| 1323 | Encoding | AGTATTATTAGGGTGAGGTGGTCGGCAGCGCAGGAT |
| | Probe 1276 | TATGCCCAGGGTTGGAGTATTATTAG |
| 1324 | Encoding | AGTATTATTAGGGTGAGGGTAGGCAGCGCAGGATTA |
| | Probe 1277 | TGCGGATAGGGTTGGAGTATTATTAG |
| 1325 | Encoding | AGTATTATTAGGGTGAGGGTAGGCAGCGCAGGATTA |
| | Probe 1278 | TGCGCATGGGTTGGAGTATTATTAG |
| 1326 | Encoding | AGTATTATTAGGGTGAGGGTAGGCAGCGCAGGATTA |
| | Probe 1279 | TGCCCAGGGTTGGAGTATTATTAG |
| 1327 | Encoding | AGTATTATTAGGGTGAGGTACGCAGCGCAGGATTAT |
| | Probe 1280 | GCGGATAGGGTTGGAGTATTATTAG |
| 1328 | Encoding | GGAATTTAGTGAGAAGGGAGTACACCCAGTATCAAC |
| | Probe 1281 | TGCTTAGGGTGTTGGAATTTAGTG |
| 1329 | Encoding | GGAATTTAGTGAGAAGGGACCGGTTCAGACTCTCGT |
| | Probe 1282 | CCAAACGGGTGTTGGAATTTAGTG |
| 1330 | Encoding | GGAATTTAGTGAGAAGGGCAGCACATCATTCAGTTG |
| | Probe 1283 | CAAAAGTGGGTGTTGGAATTTAGTG |
| 1331 | Encoding | GGAATTTAGTGAGAAGGGTCTCTTTCGGGATTAGCA |
| | Probe 1284 | TCACCAGTGGGTGTTGGAATTTAGTG |
| 1332 | Encoding | GGAATTTAGTGAGAAGGGTGGGCGGAAGAACTATG |
| | Probe 1285 | CCATCCCCGGGTGTTGGAATTTAGTG |
| 1333 | Encoding | GGAATTTAGTGAGAAGGGTGGTCGGAAGAACTATGC |
| | Probe 1286 | CATCCCCGGGTGTTGGAATTTAGTG |
| 1334 | Encoding | GGAATTTAGTGAGAAGGGACTGAAGTTCTTTAATAG |
| | Probe 1287 | TTCTACCAACGTGGGTGTTGGAATTTAGTG |
| 1335 | Encoding | GGAATTTAGTGAGAAGGGTGGAGTTCTTTAATAGTT |
| | Probe 1288 | CTACCATGGCCGGGTGTTGGAATTTAGTG |
| 1336 | Encoding | AGTATTATTAGGGTGAGGAGTACACCCAGTATCAAC |
| | Probe 1289 | TGCTTAGGGTTGGAGTATTATTAG |
| 1337 | Encoding | AGTATTATTAGGGTGAGGACCGGTTCAGACTCTCGT |
| | Probe 1290 | CCAAACGGGTTGGAGTATTATTAG |
| 1338 | Encoding | AGTATTATTAGGGTGAGGCAGCACATCATTCAGTTG |
| | Probe 1291 | CAAAAGTGGGTTGGAGTATTATTAG |
| 1339 | Encoding | AGTATTATTAGGGTGAGGTCTCTTTCGGGATTAGCAT |
| | Probe 1292 | CACCAGTGGGTTGGAGTATTATTAG |
| 1340 | Encoding | AGTATTATTAGGGTGAGGTGGGCGGAAGAACTATGC |
| | Probe 1293 | CATCCCCGGGTTGGAGTATTATTAG |
| 1341 | Encoding | AGTATTATTAGGGTGAGGTGGTCGGAAGAACTATGC |
| | Probe 1294 | CATCCCCGGGTTGGAGTATTATTAG |
| 1342 | Encoding | AGTATTATTAGGGTGAGGACTGAAGTTCTTTAATAG |
| | Probe 1295 | TTCTACCAACGTGGGTTGGAGTATTATTAG |
| 1343 | Encoding | AGTATTATTAGGGTGAGGTGGAGTTCTTTAATAGTTC |
| | Probe 1296 | TACCATGGCCGGGTTGGAGTATTATTAG |
| 1344 | Encoding | ATAAGATAGTGAGATGGGATCGGAGCTTTCTTGCAG |
| | Probe 1297 | GGTAGGCGTGGGTGATAAGATAGTG |
| 1345 | Encoding | ATAAGATAGTGAGATGGGATCGGAGCTTTCTTGCAG |
| | Probe 1298 | GGTTGGGTGGGTGATAAGATAGTG |
| 1346 | Encoding | ATAAGATAGTGAGATGGGTCTTCACATTCAACTTAT |
| | Probe 1299 | CCTCCGCGGTGGGTGATAAGATAGTG |
| 1347 | Encoding | ATAAGATAGTGAGATGGGTGCAGTCCCATTAGAGTG |
| | Probe 1300 | CTCAACGTGGGTGATAAGATAGTG |
| 1348 | Encoding | ATAAGATAGTGAGATGGGAATCTATTGACTTCGGGT |
| | Probe 1301 | GTTTGGGTGGGTGATAAGATAGTG |
| 1349 | Encoding | ATAAGATAGTGAGATGGGTCGGAGCTTTCTTGCAGG |
| | Probe 1302 | GTAGGCGTGGGTGATAAGATAGTG |
| 1350 | Encoding | ATAAGATAGTGAGATGGGTGCAGTCCCATTAGAGTG |
| | Probe 1303 | CTCTACGGTGGGTGATAAGATAGTG |
| 1351 | Encoding | ATAAGATAGTGAGATGGGATCTTCACATTCAACTTA |
| | Probe 1304 | TCCTCCGCGGTGGGTGATAAGATAGTG |
| 1352 | Encoding | AGTATTATTAGGGTGAGGATCGGAGCTTTCTTGCAG |
| | Probe 1305 | GGTAGGCGGGTTGGAGTATTATTAG |
| 1353 | Encoding | AGTATTATTAGGGTGAGGATCGGAGCTTTCTTGCAG |
| | Probe 1306 | GGTTGGTGGGTTGGAGTATTATTAG |
| 1354 | Encoding | AGTATTATTAGGGTGAGGTCTTCACATTCAACTTATC |
| | Probe 1307 | CTCCGCGTGGGTTGGAGTATTATTAG |
| 1355 | Encoding | AGTATTATTAGGGTGAGGTGCAGTCCCATTAGAGTG |
| | Probe 1308 | CTCAACGGGTTGGAGTATTATTAG |
| 1356 | Encoding | AGTATTATTAGGGTGAGGAATCTATTGACTTCGGGT |
| | Probe 1309 | GTTTGGTGGGTTGGAGTATTATTAG |
| 1357 | Encoding | AGTATTATTAGGGTGAGGTCGGAGCTTTCTTGCAGG |
| | Probe 1310 | GTAGGCGGGTTGGAGTATTATTAG |
| 1358 | Encoding | AGTATTATTAGGGTGAGGTGCAGTCCCATTAGAGTG |
| | Probe 1311 | CTCTACGTGGGTTGGAGTATTATTAG |
| 1359 | Encoding | AGTATTATTAGGGTGAGGATCTTCACATTCAACTTAT |
| | Probe 1312 | CCTCCGCGTGGGTTGGAGTATTATTAG |
| 1360 | Encoding | TTAATATGGGTAGTTGGGTGAAGTACAAACAGGATG |
| | Probe 1313 | TCCCATCCGATGTGGGTGTGTTAATATGGGT |
| 1361 | Encoding | TTAATATGGGTAGTTGGGTAGTGGTACAAACAGGAT |
| | Probe 1314 | GTCCGTAGGGTGTGTTAATATGGGT |
| 1362 | Encoding | TTAATATGGGTAGTTGGGTAGTGGTACAAACAGGAT |
| | Probe 1315 | GTCGGTGGGTGTGTTAATATGGGT |
| 1363 | Encoding | TTAATATGGGTAGTTGGGAAATACAAACAGGATGTC |
| | Probe 1316 | CCATCCGATGTGGGTGTGTTAATATGGGT |
| 1364 | Encoding | TTAATATGGGTAGTTGGGAACACAAACAGGATGTCC |
| | Probe 1317 | CATCCGATGTGGGTGTGTTAATATGGGT |
| 1365 | Encoding | TTAATATGGGTAGTTGGGCTAATCTTTGGTACAAAC |
| | Probe 1318 | AGGAACAGGGTGTGTTAATATGGGT |
| 1366 | Encoding | TTAATATGGGTAGTTGGGTGAGGAGTTGCAGTTTTG |
| | Probe 1319 | AGTGGCTGGGTGTGTTAATATGGGT |
| 1367 | Encoding | TTAATATGGGTAGTTGGGTGAAGAGTTGCAGTTTTG |
| | Probe 1320 | AGTGGCTGGGTGTGTTAATATGGGT |
| 1368 | Encoding | AGTATTATTAGGGTGAGGGAAGTACAAACAGGATGT |
| | Probe 1321 | CCCATCCGATGTGGGTTGGAGTATTATTAG |
| 1369 | Encoding | AGTATTATTAGGGTGAGGTAGTGGTACAAACAGGAT |
| | Probe 1322 | GTCCGTAGGGTTGGAGTATTATTAG |
| 1370 | Encoding | AGTATTATTAGGGTGAGGTAGTGGTACAAACAGGAT |
| | Probe 1323 | GTCGGTGGGTTGGAGTATTATTAG |
| 1371 | Encoding | AGTATTATTAGGGTGAGGAAATACAAACAGGATGTC |
| | Probe 1324 | CCATCCGATGTGGGTTGGAGTATTATTAG |
| 1372 | Encoding | AGTATTATTAGGGTGAGGAACACAAACAGGATGTCC |
| | Probe 1325 | CATCCGATGTGGGTTGGAGTATTATTAG |
| 1373 | Encoding | AGTATTATTAGGGTGAGGCTAATCTTTGGTACAAAC |
| | Probe 1326 | AGGAACAGGGTTGGAGTATTATTAG |
| 1374 | Encoding | AGTATTATTAGGGTGAGGGAGGAGTTGCAGTTTTGA |
| | Probe 1327 | GTGGCTGGGTTGGAGTATTATTAG |
| 1375 | Encoding | AGTATTATTAGGGTGAGGTGAAGAGTTGCAGTTTTG |
| | Probe 1328 | AGTGGCTGGGTTGGAGTATTATTAG |
| 1376 | Encoding | GGAATTTAGTGAGAAGGGTGGAACTTCACTCAAGAA |
| | Probe 1329 | CAGCTCAGGGTGTTGGAATTTAGTG |
| 1377 | Encoding | GGAATTTAGTGAGAAGGGCGATCTCTAAGCTCTTCT |
| | Probe 1330 | TGGGATGTGTTGGGTGTTGGAATTTAGTG |
| 1378 | Encoding | GGAATTTAGTGAGAAGGGCAACTCTGCTTCGCAGCT |
| | Probe 1331 | TTGGAAGGGTGTTGGAATTTAGTG |
| 1379 | Encoding | GGAATTTAGTGAGAAGGGTTTGGTCAGCCCCCCCCA |
| | Probe 1332 | CACGATGGGTGTTGGAATTTAGTG |
| 1380 | Encoding | GGAATTTAGTGAGAAGGGAGCGGCGCCCTCCTAAAA |
| | Probe 1333 | GGTATCGGGTGTTGGAATTTAGTG |
| 1381 | Encoding | GGAATTTAGTGAGAAGGGAATGTCCCTTAAGACAGA |
| | Probe 1334 | GGTAATGGGTGTTGGAATTTAGTG |
| 1382 | Encoding | GGAATTTAGTGAGAAGGGCCGTTCTACCTCTCAGTA |
| | Probe 1335 | CGGGATGGGTGTTGGAATTTAGTG |
| 1383 | Encoding | GGAATTTAGTGAGAAGGGAGGCACTAACTTGAGAG |
| | Probe 1336 | AGCATCGTGGGTGTTGGAATTTAGTG |
| 1384 | Encoding | ATGTATTAAGAGGAGGGAGGAACTTCACTCAAGAAC |
| | Probe 1337 | AGCTCAGAGGAGGATGTATTAAGA |
| 1385 | Encoding | ATGTATTAAGAGGAGGGACGATCTCTAAGCTCTTCT |
| | Probe 1338 | TGGGATGTGTTGAGGAGGATGTATTAAGA |
| 1386 | Encoding | ATGTATTAAGAGGAGGGACAACTCTGCTTCGCAGCT |
| | Probe 1339 | TTGGAAGAGGAGGATGTATTAAGA |
| 1387 | Encoding | ATGTATTAAGAGGAGGGATTTGGTCAGCCCCCCCCA |
| | Probe 1340 | CACGATGAGGAGGATGTATTAAGA |
| 1388 | Encoding | ATGTATTAAGAGGAGGGAAGCGGCGCCCTCCTAAAA |
| | Probe 1341 | GGTATCGAGGAGGATGTATTAAGA |
| 1389 | Encoding | ATGTATTAAGAGGAGGGAAATGTCCCTTAAGACAGA |
| | Probe 1342 | GGTAATGAGGAGGATGTATTAAGA |
| 1390 | Encoding | ATGTATTAAGAGGAGGGACCGTTCTACCTCTCAGTA |
| | Probe 1343 | CGGGATGAGGAGGATGTATTAAGA |
| 1391 | Encoding | ATGTATTAAGAGGAGGGAAGGCACTAACTTGAGAG |
| | Probe 1344 | AGCATCGGAGGAGGATGTATTAAGA |
| 1392 | Encoding | ATAAGATAGTGAGATGGGTGCGCATTGCTGGGTAAG |
| | Probe 1345 | AGTAAGGTGGGTGATAAGATAGTG |
| 1393 | Encoding | ATAAGATAGTGAGATGGGTCACTAACTTAATATTGG |
| | Probe 1346 | CAACTAGTATAGTGGGTGATAAGATAGTG |
| 1394 | Encoding | ATAAGATAGTGAGATGGGTGAAACCGTATTAGCACA |
| | Probe 1347 | AATTTCAGAGTGGGTGATAAGATAGTG |
| 1395 | Encoding | ATAAGATAGTGAGATGGGCAGATACCGTATTAGCAC |
| | Probe 1348 | AAATTTGAGGTGGGTGATAAGATAGTG |
| 1396 | Encoding | ATAAGATAGTGAGATGGGTGCAGCTTCGGCGCAGAA |
| | Probe 1349 | GGAGAGCGTGGGTGATAAGATAGTG |
| 1397 | Encoding | ATAAGATAGTGAGATGGGAATTTCGGCGCAGAAGG |
| | Probe 1350 | AGTCCTAGTGGGTGATAAGATAGTG |
| 1398 | Encoding | ATAAGATAGTGAGATGGGTGCGCATTGCTGGGTAAG |
| | Probe 1351 | AGTTAGGGTGGGTGATAAGATAGTG |
| 1399 | Encoding | ATAAGATAGTGAGATGGGATCATTCCACTTTCCTCT |
| | Probe 1352 | ACTGGTGGTGGGTGATAAGATAGTG |
| 1400 | Encoding | ATGTATTAAGAGGAGGGATGCGCATTGCTGGGTAAG |
| | Probe 1353 | AGTAAGGAGGAGGATGTATTAAGA |
| 1401 | Encoding | ATGTATTAAGAGGAGGGATCACTAACTTAATATTGG |
| | Probe 1354 | CAACTAGTATAGAGGAGGATGTATTAAGA |
| 1402 | Encoding | ATGTATTAAGAGGAGGGAGAAACCGTATTAGCACA |
| | Probe 1355 | AATTTCAGAGAGGAGGATGTATTAAGA |
| 1403 | Encoding | ATGTATTAAGAGGAGGGACAGATACCGTATTAGCAC |
| | Probe 1356 | AAATTTGAGGAGGAGGATGTATTAAGA |
| 1404 | Encoding | ATGTATTAAGAGGAGGGAGCAGCTTCGGCGCAGAA |
| | Probe 1357 | GGAGAGCGAGGAGGATGTATTAAGA |
| 1405 | Encoding | ATGTATTAAGAGGAGGGAAATTTCGGCGCAGAAGG |
| | Probe 1358 | AGTCCTAGAGGAGGATGTATTAAGA |
| 1406 | Encoding | ATGTATTAAGAGGAGGGAGCGCATTGCTGGGTAAGA |
| | Probe 1359 | GTTAGGGAGGAGGATGTATTAAGA |
| 1407 | Encoding | ATGTATTAAGAGGAGGGAATCATTCCACTTTCCTCT |
| | Probe 1360 | ACTGGTGGAGGAGGATGTATTAAGA |
| 1408 | Encoding | TTAATATGGGTAGTTGGGATCTCAATTTCTTGACGTT |
| | Probe 1361 | ATCCGAGTGGGTGTGTTAATATGGGT |
| 1409 | Encoding | TTAATATGGGTAGTTGGGCAATTATGCGGTTCCTGG |
| | Probe 1362 | GTTGTCGTGGGTGTGTTAATATGGGT |
| 1410 | Encoding | TTAATATGGGTAGTTGGGCTTAACTCCGCTTTACACG |
| | Probe 1363 | GCCACAGGGTGTGTTAATATGGGT |
| 1411 | Encoding | TTAATATGGGTAGTTGGGTGTTAGCGCTCATCGTTTA |
| | Probe 1364 | CACGCGGGTGTGTTAATATGGGT |
| 1412 | Encoding | TTAATATGGGTAGTTGGGTGGCACTTCCTTCTTCCCT |
| | Probe 1365 | GCACTGGGTGTGTTAATATGGGT |
| 1413 | Encoding | TTAATATGGGTAGTTGGGTGGCAATTCCTTGCCGAC |
| | Probe 1366 | ACCATCGGGTGTGTTAATATGGGT |
| 1414 | Encoding | TTAATATGGGTAGTTGGGCAACTTCACTCTGTTTCAG |
| | Probe 1367 | CCTAAGGGTGTGTTAATATGGGT |
| 1415 | Encoding | TTAATATGGGTAGTTGGGAACGATAAATCTTTTCTCT |
| | Probe 1368 | CGCCACGTACGGGTGTGTTAATATGGGT |
| 1416 | Encoding | ATGTATTAAGAGGAGGGAATCTCAATTTCTTGACGT |
| | Probe 1369 | TATCCGAGGAGGAGGATGTATTAAGA |
| 1417 | Encoding | ATGTATTAAGAGGAGGGACAATTATGCGGTTCCTGG |
| | Probe 1370 | GTTGTCGGAGGAGGATGTATTAAGA |
| 1418 | Encoding | ATGTATTAAGAGGAGGGACTTAACTCCGCTTTACAC |
| | Probe 1371 | GGCCACAGAGGAGGATGTATTAAGA |
| 1419 | Encoding | ATGTATTAAGAGGAGGGATGTTAGCGCTCATCGTTT |
| | Probe 1372 | ACACGCGAGGAGGATGTATTAAGA |
| 1420 | Encoding | ATGTATTAAGAGGAGGGATGGCACTTCCTTCTTCCCT |
| | Probe 1373 | GCACTGAGGAGGATGTATTAAGA |
| 1421 | Encoding | ATGTATTAAGAGGAGGGATGGCAATTCCTTGCCGAC |
| | Probe 1374 | ACCATCGAGGAGGATGTATTAAGA |
| 1422 | Encoding | ATGTATTAAGAGGAGGGACAACTTCACTCTGTTTCA |
| | Probe 1375 | GCCTAAGAGGAGGATGTATTAAGA |
| 1423 | Encoding | ATGTATTAAGAGGAGGGAAACGATAAATCTTTTCTC |
| | Probe 1376 | TCGCCACGTACGAGGAGGATGTATTAAGA |
| 1424 | Encoding | GGTAATTGAGTAGAAGGGCGGTAAATCTTTTCACAC |
| | Probe 1377 | CATGCGTAGGGATGAGGTAATTGAGT |
| 1425 | Encoding | GGTAATTGAGTAGAAGGGTGCCCCGAAGGATTGTTT |
| | Probe 1378 | TACTACGGGATGAGGTAATTGAGT |
| 1426 | Encoding | GGTAATTGAGTAGAAGGGTGACCCGTAGGAAAAGA |
| | Probe 1379 | CACATTACACAGGGATGAGGTAATTGAGT |
| 1427 | Encoding | GGTAATTGAGTAGAAGGGCGGACAGCTCTGCTTCCC |
| | Probe 1380 | TTTCAAGGGATGAGGTAATTGAGT |
| 1428 | Encoding | GGTAATTGAGTAGAAGGGTGGAGAGTTATCCTCGGC |
| | Probe 1381 | TGTCGGAGGGATGAGGTAATTGAGT |
| 1429 | Encoding | GGTAATTGAGTAGAAGGGACGATAAATCTTTTCACA |
| | Probe 1382 | CCATGCGTAGGGATGAGGTAATTGAGT |
| 1430 | Encoding | GGTAATTGAGTAGAAGGGTGCCCCGAAGGATTGTTT |
| | Probe 1383 | TACAACGTGGGATGAGGTAATTGAGT |
| 1431 | Encoding | GGTAATTGAGTAGAAGGGACACGTAGGAAAAGACA |
| | Probe 1384 | CATTACACAGGGATGAGGTAATTGAGT |
| 1432 | Encoding | ATGTATTAAGAGGAGGGACGGTAAATCTTTTCACAC |
| | Probe 1385 | CATGCGTAGAGGAGGATGTATTAAGA |
| 1433 | Encoding | ATGTATTAAGAGGAGGGATGCCCCGAAGGATTGTTT |
| | Probe 1386 | TACTACGAGGAGGATGTATTAAGA |
| 1434 | Encoding | ATGTATTAAGAGGAGGGAGACCCGTAGGAAAAGAC |
| | Probe 1387 | ACATTACACAGAGGAGGATGTATTAAGA |
| 1435 | Encoding | ATGTATTAAGAGGAGGGACGGACAGCTCTGCTTCCC |
| | Probe 1388 | TTTCAAGAGGAGGATGTATTAAGA |
| 1436 | Encoding | ATGTATTAAGAGGAGGGAGGAGAGTTATCCTCGGCT |
| | Probe 1389 | GTCGGAGAGGAGGATGTATTAAGA |
| 1437 | Encoding | ATGTATTAAGAGGAGGGAACGATAAATCTTTTCACA |
| | Probe 1390 | CCATGCGTAGAGGAGGATGTATTAAGA |
| 1438 | Encoding | ATGTATTAAGAGGAGGGATGCCCCGAAGGATTGTTT |
| | Probe 1391 | TACAACGGAGGAGGATGTATTAAGA |
| 1439 | Encoding | ATGTATTAAGAGGAGGGAACACGTAGGAAAAGACA |
| | Probe 1392 | CATTACACAGAGGAGGATGTATTAAGA |
| 1440 | Encoding | GTAATAGATATGAGGGTGTTTCATGCGACTTAGTTG |
| | Probe 1393 | CATATATGGGAGGGTAATAGATAT |
| 1441 | Encoding | GTAATAGATATGAGGGTGTCGTGCGACTTAGTTGCA |
| | Probe 1394 | TTAACGTGGGAGGGTAATAGATAT |
| 1442 | Encoding | GTAATAGATATGAGGGTGGCGTTTTGCCTCTCTTTGT |
| | Probe 1395 | TGTGGTGGGAGGGTAATAGATAT |
| 1443 | Encoding | GTAATAGATATGAGGGTGTTCATGCGACTTAGTTGC |
| | Probe 1396 | ATTTACTGGGAGGGTAATAGATAT |
| 1444 | Encoding | GTAATAGATATGAGGGTGAGCGTTTTGCCTCTCTTTG |
| | Probe 1397 | TTGTGGTGGGAGGGTAATAGATAT |
| 1445 | Encoding | GTAATAGATATGAGGGTGAGCGTTTTGCCTCTCTTTG |
| | Probe 1398 | TTCTGTGGGAGGGTAATAGATAT |
| 1446 | Encoding | GTAATAGATATGAGGGTGGAGGGTTTTGCCTCTCTTT |
| | Probe 1399 | GTACTTGGGAGGGTAATAGATAT |
| 1447 | Encoding | GTAATAGATATGAGGGTGGTTCCATGCGACTTAGTT |
| | Probe 1400 | GCAAATTGGGAGGGTAATAGATAT |
| 1448 | Encoding | TAGAATTAGAGAGATGGGTTTCATGCGACTTAGTTG |
| | Probe 1401 | CATATAGGTGGAGTAGAATTAGAG |
| 1449 | Encoding | TAGAATTAGAGAGATGGGTCGTGCGACTTAGTTGCA |
| | Probe 1402 | TTAACGGGTGGAGTAGAATTAGAG |
| 1450 | Encoding | TAGAATTAGAGAGATGGGTGCGTTTTGCCTCTCTTTG |
| | Probe 1403 | TTGTGGTGGTGGAGTAGAATTAGAG |
| 1451 | Encoding | TAGAATTAGAGAGATGGGTTCATGCGACTTAGTTGC |
| | Probe 1404 | ATTTACGGTGGAGTAGAATTAGAG |
| 1452 | Encoding | TAGAATTAGAGAGATGGGAGCGTTTTGCCTCTCTTT |
| | Probe 1405 | GTTGTGGTGGTGGAGTAGAATTAGAG |
| 1453 | Encoding | TAGAATTAGAGAGATGGGAGCGTTTTGCCTCTCTTT |
| | Probe 1406 | GTTCTGGGTGGAGTAGAATTAGAG |
| 1454 | Encoding | TAGAATTAGAGAGATGGGTGAGGGTTTTGCCTCTCT |
| | Probe 1407 | TTGTACTGGTGGAGTAGAATTAGAG |
| 1455 | Encoding | TAGAATTAGAGAGATGGGTGTTCCATGCGACTTAGT |
| | Probe 1408 | TGCAAATGGTGGAGTAGAATTAGAG |
| 1456 | Encoding | GGAATTTAGTGAGAAGGGTGGTAGAATAGGAATCA |
| | Probe 1409 | CTAGGTTTCTAGTGGGTGTTGGAATTTAGTG |
| 1457 | Encoding | GGAATTTAGTGAGAAGGGCGGTAGAATAGGAATCA |
| | Probe 1410 | CTAGGTTTCTAGTGGGTGTTGGAATTTAGTG |
| 1458 | Encoding | GGAATTTAGTGAGAAGGGTGGCACTAGAATAGGAA |
| | Probe 1411 | TCACTAGGTAAGTGGGTGTTGGAATTTAGTG |
| 1459 | Encoding | GGAATTTAGTGAGAAGGGTGGCACTAGAATAGGAA |
| | Probe 1412 | TCACTAGGAAAGGGTGTTGGAATTTAGTG |
| 1460 | Encoding | GGAATTTAGTGAGAAGGGCAGCCACTAGAATAGGA |
| | Probe 1413 | ATCACTTCCGGGTGTTGGAATTTAGTG |
| 1461 | Encoding | GGAATTTAGTGAGAAGGGTGCAGCCACTAGAATAG |
| | Probe 1414 | GAATCAGATGGGTGTTGGAATTTAGTG |
| 1462 | Encoding | GGAATTTAGTGAGAAGGGTGCGCTAGAATAGGAATC |
| | Probe 1415 | ACTAGGTAAGTGGGTGTTGGAATTTAGTG |
| 1463 | Encoding | GGAATTTAGTGAGAAGGGTGGTAGAATAGGAATCA |
| | Probe 1416 | CTAGGTTTCAAGGTGGGTGTTGGAATTTAGTG |
| 1464 | Encoding | TAGAATTAGAGAGATGGGTGGTAGAATAGGAATCA |
| | Probe 1417 | CTAGGTTTCTAGGGTGGAGTAGAATTAGAG |
| 1465 | Encoding | TAGAATTAGAGAGATGGGCGGTAGAATAGGAATCA |
| | Probe 1418 | CTAGGTTTCTAGGGTGGAGTAGAATTAGAG |
| 1466 | Encoding | TAGAATTAGAGAGATGGGTGGCACTAGAATAGGAA |
| | Probe 1419 | TCACTAGGTAAGGGTGGAGTAGAATTAGAG |
| 1467 | Encoding | TAGAATTAGAGAGATGGGTGGCACTAGAATAGGAA |
| | Probe 1420 | TCACTAGGAAAGGTGGAGTAGAATTAGAG |
| 1468 | Encoding | TAGAATTAGAGAGATGGGCAGCCACTAGAATAGGA |
| | Probe 1421 | ATCACTTCCGGTGGAGTAGAATTAGAG |
| 1469 | Encoding | TAGAATTAGAGAGATGGGTGCAGCCACTAGAATAG |
| | Probe 1422 | GAATCAGATGGTGGAGTAGAATTAGAG |
| 1470 | Encoding | TAGAATTAGAGAGATGGGTGCGCTAGAATAGGAATC |
| | Probe 1423 | ACTAGGTAAGGGTGGAGTAGAATTAGAG |
| 1471 | Encoding | TAGAATTAGAGAGATGGGTGGTAGAATAGGAATCA |
| | Probe 1424 | CTAGGTTTCAAGGTGGTGGAGTAGAATTAGAG |
| 1472 | Encoding | ATAAGATAGTGAGATGGGAGGTAGGAAGGGCGACA |
| | Probe 1425 | TTACAGCGTGGGTGATAAGATAGTG |
| 1473 | Encoding | ATAAGATAGTGAGATGGGTCACCAAAGCAGTCCACA |
| | Probe 1426 | GGTTCTCGTGGGTGATAAGATAGTG |
| 1474 | Encoding | ATAAGATAGTGAGATGGGAGTCAAATCACTTCTCCT |
| | Probe 1427 | CCCCTTGTGGGTGATAAGATAGTG |
| 1475 | Encoding | ATAAGATAGTGAGATGGGCGACTCCGGTTAGGGTTG |
| | Probe 1428 | GGTGTGGTGGGTGATAAGATAGTG |
| 1476 | Encoding | ATAAGATAGTGAGATGGGCACCATCCTTGATGCTGG |
| | Probe 1429 | CTAGACGTGGGTGATAAGATAGTG |
| 1477 | Encoding | ATAAGATAGTGAGATGGGCAACAAAGCAGTCCACA |
| | Probe 1430 | GGTTCTCGTGGGTGATAAGATAGTG |
| 1478 | Encoding | ATAAGATAGTGAGATGGGAGGCGCTCAGTCAAATCA |
| | Probe 1431 | CTTGAGGTGGGTGATAAGATAGTG |
| 1479 | Encoding | ATAAGATAGTGAGATGGGCAGGTAGGAAGGGCGAC |
| | Probe 1432 | ATTAGAGGTGGGTGATAAGATAGTG |
| 1480 | Encoding | TAGAATTAGAGAGATGGGAGGTAGGAAGGGCGACA |
| | Probe 1433 | TTACAGCGGTGGAGTAGAATTAGAG |
| 1481 | Encoding | TAGAATTAGAGAGATGGGTCACCAAAGCAGTCCACA |
| | Probe 1434 | GGTTCTCGGTGGAGTAGAATTAGAG |
| 1482 | Encoding | TAGAATTAGAGAGATGGGAGTCAAATCACTTCTCCT |
| | Probe 1435 | CCCCTTGGTGGAGTAGAATTAGAG |
| 1483 | Encoding | TAGAATTAGAGAGATGGGCGACTCCGGTTAGGGTTG |
| | Probe 1436 | GGTGTGGGTGGAGTAGAATTAGAG |
| 1484 | Encoding | TAGAATTAGAGAGATGGGCACCATCCTTGATGCTGG |
| | Probe 1437 | CTAGACGGTGGAGTAGAATTAGAG |
| 1485 | Encoding | TAGAATTAGAGAGATGGGCAACAAAGCAGTCCACA |
| | Probe 1438 | GGTTCTCGGTGGAGTAGAATTAGAG |
| 1486 | Encoding | TAGAATTAGAGAGATGGGAGGCGCTCAGTCAAATCA |
| | Probe 1439 | CTTGAGGGTGGAGTAGAATTAGAG |
| 1487 | Encoding | TAGAATTAGAGAGATGGGCAGGTAGGAAGGGCGAC |
| | Probe 1440 | ATTAGAGGGTGGAGTAGAATTAGAG |
| 1488 | Encoding | TTAATATGGGTAGTTGGGCAGGCGACTTCGTGGTCT |
| | Probe 1441 | TATGGCCGGGTGTGTTAATATGGGT |
| 1489 | Encoding | TTAATATGGGTAGTTGGGTGCGGAGCTTTTACCCCA |
| | Probe 1442 | AAGTCTACGGGTGTGTTAATATGGGT |
| 1490 | Encoding | TTAATATGGGTAGTTGGGTGGCGGAGCTTTTACCCC |
| | Probe 1443 | AAAGTGTAGGGTGTGTTAATATGGGT |
| 1491 | Encoding | TTAATATGGGTAGTTGGGTGGAAGTCATGCGACTTC |
| | Probe 1444 | GTGGAGAGGGTGTGTTAATATGGGT |
| 1492 | Encoding | TTAATATGGGTAGTTGGGTGGAAAGTCATGCGACTT |
| | Probe 1445 | CGTGCAGTGGGTGTGTTAATATGGGT |
| 1493 | Encoding | TTAATATGGGTAGTTGGGTGCGGAGCTTTTACCCCA |
| | Probe 1446 | AAGTGTAGGGTGTGTTAATATGGGT |
| 1494 | Encoding | TTAATATGGGTAGTTGGGAGTCGACTTCGTGGTCTTA |
| | Probe 1447 | TGGCCGGGTGTGTTAATATGGGT |
| 1495 | Encoding | TTAATATGGGTAGTTGGGTGGCGGAGCTTTTACCCC |
| | Probe 1448 | AAAGAGTGGGTGTGTTAATATGGGT |
| 1496 | Encoding | TAGAATTAGAGAGATGGGCAGGCGACTTCGTGGTCT |
| | Probe 1449 | TATGGCCGGTGGAGTAGAATTAGAG |
| 1497 | Encoding | TAGAATTAGAGAGATGGGTGCGGAGCTTTTACCCCA |
| | Probe 1450 | AAGTCTACGGTGGAGTAGAATTAGAG |
| 1498 | Encoding | TAGAATTAGAGAGATGGGTGGCGGAGCTTTTACCCC |
| | Probe 1451 | AAAGTGTAGGTGGAGTAGAATTAGAG |
| 1499 | Encoding | TAGAATTAGAGAGATGGGTGGAAGTCATGCGACTTC |
| | Probe 1452 | GTGGAGAGGTGGAGTAGAATTAGAG |
| 1500 | Encoding | TAGAATTAGAGAGATGGGTGGAAAGTCATGCGACTT |
| | Probe 1453 | CGTGCAGGGTGGAGTAGAATTAGAG |
| 1501 | Encoding | TAGAATTAGAGAGATGGGTGCGGAGCTTTTACCCCA |
| | Probe 1454 | AAGTGTAGGTGGAGTAGAATTAGAG |
| 1502 | Encoding | TAGAATTAGAGAGATGGGAGTCGACTTCGTGGTCTT |
| | Probe 1455 | ATGGCCGGTGGAGTAGAATTAGAG |
| 1503 | Encoding | TAGAATTAGAGAGATGGGTGGCGGAGCTTTTACCCC |
| | Probe 1456 | AAAGAGTGGTGGAGTAGAATTAGAG |
| 1504 | Encoding | GGTAATTGAGTAGAAGGGACAGTTACAGTCTAGCAA |
| | Probe 1457 | CCCCGGTGGGATGAGGTAATTGAGT |
| 1505 | Encoding | GGTAATTGAGTAGAAGGGTGCACACTAGGAATTCCG |
| | Probe 1458 | GTTGAGGTGGGATGAGGTAATTGAGT |
| 1506 | Encoding | GGTAATTGAGTAGAAGGGCAAAGTAGTAGTTCCAAG |
| | Probe 1459 | GTTGTCGTGGGATGAGGTAATTGAGT |
| 1507 | Encoding | GGTAATTGAGTAGAAGGGACAGTTACAGTCTAGCAA |
| | Probe 1460 | CCCGGGAGGGATGAGGTAATTGAGT |
| 1508 | Encoding | GGTAATTGAGTAGAAGGGAACCCTTGGAGTTACGCT |
| | Probe 1461 | ACTTTGTGGGATGAGGTAATTGAGT |
| 1509 | Encoding | GGTAATTGAGTAGAAGGGCAGTTACAGTCTAGCAAC |
| | Probe 1462 | CCGGGAGGGATGAGGTAATTGAGT |
| 1510 | Encoding | GGTAATTGAGTAGAAGGGTGGTGTTGAGCCTTGGAG |
| | Probe 1463 | TTACCGAGGGATGAGGTAATTGAGT |
| 1511 | Encoding | GGTAATTGAGTAGAAGGGTGTTGTTTTAGTAGTAGT |
| | Probe 1464 | TCCAAGGAACGGGATGAGGTAATTGAGT |
| 1512 | Encoding | TAGAATTAGAGAGATGGGACAGTTACAGTCTAGCAA |
| | Probe 1465 | CCCCGGTGGTGGAGTAGAATTAGAG |
| 1513 | Encoding | TAGAATTAGAGAGATGGGTGCACACTAGGAATTCCG |
| | Probe 1466 | GTTGAGGTGGTGGAGTAGAATTAGAG |
| 1514 | Encoding | TAGAATTAGAGAGATGGGCAAAGTAGTAGTTCCAAG |
| | Probe 1467 | GTTGTCGGGTGGAGTAGAATTAGAG |
| 1515 | Encoding | TAGAATTAGAGAGATGGGACAGTTACAGTCTAGCAA |
| | Probe 1468 | CCCGGGAGGTGGAGTAGAATTAGAG |
| 1516 | Encoding | TAGAATTAGAGAGATGGGAACCCTTGGAGTTACGCT |
| | Probe 1469 | ACTTTGGGTGGAGTAGAATTAGAG |
| 1517 | Encoding | TAGAATTAGAGAGATGGGCAGTTACAGTCTAGCAAC |
| | Probe 1470 | CCGGGAGGTGGAGTAGAATTAGAG |
| 1518 | Encoding | TAGAATTAGAGAGATGGGTGGTGTTGAGCCTTGGAG |
| | Probe 1471 | TTACCGAGGTGGAGTAGAATTAGAG |
| 1519 | Encoding | TAGAATTAGAGAGATGGGTGTTGTTTTAGTAGTAGT |
| | Probe 1472 | TCCAAGGAACGGTGGAGTAGAATTAGAG |
| 1520 | Encoding | TGTATAGGATTAGAAGGGCGACTTGATAGGTACAGT |
| | Probe 1473 | CTTTTTTGAAGGGTGAGTGTATAGGATT |
| 1521 | Encoding | TGTATAGGATTAGAAGGGTGTGACTAGTTAATCAGG |
| | Probe 1474 | CGCATCCGGGTGAGTGTATAGGATT |
| 1522 | Encoding | TGTATAGGATTAGAAGGGTACCCGAAATACTATCTA |
| | Probe 1475 | CTTTCATACTAGGGTGAGTGTATAGGATT |
| 1523 | Encoding | TGTATAGGATTAGAAGGGAAGTTCTCTCTGTAATAG |
| | Probe 1476 | CCATTCATGGGTGAGTGTATAGGATT |
| 1524 | Encoding | TGTATAGGATTAGAAGGGTCGTCTTGATAGGTACAG |
| | Probe 1477 | TCTTTTTTGAAGGGTGAGTGTATAGGATT |
| 1525 | Encoding | TGTATAGGATTAGAAGGGCAAGCTTGACCTTGCGGT |
| | Probe 1478 | TTCCGAGGGTGAGTGTATAGGATT |
| 1526 | Encoding | TGTATAGGATTAGAAGGGCGCAGGCCATCTATTAGT |
| | Probe 1479 | GGAAACGGGTGAGTGTATAGGATT |
| 1527 | Encoding | TGTATAGGATTAGAAGGGCCGAAGGCCATCTATTAG |
| | Probe 1480 | TGGAAACGGGTGAGTGTATAGGATT |
| 1528 | Encoding | TATAGTTATGGAGAAGGGCGACTTGATAGGTACAGT |
| | Probe 1481 | CTTTTTTGAAGGAAGGGTATAGTTATGG |
| 1529 | Encoding | TATAGTTATGGAGAAGGGTGTGACTAGTTAATCAGG |
| | Probe 1482 | CGCATCCGGAAGGGTATAGTTATGG |
| 1530 | Encoding | TATAGTTATGGAGAAGGGTACCCGAAATACTATCTA |
| | Probe 1483 | CTTTCATACTAGGAAGGGTATAGTTATGG |
| 1531 | Encoding | TATAGTTATGGAGAAGGGAAGTTCTCTCTGTAATAG |
| | Probe 1484 | CCATTCATGGAAGGGTATAGTTATGG |
| 1532 | Encoding | TATAGTTATGGAGAAGGGTCGTCTTGATAGGTACAG |
| | Probe 1485 | TCTTTTTTGAAGGAAGGGTATAGTTATGG |
| 1533 | Encoding | TATAGTTATGGAGAAGGGCAAGCTTGACCTTGCGGT |
| | Probe 1486 | TTCCGAGGAAGGGTATAGTTATGG |
| 1534 | Encoding | TATAGTTATGGAGAAGGGCGCAGGCCATCTATTAGT |
| | Probe 1487 | GGAAACGGAAGGGTATAGTTATGG |
| 1535 | Encoding | TATAGTTATGGAGAAGGGCCGAAGGCCATCTATTAG |
| | Probe 1488 | TGGAAACGGAAGGGTATAGTTATGG |
| 1536 | Encoding | ATGGAAGTAGTAGAAGGGTAAACATCTGACTTGACA |
| | Probe 1489 | GACCCGGTGGGATGTATGGAAGTAGT |
| 1537 | Encoding | ATGGAAGTAGTAGAAGGGTGGACTCTACAAGACTCT |
| | Probe 1490 | AGCCTCGGTGGGATGTATGGAAGTAGT |
| 1538 | Encoding | ATGGAAGTAGTAGAAGGGATACCCTCTGTCAGGCAG |
| | Probe 1491 | ATCAGGTGGGATGTATGGAAGTAGT |
| 1539 | Encoding | ATGGAAGTAGTAGAAGGGTAGCCTCTGTCAGGCAGA |
| | Probe 1492 | TCCGGTGGGATGTATGGAAGTAGT |
| 1540 | Encoding | ATGGAAGTAGTAGAAGGGCCAATCTGACAGCGAGA |
| | Probe 1493 | GGCCGCTGGGATGTATGGAAGTAGT |
| 1541 | Encoding | ATGGAAGTAGTAGAAGGGTGCAATTGCTGAGGTTAT |
| | Probe 1494 | TAACCAGTGGGATGTATGGAAGTAGT |
| 1542 | Encoding | ATGGAAGTAGTAGAAGGGTCGCTCCTCAAGGGAAC |
| | Probe 1495 | AACCAGGTGGGATGTATGGAAGTAGT |
| 1543 | Encoding | ATGGAAGTAGTAGAAGGGTCCTCTCTGCCAAATTCC |
| | Probe 1496 | GTGCTAGGGATGTATGGAAGTAGT |
| 1544 | Encoding | TATAGTTATGGAGAAGGGTAAACATCTGACTTGACA |
| | Probe 1497 | GACCCGGTGGAAGGGTATAGTTATGG |
| 1545 | Encoding | TATAGTTATGGAGAAGGGTGGACTCTACAAGACTCT |
| | Probe 1498 | AGCCTCGGTGGAAGGGTATAGTTATGG |
| 1546 | Encoding | TATAGTTATGGAGAAGGGATACCCTCTGTCAGGCAG |
| | Probe 1499 | ATCAGGTGGAAGGGTATAGTTATGG |
| 1547 | Encoding | TATAGTTATGGAGAAGGGTAGCCTCTGTCAGGCAGA |
| | Probe 1500 | TCCGGTGGAAGGGTATAGTTATGG |
| 1548 | Encoding | TATAGTTATGGAGAAGGGCCAATCTGACAGCGAGAG |
| | Probe 1501 | GCCGCTGGAAGGGTATAGTTATGG |
| 1549 | Encoding | TATAGTTATGGAGAAGGGTGCAATTGCTGAGGTTAT |
| | Probe 1502 | TAACCAGTGGAAGGGTATAGTTATGG |
| 1550 | Encoding | TATAGTTATGGAGAAGGGTCGCTCCTCAAGGGAACA |
| | Probe 1503 | ACCAGGTGGAAGGGTATAGTTATGG |
| 1551 | Encoding | TATAGTTATGGAGAAGGGTCCTCTCTGCCAAATTCC |
| | Probe 1504 | GTGCTAGGAAGGGTATAGTTATGG |
| 1552 | Encoding | GGATAGAGTATAGTTGGGTTCCTGACGGCTTTACCC |
| | Probe 1505 | ATCATAGTGGATGGAGGATAGAGTAT |
| 1553 | Encoding | GGATAGAGTATAGTTGGGTCCTGACGGCTTTACCCA |
| | Probe 1506 | TCATAGTGGATGGAGGATAGAGTAT |
| 1554 | Encoding | GGATAGAGTATAGTTGGGAGGAAGGCCTGACGGCTT |
| | Probe 1507 | TACGGTGGATGGAGGATAGAGTAT |
| 1555 | Encoding | GGATAGAGTATAGTTGGGTTCCTGACGGCTTTACCC |
| | Probe 1508 | ATCAAAGGGATGGAGGATAGAGTAT |
| 1556 | Encoding | GGATAGAGTATAGTTGGGCCGGACGGCTTTACCCAT |
| | Probe 1509 | CATAGTGGATGGAGGATAGAGTAT |
| 1557 | Encoding | GGATAGAGTATAGTTGGGCAGGAAGGCCTGACGGCT |
| | Probe 1510 | TTAAGGTGGATGGAGGATAGAGTAT |
| 1558 | Encoding | GGATAGAGTATAGTTGGGTTCCTGACGGCTTTACCC |
| | Probe 1511 | ATCTAAGGATGGAGGATAGAGTAT |
| 1559 | Encoding | GGATAGAGTATAGTTGGGTCCTGACGGCTTTACCCA |
| | Probe 1512 | TCAAAGGGATGGAGGATAGAGTAT |
| 1560 | Encoding | TATAGTTATGGAGAAGGGTTCCTGACGGCTTTACCC |
| | Probe 1513 | ATCATAGTGGAAGGGTATAGTTATGG |
| 1561 | Encoding | TATAGTTATGGAGAAGGGTCCTGACGGCTTTACCCA |
| | Probe 1514 | TCATAGTGGAAGGGTATAGTTATGG |
| 1562 | Encoding | TATAGTTATGGAGAAGGGAGGAAGGCCTGACGGCTT |
| | Probe 1515 | TACGGTGGAAGGGTATAGTTATGG |
| 1563 | Encoding | TATAGTTATGGAGAAGGGTTCCTGACGGCTTTACCC |
| | Probe 1516 | ATCAAAGGGAAGGGTATAGTTATGG |
| 1564 | Encoding | TATAGTTATGGAGAAGGGCCGGACGGCTTTACCCAT |
| | Probe 1517 | CATAGTGGAAGGGTATAGTTATGG |
| 1565 | Encoding | TATAGTTATGGAGAAGGGCAGGAAGGCCTGACGGCT |
| | Probe 1518 | TTAAGGTGGAAGGGTATAGTTATGG |
| 1566 | Encoding | TATAGTTATGGAGAAGGGTTCCTGACGGCTTTACCC |
| | Probe 1519 | ATCTAAGGAAGGGTATAGTTATGG |
| 1567 | Encoding | TATAGTTATGGAGAAGGGTCCTGACGGCTTTACCCA |
| | Probe 1520 | TCAAAGGGAAGGGTATAGTTATGG |
| 1568 | Encoding | AATGATATGTTGAGTGGGAGCCCTGTCCACAGAGGT |
| | Probe 1521 | TTAGTTGTGGTGGAATGATATGTT |
| 1569 | Encoding | AATGATATGTTGAGTGGGAGGCACTGTTCGAGTGGA |
| | Probe 1522 | ACATCAGTGGTGGAATGATATGTT |
| 1570 | Encoding | AATGATATGTTGAGTGGGTCGATTTCTCCTTTGATAA |
| | Probe 1523 | CAGAATCTACGTGGTGGAATGATATGTT |
| 1571 | Encoding | AATGATATGTTGAGTGGGTGGTCTTCGTGTCTCCGA |
| | Probe 1524 | AGACTCGTGGTGGAATGATATGTT |
| 1572 | Encoding | AATGATATGTTGAGTGGGAGGCACGGAAGGGTTCAT |
| | Probe 1525 | CCCAGGGTGGTGGAATGATATGTT |
| 1573 | Encoding | AATGATATGTTGAGTGGGAGGCACTGTTCGAGTGGA |
| | Probe 1526 | ACAAGTAGGGTGGTGGAATGATATGTT |
| 1574 | Encoding | AATGATATGTTGAGTGGGTAACCGTAGTATGCTGAC |
| | Probe 1527 | CTAGCTGTGGTGGAATGATATGTT |
| 1575 | Encoding | AATGATATGTTGAGTGGGTTTCAGTTTCAAAAGCAG |
| | Probe 1528 | GTTTACGTGGTGGAATGATATGTT |
| 1576 | Encoding | GATAAGTAAGTAGGGATGAGCCCTGTCCACAGAGGT |
| | Probe 1529 | TTAGTTGGTGGAGGATAAGTAAGT |
| 1577 | Encoding | GATAAGTAAGTAGGGATGAGGCACTGTTCGAGTGGA |
| | Probe 1530 | ACATCAGGTGGAGGATAAGTAAGT |
| 1578 | Encoding | GATAAGTAAGTAGGGATGTCGATTTCTCCTTTGATA |
| | Probe 1531 | ACAGAATCTACGGTGGAGGATAAGTAAGT |
| 1579 | Encoding | GATAAGTAAGTAGGGATGTGGTCTTCGTGTCTCCGA |
| | Probe 1532 | AGACTCGGTGGAGGATAAGTAAGT |
| 1580 | Encoding | GATAAGTAAGTAGGGATGAGGCACGGAAGGGTTCA |
| | Probe 1533 | TCCCAGGTGGTGGAGGATAAGTAAGT |
| 1581 | Encoding | GATAAGTAAGTAGGGATGAGGCACTGTTCGAGTGGA |
| | Probe 1534 | ACAAGTAGGTGGTGGAGGATAAGTAAGT |
| 1582 | Encoding | GATAAGTAAGTAGGGATGTAACCGTAGTATGCTGAC |
| | Probe 1535 | CTAGCTGGTGGAGGATAAGTAAGT |
| 1583 | Encoding | GATAAGTAAGTAGGGATGTTTCAGTTTCAAAAGCAG |
| | Probe 1536 | GTTTACGGTGGAGGATAAGTAAGT |
| 1584 | Encoding | ATGGAAGTAGTAGAAGGGACGTGGTCCGTAGACATT |
| | Probe 1537 | ATGCCCAGGGATGTATGGAAGTAGT |
| 1585 | Encoding | ATGGAAGTAGTAGAAGGGTACTTCATCCGATAGTGC |
| | Probe 1538 | AAGCAGTGGGATGTATGGAAGTAGT |
| 1586 | Encoding | ATGGAAGTAGTAGAAGGGATGCCCTAAGGCCTTCTT |
| | Probe 1539 | CATAGTGTGGGATGTATGGAAGTAGT |
| 1587 | Encoding | ATGGAAGTAGTAGAAGGGAAACATCTGACTTAATTG |
| | Probe 1540 | ACCGGGAGGGATGTATGGAAGTAGT |
| 1588 | Encoding | ATGGAAGTAGTAGAAGGGTGCCCAAGACCACAACC |
| | Probe 1541 | TCTAAATCCTGTGGGATGTATGGAAGTAGT |
| 1589 | Encoding | ATGGAAGTAGTAGAAGGGTGAGCTATCTCTAAAGGA |
| | Probe 1542 | TTCGCTCCTGGGATGTATGGAAGTAGT |
| 1590 | Encoding | ATGGAAGTAGTAGAAGGGCGTTGTCTCAGCGTTCCC |
| | Probe 1543 | GAACCGTGGGATGTATGGAAGTAGT |
| 1591 | Encoding | ATGGAAGTAGTAGAAGGGTGCCTACGACAGACTTTA |
| | Probe 1544 | TGAGTTGGCGGGATGTATGGAAGTAGT |
| 1592 | Encoding | GATAAGTAAGTAGGGATGACGTGGTCCGTAGACATT |
| | Probe 1545 | ATGCCCAGGTGGAGGATAAGTAAGT |
| 1593 | Encoding | GATAAGTAAGTAGGGATGTACTTCATCCGATAGTGC |
| | Probe 1546 | AAGCAGGGTGGAGGATAAGTAAGT |
| 1594 | Encoding | GATAAGTAAGTAGGGATGATGCCCTAAGGCCTTCTT |
| | Probe 1547 | CATAGTGGGTGGAGGATAAGTAAGT |
| 1595 | Encoding | GATAAGTAAGTAGGGATGAAACATCTGACTTAATTG |
| | Probe 1548 | ACCGGGAGGTGGAGGATAAGTAAGT |
| 1596 | Encoding | GATAAGTAAGTAGGGATGGCCCAAGACCACAACCTC |
| | Probe 1549 | TAAATCCTGGGTGGAGGATAAGTAAGT |
| 1597 | Encoding | GATAAGTAAGTAGGGATGGAGCTATCTCTAAAGGAT |
| | Probe 1550 | TCGCTCCTGGTGGAGGATAAGTAAGT |
| 1598 | Encoding | GATAAGTAAGTAGGGATGCGTTGTCTCAGCGTTCCC |
| | Probe 1551 | GAACCGGGTGGAGGATAAGTAAGT |
| 1599 | Encoding | GATAAGTAAGTAGGGATGGCCTACGACAGACTTTAT |
| | Probe 1552 | GAGTTGGCGGTGGAGGATAAGTAAGT |
| 1600 | Encoding | GGATAGAGTATAGTTGGGTGGAAGGGAACAGGGCG |
| | Probe 1553 | TTGCCGGAGGATGGAGGATAGAGTAT |
| 1601 | Encoding | GGATAGAGTATAGTTGGGTGGAAAGGGAACAGGGC |
| | Probe 1554 | GTTGCAGGTGGATGGAGGATAGAGTAT |
| 1602 | Encoding | GGATAGAGTATAGTTGGGCGAGAAGGGAACAGGGC |
| | Probe 1555 | GTTGAGGTGGATGGAGGATAGAGTAT |
| 1603 | Encoding | GGATAGAGTATAGTTGGGTTCAACAGGGCGTTGCCC |
| | Probe 1556 | CTGGCAGGATGGAGGATAGAGTAT |
| 1604 | Encoding | GGATAGAGTATAGTTGGGTGCGCGAAGGGAACAGG |
| | Probe 1557 | GCGTTCGGTGGATGGAGGATAGAGTAT |
| 1605 | Encoding | GGATAGAGTATAGTTGGGCTTGAACAGGGCGTTGCC |
| | Probe 1558 | CCTCGCGGATGGAGGATAGAGTAT |
| 1606 | Encoding | GGATAGAGTATAGTTGGGTGCACGAAGGGAACAGG |
| | Probe 1559 | GCGTTGAGGTGGATGGAGGATAGAGTAT |
| 1607 | Encoding | GGATAGAGTATAGTTGGGCGGGAAGGGAACAGGGC |
| | Probe 1560 | GTTGCAGGTGGATGGAGGATAGAGTAT |
| 1608 | Encoding | GATAAGTAAGTAGGGATGGGAAGGGAACAGGGCGT |
| | Probe 1561 | TGCCGGAGGTGGAGGATAAGTAAGT |
| 1609 | Encoding | GATAAGTAAGTAGGGATGGGAAAGGGAACAGGGCG |
| | Probe 1562 | TTGCAGGTGGTGGAGGATAAGTAAGT |
| 1610 | Encoding | GATAAGTAAGTAGGGATGCGAGAAGGGAACAGGGC |
| | Probe 1563 | GTTGAGGTGGTGGAGGATAAGTAAGT |
| 1611 | Encoding | GATAAGTAAGTAGGGATGTTCAACAGGGCGTTGCCC |
| | Probe 1564 | CTGGCAGGTGGAGGATAAGTAAGT |
| 1612 | Encoding | GATAAGTAAGTAGGGATGGCGCGAAGGGAACAGGG |
| | Probe 1565 | CGTTCGGTGGTGGAGGATAAGTAAGT |
| 1613 | Encoding | GATAAGTAAGTAGGGATGCTTGAACAGGGCGTTGCC |
| | Probe 1566 | CCTCGCGGTGGAGGATAAGTAAGT |
| 1614 | Encoding | GATAAGTAAGTAGGGATGGCACGAAGGGAACAGGG |
| | Probe 1567 | CGTTGAGGTGGTGGAGGATAAGTAAGT |
| 1615 | Encoding | GATAAGTAAGTAGGGATGCGGGAAGGGAACAGGGC |
| | Probe 1568 | GTTGCAGGTGGTGGAGGATAAGTAAGT |
| 1616 | Encoding | TGTAATAGTAAGGAGGGAGAAGACCGTAATCTTCCC |
| | Probe 1569 | TTCACAGGGTGAGTGTAATAGTAA |
| 1617 | Encoding | TGTAATAGTAAGGAGGGATCTTTCCGACCGTAATCT |
| | Probe 1570 | TCCGAAGGGTGAGTGTAATAGTAA |
| 1618 | Encoding | TGTAATAGTAAGGAGGGATGATGCACAGATCTTCCG |
| | Probe 1571 | ACCCATGGGTGAGTGTAATAGTAA |
| 1619 | Encoding | TGTAATAGTAAGGAGGGAAGACGACCGTAATCTTCC |
| | Probe 1572 | CTTCACAGGGTGAGTGTAATAGTAA |
| 1620 | Encoding | TGTAATAGTAAGGAGGGAATTTTCCCTTCTGTACAC |
| | Probe 1573 | CCGTAGCGGGTGAGTGTAATAGTAA |
| 1621 | Encoding | TGTAATAGTAAGGAGGGAGTGATGCACAGATCTTCC |
| | Probe 1574 | GACCCATGGGTGAGTGTAATAGTAA |
| 1622 | Encoding | TGTAATAGTAAGGAGGGAGTCCTTCCGACCGTAATC |
| | Probe 1575 | TTCCGAAGGGTGAGTGTAATAGTAA |
| 1623 | Encoding | TGTAATAGTAAGGAGGGAATTTTCCCTTCTGTACAC |
| | Probe 1576 | CCGAAGTGGGTGAGTGTAATAGTAA |
| 1624 | Encoding | GATAAGTAAGTAGGGATGGAAGACCGTAATCTTCCC |
| | Probe 1577 | TTCACAGGTGGAGGATAAGTAAGT |
| 1625 | Encoding | GATAAGTAAGTAGGGATGTCTTTCCGACCGTAATCT |
| | Probe 1578 | TCCGAAGGTGGAGGATAAGTAAGT |
| 1626 | Encoding | GATAAGTAAGTAGGGATGTGATGCACAGATCTTCCG |
| | Probe 1579 | ACCCATGGTGGAGGATAAGTAAGT |
| 1627 | Encoding | GATAAGTAAGTAGGGATGAGACGACCGTAATCTTCC |
| | Probe 1580 | CTTCACAGGTGGAGGATAAGTAAGT |
| 1628 | Encoding | GATAAGTAAGTAGGGATGATTTTCCCTTCTGTACAC |
| | Probe 1581 | CCGTAGCGGTGGAGGATAAGTAAGT |
| 1629 | Encoding | GATAAGTAAGTAGGGATGGTGATGCACAGATCTTCC |
| | Probe 1582 | GACCCATGGTGGAGGATAAGTAAGT |
| 1630 | Encoding | GATAAGTAAGTAGGGATGGTCCTTCCGACCGTAATC |
| | Probe 1583 | TTCCGAAGGTGGAGGATAAGTAAGT |
| 1631 | Encoding | GATAAGTAAGTAGGGATGATTTTCCCTTCTGTACAC |
| | Probe 1584 | CCGAAGGGTGGAGGATAAGTAAGT |
| 1632 | Encoding | AATGATATGTTGAGTGGGCGTCTGTTTCCTGTTACCG |
| | Probe 1585 | TTGCTGTGGTGGAATGATATGTT |
| 1633 | Encoding | AATGATATGTTGAGTGGGCGGTCGTCAGCGAAACAG |
| | Probe 1586 | CAACGAGTGGTGGAATGATATGTT |
| 1634 | Encoding | AATGATATGTTGAGTGGGTGTCAAACAGCAAGCTGT |
| | Probe 1587 | TTCCACAGTGGTGGAATGATATGTT |
| 1635 | Encoding | AATGATATGTTGAGTGGGTTGCAAGCTGTTTCCTGTT |
| | Probe 1588 | ACGCAGTGGTGGAATGATATGTT |
| 1636 | Encoding | AATGATATGTTGAGTGGGAGTGAAACAGCAAGCTGT |
| | Probe 1589 | TTCGACGTGGTGGAATGATATGTT |
| 1637 | Encoding | AATGATATGTTGAGTGGGTGTAAGCTGTTTCCTGTTA |
| | Probe 1590 | CCCAAGTGGTGGAATGATATGTT |
| 1638 | Encoding | AATGATATGTTGAGTGGGCGTCTGTTTCCTGTTACCG |
| | Probe 1591 | TTCCTGGTGGTGGAATGATATGTT |
| 1639 | Encoding | AATGATATGTTGAGTGGGTGGTCGTCAGCGAAACAG |
| | Probe 1592 | CAAGGACGTGGTGGAATGATATGTT |
| 1640 | Encoding | AGTATTATTAGGGTGAGGCGTCTGTTTCCTGTTACCG |
| | Probe 1593 | TTGCTGGGTTGGAGTATTATTAG |
| 1641 | Encoding | AGTATTATTAGGGTGAGGCGGTCGTCAGCGAAACAG |
| | Probe 1594 | CAACGAGGGTTGGAGTATTATTAG |
| 1642 | Encoding | AGTATTATTAGGGTGAGGGTCAAACAGCAAGCTGTT |
| | Probe 1595 | TCCACAGGGTTGGAGTATTATTAG |
| 1643 | Encoding | AGTATTATTAGGGTGAGGTTGCAAGCTGTTTCCTGTT |
| | Probe 1596 | ACGCAGGGTTGGAGTATTATTAG |
| 1644 | Encoding | AGTATTATTAGGGTGAGGAGTGAAACAGCAAGCTGT |
| | Probe 1597 | TTCGACGGGTTGGAGTATTATTAG |
| 1645 | Encoding | AGTATTATTAGGGTGAGGTGTAAGCTGTTTCCTGTTA |
| | Probe 1598 | CCCAAGGGTTGGAGTATTATTAG |
| 1646 | Encoding | AGTATTATTAGGGTGAGGCGTCTGTTTCCTGTTACCG |
| | Probe 1599 | TTCCTGTGGGTTGGAGTATTATTAG |
| 1647 | Encoding | AGTATTATTAGGGTGAGGTGGTCGTCAGCGAAACAG |
| | Probe 1600 | CAAGGACGGGTTGGAGTATTATTAG |
| 1648 | Encoding | TGTATAGGATTAGAAGGGAGACATACTCTAGCTCGT |
| | Probe 1601 | CAGAAAGGGTGAGTGTATAGGATT |
| 1649 | Encoding | TGTATAGGATTAGAAGGGTTTGCAAAGTATTAATTT |
| | Probe 1602 | ACTGCCCTAGGTGGGTGAGTGTATAGGATT |
| 1650 | Encoding | TGTATAGGATTAGAAGGGTTTAGCAAAGTATTAATT |
| | Probe 1603 | TACTGCCCAAGTGGGTGAGTGTATAGGATT |
| 1651 | Encoding | TGTATAGGATTAGAAGGGATGTAGCTCGTCAGTTTT |
| | Probe 1604 | GAAACGTGGGTGAGTGTATAGGATT |
| 1652 | Encoding | TGTATAGGATTAGAAGGGTTTAGCAAAGTATTAATT |
| | Probe 1605 | TACTGCCGAAGGGTGAGTGTATAGGATT |
| 1653 | Encoding | TGTATAGGATTAGAAGGGATGTAGCTCGTCAGTTTT |
| | Probe 1606 | GAATCGTGGGTGAGTGTATAGGATT |
| 1654 | Encoding | TGTATAGGATTAGAAGGGTTTGCAAAGTATTAATTT |
| | Probe 1607 | ACTGCCCAAGTGGGTGAGTGTATAGGATT |
| 1655 | Encoding | TGTATAGGATTAGAAGGGTGAAGCTCGTCAGTTTTG |
| | Probe 1608 | AATCGTGGGTGAGTGTATAGGATT |
| 1656 | Encoding | AGTATTATTAGGGTGAGGAGACATACTCTAGCTCGT |
| | Probe 1609 | CAGAAAGGGTTGGAGTATTATTAG |
| 1657 | Encoding | AGTATTATTAGGGTGAGGTTTGCAAAGTATTAATTT |
| | Probe 1610 | ACTGCCCTAGGTGGGTTGGAGTATTATTAG |
| 1658 | Encoding | AGTATTATTAGGGTGAGGTTTAGCAAAGTATTAATT |
| | Probe 1611 | TACTGCCCAAGTGGGTTGGAGTATTATTAG |
| 1659 | Encoding | AGTATTATTAGGGTGAGGATGTAGCTCGTCAGTTTT |
| | Probe 1612 | GAAACGTGGGTTGGAGTATTATTAG |
| 1660 | Encoding | AGTATTATTAGGGTGAGGTTTAGCAAAGTATTAATT |
| | Probe 1613 | TACTGCCGAAGGGTTGGAGTATTATTAG |
| 1661 | Encoding | AGTATTATTAGGGTGAGGATGTAGCTCGTCAGTTTT |
| | Probe 1614 | GAATCGTGGGTTGGAGTATTATTAG |
| 1662 | Encoding | AGTATTATTAGGGTGAGGTTTGCAAAGTATTAATTT |
| | Probe 1615 | ACTGCCCAAGTGGGTTGGAGTATTATTAG |
| 1663 | Encoding | AGTATTATTAGGGTGAGGTGAAGCTCGTCAGTTTTG |
| | Probe 1616 | AATCGTGGGTTGGAGTATTATTAG |
| 1664 | Encoding | GGATAGAGTATAGTTGGGTGCAGCCAGTAAACTGGC |
| | Probe 1617 | AGAAAGGTGGATGGAGGATAGAGTAT |
| 1665 | Encoding | GGATAGAGTATAGTTGGGTGTGTAGACAACTGCCTC |
| | Probe 1618 | CCTTCGCGGATGGAGGATAGAGTAT |
| 1666 | Encoding | GGATAGAGTATAGTTGGGTGCCTGGCAACTGGACGT |
| | Probe 1619 | AGGCCAGGATGGAGGATAGAGTAT |
| 1667 | Encoding | GGATAGAGTATAGTTGGGAGATCCCGTTCGCTACCC |
| | Probe 1620 | ACGGAAGGATGGAGGATAGAGTAT |
| 1668 | Encoding | GGATAGAGTATAGTTGGGTCGGAGCATTGTTAAGAG |
| | Probe 1621 | GCCAGAGGATGGAGGATAGAGTAT |
| 1669 | Encoding | GGATAGAGTATAGTTGGGTGGCACTAACCTTTCCTA |
| | Probe 1622 | ATTTCCACGGCGGATGGAGGATAGAGTAT |
| 1670 | Encoding | GGATAGAGTATAGTTGGGTGTGGACTTAAGCGCCCA |
| | Probe 1623 | CCTAGCGGGATGGAGGATAGAGTAT |
| 1671 | Encoding | GGATAGAGTATAGTTGGGCCACGTCATACACAAAAC |
| | Probe 1624 | TATTCGCAAACGGATGGAGGATAGAGTAT |
| 1672 | Encoding | AGTATTATTAGGGTGAGGGCAGCCAGTAAACTGGCA |
| | Probe 1625 | GAAAGGTGGGTTGGAGTATTATTAG |
| 1673 | Encoding | AGTATTATTAGGGTGAGGGTGTAGACAACTGCCTCC |
| | Probe 1626 | CTTCGCGGGTTGGAGTATTATTAG |
| 1674 | Encoding | AGTATTATTAGGGTGAGGTGCCTGGCAACTGGACGT |
| | Probe 1627 | AGGCCAGGGTTGGAGTATTATTAG |
| 1675 | Encoding | AGTATTATTAGGGTGAGGAGATCCCGTTCGCTACCC |
| | Probe 1628 | ACGGAAGGGTTGGAGTATTATTAG |
| 1676 | Encoding | AGTATTATTAGGGTGAGGTCGGAGCATTGTTAAGAG |
| | Probe 1629 | GCCAGAGGGTTGGAGTATTATTAG |
| 1677 | Encoding | AGTATTATTAGGGTGAGGTGGCACTAACCTTTCCTA |
| | Probe 1630 | ATTTCCACGGCGGGTTGGAGTATTATTAG |
| 1678 | Encoding | AGTATTATTAGGGTGAGGGTGGACTTAAGCGCCCAC |
| | Probe 1631 | CTAGCGTGGGTTGGAGTATTATTAG |
| 1679 | Encoding | AGTATTATTAGGGTGAGGCCACGTCATACACAAAAC |
| | Probe 1632 | TATTCGCAAACGGGTTGGAGTATTATTAG |
| 1680 | Encoding | TGTAATAGTAAGGAGGGAAGTGGATTGCTCCTTTGA |
| | Probe 1633 | TTATCTTCGGGTGAGTGTAATAGTAA |
| 1681 | Encoding | TGTAATAGTAAGGAGGGAGAGCTACCGTCATCATCT |
| | Probe 1634 | TCAGTCGGGTGAGTGTAATAGTAA |
| 1682 | Encoding | TGTAATAGTAAGGAGGGAGAGCCTCGTTAGCGGGAT |
| | Probe 1635 | GTCTTCGGGTGAGTGTAATAGTAA |
| 1683 | Encoding | TGTAATAGTAAGGAGGGACTAACAAGAATCAATAG |
| | Probe 1636 | CAAGCATTGGGTGAGTGTAATAGTAA |
| 1684 | Encoding | TGTAATAGTAAGGAGGGAAGATACCGTCATCATCTT |
| | Probe 1637 | CACTCTGGGTGAGTGTAATAGTAA |
| 1685 | Encoding | TGTAATAGTAAGGAGGGAGCTTGGGACCATTTTTAG |
| | Probe 1638 | GGTAAAGGGTGAGTGTAATAGTAA |
| 1686 | Encoding | TGTAATAGTAAGGAGGGACTAACAAGAATCAATAG |
| | Probe 1639 | CAAGCTTTTGGGTGAGTGTAATAGTAA |
| 1687 | Encoding | TGTAATAGTAAGGAGGGAGTCGATTGCTCCTTTGAT |
| | Probe 1640 | TATCATCGTGGGTGAGTGTAATAGTAA |
| 1688 | Encoding | AGTATTATTAGGGTGAGGAGTGGATTGCTCCTTTGA |
| | Probe 1641 | TTATCTTCGGGTTGGAGTATTATTAG |
| 1689 | Encoding | AGTATTATTAGGGTGAGGGAGCTACCGTCATCATCT |
| | Probe 1642 | TCAGTCGGGTTGGAGTATTATTAG |
| 1690 | Encoding | AGTATTATTAGGGTGAGGGAGCCTCGTTAGCGGGAT |
| | Probe 1643 | GTCTTCGGGTTGGAGTATTATTAG |
| 1691 | Encoding | AGTATTATTAGGGTGAGGCTAACAAGAATCAATAGC |
| | Probe 1644 | AAGCATTGGGTTGGAGTATTATTAG |
| 1692 | Encoding | AGTATTATTAGGGTGAGGAGATACCGTCATCATCTT |
| | Probe 1645 | CACTCTGGGTTGGAGTATTATTAG |
| 1693 | Encoding | AGTATTATTAGGGTGAGGGCTTGGGACCATTTTTAG |
| | Probe 1646 | GGTAAAGGGTTGGAGTATTATTAG |
| 1694 | Encoding | AGTATTATTAGGGTGAGGCTAACAAGAATCAATAGC |
| | Probe 1647 | AAGCTTTTGGGTTGGAGTATTATTAG |
| 1695 | Encoding | AGTATTATTAGGGTGAGGGTCGATTGCTCCTTTGATT |
| | Probe 1648 | ATCATCGTGGGTTGGAGTATTATTAG |
| 1696 | Encoding | AATGATATGTTGAGTGGGCCGCTCCTATAGCATGAG |
| | Probe 1649 | GCCTACGGTGGTGGAATGATATGTT |
| 1697 | Encoding | AATGATATGTTGAGTGGGTGGATCGTAGCAACTAGA |
| | Probe 1650 | GACAAGCCAGTGGTGGAATGATATGTT |
| 1698 | Encoding | AATGATATGTTGAGTGGGTCGCTGTGTCCACTTTCTC |
| | Probe 1651 | TTTCCTCGTGGTGGAATGATATGTT |
| 1699 | Encoding | AATGATATGTTGAGTGGGAAACATCGGTCTTGCACA |
| | Probe 1652 | ACCCGGGTGGTGGAATGATATGTT |
| 1700 | Encoding | AATGATATGTTGAGTGGGTAGGCAAGCTAGATCATG |
| | Probe 1653 | CTGCGCAGTGGTGGAATGATATGTT |
| 1701 | Encoding | AATGATATGTTGAGTGGGTAGGCAAGCTAGATCATG |
| | Probe 1654 | CTGGGCGTGGTGGAATGATATGTT |
| 1702 | Encoding | AATGATATGTTGAGTGGGTAGCACCTAATATTAGTA |
| | Probe 1655 | AGTGCGTAAGTGGTGGAATGATATGTT |
| 1703 | Encoding | AATGATATGTTGAGTGGGCTGCGATGCATTTTCTGG |
| | Probe 1656 | GATATCGTGGTGGAATGATATGTT |
| 1704 | Encoding | ATGTATTAAGAGGAGGGACCGCTCCTATAGCATGAG |
| | Probe 1657 | GCCTACGGAGGAGGATGTATTAAGA |
| 1705 | Encoding | ATGTATTAAGAGGAGGGAGGATCGTAGCAACTAGA |
| | Probe 1658 | GACAAGCCAGAGGAGGATGTATTAAGA |
| 1706 | Encoding | ATGTATTAAGAGGAGGGATCGCTGTGTCCACTTTCT |
| | Probe 1659 | CTTTCCTCGAGGAGGATGTATTAAGA |
| 1707 | Encoding | ATGTATTAAGAGGAGGGAAAACATCGGTCTTGCACA |
| | Probe 1660 | ACCCGGGAGGAGGATGTATTAAGA |
| 1708 | Encoding | ATGTATTAAGAGGAGGGATAGGCAAGCTAGATCATG |
| | Probe 1661 | CTGCGCAGAGGAGGATGTATTAAGA |
| 1709 | Encoding | ATGTATTAAGAGGAGGGATAGGCAAGCTAGATCATG |
| | Probe 1662 | CTGGGCGAGGAGGATGTATTAAGA |
| 1710 | Encoding | ATGTATTAAGAGGAGGGATAGCACCTAATATTAGTA |
| | Probe 1663 | AGTGCGTAAGAGGAGGATGTATTAAGA |
| 1711 | Encoding | ATGTATTAAGAGGAGGGACTGCGATGCATTTTCTGG |
| | Probe 1664 | GATATCGAGGAGGATGTATTAAGA |
| 1712 | Encoding | TGTATAGGATTAGAAGGGTTTGCCTTTCAACTTTCTT |
| | Probe 1665 | CCATGGCCGGGTGAGTGTATAGGATT |
| 1713 | Encoding | TGTATAGGATTAGAAGGGTGGTCGGAAAATAGTGTT |
| | Probe 1666 | ATACGGATAGGGTGAGTGTATAGGATT |
| 1714 | Encoding | TGTATAGGATTAGAAGGGAGGGCGGAAAATAGTGTT |
| | Probe 1667 | ATACGCATGGGTGAGTGTATAGGATT |
| 1715 | Encoding | TGTATAGGATTAGAAGGGTGCTGGGAAGCTCTATCT |
| | Probe 1668 | CTAGACACGGGTGAGTGTATAGGATT |
| 1716 | Encoding | TGTATAGGATTAGAAGGGTGTATACTCTCATCCTTGT |
| | Probe 1669 | TCTTCAGAGGGTGAGTGTATAGGATT |
| 1717 | Encoding | TGTATAGGATTAGAAGGGAGGGCGGAAAATAGTGTT |
| | Probe 1670 | ATACGGATAGGGTGAGTGTATAGGATT |
| 1718 | Encoding | TGTATAGGATTAGAAGGGAAAGAGATTAGCTTAGCC |
| | Probe 1671 | TCGGCTGGGTGAGTGTATAGGATT |
| 1719 | Encoding | TGTATAGGATTAGAAGGGTAAACTCTCATCCTTGTTC |
| | Probe 1672 | TTCAGAGGGTGAGTGTATAGGATT |
| 1720 | Encoding | ATGTATTAAGAGGAGGGATTTGCCTTTCAACTTTCTT |
| | Probe 1673 | CCATGGCCGAGGAGGATGTATTAAGA |
| 1721 | Encoding | ATGTATTAAGAGGAGGGAGGTCGGAAAATAGTGTTA |
| | Probe 1674 | TACGGATAGAGGAGGATGTATTAAGA |
| 1722 | Encoding | ATGTATTAAGAGGAGGGAAGGGCGGAAAATAGTGT |
| | Probe 1675 | TATACGCATGAGGAGGATGTATTAAGA |
| 1723 | Encoding | ATGTATTAAGAGGAGGGAGCTGGGAAGCTCTATCTC |
| | Probe 1676 | TAGACACGAGGAGGATGTATTAAGA |
| 1724 | Encoding | ATGTATTAAGAGGAGGGAGTATACTCTCATCCTTGT |
| | Probe 1677 | TCTTCAGAGAGGAGGATGTATTAAGA |
| 1725 | Encoding | ATGTATTAAGAGGAGGGAAGGGCGGAAAATAGTGT |
| | Probe 1678 | TATACGGATAGAGGAGGATGTATTAAGA |
| 1726 | Encoding | ATGTATTAAGAGGAGGGAAAAGAGATTAGCTTAGCC |
| | Probe 1679 | TCGGCTGAGGAGGATGTATTAAGA |
| 1727 | Encoding | ATGTATTAAGAGGAGGGATAAACTCTCATCCTTGTT |
| | Probe 1680 | CTTCAGAGAGGAGGATGTATTAAGA |
| 1728 | Encoding | ATGGAAGTAGTAGAAGGGTGTAGCTCCCGGGTGCTT |
| | Probe 1681 | ATGCCCAGGGATGTATGGAAGTAGT |
| 1729 | Encoding | ATGGAAGTAGTAGAAGGGTGCGCTAAAGCAAACAC |
| | Probe 1682 | ACTTCCTAGGTGGGATGTATGGAAGTAGT |
| 1730 | Encoding | ATGGAAGTAGTAGAAGGGTGCGCTAAAGCAAACAC |
| | Probe 1683 | ACTTCCAAGTGGGATGTATGGAAGTAGT |
| 1731 | Encoding | ATGGAAGTAGTAGAAGGGACTCGTTATTTCTCGGAT |
| | Probe 1684 | TCGGAGTGGGATGTATGGAAGTAGT |
| 1732 | Encoding | ATGGAAGTAGTAGAAGGGCGGTAAAGCAAACACAC |
| | Probe 1685 | TTCCTAGGTGGGATGTATGGAAGTAGT |
| 1733 | Encoding | ATGGAAGTAGTAGAAGGGTCTTTATTTCTCGGATTC |
| | Probe 1686 | GCTGGCGGGATGTATGGAAGTAGT |
| 1734 | Encoding | ATGGAAGTAGTAGAAGGGTGCGGTGTTCCTCCTGAT |
| | Probe 1687 | CTCTTGCGGGATGTATGGAAGTAGT |
| 1735 | Encoding | ATGGAAGTAGTAGAAGGGCGTCGCTCCCGGGTGCTT |
| | Probe 1688 | ATGGCCGGGATGTATGGAAGTAGT |
| 1736 | Encoding | ATGTATTAAGAGGAGGGAGTAGCTCCCGGGTGCTTA |
| | Probe 1689 | TGCCCAGAGGAGGATGTATTAAGA |
| 1737 | Encoding | ATGTATTAAGAGGAGGGAGCGCTAAAGCAAACACA |
| | Probe 1690 | CTTCCTAGGGAGGAGGATGTATTAAGA |
| 1738 | Encoding | ATGTATTAAGAGGAGGGAGCGCTAAAGCAAACACA |
| | Probe 1691 | CTTCCAAGGAGGAGGATGTATTAAGA |
| 1739 | Encoding | ATGTATTAAGAGGAGGGAACTCGTTATTTCTCGGAT |
| | Probe 1692 | TCGGAGGAGGAGGATGTATTAAGA |
| 1740 | Encoding | ATGTATTAAGAGGAGGGACGGTAAAGCAAACACAC |
| | Probe 1693 | TTCCTAGGGAGGAGGATGTATTAAGA |
| 1741 | Encoding | ATGTATTAAGAGGAGGGATCTTTATTTCTCGGATTCG |
| | Probe 1694 | CTGGCGAGGAGGATGTATTAAGA |
| 1742 | Encoding | ATGTATTAAGAGGAGGGAGCGGTGTTCCTCCTGATC |
| | Probe 1695 | TCTTGCGAGGAGGATGTATTAAGA |
| 1743 | Encoding | ATGTATTAAGAGGAGGGACGTCGCTCCCGGGTGCTT |
| | Probe 1696 | ATGGCCGAGGAGGATGTATTAAGA |
| 1744 | Encoding | TGTAATAGTAAGGAGGGAGGCGCTCTCATTCTTAAT |
| | Probe 1697 | ATCTTCGTAGCGGGTGAGTGTAATAGTAA |
| 1745 | Encoding | TGTAATAGTAAGGAGGGATTCTATCTCTACGCCTGT |
| | Probe 1698 | CATGTCGGGTGAGTGTAATAGTAA |
| 1746 | Encoding | TGTAATAGTAAGGAGGGAGGTCTGCACCGAATAAAT |
| | Probe 1699 | CCTAAGTGGGTGAGTGTAATAGTAA |
| 1747 | Encoding | TGTAATAGTAAGGAGGGAGTACTTTCGTCCCTGTTG |
| | Probe 1700 | ATACTTGGGTGAGTGTAATAGTAA |
| 1748 | Encoding | TGTAATAGTAAGGAGGGAAGGATGCAATCCTCGGGT |
| | Probe 1701 | TAACGGTGGGTGAGTGTAATAGTAA |
| 1749 | Encoding | TGTAATAGTAAGGAGGGACAATGTGATTTGCTTAAC |
| | Probe 1702 | GTCCGGTGGGTGAGTGTAATAGTAA |
| 1750 | Encoding | TGTAATAGTAAGGAGGGAAGTGACTTCGGGTGCTTC |
| | Probe 1703 | CAAGAGTGGGTGAGTGTAATAGTAA |
| 1751 | Encoding | TGTAATAGTAAGGAGGGAGTAGGGATTCCTCCCCGA |
| | Probe 1704 | CACAATGGGTGAGTGTAATAGTAA |
| 1752 | Encoding | ATGTATTAAGAGGAGGGAGGCGCTCTCATTCTTAAT |
| | Probe 1705 | ATCTTCGTAGCGAGGAGGATGTATTAAGA |
| 1753 | Encoding | ATGTATTAAGAGGAGGGATTCTATCTCTACGCCTGT |
| | Probe 1706 | CATGTCGAGGAGGATGTATTAAGA |
| 1754 | Encoding | ATGTATTAAGAGGAGGGAGGTCTGCACCGAATAAAT |
| | Probe 1707 | CCTAAGGAGGAGGATGTATTAAGA |
| 1755 | Encoding | ATGTATTAAGAGGAGGGAGTACTTTCGTCCCTGTTG |
| | Probe 1708 | ATACTTGAGGAGGATGTATTAAGA |
| 1756 | Encoding | ATGTATTAAGAGGAGGGAAGGATGCAATCCTCGGGT |
| | Probe 1709 | TAACGGGAGGAGGATGTATTAAGA |
| 1757 | Encoding | ATGTATTAAGAGGAGGGACAATGTGATTTGCTTAAC |
| | Probe 1710 | GTCCGGGAGGAGGATGTATTAAGA |
| 1758 | Encoding | ATGTATTAAGAGGAGGGAAGTGACTTCGGGTGCTTC |
| | Probe 1711 | CAAGAGGAGGAGGATGTATTAAGA |
| 1759 | Encoding | ATGTATTAAGAGGAGGGAGTAGGGATTCCTCCCCGA |
| | Probe 1712 | CACAATGAGGAGGATGTATTAAGA |
| 1760 | Encoding | AATGATATGTTGAGTGGGTTTTTCTCTCCAATTTGTA |
| | Probe 1713 | ACGAAGAGTGGTGGAATGATATGTT |
| 1761 | Encoding | AATGATATGTTGAGTGGGTGATGCCTCTATATAGTT |
| | Probe 1714 | GGCTGTGGTGGTGGAATGATATGTT |
| 1762 | Encoding | AATGATATGTTGAGTGGGTTATCTCTCCAATTTGTAA |
| | Probe 1715 | CGAAGAGTGGTGGAATGATATGTT |
| 1763 | Encoding | AATGATATGTTGAGTGGGAGGCTAGTATCATGTGAT |
| | Probe 1716 | ACTTATGGGTAGTGGTGGAATGATATGTT |
| 1764 | Encoding | AATGATATGTTGAGTGGGTGAACCACTAGTATCATG |
| | Probe 1717 | TGATACTATAGTGGTGGAATGATATGTT |
| 1765 | Encoding | AATGATATGTTGAGTGGGAAACTCCATATCACTACT |
| | Probe 1718 | TAGCTTAGGGTGGTGGAATGATATGTT |
| 1766 | Encoding | AATGATATGTTGAGTGGGTGCTGCACAGATTACTTA |
| | Probe 1719 | ATATAACCTTGTGTGGTGGAATGATATGTT |
| 1767 | Encoding | AATGATATGTTGAGTGGGTGTAACCACCTGTATAGA |
| | Probe 1720 | CGTCGGCGTGGTGGAATGATATGTT |
| 1768 | Encoding | TAGAATTAGAGAGATGGGTTTTTCTCTCCAATTTGTA |
| | Probe 1721 | ACGAAGAGGTGGAGTAGAATTAGAG |
| 1769 | Encoding | TAGAATTAGAGAGATGGGTGATGCCTCTATATAGTT |
| | Probe 1722 | GGCTGTGGGTGGAGTAGAATTAGAG |
| 1770 | Encoding | TAGAATTAGAGAGATGGGTTATCTCTCCAATTTGTA |
| | Probe 1723 | ACGAAGAGGTGGAGTAGAATTAGAG |
| 1771 | Encoding | TAGAATTAGAGAGATGGGAGGCTAGTATCATGTGAT |
| | Probe 1724 | ACTTATGGGTAGGTGGAGTAGAATTAGAG |
| 1772 | Encoding | TAGAATTAGAGAGATGGGTGAACCACTAGTATCATG |
| | Probe 1725 | TGATACTATAGGTGGAGTAGAATTAGAG |
| 1773 | Encoding | TAGAATTAGAGAGATGGGAAACTCCATATCACTACT |
| | Probe 1726 | TAGCTTAGGTGGTGGAGTAGAATTAGAG |
| 1774 | Encoding | TAGAATTAGAGAGATGGGTGCTGCACAGATTACTTA |
| | Probe 1727 | ATATAACCTTGTGGTGGAGTAGAATTAGAG |
| 1775 | Encoding | TAGAATTAGAGAGATGGGTGTAACCACCTGTATAGA |
| | Probe 1728 | CGTCGGCGGTGGAGTAGAATTAGAG |
| 1776 | Encoding | ATGGAAGTAGTAGAAGGGTGCACCGGGAGCCTTTGG |
| | Probe 1729 | CACGGTGGGATGTATGGAAGTAGT |
| 1777 | Encoding | ATGGAAGTAGTAGAAGGGTGGCTCGGCTTTTCACCC |
| | Probe 1730 | CGAAGGTGGGATGTATGGAAGTAGT |
| 1778 | Encoding | ATGGAAGTAGTAGAAGGGAAACTTCCGACTTGTATT |
| | Probe 1731 | GCCCAGTGGGATGTATGGAAGTAGT |
| 1779 | Encoding | ATGGAAGTAGTAGAAGGGTGCGCTCAGTCAATTAAC |
| | Probe 1732 | ATTCCAAGGTGGGATGTATGGAAGTAGT |
| 1780 | Encoding | ATGGAAGTAGTAGAAGGGTGGCAACTTCCTCTTAAT |
| | Probe 1733 | TGCTTCCGAGGGATGTATGGAAGTAGT |
| 1781 | Encoding | ATGGAAGTAGTAGAAGGGAGCAGCTCCCTGCTTTCG |
| | Probe 1734 | CTTCCCGGGATGTATGGAAGTAGT |
| 1782 | Encoding | ATGGAAGTAGTAGAAGGGACGAGCTTTCTCTGTTTG |
| | Probe 1735 | CTAGACGGGATGTATGGAAGTAGT |
| 1783 | Encoding | ATGGAAGTAGTAGAAGGGACGCGTAGGGAACAGAA |
| | Probe 1736 | TGTTTGAGGGATGTATGGAAGTAGT |
| 1784 | Encoding | TAGAATTAGAGAGATGGGTGCACCGGGAGCCTTTGG |
| | Probe 1737 | CACGGTGGTGGAGTAGAATTAGAG |
| 1785 | Encoding | TAGAATTAGAGAGATGGGTGGCTCGGCTTTTCACCC |
| | Probe 1738 | CGAAGGTGGTGGAGTAGAATTAGAG |
| 1786 | Encoding | TAGAATTAGAGAGATGGGAAACTTCCGACTTGTATT |
| | Probe 1739 | GCCCAGGGTGGAGTAGAATTAGAG |
| 1787 | Encoding | TAGAATTAGAGAGATGGGTGCGCTCAGTCAATTAAC |
| | Probe 1740 | ATTCCAAGGTGGTGGAGTAGAATTAGAG |
| 1788 | Encoding | TAGAATTAGAGAGATGGGTGGCAACTTCCTCTTAAT |
| | Probe 1741 | TGCTTCCGAGGTGGAGTAGAATTAGAG |
| 1789 | Encoding | TAGAATTAGAGAGATGGGAGCAGCTCCCTGCTTTCG |
| | Probe 1742 | CTTCCCGGTGGAGTAGAATTAGAG |
| 1790 | Encoding | TAGAATTAGAGAGATGGGACGAGCTTTCTCTGTTTG |
| | Probe 1743 | CTAGACGGTGGAGTAGAATTAGAG |
| 1791 | Encoding | TAGAATTAGAGAGATGGGACGCGTAGGGAACAGAA |
| | Probe 1744 | TGTTTGAGGTGGAGTAGAATTAGAG |
| 1792 | Encoding | GGATAGAGTATAGTTGGGCGTCCCCTCTGTAAGCGG |
| | Probe 1745 | ATTAGAGGATGGAGGATAGAGTAT |
| 1793 | Encoding | GGATAGAGTATAGTTGGGCCACCGTCAAATTTCTCT |
| | Probe 1746 | TTCTAGAGGATGGAGGATAGAGTAT |
| 1794 | Encoding | GGATAGAGTATAGTTGGGTGTGCCTATCGGCAACAC |
| | Probe 1747 | TTAGATGGGATGGAGGATAGAGTAT |
| 1795 | Encoding | GGATAGAGTATAGTTGGGTGCGTGTCTCCATAACTT |
| | Probe 1748 | CGCACCCGGATGGAGGATAGAGTAT |
| 1796 | Encoding | GGATAGAGTATAGTTGGGAAGCCATTTTATCAATGG |
| | Probe 1749 | CAGTGATGGATGGAGGATAGAGTAT |
| 1797 | Encoding | GGATAGAGTATAGTTGGGTCTGCTCCCTCATTTCTGT |
| | Probe 1750 | TACCGGGATGGAGGATAGAGTAT |
| 1798 | Encoding | GGATAGAGTATAGTTGGGTGTCTAAAATGCTTTTTCC |
| | Probe 1751 | ATTGTGGAGGATGGAGGATAGAGTAT |
| 1799 | Encoding | GGATAGAGTATAGTTGGGTGAAGTTCATCAGTATCT |
| | Probe 1752 | TTTGCCCATGGGATGGAGGATAGAGTAT |
| 1800 | Encoding | TAGAATTAGAGAGATGGGCGTCCCCTCTGTAAGCGG |
| | Probe 1753 | ATTAGAGGTGGAGTAGAATTAGAG |
| 1801 | Encoding | TAGAATTAGAGAGATGGGCCACCGTCAAATTTCTCT |
| | Probe 1754 | TTCTAGAGGTGGAGTAGAATTAGAG |
| 1802 | Encoding | TAGAATTAGAGAGATGGGTGTGCCTATCGGCAACAC |
| | Probe 1755 | TTAGATGGGTGGAGTAGAATTAGAG |
| 1803 | Encoding | TAGAATTAGAGAGATGGGTGCGTGTCTCCATAACTT |
| | Probe 1756 | CGCACCCGGTGGAGTAGAATTAGAG |
| 1804 | Encoding | TAGAATTAGAGAGATGGGAAGCCATTTTATCAATGG |
| | Probe 1757 | CAGTGATGGTGGAGTAGAATTAGAG |
| 1805 | Encoding | TAGAATTAGAGAGATGGGTCTGCTCCCTCATTTCTGT |
| | Probe 1758 | TACCGGGTGGAGTAGAATTAGAG |
| 1806 | Encoding | TAGAATTAGAGAGATGGGTGTCTAAAATGCTTTTTC |
| | Probe 1759 | CATTGTGGAGGTGGAGTAGAATTAGAG |
| 1807 | Encoding | TAGAATTAGAGAGATGGGTGAAGTTCATCAGTATCT |
| | Probe 1760 | TTTGCCCATGGGTGGAGTAGAATTAGAG |

Although the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of the present disclosure. All such modifications are intended to be within the scope of the claims appended hereto.

What is claimed is:

1. A method of characterizing a microbial cell from a biological sample, the method comprising
a) directly inoculating the microbe onto a device;
b) identifying the microbe; and
c) detecting susceptibility to one or more antimicrobial agents.

2. A method of characterizing a microbial cell from a biological sample, the method comprising
a) directly inoculating the microbe onto a device;
b) identifying the microbe; and
c) detecting future susceptibility to one or more antimicrobial agents.

3. The method of claim 1, wherein the sample is not subjected to culturing before the microbe is inoculated onto the device.

4. The method of claim 1, wherein the microbe in the sample is cultured for one or more cell divisions before it is inoculated onto the device.

5. The method of claim 1, wherein the microbe is identified by in situ hybridization.

6. The method of claim 5, wherein the microbe is identified by fluorescence in situ hybridization (FISH).

7. The method of claim 5, wherein the fluorescence in situ hybridization is high-phylogenetic-resolution fluorescence in situ hybridization (HiPR-FISH).

8. The method of claim 5, wherein the microbe is further characterized via live-cell imaging or dynamic calculation while in situ hybridization is performed.

9. The method of claim 1, wherein the microbe is identified by hybridization of a bar-coded probe a 16S ribosomal RNA sequence in the microbe, 5S ribosomal RNA sequence in the microbe, and/or 23 S ribosomal RNA sequence in the microbe.

10. The method of claim 6, wherein the in situ hybridization is multiplexed.

11. The method of claim 1, wherein the susceptibility to one or more microbial agents is determined by measuring the minimum inhibitory concentration of the microbe when exposed to an antimicrobial agent.

12. The method of claim 1, wherein the susceptibility to one or more microbial agents is determined by measuring microbial cell metabolism when the microbe is exposed to an antimicrobial agent.

13. The method of claim 12, wherein microbial cell metabolism is measured by determining the concentration of dissolved carbon dioxide, oxygen consumption of microbes in the sample, expression of genes involved in cell division and/or growth, or expression of stress response genes.

14. The method of claim 1, wherein microbial cell susceptibility is determined by a live/dead stain.

15. The method of claim 1, wherein microbial cell susceptibility is determined by cell number.

16. The method of claim 1, wherein microbial cell susceptibility is determined by detecting the presence or absence of one or more antimicrobial genes in the microbial cell.

17. The method of claim 1, wherein microbial cell susceptibility is determined by detecting the presence or absence of one or more gene mutations associated with the development of antimicrobial resistance or susceptibility in the microbial cell.

18. The method of claim 2, wherein future microbial cell susceptibility is determined by detecting the presence or absence of one or more antimicrobial genes in the microbial cell.

19. The method of claim 2, wherein future microbial cell susceptibility is determined by detecting the presence or absence of one or more gene mutations associated with the development of antimicrobial resistance or susceptibility in the microbial cell.

20. The method of claim 17, wherein the one or more gene mutations associated with the development of antimicrobial resistance or susceptibility is selected from deletions, duplications, single nucleotide polymorphisms (SNPs), frame-shift mutations, inversions, insertions, and/or nucleotide substitutions.

21. The method of claim 16, wherein the one or more antimicrobial genes is selected from: genes encoding multidrug resistance proteins (e.g. PDR1, PDR3, PDR7, PDR9), ABC transporters (e.g. SNQ2, STE6, PDR5, PDR10, PDR11, YOR1), membrane associated transporters (GAS1, D4405), soluble proteins (e.g. G3PD), RNA polymerase, rpoB, gyrA, gyrB, 16S RNA, 23S rRNA, NADPH nitroreductase, sul2, strAB, tetAR, aac3-iid, aph, sph, cmy-2, floR, tetB; aadA, aac3-VIa, and sul1.

22. The method of claim 16, wherein the presence or absence of one or more antimicrobial genes, or the gene mutation associated with the development of antimicrobial resistance or susceptibility in the microbial cell is detected using in situ hybridization.

23. The method of claim 22, wherein the presence or absence of one or more antimicrobial genes, or the gene mutation associated with the development of antimicrobial resistance or susceptibility in the microbial cell is detected using fluorescence in situ hybridization (FISH).

24. The method of claim 23, wherein the fluorescence in situ hybridization is high-phylogenetic-resolution fluorescence in situ hybridization (HiPR-FISH).

25. The method claim 1, wherein the identification of the microbial cell and the detection of susceptibility or future susceptibility to one or more antimicrobial agents occurs sequentially.

26. The method of claim 1, wherein the identification of the microbial cell and the detection of susceptibility or future susceptibility to one or more antimicrobial agents occurs simultaneously.

27. The method of claim 1, wherein the identification of the microbial cell and the detection of susceptibility or future susceptibility to one or more antimicrobial agents occurs in parallel.

28. The method of claim 1, wherein the biological sample is obtained from a patient.

29. The method of claim 1, wherein the biological sample is obtained from a patient diagnosed with or believed to be suffering from an infection or disorder.

30. The method of claim 29, wherein the disease or disorder is an infection.

31. The method of claim 30, wherein the infection is a bacterial, viral, fungal, or parasitic infections.

32. The method of claim 31, wherein the bacterial infection is selected from Mycobacterium, Streptococcus, Staphylococcus, Shigella, Campylobacter, Salmonella, Clostridium, Corynebacterium, Pseudomonas, Neisseria, Listeria, Vibrio, Bordetella, E. coli (including pathogenic E. coli), Pseudomonas aeruginosa, Enterobacter cloacae, Mycobacterium tuberculosis, Staphylococcus aureus, Helicobacter pylori, Legionella, Acinetobacter baumannii, Citrobacter freundii, Citrobacter koseri, Enterobacter cloacae, Enterococcus faecalis, Enterococcus faecium, Escherichia coli, Klebsiella oxytoca, Klebsiella pneumoniae, Proteus mirabilis, Proteus vulgaris, Pseudomonas aeruginosa, Serratia marcescens, Staphylococcus aureus, Staphylococcus saprophyticus, and Streptococcus agalactiae, or a combination thereof.

33. The method of claim 31, wherein the viral infection is selected from Helicobacter pylori, infectious haematopoietic necrosis virus (IHNV), Parvovirus B19, Herpes Simplex Virus, Varicella-zoster virus, Cytomegalovirus, Epstein-Barr virus, Hepatitis A virus, Hepatitis B virus, Hepatitis C virus, Hepatitis D virus, Hepatitis E virus, Measles virus, Mumps virus, Rubella virus, Human Immunodeficiency Virus (HIV), Influenza virus, Rhinovirus, Rotavirus A, Rotavirus B, Rotavirus C, Respiratory Syncytial Virus (RSV), Varicella zoster, Poliovirus, Norovirus, Zika Virus, Dengue Virus, Rabies Virus, Newcastle Disease Virus, and White Spot Syndrome Virus, or a combination thereof.

34. The method of claim 31, wherein the fungal infection is selected from Aspergillus, Candida, Pneumocystis, Blastomyces, Coccidioides, Cryptococcus, and Histoplasma, or a combination thereof.

35. The method of claim 31, wherein the parasitic infection is selected from Plasmodium (i.e. P. falciparum, P. malariae, P. ovale, P. knowlesi, and P. vivax), Trypanosoma, Toxoplasma, Giardia, and Leishmania, Cryptosporidium, helminthic parasites: Trichuris spp. (whipworms), Enterobius spp. (pinworms), Ascaris spp. (roundworms), Ancylostoma spp. and Necator spp. (hookworms), Strongyloides spp. (threadworms), Dracunculus spp. (Guinea worms), Onchocerca spp. and Wuchereria spp. (filarial worms), Taenia spp., Echinococcus spp., and Diphyllobothrium spp. (human and animal cestodes), Fasciola spp. (liver flukes) and Schistosoma spp. (blood flukes), or a combination thereof.

36. The method of claim 1, wherein the biological sample is selected from bronchoalveolar lavage fluid (BAL), blood, serum, plasma, urine, cerebrospinal fluid, pleural fluid, synovial fluid, ocular fluid, peritoneal fluid, amniotic fluid, gastric fluid, lymph fluid, interstitial fluid, tissue homogenate, cell extracts, saliva, sputum, stool, physiological secretions, tears, mucus, sweat, milk, semen, seminal fluid, vaginal secretions, fluid from ulcers and other surface eruptions, blisters, and abscesses, and extracts of tissues including biopsies of normal, malignant, and suspect tissues or any other constituents of the body which may contain the microorganism of interest.

37. The method of claim 1, wherein the biological sample is a human oral microbiome sample.

38. The method of claim 1, wherein the biological sample is a whole organism.

39. A method for analyzing a sample, comprising:
contacting at least one encoding probe with the sample to produce a first complex, wherein each encoding probe comprises a targeting sequence, a first landing pad sequence, and a second landing pad sequence;
adding at least one first emissive readout probe to the first complex, wherein the first emissive readout probe comprises a label and a sequence complementary to the first landing pad sequence;
acquiring one or more emission spectra from the first emissive readout probe;
adding an exchange probe to the sample, wherein the exchange probe comprises a 100% complementary sequence to the first emissive readout probe sequence,
hybridizing the exchange probe to the first emissive readout probe to form a second complex;
removing the second complex from the sample,
adding at least one second emissive readout probe to the first complex, wherein the second emissive readout probe comprises a label and a sequence complementary to the second landing pad sequence;
acquiring one or more emission spectra from the second emissive readout probe;
repeating the aforementioned steps for at least one different encoding probe;
determining the spectra of “signal” (e.g., puncta, blobs) and assigning them to a species of interest; and
decoding the spectra into a single, targeted transcript through means of signal deconvolution, error correction, comparison to reference standards.

40. A method for analyzing a sample, comprising:
generating a set of probes, wherein each probe comprises:
(i) a targeting sequence;
(ii) a first landing pad sequence; and
(iii) a second landing pad sequence;
contacting the set of probes with the sample to permit hybridization of the probes to nucleotides present in the sample to produce a complex;
adding a first set of emissive readout probes to the complex, wherein each emissive readout probe comprises:
(i) a label, and
(ii) a sequence complementary to the first or second landing pad sequence;
acquiring one or more emission spectra from the first emissive readout probe;
adding a set of exchange probes to the sample, wherein each exchange probe comprises a 100% complementary sequence to the first emissive readout probe sequences,
hybridizing the exchange probes to the first emissive readout probes to form a second complex;
removing the second complex from the sample,
adding a second set of emissive readout probes to the complex, wherein each emissive readout probe comprises:
(i) a label, and
(ii) a sequence complementary to the first or second landing pad sequence;
acquiring one or more emission spectra from the second emissive readout probe;
determining the spectra of “signal” (e.g., puncta, blobs) and assigning them to a species of interest; and
decoding the spectra into a single, targeted transcript through means of signal deconvolution, error correction, comparison to reference standards.

41. The method of claim 39, wherein the sample is at least one of a cell, a cell suspension, a tissue biopsy, a tissue specimen, urine, stool, blood, serum, plasma, bone biopsies, bone marrow, respiratory specimens, sputum, induced sputum, tracheal aspirates, bronchoalveolar lavage fluid, sweat, saliva, tears, ocular fluid, cerebral spinal fluid, pericardial fluid, pleural fluid, peritoneal fluid, placenta, amnion, pus, nasal swabs, nasopharyngeal swabs, oropharyngeal swabs, ocular swabs, skin swabs, wound swabs, mucosal swabs, buccal swabs, vaginal swabs, vulvar swabs, nails, nail scrapings, hair follicles, corneal scrapings, gavage fluids, gargle fluids, abscess fluids, wastewater, or plant biopsies.

42. The method of claim 41, wherein the sample is a cell.

43. The method of claim 42, wherein the cell is a bacterial or eukaryotic cell.

44. The method of claim 41, wherein the sample comprises a plurality of cells.

45. The method of claim 44, wherein each cell comprises a specific targeting sequence.

46. The method of claim 39, wherein the targeting sequence targets at least one of messenger RNA (mRNA), micro RNA (miRNA), long non-coding RNA (lncRNA), ribosomal RNA (rRNA), small interfering RNA (siRNA), transfer RNA (tRNA), Crispr RNA (crRNA), trans-activating crispr RNA (tracrRNA), mitochondria RNA, Intronic RNA, viral mRNA, viral genomic RNA, environmental RNA, double-stranded RNA (dsRNA), small nuclear RNA (snRNA), small nucleolar (snoRNA), piwi-interacting RNA (piRNA), genomic DNA, synthetic DNA, DNA, plasmid DNA, a plasmid, viral DNA, retroviral DNA, environmental DNA, extracellular DNA, a protein, a small molecule, or an antigenic target.

47. The method of claim 46, wherein the target is mRNA.

48. The method of claim 46, wherein the target is rRNA.

49. The method of claim 46, wherein the target is mRNA and rRNA.

50. The method of claim 39, wherein the at least one encoding probe comprises the first landing pad sequence on the 5′ end, and the second landing pad sequence on the 3′ end.

51. The method of claim 39, wherein the at least one encoding probe comprises the first landing pad sequence on the 3′ end, and the second landing pad sequence on the 5′ end.

52. The method of claim 50, wherein the first landing pad sequence and the second landing pad sequences have different sequences.

53. The method of claim 39, wherein the at least one first or second emissive readout probe comprises a label on the 5′ or 3′ end.

54. The method of claim 39, wherein the label is Alexa Fluor 350, Alexa Fluor 405, Alexa Fluor 430, Alexa Fluor 488, Alexa Fluor 514, Alexa Fluor 532, Alexa Fluor 546, Alexa Fluor 555, Alexa Fluor 561, Alexa Fluor 568, Alexa Fluor 594, Alexa Fluor 610, Alexa Fluor 633, Alexa Fluor 635, Alexa Fluor 647, Alexa Fluor 647-R-phycoerythrin, Alexa Fluor 660, Alexa Fluor 680, Alexa Fluor 680-allophycocyanin, Alexa Fluor 700, Alexa Fluor 750, Alexa Fluor 790, Alexa Fluor Plus 405, Alexa Fluor Plus 488, Alexa Fluor Plus 555, Alexa Fluor Plus 594, Alexa Fluor Plus 647, Alexa Fluor Plus 680, Alexa Fluor Plus 750, Alexa Fluor Plus 800, Pacific Blue, Pacific Green, Rhodamine Red X, DyLight 485-LS, DyLight-510-LS, DyLight 515-LS, DyLight 521-LS, Hydroxycoumarin, methoxycoumarin, Cy2, FAM, Fluorescein FITC, R-phycoerythrin (PE), Tamara, Cy3.5 581, Rox, Red 613, Texas Red, Cy5, Cy5.5, Cy7, Allophycocyanin, ATTO 430LS, ATTO 490LS, ATTO 390, ATTO 425, Cyan 500 NHS-Ester, ATTO 465, ATTO 488, ATTO 495, ATTO Rho110, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 643, ATTO 647, ATTO 647N, ATTO 655, ATTO Oxa12, ATTO 665, ATTO 680, ATTO 700, ATTO 725, ATTO 740.

55. The method of claim 39, wherein the one or more emission spectra of the first and/or second emissive readout probe is acquired via widefield microscopy, point scanning confocal microscopy, spinning disk confocal microscopy, lattice lightsheet microscopy, or light field microscopy.

56. The method of claim 55, wherein the detection strategy used is channel, spectral, channel and fluorescence lifetime, or spectral and fluorescence lifetime.

57. The method of claim 39, wherein the sample is on an analyzing platform, wherein the analyzing platform is a microscope slide, at least one chamber, at least one microfluidic device, at least one well, at least one plate, or at least one filter membrane.

58. The method of claim 39, wherein adding an exchange probe to the sample, hybridizing the exchange probe to the first emissive readout probe, and removing the second complex from the sample are performed in the same step.

59. The method of claim 39, wherein hybridizing the exchange probe to the first or second emissive readout probe results in de-hybridization of the first or second emissive readout probe from the first or second landing pad sequence.

60. The method of claim 58, wherein the step is achieved within 1 hour.

61. The method of claim 58, wherein the step is achieved overnight.

62. The method of claim 39, wherein the emissive readout probe sequence is at least 5 nucleotides longer than the first or second landing pad sequences.

63. A construct comprising:
a targeting sequence that is a region of interest on a nucleotide;
a first landing pad sequence;
a second landing pad sequence, wherein the second landing pad sequence is different from the first landing pad sequence;
a first emissive readout probe comprising a first label and a sequence complimentary to the first landing pad sequence;
an exchange probe comprising a 100% complementary sequence to the first emissive readout probe sequences; and
a second emissive readout probe comprising a second label and a sequence complimentary to the second landing pad sequence.

64. A library of constructs comprising a plurality of barcoded probes, wherein each barcoded probe comprises:
a targeting sequence that is a region of interest on a nucleotide;
a first landing pad sequence;
a second landing pad sequence, wherein the second landing pad sequence is different from the first landing pad sequence;
a first emissive readout probe comprising a first label and a sequence complimentary to the first landing pad sequence;
an exchange probe comprising a 100% complementary sequence to the first emissive readout probe sequences; and
a second emissive readout probe comprising a second label and a sequence complimentary to the second landing pad sequence.

65. The construct of claim 63, wherein the first emissive readout probe sequence is at least 5 nucleotides longer than the first landing pad sequence.

66. The construct of claim 63, wherein the second emissive readout probe sequence is at least 5 nucleotides longer than the second landing pad sequence.

67. The construct of claim 63, wherein the first landing pad sequence and the second landing pad sequences have different sequences.

68. The construct of claim 63, wherein the first emissive readout probe comprises the first label on the 5′ or 3′ end.

69. The construct of claim 63, wherein the second emissive readout probe comprises the second label on the 5′ or 3′ end.

70. The construct of claim 63, wherein the first or second label is each Alexa Fluor 350, Alexa Fluor 405, Alexa Fluor 430, Alexa Fluor 488, Alexa Fluor 514, Alexa Fluor 532, Alexa Fluor 546, Alexa Fluor 555, Alexa Fluor 561, Alexa Fluor 568, Alexa Fluor 594, Alexa Fluor 610, Alexa Fluor 633, Alexa Fluor 635, Alexa Fluor 647, Alexa Fluor 647-R-phycoerythrin, Alexa Fluor 660, Alexa Fluor 680, Alexa Fluor 680-allophycocyanin, Alexa Fluor 700, Alexa Fluor 750, Alexa Fluor 790, Alexa Fluor Plus 405, Alexa Fluor Plus 488, Alexa Fluor Plus 555, Alexa Fluor Plus 594, Alexa Fluor Plus 647, Alexa Fluor Plus 680, Alexa Fluor Plus 750, Alexa Fluor Plus 800, Pacific Blue, Pacific Green, Rhodamine Red X, DyLight 485-LS, DyLight-510-LS, DyLight 515-LS, DyLight 521-LS, Hydroxycoumarin, methoxycoumarin, Cy2, FAM, Fluorescein FITC, R-phycoerythrin (PE), Tamara, Cy3.5 581, Rox, Red 613, Texas Red, Cy5, Cy5.5, Cy7, Allophycocyanin, ATTO 430LS, ATTO 490LS, ATTO 390, ATTO 425, Cyan 500 NHS-Ester, ATTO 465, ATTO 488, ATTO 495, ATTO Rho110, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 643, ATTO 647, ATTO 647N, ATTO 655, ATTO Oxa12, ATTO 665, ATTO 680, ATTO 700, ATTO 725, or ATTO 740.

71. A method for analyzing a bacterial sample, comprising:
contacting at least one encoding probe with the sample to produce a first complex, wherein each encoding probe comprises a targeting sequence, a first landing pad sequence, and a second landing pad sequence;
adding at least one first emissive readout probe to the first complex, wherein the first emissive readout probe comprises a label and a sequence complementary to the first landing pad sequence;
detecting the first emissive readout probe with a confocal microscope;
adding an exchange probe to the sample, wherein the exchange probe comprises a 100% complementary sequence to the first emissive readout probe sequence,
hybridizing the exchange probe to the first emissive readout probe to form a second complex;
removing the second complex from the sample,
adding at least one second emissive readout probe to the first complex, wherein the second emissive readout probe comprises a label and a sequence complementary to the second landing pad sequence;
detecting the second emissive readout probe with a confocal microscope;
repeating the aforementioned steps for at least one different encoding probe;
determining the spectra of “signal” (e.g., puncta, blobs) and assigning them to a bacterium; and
decoding the spectra into a single, targeted transcript through means of signal deconvolution, error correction, comparison to reference standards.

72. A method for analyzing a bacterial sample, comprising:
generating a set of probes, wherein each probe comprises:
(iv) a targeting sequence;
(v) a first landing pad sequence; and
(vi) a second landing pad sequence;
contacting the set of probes with the sample to permit hybridization of the probes to nucleotides present in the sample to produce a complex;
adding a first set of emissive readout probes to the complex, wherein each emissive readout probe comprises:
(i) a label, and
(ii) a sequence complementary to the first or second landing pad sequence;
detecting the first set of emissive readout probes in the sample with a confocal microscope;
adding a set of exchange probes to the sample, wherein each exchange probe comprises a 100% complementary sequence to the first emissive readout probe sequences,
hybridizing the exchange probes to the first emissive readout probes to form a second complex;
removing the second complex from the sample,
adding a second set of emissive readout probes to the complex, wherein each emissive readout probe comprises:
(i) a label, and
(ii) a sequence complementary to the first or second landing pad sequence;
detecting the second set of emissive readout probes in the sample with a confocal microscope;
determining the spectra of “signal” (e.g., puncta, blobs) and assigning them to a bacterium; and
decoding the spectra into a single, targeted transcript through means of signal deconvolution, error correction, comparison to reference standards.
